(19) United States
(12) Patent Application Publication
(10) Pub. No.: US 2017/0137410 A1
(43) Pub. Date: May 18, 2017

(54) FUSED CYCLOALKYL-PYRIMIDINE COMPOUNDS AND USES THEREOF

(71) Applicant: Medivation Technologies, Inc., San Francisco, CA (US)

(72) Inventors: Sarvajit CHAKRAVARTY, Mountain View, CA (US); Roopa RAI, San Carlos, CA (US); Michael John GREEN, Half Moon Bay, CA (US); Amantullah ANSARI, Noida (IN); Anil Kumar AGARWAL, Noida (IN)

(73) Assignee: Medivation Technologies, Inc, San Francisco, CA (US)

(21) Appl. No.: 15/323,240

(22) PCT Filed: Jun. 26, 2015
(86) PCT No.: PCT/US15/38140

§ 371 Date: Dec. 30, 2016

Related U.S. Application Data
(60) Provisional application No. 62/020,303, filed on Jul. 2, 2014.

(51) Int. Cl. C07D 403/14; C07D 405/14; C07D 417/14; C07D 413/14; C07D 401/14; C07D 409/14

(52) U.S. Cl. CPC C07D 403/14; C07D 401/14; C07D 409/14; C07D 417/14; C07D 413/14; C07D 405/14

(57) ABSTRACT

Fused cycloalkyl-pyrimidine compounds that are kinase inhibitors, such as multi-kinase inhibitors, are provided. The compounds may be used in a method of treating cancer. Pharmaceutical compositions containing a fused cycloalkyl-pyrimidine compound and a pharmaceutically acceptable carrier are also provided, as are kits containing a fused cycloalkyl-pyrimidine compound or salt thereof and instructions for use, e.g., in a method of treating cancer.

### CROSS-REFERENCE TO RELATED APPLICATIONS

This application claims priority to U.S. Provisional Patent Application No. 62/020,303 filed Jul. 2, 2014, the disclosure of which is incorporated herein by reference in their entireties.

### BACKGROUND OF THE INVENTION

Protein kinases play key functions in cell signal transduction by phosphorylation of tyrosine, serine or threonine residues of proteins. They have become very attractive targets for therapeutic interventions in many disease states such as cancer, inflammation, arthritis and diabetes. Receptor protein tyrosine kinases have become compelling targets for cancer chemotherapy.

The FMS-like tyrosine kinase 3 (FLT3) belongs to the type III class of receptor tyrosine kinase. In the normal hematopoietic environment, FLT3 expression is predominantly found in CD34 positive cells, and appears to play an integral role in early hematopoiesis and reconstitution of multi-lineage myeloid precursors. Fathi A. T. et al. Am. J. Blood. Res. 2011, 1(2):175-89; Small D. et al. Proc. Natl. Acad. Sci. 1994, 91(2):459-63; Broxmeyer H. E. et al. Exp. Hematol. 1995, 23(10):1121-9. Upon binding of FLT3 ligand (FL), the receptor dimerizes and the inner leaflet of the membrane is auto-phosphorylated, which then leads to activation of the tyrosine kinase and subsequent downstream signaling, with significant mediators being PI3-kinase, AKT, MAP kinase, and STAT5. Fathi A. T. et al. Am. J. Blood. Res. 2011, 1(2):175-89; Dosil M. et al. Mol. Cell. Biol. 1993, 13(10):6572-85. Acute myeloid leukemia (AML) and B-cell acute lymphocytic leukemia (ALL) were found to express FLT3 and its mutants most frequently at various levels. Xu J. et al. Bioorg. Med. Chem. 2014, 22(23):6625-37; Rosnet O. et al. Leukemia 1996, 10(2):238-48. Although some AML cell lines exhibit overexpression of wild-type FLT3, 30% of de novo AML patients display activating mutations in FLT3, with 23% being hyperactive ITD mutations (FLT3-ITD). The ITD mutations have been uniformly associated with significantly poor prognosis, including increased aggressiveness as well as more frequent and rapid relapse. The presence of activating FLT3 mutations and the correlation to poor prognosis indicates that FLT3 is a driver of disease in AML. Zarrinkar P. P. et al. Blood 2009, 114(14):2984-92; Fathi A. T. et al. Am. J. Blood. Res. 2011, 1(2):175-89.

FLT3 inhibitors may find use in treating cancers such as acute myeloid leukemia and acute lymphoblastic leukemia. A compound which exhibits high selectivity for FLT3 against other kinases and the ability to afford substantial and sustained inhibition of FLT3 is particularly desirable. See Zarrinkar P. P. et al. Blood 2009, 114(2):2984-92; Fathi A. T. et al. Am. J. Blood. Res. 2011, 1(2):175-89, Li C. et al. Mol. Cancer Ther. 2015, 14(2):375-83; Smith B. D. et al. Blood 2004, 103(10):3669-76; Kantarjian H. et al. Blood 2010, 116(22):4422-9; Schiller G. J. et al. Blood 2010, 116(22):4386-7; Keng M. K. et al. Clin. Adv. Hematol. Oncol. 2013, 11(10):646-55; Levis M. et al. Blood 2002, 99(11):3885-91; Pratz K. W. et al. Blood 2009, 113(17):3938-46; DeAngelo D. J. et al. Blood 2006, 108(12):3674-81; Fiedler W. et al. Blood 2005, 105(3):986-93; Knapper S. et al. Blood 2006, 108(10):3262-70; Levis M. et al. Blood 2006, 108(10):3477-83; Stone R. M. et al. Blood 2005, 105(1):54-60 and Knapper S. et al. Blood 2006; 108(10):3494-503.

Tropomyosin-receptor kinase (Trk) receptors are a family of receptor tyrosine kinases that regulates synaptic strength and plasticity in the mammalian nervous system. Trk receptor signaling activates several small G proteins, including Ras, Rap-1, and the Cdc-42-Rac-Rho family, as well as pathways regulated by MAP kinase, PI 3-kinase and phospholipase-C-γ (PLC-γ). Huang, et al. Ann. Rev. Biochem. 72:609-642 (2003). Over-expression of Trk A and Trk C has been associated with poor prognosis in pancreatic cancer and colon cancer. Trk B is believed to be an attractive target for treatment of neuroblastoma, pancreatic cancer and colon cancer. See Sakamoto Y. et al. Oncol Rep. 2001, 8(3):477-84; Ma J. et al. J. Gastroenterol Hepatol. 2008, 23(12):1852-9; Dang C. et al. J. Gastroenterol Hepatol. 2006, 21(5):850-8; Okada Y. et al. Clin. Exp. Metastasis 2004, 21(4):285-92; Liu D. et al. Oncol. Rep. 2007, 18(3):673-7; Miknyoczki S. J. et al. Int. J. Cancer 1999, 81(3):417-27; Sasahira T. et al. Hum. Pathol. 2013, 44(6):1098-106; Asgharzadeh et al. J. Natl. Cancer Inst. 2006, 98(17):1193-203; Nakagawara A. et al. Mol. Cell. Biol. 1994, 14(1):759-67; Brodeur G. M. et al. Clin. Cancer Res. 2009, 15(10):3244-50; Ho R. et al. Cancer Res. 2002, 62(22):6462-6; Matsumoto K. et al. Cancer Res. 1995, 55(8):1798-806; Sclabas G. M. et al. Clin. Cancer Res. 2005, 11(2 Pt 1):440-9; Li Z. et al. Cancer Res. 2009, 69(19):7851-9; Sasahira T. et al. Hum. Pathol. 2013, 44(6):1098-106; Akil H. et al. PLoS One. 2011, 6(9); and Yu Y. et al. APMIS. 2010, 118(3):188-95.

Other protein kinases including those detailed herein are also important targets for treatment of conditions or disorders associated with protein kinases, such as cancer. A number of approved cancer therapeutics may function by targeting protein kinases. However, cancer remains a prevalent disease and there remains a need for new cancer therapeutics.

### BRIEF SUMMARY OF THE INVENTION

Fused cycloalkyl-pyrimidine compounds of the general Formula (I) are described as new kinase modulators, such as modulators of any one or more of the kinases in Examples B1 to B7. Certain fused cycloalkyl-pyrimidine compounds of the general Formula (I) in one aspect are multi-kinase modulators in that they are capable of modulating more than one kinase. However, it is also understood that certain compounds may be selective kinase modulators, such as a selective FLT3 inhibitor. Certain fused cycloalkyl-pyrimidine compounds of the general Formula (I) are described as new tropomyosin-receptor kinase receptor modulators (Trk modulators). In another aspect, the fused cycloalkyl-pyrimidine compounds of the general Formula (I) are described as new FMS-like tyrosine kinase (e.g., FLT3) inhibitors. Certain compounds are selective kinase modulators, such as compounds that modulate Trk and/or FLT3 to a greater extent (e.g., greater than any one of 2 fold or 3 fold or 5 fold or 10 fold or 20 fold or more) than they modulate insulin-like growth factor (IgF), such as IgF-1R. Certain compounds provided herein are Trk and/or FLT3 modulators but exhibit little to no ability to modulate IgF. Other compounds are also detailed herein. Compositions and kits comprising a compound are provided, as are methods of using and making the compounds. Compounds of the invention may also find use in treating of cancer. Compounds of the invention may also find use in treating diseases and/or conditions in which modulation of a kinase (e.g., one or more of the kinases in Examples B1 to B7) may be implicated in therapy. Compounds of the invention may also find use in treating diseases and/or conditions in which modulation of tropomyosin-receptor kinase (Trk) receptors may be implicated in therapy. Compounds of the invention may also find use in treating diseases and/or conditions in which inhibition of FMS-like tyrosine kinase (e.g., FLT3) may be implicated in therapy. Compounds disclosed herein may find use in the methods disclosed herein, including use in treating, preventing, delaying the onset and/or delaying the development of cancer in an individual in need thereof, such as a human.

In one variation, provided are compounds of the Formula (I):

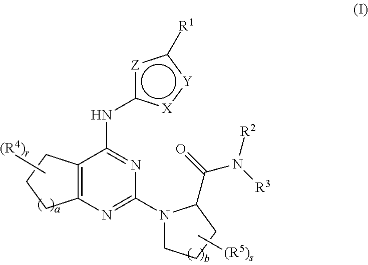

wherein:
X is N;
Y is NH or CH; and
Z is N, NH, N(C₁₋₄alkyl), or CH;
wherein the X-, Y-, and Z-containing ring is a 5-membered heteroaryl with at least two nitrogen ring atoms;
R¹ is C₃₋₈alkyl, C₃₋₈cycloalkyl, or 3- to 8-membered heterocyclyl;
wherein the C₃₋₈alkyl of R¹ is optionally substituted with 1, 2, or 3 substituents selected from the group consisting of —OH, —OC₁₋₄alkyl, —F, —CN, —NR^{a}R^{b}, and C₃₋₈cycloalkyl; and
the C₃₋₈cycloalkyl and 3- to 8-membered heterocyclyl of R¹ are independently optionally substituted with 1, 2, 3, or 4 substituents selected from the group consisting of C₁₋₄alkyl, —OH, —OC₁₋₄alkyl, —F, —CF₃, —CN, —CO₂H, —CO₂C₁₋₄alkyl, —C(O)NR^{a}R^{b}, and —NR^{a}R^{b};
   wherein R^{a} and R^{b} are each independently H or C₁₋₄alkyl;
R² is H or C₁₋₄alkyl optionally substituted with —OH, —OC₁₋₄alkyl, —F, —CF₃, —CN, or —NR^{c}R^{d}; wherein R^{c} and R^{d} are each independently H or C₁₋₄alkyl;
R³ is (a) C₁₋₄alkyl optionally substituted with —OH, —OC₁₋₄alkyl, —F, —CF₃, —CN, or —NR^{e}R^{f}; or (b) a 5- or 6-membered heteroaryl optionally substituted with one or more substituents selected from the group consisting of C₁₋₄alkyl, —OH, —OC₁₋₄alkyl, halo, —CF₃, —CN, —CO₂H, —CO₂C₁₋₄alkyl, —C(O)NR^{e}R^{f}, and —NR^{e}R^{f};
wherein R^{e} and R^{f} are each independently H or C₁₋₄alkyl;
or R² and R³ taken together with the nitrogen to which they are attached form a 5- to 10-membered heterocyclyl ring optionally substituted with 1, 2, 3, or 4 substituents selected from the group consisting of C₁₋₄alkyl, —OH, —OC₁₋₄alkyl, oxo, halo, —CF₃, —CN, —C(O)C₁₋₄alkyl, —CO₂H, —CO₂C₁₋₄alkyl, —C(O)NR^{g}R^{h}, and —NR^{g}R^{h};
   wherein R^{g} and R^{h} are each independently H or C₁₋₄alkyl;
a and b are each independently 1 or 2;
r and s are each independently 0, 1, 2, 3, or 4; and
each R⁴ and R⁵ is independently C₁₋₄alkyl, —OH, —OC₁₋₄alkyl, —F, —CF₃, —CN, —NRⁱR^{j}, or oxo; wherein Rⁱ and R^{j} are each independently H or C₁₋₄alkyl;
   or a pharmaceutically acceptable salt thereof.

In some embodiments of Formula (I), the compound is not a compound selected from the group consisting of Compound Nos. 1X, 2X, 3X, 4X, 6X, 7X, 8X, 9X, 10X, 11X, 12X, 13X, 14X, 15X, 16X, 17X, 19X, 20X, 21X, 24X, 25X, 28X, 29X, 30X, 31X, 32X, 33X, 34X, 36X, 37X, 42X, 45X, 46X, 47X, 49X, 5X, 501X, 53X, 55X, 56X, 60X, 61X, 62X, 63X, 64X, 65X, 66X, 68X, 69X, 70X, 71X, 72X, 73X, 74X, 75X, 79X, 80X, 82X, 84X, 90X, 91X, 92X, 93X, 94X, 95X, 96X, 97X, 98X, 100X, 103X, 104X, 107X, 108X, 109X, 110X, 111X, 112X, 113X, 114X, 115X, 116X, 119X, 120X, 121X, 122X, 123X, 124X, 125X, 126X, 127X, 128X, 129X, 130X, 131X, 132X, 133X, 134X, 135X, 136X, 137X, 140X, 141X, 154X, 155X, 156X, 157X, 158X, 159X, 168X, 169X, 170X, 171X, 174X, 175X, 176X, 177X, 178X, 179X, 180X, 181X, 182X, 183X, 184X and 185X in Table X, and pharmaceutically acceptable salts thereof.

TABLE X

| Cpd. | | |
|---|---|---|
| No. | Structure | Compound Name |
| 1X | 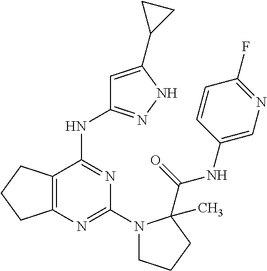 [SMILES: CC1(C(=O)Nc2ccc(F)nc2)CCCN1c1nc2c(c(Nc3cc(C4CC4)[nH]n3)n1)CCC2] | 1-(4-(5-cyclopropyl-1H-pyrazol-3- ylamino)-6,7-dihydro-5H- cyclopenta[d]pyrimidin-2-yl)-N-(6- fluoropyridin-3-yl)-2-methylpyrrolidine-2- carboxamide |
| 2X | 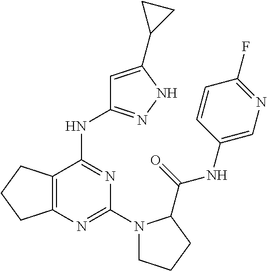 [SMILES: O=C(Nc1ccc(F)nc1)C1CCCN1c1nc2c(c(Nc3cc(C4CC4)[nH]n3)n1)CCC2] | 1-(4-(5-cyclopropyl-1H-pyrazol-3- ylamino)-6,7-dihydro-5H- cyclopenta[d]pyrimidin-2-yl)-N-(6- fluoropyridin-3-yl)pyrrolidine-2- carboxamide |
| 3X | 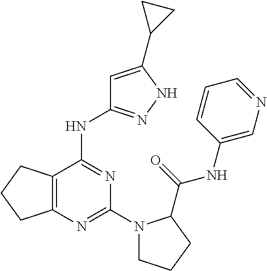 [SMILES: O=C(Nc1cccnc1)C1CCCN1c1nc2c(c(Nc3cc(C4CC4)[nH]n3)n1)CCC2] | 1-(4-(5-cyclopropyl-1H-pyrazol-3- ylamino)-6,7-dihydro-5H- cyclopenta[d]pyrimidin-2-yl)-N-(pyridin-3- yl)pyrrolidine-2-carboxamide |
| 4X | 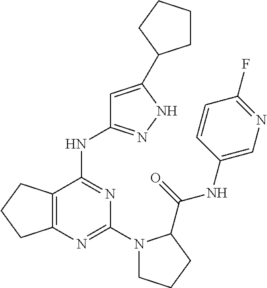 [SMILES: O=C(Nc1ccc(F)nc1)C1CCCN1c1nc2c(c(Nc3cc(C4CCCC4)[nH]n3)n1)CCC2] | 1-(4-(5-cyclopentyl-1H-pyrazol-3- ylamino)-6,7-dihydro-5H- cyclopenta[d]pyrimidin-2-yl)-N-(6- fluoropyridin-3-yl)pyrrolidine-2- carboxamide |
| 6X | 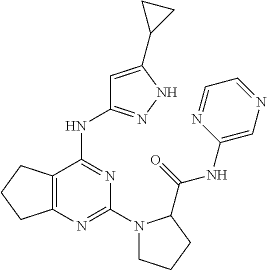 [SMILES: O=C(Nc1cnccn1)C1CCCN1c1nc2c(c(Nc3cc(C4CC4)[nH]n3)n1)CCC2] | 1-(4-(5-cyclopropyl-1H-pyrazol-3- ylamino)-6,7-dihydro-5H- cyclopenta[d]pyrimidin-2-yl)-N-(pyrazin- 2-yl)pyrrolidine-2-carboxamide |
| 7X | 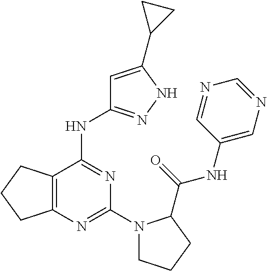 [SMILES: O=C(Nc1cncnc1)C1CCCN1c1nc2c(c(Nc3cc(C4CC4)[nH]n3)n1)CCC2] | 1-(4-(5-cyclopropyl-1H-pyrazol-3- ylamino)-6,7-dihydro-5H- cyclopenta[d]pyrimidin-2-yl)-N- (pyrimidin-5-yl)pyrrolidine-2-carboxamide |
| 8X | 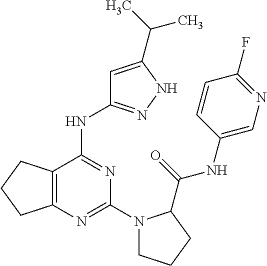 [SMILES: CC(C)c1cc(Nc2nc(N3CCCC3C(=O)Nc3ccc(F)nc3)nc3c2CCC3)n[nH]1] | N-(6-fluoropyridin-3-yl)-1-(4-(5-isopropyl- 1H-pyrazol-3-ylamino)-6,7-dihydro-5H- cyclopenta[d]pyrimidin-2-yl)pyrrolidine-2- carboxamide |
| 9X | 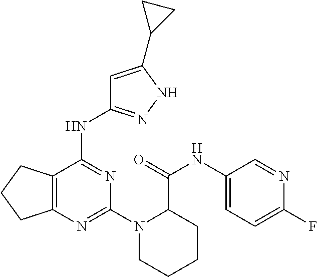 [SMILES: O=C(Nc1ccc(F)nc1)C1CCCCN1c1nc2c(c(Nc3cc(C4CC4)[nH]n3)n1)CCC2] | 1-(4-(5-cyclopropyl-1H-pyrazol-3- ylamino)-6,7-dihydro-5H- cyclopenta[d]pyrimidin-2-yl)-N-(6- fluoropyridin-3-yl)piperidine-2- carboxamide |
| 10X | 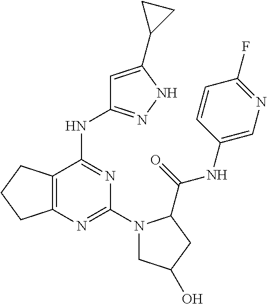 [SMILES: O=C(Nc1ccc(F)nc1)C1CC(O)CN1c1nc2c(c(Nc3cc(C4CC4)[nH]n3)n1)CCC2] | 1-(4-(5-cyclopropyl-1H-pyrazol-3- ylamino)-6,7-dihydro-5H- cyclopenta[d]pyrimidin-2-yl)-N-(6- fluoropyridin-3-yl)-4-hydroxypyrrolidine- 2-carboxamide |
| 11X | 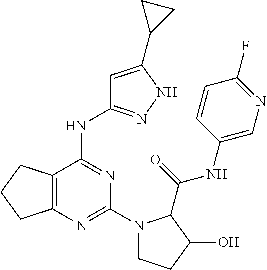 [SMILES: O=C(Nc1ccc(F)nc1)C1C(O)CCN1c1nc2c(c(Nc3cc(C4CC4)[nH]n3)n1)CCC2] | 1-(4-(5-cyclopropyl-1H-pyrazol-3- ylamino)-6,7-dihydro-5H- cyclopenta[d]pyrimidin-2-yl)-N-(6- fluoropyridin-3-yl)-3-hydroxypyrrolidine- 2-carboxamide |
| 12X | 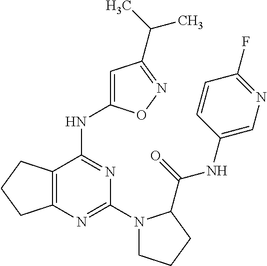 [SMILES: CC(C)c1cc(Nc2nc(N3CCCC3C(=O)Nc3ccc(F)nc3)nc3c2CCC3)on1] | N-(6-fluoropyridin-3-yl)-1-(4-(3- isopropylisoxazol-5-ylamino)-6,7-dihydro- 5H-cyclopenta[d]pyrimidin-2- yl)pyrrolidine-2-carboxamide |
| 13X | 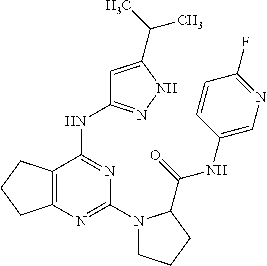 [SMILES: CC(C)c1cc(Nc2nc(N3CCCC3C(=O)Nc3ccc(F)nc3)nc3c2CCC3)n[nH]1] | N-(6-fluoropyridin-3-yl)-1-(4-(5-isopropyl- 1H-pyrazol-3-ylamino)-5,6,7,8- tetrahydroquinazolin-2-yl)pyrrolidine-2- carboxamide |
| 14X | 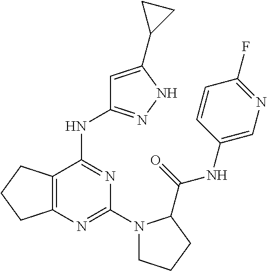 [SMILES: O=C(Nc1ccc(F)nc1)C1CCCN1c1nc2c(c(Nc3cc(C4CC4)[nH]n3)n1)CCC2] | 1-(4-(5-cyclopropyl-1H-pyrazol-3- ylamino)-5,6,7,8-tetrahydroquinazolin-2- yl)-N-(6-fluoropyridin-3-yl)pyrrolidine-2- carboxamide |
| 15X | 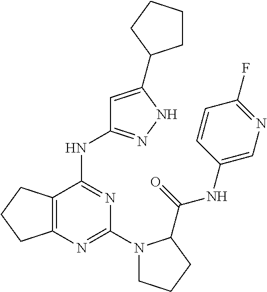 [SMILES: O=C(Nc1ccc(F)nc1)C1CCCN1c1nc2c(c(Nc3cc(C4CCCC4)[nH]n3)n1)CCC2] | 1-(4-(5-cyclopentyl-1H-pyrazol-3- ylamino)-5,6,7,8-tetrahydroquinazolin-2- yl)-N-(6-fluoropyridin-3-yl)pyrrolidine-2- carboxamide |
| 16X | 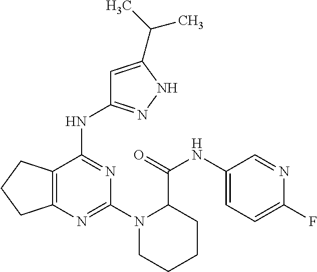 [SMILES: CC(C)c1cc(Nc2nc(N3CCCCC3C(=O)Nc3ccc(F)nc3)nc3c2CCC3)n[nH]1] | N-(6-fluoropyridin-3-yl)-1-(4-(5-isopropyl- 1H-pyrazol-3-ylamino)-6,7-dihydro-5H- cyclopenta[d]pyrimidin-2-yl)piperidine-2- carboxamide |
| 17X | 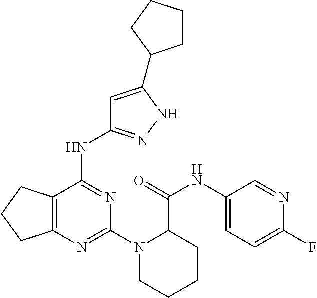 [SMILES: O=C(Nc1ccc(F)nc1)C1CCCCN1c1nc2c(c(Nc3cc(C4CCCC4)[nH]n3)n1)CCC2] | 1-(4-(5-cyclopentyl-1H-pyrazol-3- ylamino)-6,7-dihydro-5H- cyclopenta[d]pyrimidin-2-yl)-N-(6- fluoropyridin-3-yl)piperidine-2- carboxamide |
| 19X | 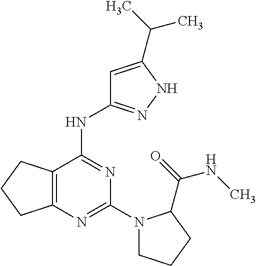 [SMILES: CNC(=O)C1CCCN1c1nc2c(c(Nc3cc(C(C)C)[nH]n3)n1)CCC2] | 1-(4-(5-isopropyl-1H-pyrazol-3-ylamino)- 6,7-dihydro-5H-cyclopenta[d]pyrimidin-2- yl)-N-methylpyrrolidine-2-carboxamide |
| 20X | 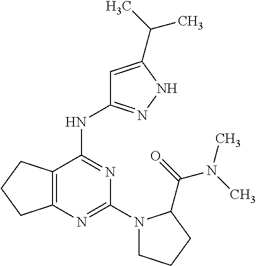 [SMILES: CC(C)c1cc(Nc2nc(N3CCCC3C(=O)N(C)C)nc3c2CCC3)n[nH]1] | 1-(4-(5-isopropyl-1H-pyrazol-3-ylamino)- 6,7-dihydro-5H-cyclopenta[d]pyrimidin-2- yl)-N,N-dimethylpyrrolidine-2- carboxamide |
| 21X | 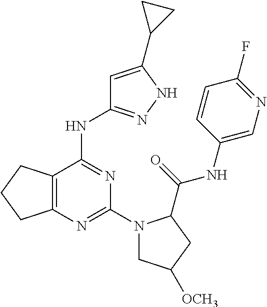 [SMILES: COC1CC(C(=O)Nc2ccc(F)nc2)N(c2nc3c(c(Nc4cc(C5CC5)[nH]n4)n2)CCC3)C1] | 1-(4-(5-cyclopropyl-1H-pyrazol-3- ylamino)-6,7-dihydro-5H- cyclopenta[d]pyrimidin-2-yl)-N-(6- fluoropyridin-3-yl)-4-methoxypyrrolidine- 2-carboxamide |
| 24X | 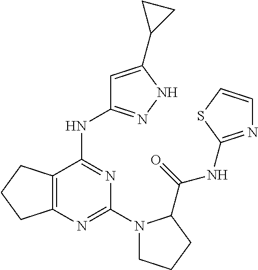 [SMILES: O=C(Nc1nccs1)C1CCCN1c1nc2c(c(Nc3cc(C4CC4)[nH]n3)n1)CCC2] | 1-(4-((5-cyclopropyl-1H-pyrazol-3- yl)amino)-6,7-dihydro-5H- cyclopenta[d]pyrimidin-2-yl)-N-(thiazol-2- yl)pyrrolidine-2-carboxamide |
| 25X | 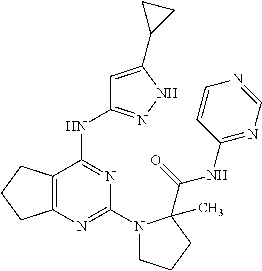 [SMILES: CC1(C(=O)Nc2ccncn2)CCCN1c1nc2c(c(Nc3cc(C4CC4)[nH]n3)n1)CCC2] | 1-(4-((5-cyclopropyl-1H-pyrazol-3- yl)amino)-6,7-dihydro-5H- cyclopenta[d]pyrimidin-2-yl)-2-methyl-N- (pyrimidin-4-yl)pyrrolidine-2-carboxamide |
| 28X | 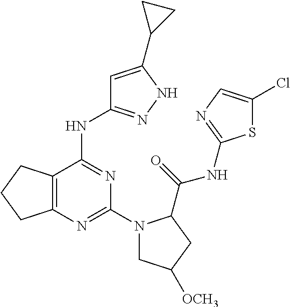 [SMILES: COC1CC(C(=O)Nc2ncc(Cl)s2)N(c2nc3c(c(Nc4cc(C5CC5)[nH]n4)n2)CCC3)C1] | N-(5-chlorothiazol-2-yl)-1-(4-((5- cyclopropyl-1H-pyrazol-3-yl)amino)-6,7- dihydro-5H-cyclopenta[d]pyrimidin-2-yl)- 4-methoxypyrrolidine-2-carboxamide |
| 29X | 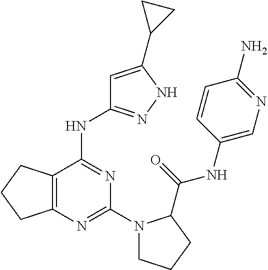 [SMILES: Nc1ccc(NC(=O)C2CCCN2c2nc3c(c(Nc4cc(C5CC5)[nH]n4)n2)CCC3)cn1] | N-(6-aminopyridin-3-yl)-1-(4-((5- cyclopropyl-1H-pyrazol-3-yl)amino)-6,7- dihydro-5H-cyclopenta[d]pyrimidin-2- yl)pyrrolidine-2-carboxamide |
| 30X | 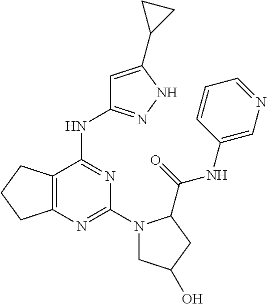 [SMILES: O=C(Nc1cccnc1)C1CC(O)CN1c1nc2c(c(Nc3cc(C4CC4)[nH]n3)n1)CCC2] | 1-(4-((5-cyclopropyl-1H-pyrazol-3- yl)amino)-6,7-dihydro-5H- cyclopenta[d]pyrimidin-2-yl)-4-hydroxy- N-(pyridin-3-yl)pyrrolidine-2-carboxamide |
| 31X | 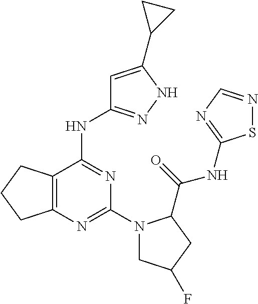 [SMILES: O=C(Nc1ncns1)C1CC(F)CN1c1nc2c(c(Nc3cc(C4CC4)[nH]n3)n1)CCC2] | 1-(4-((5-cyclopropyl-1H-pyrazol-3- yl)amino)-6,7-dihydro-5H- cyclopenta[d]pyrimidin-2-yl)-4-fluoro-N- (1,2,4-thiadiazol-5-yl)pyrrolidine-2- carboxamide |
| 32X | 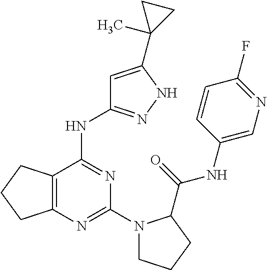 [SMILES: CC1(c2cc(Nc3nc(N4CCCC4C(=O)Nc4ccc(F)nc4)nc4c3CCC4)n[nH]2)CC1] | N-(6-fluoropyridin-3-yl)-1-(4-((5-(1- methylcyclopropyl)-1H-pyrazol-3- yl)amino)-6,7-dihydro-5H- cyclopenta[d]pyrimidin-2-yl)pyrrolidine-2- carboxamide |
| 33X | 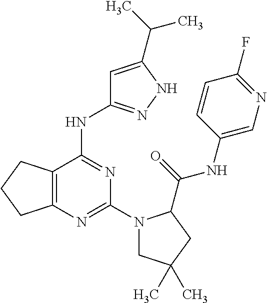 [SMILES: CC(C)c1cc(Nc2nc(N3CC(C)(C)CC3C(=O)Nc3ccc(F)nc3)nc3c2CCC3)n[nH]1] | N-(6-fluoropyridin-3-yl)-1-(4-((5- isopropyl-1H-pyrazol-3-yl)amino)-6,7- dihydro-5H-cyclopenta[d]pyrimidin-2-yl)- 4,4-dimethylpyrrolidine-2-carboxamide |
| 34X | 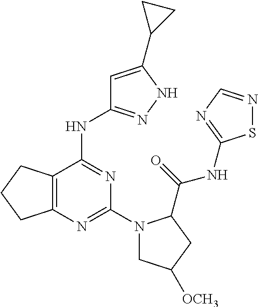 [SMILES: COC1CC(C(=O)Nc2ncns2)N(c2nc3c(c(Nc4cc(C5CC5)[nH]n4)n2)CCC3)C1] | 1-(4-((5-cyclopropyl-1H-pyrazol-3- yl)amino)-6,7-dihydro-5H- cyclopenta[d]pyrimidin-2-yl)-4-methoxy- N-(1,2,4-thiadiazol-5-yl)pyrrolidine-2- carboxamide |
| 36X | 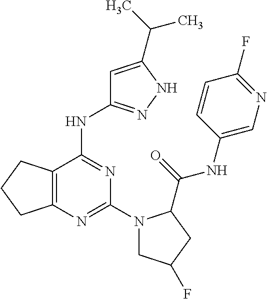 [SMILES: CC(C)c1cc(Nc2nc(N3CC(F)CC3C(=O)Nc3ccc(F)nc3)nc3c2CCC3)n[nH]1] | 4-fluoro-N-(6-fluoropyridin-3-yl)-1-(4-((5- isopropyl-1H-pyrazol-3-yl)amino)-6,7- dihydro-5H-cyclopenta[d]pyrimidin-2- yl)pyrrolidine-2-carboxamide |
| 37X | 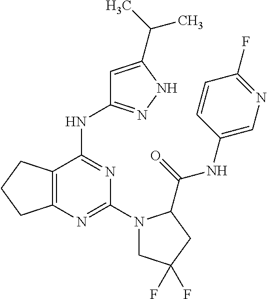 [SMILES: CC(C)c1cc(Nc2nc(N3CC(F)(F)CC3C(=O)Nc3ccc(F)nc3)nc3c2CCC3)n[nH]1] | 4,4-difluoro-N-(6-fluoropyridin-3-yl)-1-(4- ((5-isopropyl-1H-pyrazol-3-yl)amino)-6,7- dihydro-5H-cyclopenta[d]pyrimidin-2- yl)pyrrolidine-2-carboxamide |
| 42X | 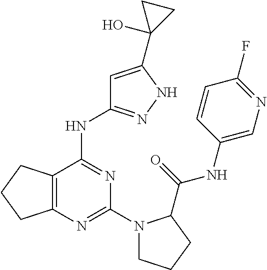 [SMILES: O=C(Nc1ccc(F)nc1)C1CCCN1c1nc2c(c(Nc3cc(C4(O)CC4)[nH]n3)n1)CCC2] | N-(6-fluoropyridin-3-yl)-1-(4-((5-(1- hydroxycyclopropyl)-1H-pyrazol-3- yl)amino)-6,7-dihydro-5H- cyclopenta[d]pyrimidin-2-yl)pyrrolidine-2- carboxamide |
| 45X | 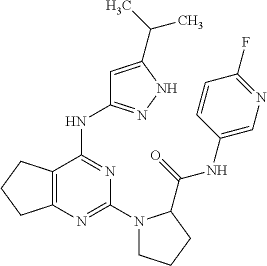 [SMILES: CC(C)c1cc(Nc2nc(N3CCCC3C(=O)Nc3ccc(F)nc3)nc3c2CCC3)n[nH]1] | N-(6-fluoropyridin-3-yl)-1-(4-((2- isopropyl-1H-imidazol-5-yl)amino)-6,7- dihydro-5H-cyclopenta[d]pyrimidin-2- yl)pyrrolidine-2-carboxamide |
| 46X | 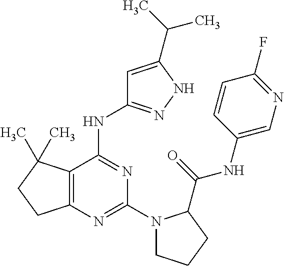 [SMILES: CC(C)c1cc(Nc2nc(N3CCCC3C(=O)Nc3ccc(F)nc3)nc3c2C(C)(C)CC3)n[nH]1] | N-(6-fluoropyridin-3-yl)-1-(4-((5- isopropyl-1H-pyrazol-3-yl)amino)-5,5- dimethyl-6,7-dihydro-5H- cyclopenta[d]pyrimidin-2-yl)pyrrolidine-2- carboxamide |
| 47X | 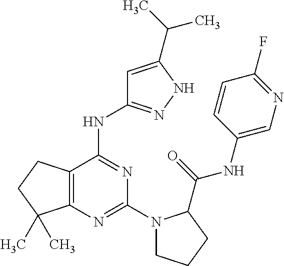 [SMILES: CC(C)c1cc(Nc2nc(N3CCCC3C(=O)Nc3ccc(F)nc3)nc3c2CCC3(C)C)n[nH]1] | N-(6-fluoropyridin-3-yl)-1-(4-((5- isopropyl-1H-pyrazol-3-yl)amino)-7,7- dimethyl-6,7-dihydro-5H- cyclopenta[d]pyrimidin-2-yl)pyrrolidine-2- carboxamide |
| 49X | 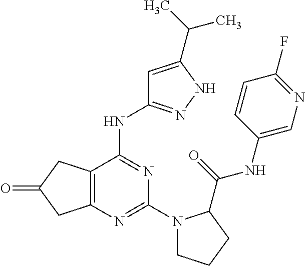 [SMILES: CC(C)c1cc(Nc2nc(N3CCCC3C(=O)Nc3ccc(F)nc3)nc3c2CC(=O)C3)n[nH]1] | N-(6-fluoropyridin-3-yl)-1-(4-((5- isopropyl-1H-pyrazol-3-yl)amino)-6-oxo- 6,7-dihydro-5H-cyclopenta[d]pyrimidin-2- yl)pyrrolidine-2-carboxamide |
| 50X | 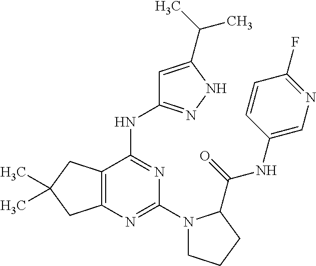 [SMILES: CC(C)c1cc(Nc2nc(N3CCCC3C(=O)Nc3ccc(F)nc3)nc3c2CC(C)(C)C3)n[nH]1] | N-(6-fluoropyridin-3-yl)-1-(4-((5- isopropyl-1H-pyrazol-3-yl)amino)-6,6- dimethyl-6,7-dihydro-5H- cyclopenta[d]pyrimidin-2-yl)pyrrolidine-2- carboxamide |
| 51X | 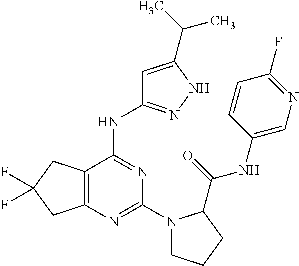 [SMILES: CC(C)c1cc(Nc2nc(N3CCCC3C(=O)Nc3ccc(F)nc3)nc3c2CC(F)(F)C3)n[nH]1] | 1-(6,6-difluoro-4-((5-isopropyl-1H- pyrazol-3-yl)amino)-6,7-dihydro-5H- cyclopenta[d]pyrimidin-2-yl)-N-(6- fluoropyridin-3-yl)pyrrolidine-2- carboxamide |
| 53X | 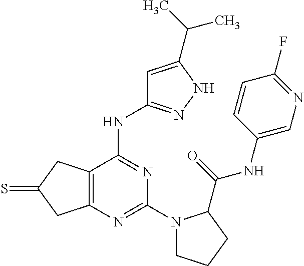 [SMILES: CC(C)c1cc(Nc2nc(N3CCCC3C(=O)Nc3ccc(F)nc3)nc3c2CC(=S)C3)n[nH]1] | N-(6-fluoropyridin-3-yl)-1-(4-((5- isopropyl-1H-pyrazol-3-yl)amino)-6- thioxo-6,7-dihydro-5H- cyclopenta[d]pyrimidin-2-yl)pyrrolidine-2- carboxamide |
| 55X | 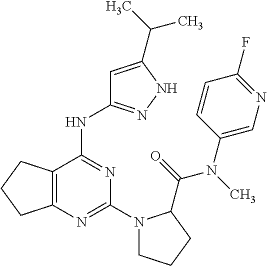 [SMILES: CC(C)c1cc(Nc2nc(N3CCCC3C(=O)N(C)c3ccc(F)nc3)nc3c2CCC3)n[nH]1] | N-(6-fluoropyridin-3-yl)-1-(4-((5- isopropyl-1H-pyrazol-3-yl)amino)-6,7- dihydro-5H-cyclopenta[d]pyrimidin-2-yl)- N-methylpyrrolidine-2-carboxamide |
| 56X | 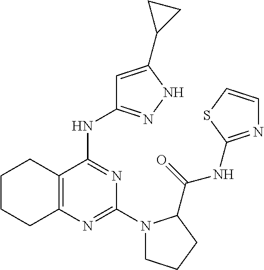 [SMILES: O=C(Nc1nccs1)C1CCCN1c1nc2c(c(Nc3cc(C4CC4)[nH]n3)n1)CCCC2] | 1-(4-((5-cyclopropyl-1H-pyrazol-3- yl)amino)-5,6,7,8-tetrahydroquinazolin-2- yl)-N-(thiazol-2-yl)pyrrolidine-2- carboxamide |
| 60X | 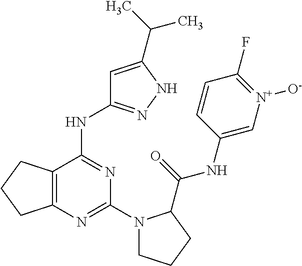 [SMILES: CC(C)c1cc(Nc2nc(N3CCCC3C(=O)Nc3ccc(F)[n+]([O-])c3)nc3c2CCC3)n[nH]1] | 2-fluoro-5-(1-(4-((5-isopropyl-1H-pyrazol- 3-yl)amino)-6,7-dihydro-5H- cyclopenta[d]pyrimidin-2-yl)pyrrolidine-2- carboxamido)pyridine 1-oxide |
| 61X | 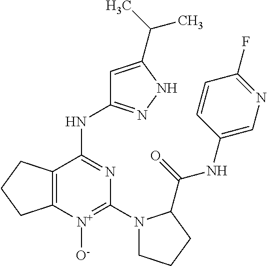 [SMILES: CC(C)c1cc(Nc2nc(N3CCCC3C(=O)Nc3ccc(F)nc3)[n+]([O-])c3c2CCC3)n[nH]1] | 2-(2-((6-fluoropyridin-3- yl)carbamoyl)pyrrolidin-1-yl)-4-((5- isopropyl-1H-pyrazol-3-yl)amino)-6,7- dihydro-5H-cyclopenta[d]pyrimidine 1- oxide |
| 62X | 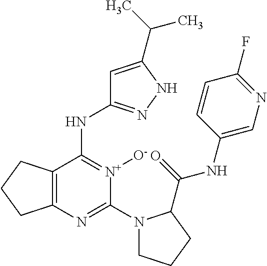 [SMILES: CC(C)c1cc(Nc2c3c(nc(N4CCCC4C(=O)Nc4ccc(F)nc4)[n+]2[O-])CCC3)n[nH]1] | 2-(2-((6-fluoropyridin-3- yl)carbamoyl)pyrrolidin-1-yl)-4-((5- isopropyl-1H-pyrazol-3-yl)amino)-6,7- dihydro-5H-cyclopenta[d]pyrimidine 3- oxide |
| 63X | 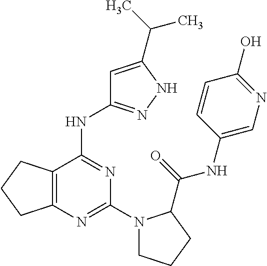 [SMILES: CC(C)c1cc(Nc2nc(N3CCCC3C(=O)Nc3ccc(O)nc3)nc3c2CCC3)n[nH]1] | N-(6-hydroxypyridin-3-yl)-1-(4-((5- isopropyl-1H-pyrazol-3-yl)amino)-6,7- dihydro-5H-cyclopenta[d]pyrimidin-2- yl)pyrrolidine-2-carboxamide |
| 64X | 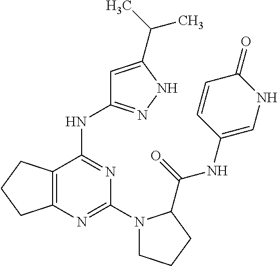 [SMILES: CC(C)c1cc(Nc2nc(N3CCCC3C(=O)Nc3ccc(=O)[nH]c3)nc3c2CCC3)n[nH]1] | 1-(4-((5-isopropyl-1H-pyrazol-3- yl)amino)-6,7-dihydro-5H- cyclopenta[d]pyrimidin-2-yl)-N-(6-oxo- 1,6-dihydropyridin-3-yl)pyrrolidine-2- carboxamide |
| 65X | 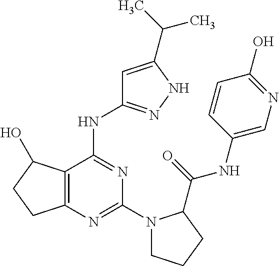 [SMILES: CC(C)c1cc(Nc2nc(N3CCCC3C(=O)Nc3ccc(O)nc3)nc3c2C(O)CC3)n[nH]1] | 1-(5-hydroxy-4-((5-isopropyl-1H-pyrazol- 3-yl)amino)-6,7-dihydro-5H- cyclopenta[d]pyrimidin-2-yl)-N-(6- hydroxypyridin-3-yl)pyrrolidine-2- carboxamide |
| 66X | 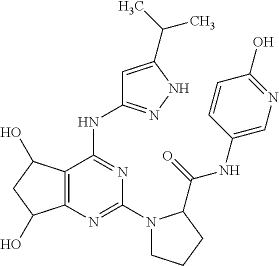 [SMILES: CC(C)c1cc(Nc2nc(N3CCCC3C(=O)Nc3ccc(O)nc3)nc3c2C(O)CC3O)n[nH]1] | 1-(5,7-dihydroxy-4-((5-isopropyl-1H- pyrazol-3-yl)amino)-6,7-dihydro-5H- cyclopenta[d]pyrimidin-2-yl)-N-(6- hydroxypyridin-3-yl)pyrrolidine-2- carboxamide |
| 68X | 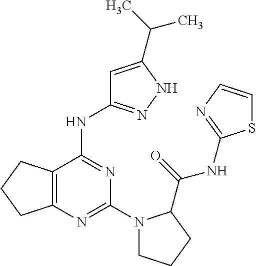 [SMILES: CC(C)c1cc(Nc2nc(N3CCCC3C(=O)Nc3nccs3)nc3c2CCC3)n[nH]1] | 1-(4-(5-isopropyl-1H-pyrazol-3-ylamino)- 6,7-dihydro-5H-cyclopenta[d]pyrimidin-2- yl)-N-(thiazol-2-yl)pyrrolidine-2- carboxamide |
| 69X | 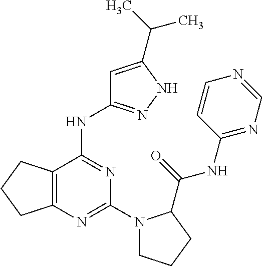 [SMILES: CC(C)c1cc(Nc2nc(N3CCCC3C(=O)Nc3ccncn3)nc3c2CCC3)n[nH]1] | 1-(4-(5-isopropyl-1H-pyrazol-3-ylamino)- 6,7-dihydro-5H-cyclopenta[d]pyrimidin-2- yl)-N-(pyrimidin-4-yl)pyrrolidine-2- carboxamide |
| 70X | 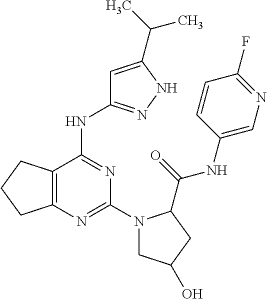 [SMILES: CC(C)c1cc(Nc2nc(N3CC(O)CC3C(=O)Nc3ccc(F)nc3)nc3c2CCC3)n[nH]1] | N-(6-fluoropyridin-3-yl)-4-hydroxy-1-(4- (5-isopropyl-1H-pyrazol-3-ylamino)-6,7- dihydro-5H-cyclopenta[d]pyrimidin-2- yl)pyrrolidine-2-carboxamide |
| 71X | 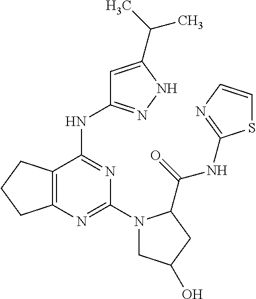 [SMILES: CC(C)c1cc(Nc2nc(N3CC(O)CC3C(=O)Nc3nccs3)nc3c2CCC3)n[nH]1] | 4-hydroxy-1-(4-(5-isopropyl-1H-pyrazol-3- ylamino)-6,7-dihydro-5H- cyclopenta[d]pyrimidin-2-yl)-N-(thiazol-2- yl)pyrrolidine-2-carboxamide |
| 72X | 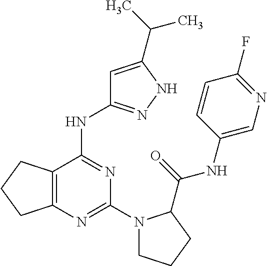 [SMILES: CC(C)c1cc(Nc2nc(N3CCCC3C(=O)Nc3ccc(F)nc3)nc3c2CCC3)n[nH]1] | N-(6-aminopyridin-3-yl)-1-(4-(5-isopropyl- 1H-pyrazol-3-ylamino)-6,7-dihydro-5H- cyclopenta[d]pyrimidin-2-yl)pyrrolidine-2- carboxamide |
| 73X | 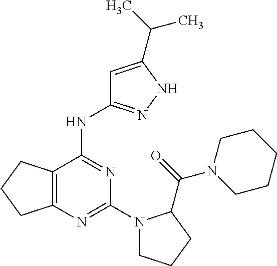 [SMILES: CC(C)c1cc(Nc2nc(N3CCCC3C(=O)N3CCCCC3)nc3c2CCC3)n[nH]1] | (1-(4-(5-isopropyl-1H-pyrazol-3-ylamino)- 6,7-dihydro-5H-cyclopenta[d]pyrimidin-2- yl)pyrrolidin-2-yl)(piperidin-1- yl)methanone |
| 74X | 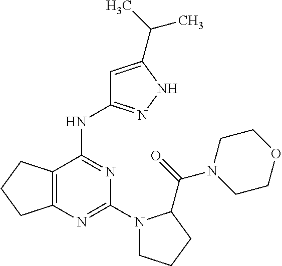 [SMILES: CC(C)c1cc(Nc2nc(N3CCCC3C(=O)N3CCOCC3)nc3c2CCC3)n[nH]1] | (1-(4-(5-isopropyl-1H-pyrazol-3-ylamino)- 6,7-dihydro-5H-cyclopenta[d]pyrimidin-2- yl)pyrrolidin-2-yl)(morpholino)methanone |
| 75X | 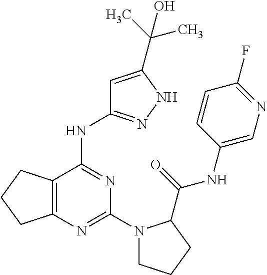 [SMILES: CC(C)(O)c1cc(Nc2nc(N3CCCC3C(=O)Nc3ccc(F)nc3)nc3c2CCC3)n[nH]1] | N-(6-fluoropyridin-3-yl)-1-(4-(5-(2- hydroxypropan-2-yl)-1H-pyrazol-3- ylamino)-6,7-dihydro-5H- cyclopenta[d]pyrimidin-2-yl)pyrrolidine-2- carboxamide |
| 79X | 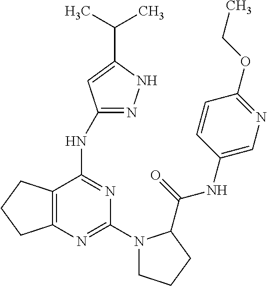 [SMILES: CCOc1ccc(NC(=O)C2CCCN2c2nc3c(c(Nc4cc(C(C)C)[nH]n4)n2)CCC3)cn1] | N-(6-ethoxypyridin-3-yl)-1-(4-(5- isopropyl-1H-pyrazol-3-ylamino)-6,7- dihydro-5H-cyclopenta[d]pyrimidin-2- yl)pyrrolidine-2-carboxamide |
| 80X | 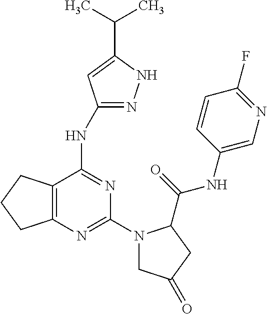 [SMILES: CC(C)c1cc(Nc2nc(N3CC(=O)CC3C(=O)Nc3ccc(F)nc3)nc3c2CCC3)n[nH]1] | N-(6-fluoropyridin-3-yl)-1-(4-(5-isopropyl- 1H-pyrazol-3-ylamino)-6,7-dihydro-5H- cyclopenta[d]pyrimidin-2-yl)-4- oxopyrrolidine-2-carboxamide |
| 82X | 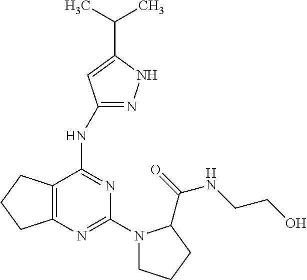 [SMILES: CC(C)c1cc(Nc2nc(N3CCCC3C(=O)NCCO)nc3c2CCC3)n[nH]1] | N-(2-hydroxyethyl)-1-(4-(5-isopropyl- 1H-pyrazol-3-ylamino)-6,7-dihydro-5H- cyclopenta[d]pyrimidin-2-yl)pyrrolidine-2- carboxamide |
| 84X | 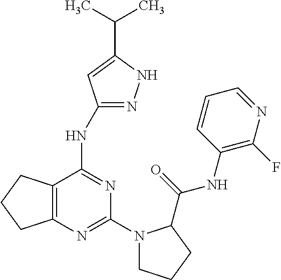 [SMILES: CC(C)c1cc(Nc2nc(N3CCCC3C(=O)Nc3cccnc3F)nc3c2CCC3)n[nH]1] | N-(2-fluoropyridin-3-yl)-1-(4-(5-isopropyl- 1H-pyrazol-3-ylamino)-6,7-dihydro-5H- cyclopenta[d]pyrimidin-2-yl)pyrrolidine-2- carboxamide |
| 90X | 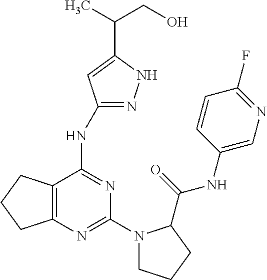 [SMILES: CC(CO)c1cc(Nc2nc(N3CCCC3C(=O)Nc3ccc(F)nc3)nc3c2CCC3)n[nH]1] | N-(6-fluoropyridin-3-yl)-1-(4-(5-(1- hydroxypropan-2-yl)-1H-pyrazol-3- ylamino)-6,7-dihydro-5H- cyclopenta[d]pyrimidin-2-yl)pyrrolidine-2- carboxamide |
| 91X | 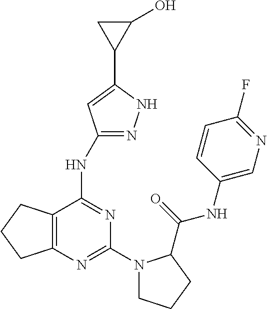 [SMILES: O=C(Nc1ccc(F)nc1)C1CCCN1c1nc2c(c(Nc3cc(C4CC4O)[nH]n3)n1)CCC2] | N-(6-fluoropyridin-3-yl)-1-(4-(5-(2- hydroxycyclopropyl)-1H-pyrazol-3- ylamino)-6,7-dihydro-5H- cyclopenta[d]pyrimidin-2-yl)pyrrolidine-2- carboxamide |
| 92X | 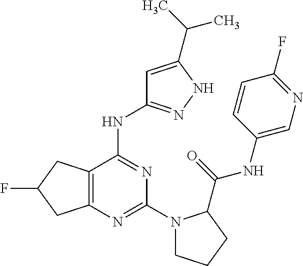 [SMILES: CC(C)c1cc(Nc2nc(N3CCCC3C(=O)Nc3ccc(F)nc3)nc3c2CC(F)C3)n[nH]1] | 1-(6-fluoro-4-(5-isopropyl-1H-pyrazol-3- ylamino)-6,7-dihydro-5H- cyclopenta[d]pyrimidin-2-yl)-N-(6- fluoropyridin-3-yl)pyrrolidine-2- carboxamide |
| 93X | 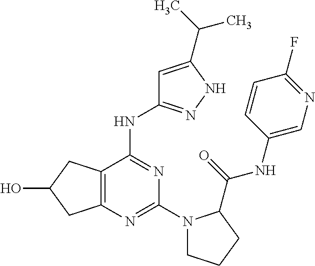 [SMILES: CC(C)c1cc(Nc2nc(N3CCCC3C(=O)Nc3ccc(F)nc3)nc3c2CC(O)C3)n[nH]1] | N-(6-fluoropyridin-3-yl)-1-(6-hydroxy-4- (5-isopropyl-1H-pyrazol-3-ylamino)-6,7- dihydro-5H-cyclopenta[d]pyrimidin-2- yl)pyrrolidine-2-carboxamide |
| 94X | 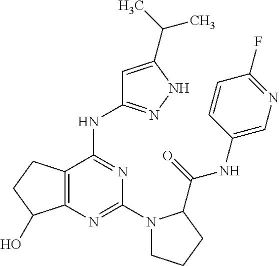 [SMILES: CC(C)c1cc(Nc2nc(N3CCCC3C(=O)Nc3ccc(F)nc3)nc3c2CCC3O)n[nH]1] | N-(6-fluoropyridin-3-yl)-1-(7-hydroxy-4- (5-isopropyl-1H-pyrazol-3-ylamino)-6,7- dihydro-5H-cyclopenta[d]pyrimidin-2- yl)pyrrolidine-2-carboxamide |
| 95X | 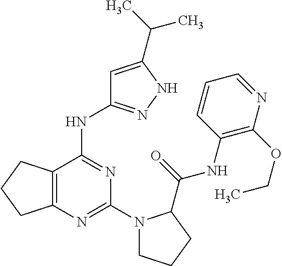 [SMILES: CCOc1ncccc1NC(=O)C1CCCN1c1nc2c(c(Nc3cc(C(C)C)[nH]n3)n1)CCC2] | N-(2-ethoxypyridin-3-yl)-1-(4-(5- isopropyl-1H-pyrazol-3-ylamino)-6,7- dihydro-5H-cyclopenta[d]pyrimidin-2- yl)pyrrolidine-2-carboxamide |
| 96X | 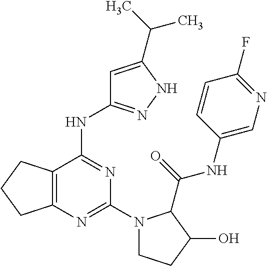 [SMILES: CC(C)c1cc(Nc2nc(N3CCC(O)C3C(=O)Nc3ccc(F)nc3)nc3c2CCC3)n[nH]1] | N-(6-fluoropyridin-3-yl)-3-hydroxy-1-(4- ((5-isopropyl-1H-pyrazol-3-yl)amino)-6,7- dihydro-5H-cyclopenta[d]pyrimidin-2- yl)pyrrolidine-2-carboxamide |
| 97X | 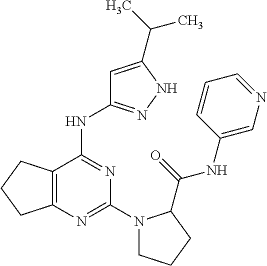 [SMILES: CC(C)c1cc(Nc2nc(N3CCCC3C(=O)Nc3cccnc3)nc3c2CCC3)n[nH]1] | 1-(4-(5-isopropyl-1H-pyrazol-3-ylamino)- 6,7-dihydro-5H-cyclopenta[d]pyrimidin-2- yl)-N-(pyridin-3-yl)pyrrolidine-2- carboxamide |
| 98X | 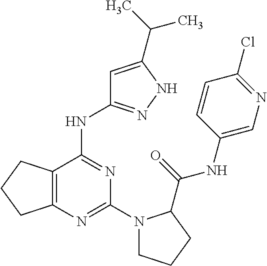 [SMILES: CC(C)c1cc(Nc2nc(N3CCCC3C(=O)Nc3ccc(Cl)nc3)nc3c2CCC3)n[nH]1] | N-(6-chloropyridin-3-yl)-1-(4-(5- isopropyl-1H-pyrazol-3-ylamino)-6,7- dihydro-5H-cyclopenta[d]pyrimidin-2- yl)pyrrolidine-2-carboxamide |
| 100X | 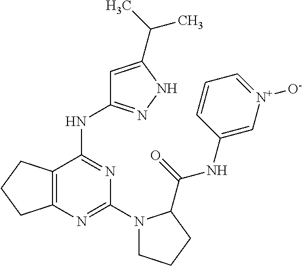 [SMILES: CC(C)c1cc(Nc2nc(N3CCCC3C(=O)Nc3ccc[n+]([O-])c3)nc3c2CCC3)n[nH]1] | 3-(1-(4-(5-isopropyl-1H-pyrazol-3- ylamino)-6,7-dihydro-5H- cyclopenta[d]pyrimidin-2-yl)pyrrolidine-2- carboxamido)pyridine 1-oxide |
| 103X | 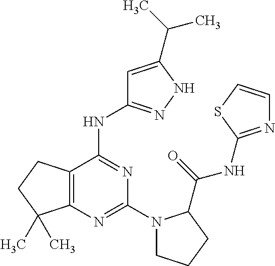 [SMILES: CC(C)c1cc(Nc2nc(N3CCCC3C(=O)Nc3nccs3)nc3c2CCC3(C)C)n[nH]1] | 1-(4-(5-isopropyl-1H-pyrazol-3-ylamino)- 7,7-dimethyl-6,7-dihydro-5H- cyclopenta[d]pyrimidin-2-yl)-N-(thiazol-2- yl)pyrrolidine-2-carboxamide |
| 104X | 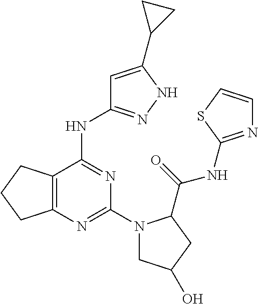 [SMILES: O=C(Nc1nccs1)C1CC(O)CN1c1nc2c(c(Nc3cc(C4CC4)[nH]n3)n1)CCC2] | 1-(4-(5-cyclopropyl-1H-pyrazol-3- ylamino)-6,7-dihydro-5H- cyclopenta[d]pyrimidin-2-yl)-4-hydroxy- N-(thiazol-2-yl)pyrrolidine-2-carboxamide |
| 107X | 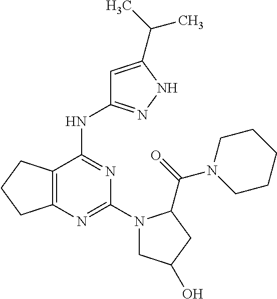 [SMILES: CC(C)c1cc(Nc2nc(N3CC(O)CC3C(=O)N3CCCCC3)nc3c2CCC3)n[nH]1] | (4-hydroxy-1-(4-(5-isopropyl-1H-pyrazol- 3-ylamino)-6,7-dihydro-5H- cyclopenta[d]pyrimidin-2-yl)pyrrolidin-2- yl)(piperidin-1-yl)methanone |
| 108X | 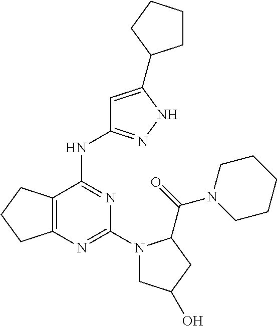 [SMILES: O=C(C1CC(O)CN1c1nc2c(c(Nc3cc(C4CCCC4)[nH]n3)n1)CCC2)N1CCCCC1] | (1-(4-(5-cyclopentyl-1H-pyrazol-3- ylamino)-6,7-dihydro-5H- cyclopenta[d]pyrimidin-2-yl)-4- hydroxypyrrolidin-2-yl)(piperidin-1- yl)methanone |
| 109X | 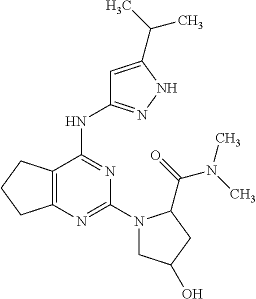 [SMILES: CC(C)c1cc(Nc2nc(N3CC(O)CC3C(=O)N(C)C)nc3c2CCC3)n[nH]1] | 4-hydroxy-1-(4-(5-isopropyl-1H-pyrazol-3- ylamino)-6,7-dihydro-5H- cyclopenta[d]pyrimidin-2-yl)-N,N- dimethylpyrrolidine-2-carboxamide |
| 110X | 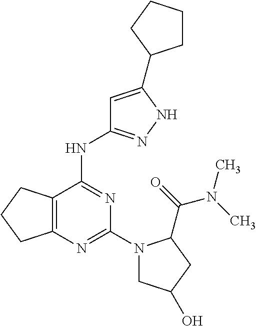 [SMILES: CN(C)C(=O)C1CC(O)CN1c1nc2c(c(Nc3cc(C4CCCC4)[nH]n3)n1)CCC2] | 1-(4-(5-cyclopentyl-1H-pyrazol-3- ylamino)-6,7-dihydro-5H- cyclopenta[d]pyrimidin-2-yl)-4-hydroxy- N,N-dimethylpyrrolidine-2-carboxamide |
| 111X | 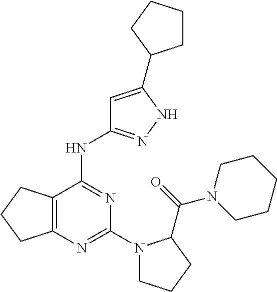 [SMILES: O=C(C1CCCN1c1nc2c(c(Nc3cc(C4CCCC4)[nH]n3)n1)CCC2)N1CCCCC1] | (1-(4-(5-cyclopentyl-1H-pyrazol-3- ylamino)-6,7-dihydro-5H- cyclopenta[d]pyrimidin-2-yl)pyrrolidin-2- yl)(piperidin-1-yl)methanone |
| 112X | 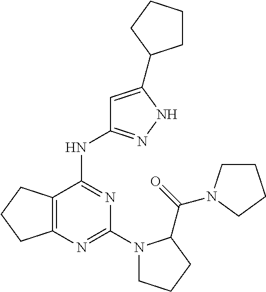 [SMILES: O=C(C1CCCN1c1nc2c(c(Nc3cc(C4CCCC4)[nH]n3)n1)CCC2)N1CCCC1] | (1-(4-(5-cyclopentyl-1H-pyrazol-3- ylamino)-6,7-dihydro-5H- cyclopenta[d]pyrimidin-2-yl)pyrrolidin-2- yl)(pyrrolidin-1-yl)methanone |
| 113X | 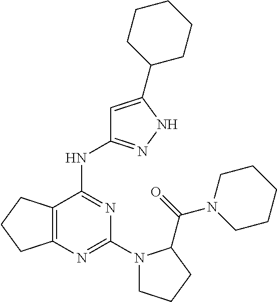 [SMILES: O=C(C1CCCN1c1nc2c(c(Nc3cc(C4CCCCC4)[nH]n3)n1)CCC2)N1CCCCC1] | (1-(4-(5-cyclohexyl-1H-pyrazol-3- ylamino)-6,7-dihydro-5H- cyclopenta[d]pyrimidin-2-yl)pyrrolidin-2- yl)(piperidin-1-yl)methanone |
| 114X | 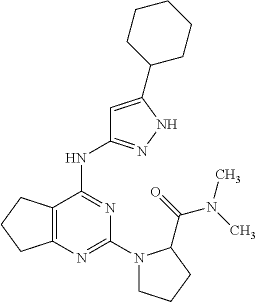 [SMILES: CN(C)C(=O)C1CCCN1c1nc2c(c(Nc3cc(C4CCCCC4)[nH]n3)n1)CCC2] | 1-(4-(5-cyclohexyl-1H-pyrazol-3- ylamino)-6,7-dihydro-5H- cyclopenta[d]pyrimidin-2-yl)-N,N- dimethylpyrrolidine-2-carboxamide |
| 115X | 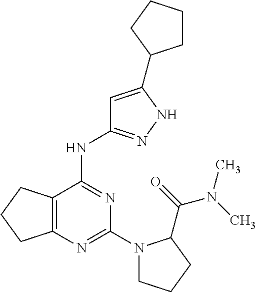 [SMILES: CN(C)C(=O)C1CCCN1c1nc2c(c(Nc3cc(C4CCCC4)[nH]n3)n1)CCC2] | 1-(4-(5-cyclopentyl-1H-pyrazol-3- ylamino)-6,7-dihydro-5H- cyclopenta[d]pyrimidin-2-yl)-N,N- dimethylpyrrolidine-2-carboxamide |
| 116X | 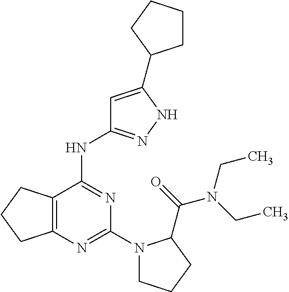 [SMILES: CCN(CC)C(=O)C1CCCN1c1nc2c(c(Nc3cc(C4CCCC4)[nH]n3)n1)CCC2] | 1-(4-(5-cyclopentyl-1H-pyrazol-3- ylamino)-6,7-dihydro-5H- cyclopenta[d]pyrimidin-2-yl)-N,N- diethylpyrrolidine-2-carboxamide |
| 119X | 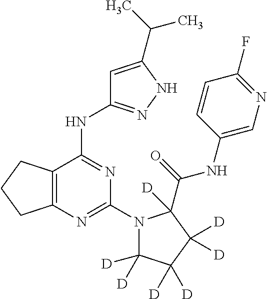 [SMILES: [2H]C1([2H])N(c2nc3c(c(Nc4cc(C(C)C)[nH]n4)n2)CCC3)C([2H])(C(=O)Nc2ccc(F)nc2)C([2H])([2H])C1([2H])[2H]] | 2,3,3,4,4,5,5-heptadeutero-N-(6- fluoropyridin-3-yl)-1-(4-(5-isopropyl-1H- pyrazol-3-ylamino)-6,7-dihydro-5H- cyclopenta[d]pyrimidin-2-yl)pyrrolidine-2- carboxamide |
| 120X | 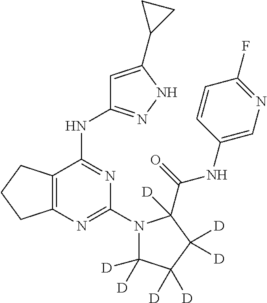 [SMILES: [2H]C1([2H])N(c2nc3c(c(Nc4cc(C5CC5)[nH]n4)n2)CCC3)C([2H])(C(=O)Nc2ccc(F)nc2)C([2H])([2H])C1([2H])[2H]] | 2,3,3,4,4,5,5-heptadeutero-1-(4-(5- cyclopropyl-1H-pyrazol-3-ylamino)-6,7- dihydro-5H-cyclopenta[d]pyrimidin-2-yl)- N-(6-fluoropyridin-3-yl)pyrrolidine-2- carboxamide |
| 121X | 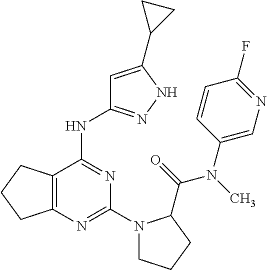 [SMILES: CN(C(=O)C1CCCN1c1nc2c(c(Nc3cc(C4CC4)[nH]n3)n1)CCC2)c1ccc(F)nc1] | 1-(4-(5-cyclopropyl-1H-pyrazol-3- ylamino)-6,7-dihydro-5H- cyclopenta[d]pyrimidin-2-yl)-N-(6- fluoropyridin-3-yl)-N-methylpyrrolidine-2- carboxamide |
| 122X | 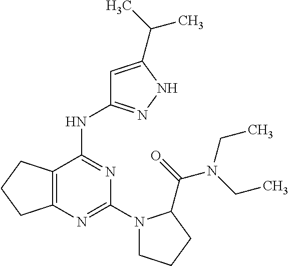 [SMILES: CCN(CC)C(=O)C1CCCN1c1nc2c(c(Nc3cc(C(C)C)[nH]n3)n1)CCC2] | N,N-diethyl-1-(4-(5-isopropyl-1H-pyrazol- 3-ylamino)-6,7-dihydro-5H- cyclopenta[d]pyrimidin-2-yl)pyrrolidine-2- carboxamide |
| 123X | 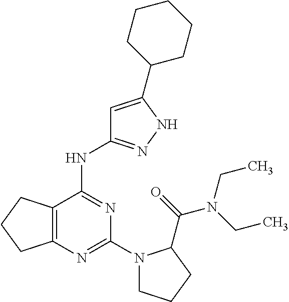 [SMILES: CCN(CC)C(=O)C1CCCN1c1nc2c(c(Nc3cc(C4CCCCC4)[nH]n3)n1)CCC2] | 1-(4-(5-cyclohexyl-1H-pyrazol-3- ylamino)-6,7-dihydro-5H- cyclopenta[d]pyrimidin-2-yl)-N,N- diethylpyrrolidine-2-carboxamide |
| 124X | 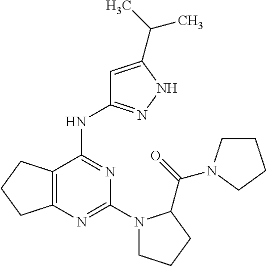 [SMILES: CC(C)c1cc(Nc2nc(N3CCCC3C(=O)N3CCCC3)nc3c2CCC3)n[nH]1] | (1-(4-(5-isopropyl-1H-pyrazol-3-ylamino)- 6,7-dihydro-5H-cyclopenta[d]pyrimidin-2- yl)pyrrolidin-2-yl)(pyrrolidin-1- yl)methanone |
| 125X | 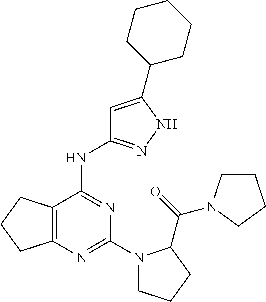 [SMILES: O=C(C1CCCN1c1nc2c(c(Nc3cc(C4CCCCC4)[nH]n3)n1)CCC2)N1CCCC1] | (1-(4-(5-cyclohexyl-1H-pyrazol-3- ylamino)-6,7-dihydro-5H- cyclopenta[d]pyrimidin-2-yl)pyrrolidin-2- yl)(pyrrolidin-1-yl)methanone |
| 126X | 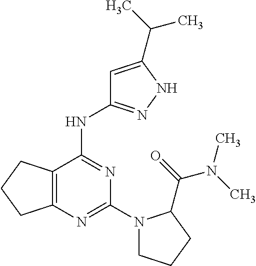 [SMILES: CC(C)c1cc(Nc2nc(N3CCCC3C(=O)N(C)C)nc3c2CCC3)n[nH]1] | 1-(4-(5-isopropyl-1H-pyrazol-3-ylamino)- 5,6,7,8-tetrahydroquinazolin-2-yl)-N,N- dimethylpyrrolidine-2-carboxamide |
| 127X | 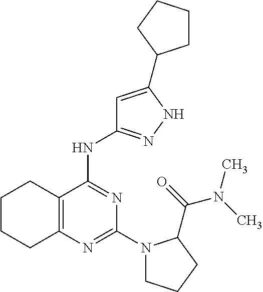 [SMILES: CN(C)C(=O)C1CCCN1c1nc2c(c(Nc3cc(C4CCCC4)[nH]n3)n1)CCCC2] | 1-(4-(5-cyclopentyl-1H-pyrazol-3- ylamino)-5,6,7,8-tetrahydroquinazolin-2- yl)-N,N-dimethylpyrrolidine-2- carboxamide |
| 128X | 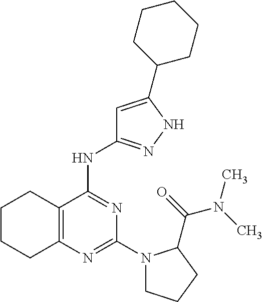 [SMILES: CN(C)C(=O)C1CCCN1c1nc2c(c(Nc3cc(C4CCCCC4)[nH]n3)n1)CCCC2] | 1-(4-(5-cyclohexyl-1H-pyrazol-3- ylamino)-5,6,7,8-tetrahydroquinazolin-2- yl)-N,N-dimethylpyrrolidine-2- carboxamide |
| 129X | 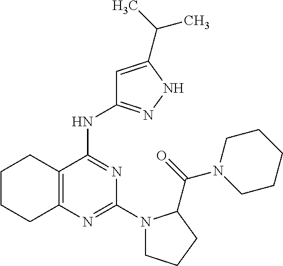 [SMILES: CC(C)c1cc(Nc2nc(N3CCCC3C(=O)N3CCCCC3)nc3c2CCCC3)n[nH]1] | (1-(4-(5-isopropyl-1H-pyrazol-3-ylamino)- 5,6,7,8-tetrahydroquinazolin-2- yl)pyrrolidin-2-yl)(piperidin-1- yl)methanone |
| 130X | 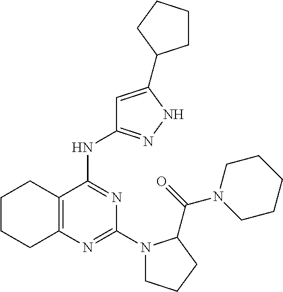 [SMILES: O=C(C1CCCN1c1nc2c(c(Nc3cc(C4CCCC4)[nH]n3)n1)CCCC2)N1CCCCC1] | (1-(4-(5-cyclopentyl-1H-pyrazol-3- ylamino)-5,6,7,8-tetrahydroquinazolin-2- yl)pyrrolidin-2-yl)(piperidin-1- yl)methanone |
| 131X | 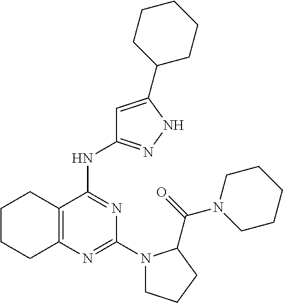 [SMILES: O=C(C1CCCN1c1nc2c(c(Nc3cc(C4CCCCC4)[nH]n3)n1)CCCC2)N1CCCCC1] | (1-(4-(5-cyclohexyl-1H-pyrazol-3- ylamino)-5,6,7,8-tetrahydroquinazolin-2- yl)pyrrolidin-2-yl)(piperidin-1- yl)methanone |
| 132X | 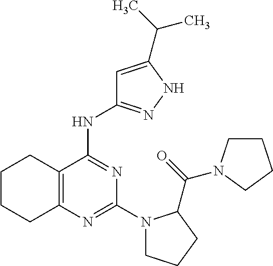 [SMILES: CC(C)c1cc(Nc2nc(N3CCCC3C(=O)N3CCCC3)nc3c2CCCC3)n[nH]1] | (1-(4-(5-isopropyl-1H-pyrazol-3-ylamino)- 5,6,7,8-tetrahydroquinazolin-2- yl)pyrrolidin-2-yl)(pyrrolidin-1- yl)methanone |
| 133X | 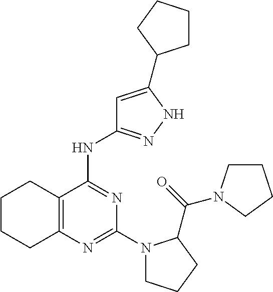 [SMILES: O=C(C1CCCN1c1nc2c(c(Nc3cc(C4CCCC4)[nH]n3)n1)CCCC2)N1CCCC1] | (1-(4-(5-cyclopentyl-1H-pyrazol-3- ylamino)-5,6,7,8-tetrahydroquinazolin-2- yl)pyrrolidin-2-yl)(pyrrolidin-1- yl)methanone |
| 134X | 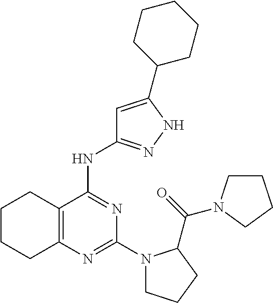 [SMILES: O=C(C1CCCN1c1nc2c(c(Nc3cc(C4CCCCC4)[nH]n3)n1)CCCC2)N1CCCC1] | (1-(4-(5-cyclohexyl-1H-pyrazol-3- ylamino)-5,6,7,8-tetrahydroquinazolin-2- yl)pyrrolidin-2-yl)(pyrrolidin-1- yl)methanone |
| 135X | 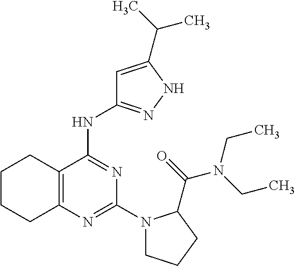 [SMILES: CCN(CC)C(=O)C1CCCN1c1nc2c(c(Nc3cc(C(C)C)[nH]n3)n1)CCCC2] | N,N-diethyl-1-(4-(5-isopropyl-1H-pyrazol- 3-ylamino)-5,6,7,8-tetrahydroquinazolin-2- yl)pyrrolidine-2-carboxamide |
| 136X | 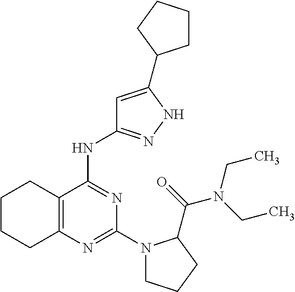 [SMILES: CCN(CC)C(=O)C1CCCN1c1nc2c(c(Nc3cc(C4CCCC4)[nH]n3)n1)CCCC2] | 1-(4-(5-cyclopentyl-1H-pyrazol-3- ylamino)-5,6,7,8-tetrahydroquinazolin-2- yl)-N,N-diethylpyrrolidine-2-carboxamide |
| 137X | 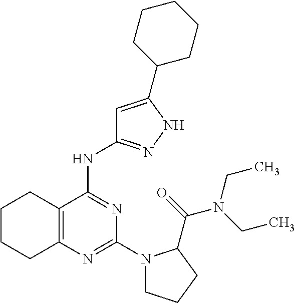 [SMILES: CCN(CC)C(=O)C1CCCN1c1nc2c(c(Nc3cc(C4CCCCC4)[nH]n3)n1)CCCC2] | 1-(4-(5-cyclohexyl-1H-pyrazol-3- ylamino)-5,6,7,8-tetrahydroquinazolin-2- yl)-N,N-diethylpyrrolidine-2-carboxamide |
| 140X | 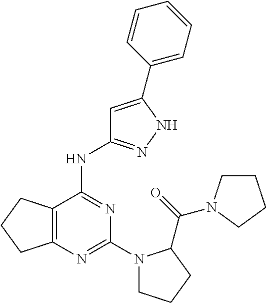 [SMILES: O=C(C1CCCN1c1nc2c(c(Nc3cc(-c4ccccc4)[nH]n3)n1)CCC2)N1CCCC1] | (1-(4-(5-phenyl-1H-pyrazol-3-ylamino)- 6,7-dihydro-5H-cyclopenta[d]pyrimidin-2- yl)pyrrolidin-2-yl)(pyrrolidin-1- yl)methanone |
| 141X | 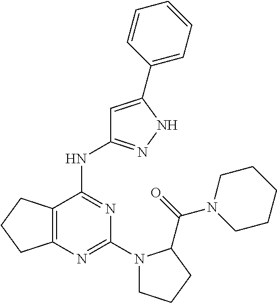 [SMILES: O=C(C1CCCN1c1nc2c(c(Nc3cc(-c4ccccc4)[nH]n3)n1)CCC2)N1CCCCC1] | (1-(4-(5-phenyl-1H-pyrazol-3-ylamino)- 6,7-dihydro-5H-cyclopenta[d]pyrimidin-2- yl)pyrrolidin-2-yl)(piperidin-1- yl)methanone |
| 154X | 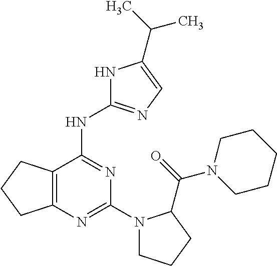 [SMILES: CC(C)c1cnc(Nc2nc(N3CCCC3C(=O)N3CCCCC3)nc3c2CCC3)[nH]1] | (1-(4-(5-isopropyl-1H-imidazol-2- ylamino)-6,7-dihydro-5H- cyclopenta[d]pyrimidin-2-yl)pyrrolidin-2- yl)(piperidin-1-yl)methanone |
| 155X | 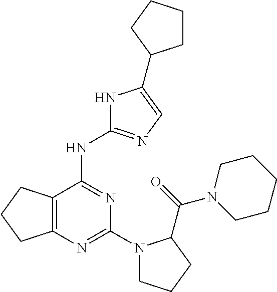 [SMILES: O=C(C1CCCN1c1nc2c(c(Nc3ncc(C4CCCC4)[nH]3)n1)CCC2)N1CCCCC1] | (1-(4-(5-cyclopentyl-1H-imidazol-2- ylamino)-6,7-dihydro-5H- cyclopenta[d]pyrimidin-2-yl)pyrrolidin-2- yl)(piperidin-1-yl)methanone |
| 156X | 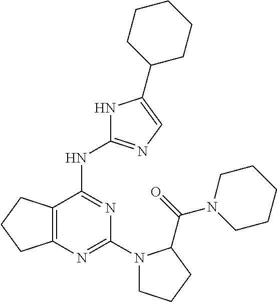 [SMILES: O=C(C1CCCN1c1nc2c(c(Nc3ncc(C4CCCCC4)[nH]3)n1)CCC2)N1CCCCC1] | (1-(4-(5-cyclohexyl-1H-imidazol-2- ylamino)-6,7-dihydro-5H- cyclopenta[d]pyrimidin-2-yl)pyrrolidin-2- yl)(piperidin-1-yl)methanone |
| 157X | 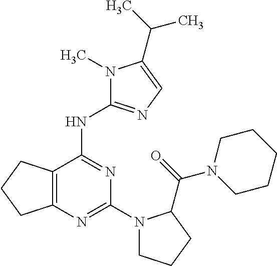 [SMILES: CC(C)c1cnc(Nc2nc(N3CCCC3C(=O)N3CCCCC3)nc3c2CCC3)n1C] | (1-(4-(5-isopropyl-1-methyl-1H-imidazol- 2-ylamino)-6,7-dihydro-5H- cyclopenta[d]pyrimidin-2-yl)pyrrolidin-2- yl)(piperidin-1-yl)methanone |
| 158X | 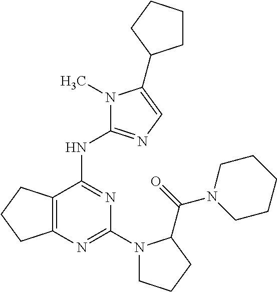 [SMILES: Cn1c(C2CCCC2)cnc1Nc1nc(N2CCCC2C(=O)N2CCCCC2)nc2c1CCC2] | (1-(4-(5-cyclopentyl-1-methyl-1H- imidazol-2-ylamino)-6,7-dihydro-5H- cyclopenta[d]pyrimidin-2-yl)pyrrolidin-2- yl)(piperidin-1-yl)methanone |
| 159X | 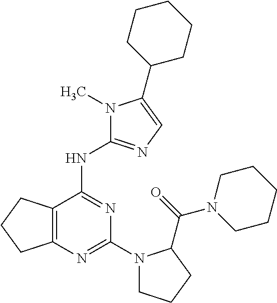 [SMILES: Cn1c(C2CCCCC2)cnc1Nc1nc(N2CCCC2C(=O)N2CCCCC2)nc2c1CCC2] | (1-(4-(5-cyclohexyl-1-methyl-1H- imidazol-2-ylamino)-6,7-dihydro-5H- cyclopenta[d]pyrimidin-2-yl)pyrrolidin-2- yl)(piperidin-1-yl)methanone |
| 168X | 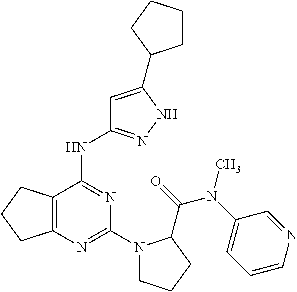 [SMILES: CN(C(=O)C1CCCN1c1nc2c(c(Nc3cc(C4CCCC4)[nH]n3)n1)CCC2)c1cccnc1] | 1-(4-(5-cyclopentyl-1H-pyrazol-3- ylamino)-6,7-dihydro-5H- cyclopenta[d]pyrimidin-2-yl)-N-methyl-N- (pyridin-3-yl)pyrrolidine-2-carboxamide |
| 169X | 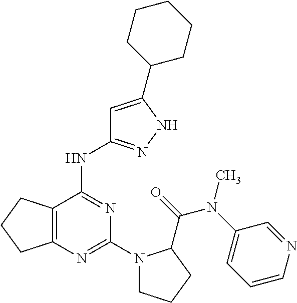 [SMILES: CN(C(=O)C1CCCN1c1nc2c(c(Nc3cc(C4CCCCC4)[nH]n3)n1)CCC2)c1cccnc1] | 1-(4-(5-cyclohexyl-1H-pyrazol-3- ylamino)-6,7-dihydro-5H- cyclopenta[d]pyrimidin-2-yl)-N-methyl-N- (pyridin-3-yl)pyrrolidine-2-carboxamide |
| 170X | 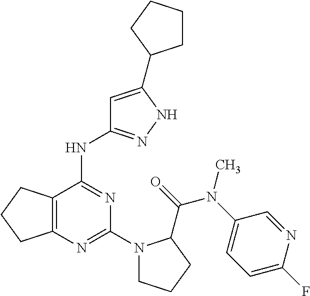 [SMILES: CN(C(=O)C1CCCN1c1nc2c(c(Nc3cc(C4CCCC4)[nH]n3)n1)CCC2)c1ccc(F)nc1] | 1-(4-(5-cyclopentyl-1H-pyrazol-3- ylamino)-6,7-dihydro-5H- cyclopenta[d]pyrimidin-2-yl)-N-(6- fluoropyridin-3-yl)-N-methylpyrrolidine-2- carboxamide |
| 171X | 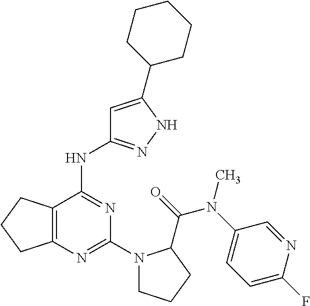 [SMILES: CN(C(=O)C1CCCN1c1nc2c(c(Nc3cc(C4CCCCC4)[nH]n3)n1)CCC2)c1ccc(F)nc1] | 1-(4-(5-cyclohexyl-1H-pyrazol-3- ylamino)-6,7-dihydro-5H- cyclopenta[d]pyrimidin-2-yl)-N-(6- fluoropyridin-3-yl)-N-methylpyrrolidine-2- carboxamide |
| 174X | 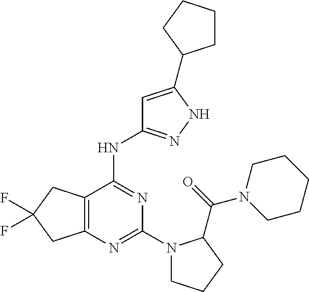 [SMILES: O=C(C1CCCN1c1nc2c(c(Nc3cc(C4CCCC4)[nH]n3)n1)CC(F)(F)C2)N1CCCCC1] | (1-(4-(5-cyclopentyl-1H-pyrazol-3- ylamino)-6,6-difluoro-6,7-dihydro-5H- cyclopenta[d]pyrimidin-2-yl)pyrrolidin-2- yl)(piperidin-1-yl)methanone |
| 175X | 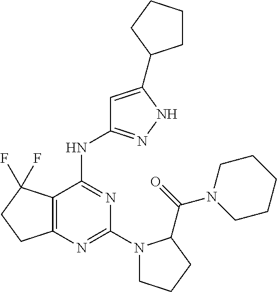 [SMILES: O=C(C1CCCN1c1nc2c(c(Nc3cc(C4CCCC4)[nH]n3)n1)C(F)(F)CC2)N1CCCCC1] | (1-(4-(5-cyclopentyl-1H-pyrazol-3- ylamino)-5,5-difluoro-6,7-dihydro-5H- cyclopenta[d]pyrimidin-2-yl)pyrrolidin-2- yl)(piperidin-1-yl)methanone |
| 176X | 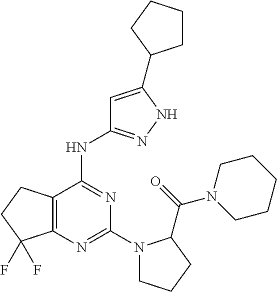 [SMILES: O=C(C1CCCN1c1nc(Nc2cc(C3CCCC3)[nH]n2)c2c(n1)C(F)(F)CC2)N1CCCCC1] | (1-(4-(5-cyclopentyl-1H-pyrazol-3- ylamino)-7,7-difluoro-6,7-dihydro-5H- cyclopenta[d]pyrimidin-2-yl)pyrrolidin-2- yl)(piperidin-1-yl)methanone |
| 177X | 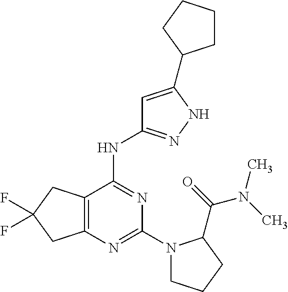 [SMILES: CN(C)C(=O)C1CCCN1c1nc2c(c(Nc3cc(C4CCCC4)[nH]n3)n1)CC(F)(F)C2] | 1-(4-(5-cyclopentyl-1H-pyrazol-3- ylamino)-6,6-difluoro-6,7-dihydro-5H- cyclopenta[d]pyrimidin-2-yl)-N,N- dimethylpyrrolidine-2-carboxamide |
| 178X | 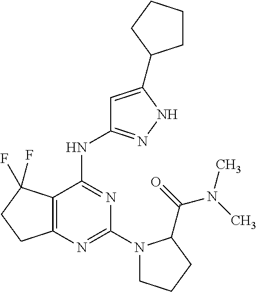 [SMILES: CN(C)C(=O)C1CCCN1c1nc2c(c(Nc3cc(C4CCCC4)[nH]n3)n1)C(F)(F)CC2] | 1-(4-(5-cyclopentyl-1H-pyrazol-3- ylamino)-5,5-difluoro-6,7-dihydro-5H- cyclopenta[d]pyrimidin-2-yl)-N,N- dimethylpyrrolidine-2-carboxamide |
| 179X | 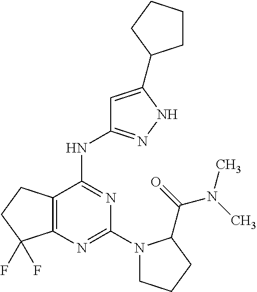 [SMILES: CN(C)C(=O)C1CCCN1c1nc(Nc2cc(C3CCCC3)[nH]n2)c2c(n1)C(F)(F)CC2] | 1-(4-(5-cyclopentyl-1H-pyrazol-3- ylamino)-7,7-difluoro-6,7-dihydro-5H- cyclopenta[d]pyrimidin-2-yl)-N,N- dimethylpyrrolidine-2-carboxamide |
| 180X | 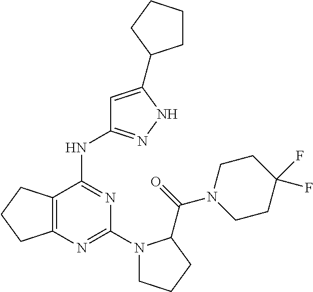 [SMILES: O=C(C1CCCN1c1nc2c(c(Nc3cc(C4CCCC4)[nH]n3)n1)CCC2)N1CCC(F)(F)CC1] | (1-(4-(5-cyclopentyl-1H-pyrazol-3- ylamino)-6,7-dihydro-5H- cyclopenta[d]pyrimidin-2-yl)pyrrolidin-2- yl)(4,4-difluoropiperidin-1-yl)methanone |
| 181X | 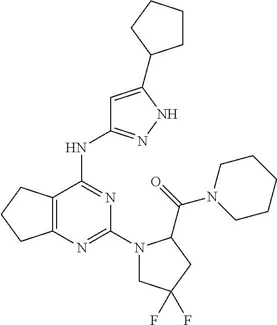 [SMILES: O=C(C1CC(F)(F)CN1c1nc2c(c(Nc3cc(C4CCCC4)[nH]n3)n1)CCC2)N1CCCCC1] | (1-(4-(5-cyclopentyl-1H-pyrazol-3- ylamino)-6,7-dihydro-5H- cyclopenta[d]pyrimidin-2-yl)-4,4- difluoropyrrolidin-2-yl)(piperidin-1- yl)methanone |
| 182X | 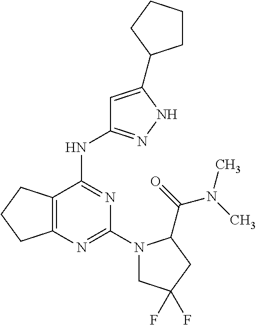 [SMILES: CN(C)C(=O)C1CC(F)(F)CN1c1nc2c(c(Nc3cc(C4CCCC4)[nH]n3)n1)CCC2] | 1-(4-(5-cyclopentyl-1H-pyrazol-3- ylamino)-6,7-dihydro-5H- cyclopenta[d]pyrimidin-2-yl)-4,4-difluoro- N,N-dimethylpyrrolidine-2-carboxamide |
| 183X | 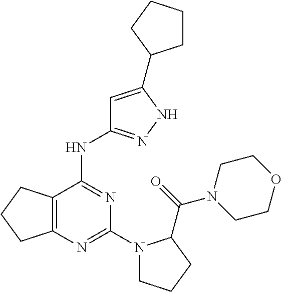 [SMILES: O=C(C1CCCN1c1nc2c(c(Nc3cc(C4CCCC4)[nH]n3)n1)CCC2)N1CCOCC1] | (1-(4-(5-cyclopentyl-1H-pyrazol-3- ylamino)-6,7-dihydro-5H- cyclopenta[d]pyrimidin-2-yl)pyrrolidin-2- yl)(morpholino)methanone |
| 184X | 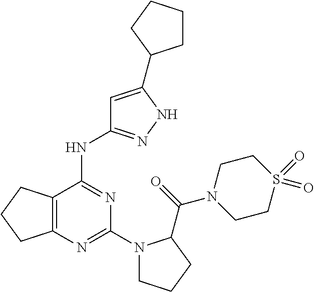 [SMILES: O=C(C1CCCN1c1nc2c(c(Nc3cc(C4CCCC4)[nH]n3)n1)CCC2)N1CCS(=O)(=O)CC1] | (1-(4-(5-cyclopentyl-1H-pyrazol-3- ylamino)-6,7-dihydro-5H- cyclopenta[d]pyrimidin-2-yl)pyrrolidin-2- yl)(1,1-dioxothiomorpholine-4- yl)methanone |
| 185X | 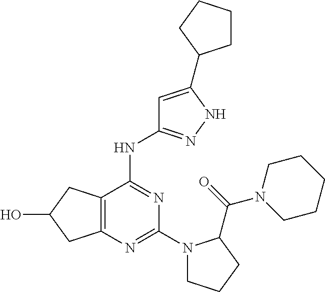 [SMILES: O=C(C1CCCN1c1nc2c(c(Nc3cc(C4CCCC4)[nH]n3)n1)CC(O)C2)N1CCCCC1] | (1-(4-(5-cyclopentyl-1H-pyrazol-3- ylamino)-6-hydroxy-6,7-dihydro-5H- cyclopenta[d]pyrimidin-2-yl)pyrrolidin-2- yl)(piperidin-1-yl)methanone |

Herein, any reference to compounds of Table X or to particular compound numbers from Table X is intended to be a reference to compounds with the names and structures shown in Table X and pharmaceutically acceptable salts thereof. To the extent a name and structure for a given compound number in Table X may disagree, a reference to that compound number is intended to refer to either the name or structure or both as shown for that compound number in Table X.

In one variation, the compound of Formula (I) is a compound that modulates (e.g., inhibits) Trk to a greater extent than it modulates IGF, such as IGF-1R. In another variation, the compound of Formula (I) is a compound that modulates (e.g., inhibits) Trk but exhibits little or no ability to modulate (e.g., inhibit) IGF, such as IGF-1R.

In another variation, the compound of Formula (I) is a compound that modulates (e.g., inhibits) FLT3 to a greater extent than it modulates IGF, such as IGF-1R. In yet another variation, the compound of Formula (I) is a compound that modulates (e.g., inhibits) FLT3 but exhibits little or no ability to modulate (e.g., inhibit) IGF, such as IGF-1R.

In another variation, the compound of Formula (I) is a compound of the Formula (II):

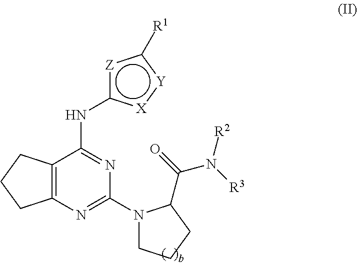

wherein X, Y, Z, R¹, R², R³, and b are as defined for Formula (I), or a pharmaceutically acceptable salt thereof. In some embodiments of Formula (II), the compound is not a compound in Table X or a pharmaceutically acceptable salt thereof. In other embodiments of Formula (II), the compound is not a compound selected from the group consisting of Compound Nos. 2X, 3X, 4X, 6X, 7X, 8X, 9X, 12X, 16X, 17X, 19X, 20X, 24X, 29X, 32X, 45X, 55X, 56X, 60X, 61X, 62X, 63X, 64X, 68X, 69X, 72X, 73X, 74X, 75X, 79X, 82X, 84X, 90X, 91X, 95X, 97X, 98X, 100X, 111X, 112X, 113X, 114X, 115X, 116X, 119X, 120X, 121X, 122X, 123X, 124X, 125X, 129X, 130X, 131X, 132X, 133X, 134X, 140X, 141X, 154X, 155X, 156X, 157X, 158X, 159X, 168X, 169X, 170X, 171X, 180X, 183X and 184X in Table X, and pharmaceutically acceptable salts thereof.

In one variation, the compound of Formula (II) is a compound that modulates (e.g., inhibits) Trk to a greater extent than it modulates IGF, such as IGF-1R. In another variation, the compound of Formula (II) is a compound that modulates (e.g., inhibits) Trk but exhibits little or no ability to modulate (e.g., inhibit) IGF, such as IGF-1R.

In another variation, the compound of Formula (II) is a compound that modulates (e.g., inhibits) FLT3 to a greater extent than it modulates IGF, such as IGF-1R. In yet another variation, the compound of Formula (II) is a compound that modulates (e.g., inhibits) FLT3 but exhibits little or no ability to modulate (e.g., inhibit) IGFm such as IGF-1R.

In one variation is provided a compound of any one of formulae (I)-(VI) wherein the compound modulates (e.g., inhibits) Trk and/or FLT3 to a greater extent than it modulates IGF, such as IGF-1R and wherein the compound has the following structural features: (1) R¹ is C₅₋₈cycloalkyl (e.g., cyclopentyl, cyclohexyl or bicyclo[3.1.0]hexan-3-yl) or 5- to 7-membered heterocyclyl (e.g., tetrahydro-2H-pyran-4-yl), each optionally substituted as described for Formula (I); (2) R² is H or C₁₋₄alkyl (e.g., methyl or ethyl), and R³ is a 5-membered heteroaryl (e.g., thiophenyl, thiazolyl, oxazolyl, pyrazolyl, imidazolyl, oxadiazolyl, thiadizaolyl, or triazolyl) optionally substituted as described for Formula (I), or R² and R³ taken together with the nitrogen to which they are attached form a 5- to 8-membered heterocyclyl (e.g., pyrrolidinyl, piperidinyl, morpholinyl, thiomorpholinyl, piperazinyl, 5-azaspiro[2.4]heptan-5-yl or 6-azaspiro[2.5]octan-6-yl) optionally substituted with C₁₋₄alkyl, —OH, —OC₁₋₄alkyl, oxo, halo, —CF₃, —CN, —C(O)C₁₋₄alkyl, —CO₂H, —CO₂C₁₋₄alkyl, —C(O)NR^{g}R^{h}, or —NR^{g}R^{h}; (3) R¹ is C₅₋₈cycloalkyl (e.g., cyclopentyl, cyclohexyl or bicyclo[3.1.0]hexan-3-yl) or 5- to 7-membered heterocyclyl (e.g., tetrahydro-2H-pyran-4-yl), each optionally substituted as described for Formula (I), R² is H or C₁₋₄alkyl (e.g., methyl or ethyl), and R³ is a 5-membered heteroaryl (e.g., thiophenyl, thiazolyl, oxazolyl, pyrazolyl, imidazolyl, oxadiazolyl, thiadizaolyl, or triazolyl) optionally substituted as described for Formula (I); or (4) R¹ is C₅₋₈cycloalkyl (e.g., cyclopentyl, cyclohexyl or bicyclo[3.1.0]hexan-3-yl) or 5- to 7-membered heterocyclyl (e.g., tetrahydro-2H-pyran-4-yl), each optionally substituted as described for Formula (I), and R² and R³ taken together with the nitrogen to which they are attached form a 5- to 8-membered heterocyclyl (e.g., pyrrolidinyl, piperidinyl, morpholinyl, thiomorpholinyl, piperazinyl, 5-azaspiro[2.4]heptan-5-yl or 6-azaspiro[2.5]octan-6-yl) optionally substituted with C₁₋₄alkyl, —OH, —OC₁₋₄alkyl, oxo, halo, —CF₃, —CN, —C(O)C₁₋₄alkyl, —CO₂H, —CO₂C₁₋₄alkyl, —C(O)NR^{g}R^{h}, or —NR^{g}R^{h}. In another variation, is provided a compound of any one of formulae (I)-(VI) wherein the compound the modulates (e.g., inhibits) Trk and/or FLT3 but exhibits little or no ability to modulate (e.g., inhibit) IGF, such as IGF-1R and wherein the compound has the following structural features: (1) R¹ is C₅₋₈cycloalkyl (e.g., cyclopentyl, cyclohexyl or bicyclo[3.1.0]hexan-3-yl) or 5- to 7-membered heterocyclyl (e.g., tetrahydro-2H-pyran-4-yl), each optionally substituted as described for Formula (I); (2) R² is H or C₁₋₄alkyl (e.g., methyl or ethyl), and R³ is a 5-membered heteroaryl (e.g., thiophenyl, thiazolyl, oxazolyl, pyrazolyl, imidazolyl, oxadiazolyl, thiadizaolyl, or triazolyl) optionally substituted as described for Formula (I), or R² and R³ taken together with the nitrogen to which they are attached form a 5- to 8-membered heterocyclyl (e.g., pyrrolidinyl, piperidinyl, morpholinyl, thiomorpholinyl, piperazinyl, 5-azaspiro[2.4]heptan-5-yl or 6-azaspiro[2.5]octan-6-yl) optionally substituted with C₁₋₄alkyl, —OH, —OC₁₋₄alkyl, oxo, halo, —CF₃, —CN, —C(O)C₁₋₄alkyl, —CO₂H, —CO₂C₁₋₄alkyl, —C(O)NR^{g}R^{h}, or —NR^{g}R^{h}; (3) R¹ is C₅₋₈cycloalkyl (e.g., cyclopentyl, cyclohexyl or bicyclo[3.1.0]hexan-3-yl) or 5- to 7-membered heterocyclyl (e.g., tetrahydro-2H-pyran-4-yl), each optionally substituted as described for Formula (I), R² is H or C₁₋₄alkyl (e.g., methyl or ethyl), and R³ is a 5-membered heteroaryl (e.g., thiophenyl, thiazolyl, oxazolyl, pyrazolyl, imidazolyl, oxadiazolyl, thiadizaolyl, or triazolyl) optionally substituted as described for Formula (I); or (4) R¹ is C₅₋₈cycloalkyl (e.g., cyclopentyl, cyclohexyl or bicyclo[3.1.0]hexan-3-yl) or 5- to 7-membered heterocyclyl (e.g., tetrahydro-2H-pyran-4-yl), each optionally substituted as described for Formula (I), and R² and R³ taken together with the nitrogen to which they are attached form a 5- to 8-membered heterocyclyl (e.g., pyrrolidinyl, piperidinyl, morpholinyl, thiomorpholinyl, piperazinyl, 5-azaspiro[2.4]heptan-5-yl or 6-azaspiro[2.5]octan-6-yl) optionally substituted with C₁₋₄alkyl, —OH, —OC₁₋₄alkyl, oxo, halo, —CF₃, —CN, —C(O)C₁₋₄alkyl, —CO₂H, —CO₂C₁₋₄alkyl, —C(O)NR^{g}R^{h}, or —NR^{g}R^{h}. Pharmaceutical compositions, kits, uses and methods (including a method of treating cancer such as breast cancer, prostate cancer, ovarian cancer, lung cancer, colon cancer, leukemia, and the like) detailed herein in one variation employ such compounds.

Further provided is a pharmaceutical composition comprising a compound of Formula (I) or any variations described herein (e.g., a compound of Formula (II)-(VI), variations thereof, or a compound in Table 1), or a salt thereof, and a pharmaceutically acceptable carrier.

In one aspect, the pharmaceutical composition comprises a compound of any one of formulae (I)-(VI) wherein the compound modulates (e.g., inhibits) Trk to a greater extent than it modulates IGF, such as IGF-1R. In another variation, the pharmaceutical composition comprises a compound of any one of formulae (I)-(VI) wherein the compound modulates (e.g., inhibits) Trk but exhibits little or no ability to modulate (e.g., inhibit) IGF, such as IGF-1R.

In one aspect, the pharmaceutical composition comprises a compound of any one of formulae (I)-(VI) wherein the compound modulates (e.g., inhibits) FLT3 to a greater extent than it modulates IGF, such as IGF-1R. In another variation, the pharmaceutical composition comprises a compound of any one of formulae (I)-(VI) wherein the compound modulates (e.g., inhibits) FLT3 but exhibits little or no ability to modulate (e.g., inhibit) IGF, such as IGF-1R.

Further provided is a method of modulating a kinase receptor by administering to an individual in need thereof a therapeutically effective amount of a compound of Formula (I) or any variations described herein (e.g., a compound of Formula (II)-(VI), variations thereof, or a compound in Table 1), or a pharmaceutically acceptable salt thereof.

Further provided is a method of treating cancer comprising administering to an individual in need thereof a therapeutically effective amount of a compound of Formula (I) or any variations described herein (e.g., a compound of Formula (II)-(VI), variations thereof, or a compound in Table 1), or a pharmaceutically acceptable salt thereof.

In any of the methods detailed herein, in one aspect the method employs use of a compound of any one of formulae (I)-(VI) wherein the compound modulates (e.g., inhibits) Trk to a greater extent than it modulates IGF, such as IGF-1R. In another variation of any of the methods detailed herein, the method employs a compound of any one of formulae (I)-(VI) wherein the compound modulates (e.g., inhibits) Trk but exhibits little or no ability to modulate IGF, such as IGF-1R.

In any of the methods detailed herein, in one aspect the method employs use of a compound of any one of formulae (I)-(VI) wherein the compound modulates (e.g., inhibits) FLT3 to a greater extent than it modulates IGF, such as IGF-1R. In another variation of any of the methods detailed herein, the method employs a compound of any one of formulae (I)-(VI) wherein the compound modulates (e.g., inhibits) FLT3 but exhibits little or no ability to modulate IGF, such as IGF-1R.

Further provided is use of a compound of Formula (I) or any variations described herein (e.g., a compound of Formula (II)-(VI), variations thereof, or a compound in Table 1), or a salt thereof, in the manufacturing of a medicament for the treatment of cancer.

Further provided is a compound of Formula (I) or any variations described herein (e.g., a compound of Formula (II)-(VI), variations thereof, or a compound in Table 1), or a pharmaceutically acceptable salt thereof, for use in a method of treating cancer.

Also provided is a kit comprising a compound of Formula (I) or any variations described herein (e.g., a compound of Formula (II)-(VI), variations thereof, or a compound in Table 1), or a pharmaceutically acceptable salt thereof.

### DETAILED DESCRIPTION OF THE INVENTION

### Definitions

For use herein, unless clearly indicated otherwise, use of the terms “a,” “an,” and the like refers to one or more.

Reference to “about” a value or parameter herein includes (and describes) embodiments that are directed to that value or parameter per se. For example, description referring to “about X” includes description of “X”.

Reference to the range of carbon atoms in the group may be designated in the “Cₘ-Cₙ” format or the “Cₘ₋ₙ” format. For example, an alkyl group having from 1 to 6 carbon atoms may be referred to as “C₁-C₆ alkyl” or “C₁₋₆alkyl”. Likewise a cycloalkyl group having from 3 to 8 ring carbon atoms may be referred to as “C₃-C₈ cycloalkyl” or “C₃₋₈cycloalkyl”.

“Alkyl” as used herein refers to a saturated linear (i.e. unbranched) or branched univalent hydrocarbon chain or combination thereof, having the number of carbon atoms designated (i.e., C₁-C₁₀ means one to ten carbon atoms). The alkyl group may be optionally substituted independently with one or more substituents described herein. Particular alkyl groups are those having 1 to 20 carbon atoms (a “C₁-C₂₀ alkyl”), having 1 to 8 carbon atoms (a “C₁-C₈ alkyl”), having 1 to 6 carbon atoms (a “C₁-C₆ alkyl”), having 2 to 6 carbon atoms (a “C₂-C₆ alkyl”), or having 1 to 4 carbon atoms (a “C₁-C₄ alkyl”). Examples of alkyl group include, but are not limited to, groups such as methyl, ethyl, n-propyl, isopropyl, n-butyl, t-butyl, isobutyl, sec-butyl, homologs and isomers of, for example, n-pentyl, n-hexyl, n-heptyl, n-octyl, and the like.

“Alkenyl” as used herein refers to an unsaturated linear (i.e. unbranched) or branched univalent hydrocarbon chain or combination thereof, having at least one site of olefinic unsaturation (i.e., having at least one moiety of the formula C═C) and having the number of carbon atoms designated (i.e., C₂-C₁₀ means two to ten carbon atoms). The alkenyl group may be optionally substituted independently with one or more substituents described herein and having “cis” or “trans” configurations, or alternatively having “E” or “Z” configurations. Particular alkenyl groups are those having 2 to 20 carbon atoms (a “C₂-C₂₀ alkenyl”), having 2 to 8 carbon atoms (a “C₂-C₈ alkenyl”), having 2 to 6 carbon atoms (a “C₂-C₆ alkenyl”), or having 2 to 4 carbon atoms (a “C₂-C₄ alkenyl”). Examples of alkenyl group include, but are not limited to, groups such as ethenyl (or vinyl), prop-1-enyl, prop-2-enyl (or allyl), 2-methylprop-1-enyl, but-1-enyl, but-2-enyl, but-3-enyl, buta-1,3-dienyl, 2-methylbuta-1,3-dienyl, homologs and isomers thereof, and the like.

“Alkynyl” as used herein refers to an unsaturated linear (i.e. unbranched) or branched univalent hydrocarbon chain or combination thereof, having at least one site of acetylenic unsaturation (i.e., having at least one moiety of the formula C≡C) and having the number of carbon atoms designated (i.e., C₂-C₁₀ means two to ten carbon atoms). The alkynyl group may be optionally substituted independently with one or more substituents described herein. Particular alkynyl groups are those having 2 to 20 carbon atoms (a “C₂-C₂₀ alkynyl”), having 2 to 8 carbon atoms (a “C₂-C₈ alkynyl”), having 2 to 6 carbon atoms (a “C₂-C₆ alkynyl”), or having 2 to 4 carbon atoms (a “C₂-C₄ alkynyl”). Examples of alkynyl group include, but are not limited to, groups such as ethynyl (or acetylenyl), prop-1-ynyl, prop-2-ynyl (or propargyl), but-1-ynyl, but-2-ynyl, but-3-ynyl, homologs and isomers thereof, and the like.

“Alkylene” as used herein refers to the same residues as alkyl, but having bivalency. Particular alkylene groups are those having 1 to 6 carbon atoms (a “C₁-C₆ alkylene”), 1 to 5 carbon atoms (a “C₁-C₅ alkylene”), 1 to 4 carbon atoms (a “C₁-C₄ alkylene”) or 1 to 3 carbon atoms (a “C₁-C₃ alkylene”). Examples of alkylene include, but are not limited to, groups such as methylene (—CH₂—), ethylene (—CH₂CH₂—), propylene (—CH₂CH₂CH₂—), butylene (—CH₂CH₂CH₂CH₂—), and the like.

“Cycloalkyl” as used herein refers to non-aromatic, saturated or unsaturated cyclic univalent hydrocarbon structures having the number of carbon atoms designated (i.e., C₃-C₁₀ means three to ten carbon atoms). Cycloalkyl can consist of one ring, such as cyclohexyl, or multiple rings, such as adamantyl, but excludes aryl groups. A cycloalkyl comprising more than one ring may be fused, spiro, or bridged, or combinations thereof. The cycloalkyl group may be optionally substituted independently with one or more substituents described herein. Particular cycloalkyl groups are those having from 3 to 12 annular carbon atoms. A preferred cycloalkyl is a cyclic hydrocarbon having from 3 to 8 annular carbon atoms (a “C₃-C₈ cycloalkyl”), or having 3 to 6 carbon atoms (a “C₃-C₆ cycloalkyl”). Examples of cycloalkyl include, but are not limited to, cyclopropyl, cyclobutyl, cyclopentyl, cyclohexyl, 1-cyclohexenyl, 3-cyclohexenyl, cycloheptyl, norbornyl, and the like.

“Aryl” as used herein refers to an unsaturated aromatic carbocyclic group having a single ring (e.g., phenyl) or multiple condensed rings (e.g., naphthyl or anthryl) which condensed rings may or may not be aromatic. The aryl group may be optionally substituted independently with one or more substituents described herein. Particular aryl groups are those having from 6 to 14 annular (i.e., ring) carbon atoms (a “C₆-C₁₄ aryl”). An aryl group having more than one ring where at least one ring is non-aromatic may be connected to the parent structure at either an aromatic ring position or at a non-aromatic ring position. In one variation, an aryl group having more than one ring where at least one ring is non-aromatic is connected to the parent structure at an aromatic ring position.

“Heteroaryl” as used herein refers to an unsaturated aromatic cyclic group having from 1 to 14 annular (i.e., ring) carbon atoms and at least one annular heteroatom, including but not limited to heteroatoms such as nitrogen, oxygen and sulfur. A heteroaryl group may have a single ring (e.g., pyridyl, furyl) or multiple condensed rings (e.g., indolizinyl, benzothienyl) which condensed rings may or may not be aromatic. The heteroaryl group may be optionally substituted independently with one or more substituents described herein. Particular heteroaryl groups are 5- to 14-membered rings having 1 to 12 annular (i.e., ring) carbon atoms and 1 to 6 annular (i.e., ring) heteroatoms independently selected from nitrogen, oxygen and sulfur; 5- to 10-membered rings having 1 to 8 annular carbon atoms and 1 to 4 annular heteroatoms independently selected from nitrogen, oxygen and sulfur; and 5-, 6- or 7-membered rings having 1 to 5 annular carbon atoms and 1 to 4 annular heteroatoms independently selected from nitrogen, oxygen and sulfur. In one variation, heteroaryl includes monocyclic aromatic 5-, 6- or 7-membered rings having from 1 to 6 annular carbon atoms and 1 to 4 annular heteroatoms independently selected from nitrogen, oxygen and sulfur. In another variation, heteroaryl includes polycyclic aromatic rings having from 1 to 12 annular carbon atoms and 1 to 6 annular heteroatoms independently selected from nitrogen, oxygen and sulfur. A heteroaryl group having more than one ring where at least one ring is non-aromatic may be connected to the parent structure at either an aromatic ring position or at a non-aromatic ring position. In one variation, a heteroaryl group having more than one ring where at least one ring is non-aromatic is connected to the parent structure at an aromatic ring position.

“Heterocycle”, “heterocyclic”, or “heterocyclyl” as used herein refers to a saturated or an unsaturated non-aromatic cyclic group having a single ring or multiple condensed rings, and having from 1 to 14 annular (i.e., ring) carbon atoms and from 1 to 6 annular (i.e., ring) heteroatoms, such as nitrogen, sulfur or oxygen, and the like. A heterocycle comprising more than one ring may be fused, spiro or bridged, or any combination thereof. In fused ring systems, one or more of the fused rings can be cycloalkyl. The heterocyclyl group may be optionally substituted independently with one or more substituents described herein. Particular heterocyclyl groups are 3- to 14-membered rings having 1 to 13 annular carbon atoms and 1 to 6 annular heteroatoms independently selected from nitrogen, oxygen and sulfur; 3- to 12-membered rings having 1 to 11 annular carbon atoms and 1 to 6 annular heteroatoms independently selected from nitrogen, oxygen and sulfur; 3- to 10-membered rings having 1 to 9 annular carbon atoms and 1 to 4 annular heteroatoms independently selected from nitrogen, oxygen and sulfur; 3- to 8-membered rings having 1 to 7 annular carbon atoms and 1 to 4 annular heteroatoms independently selected from nitrogen, oxygen and sulfur; and -3 to 6-membered rings having 1 to 5 annular carbon atoms and 1 to 4 annular heteroatoms independently selected from nitrogen, oxygen and sulfur. In one variation, heterocyclyl includes monocyclic 3-, 4-, 5-, 6- or 7-membered rings having from 1 to 2, 1 to 3, 1 to 4, 1 to 5 or 1 to 6 annular carbon atoms and 1 to 2, 1 to 3 or 1 to 4 annular heteroatoms independently selected from nitrogen, oxygen and sulfur. In another variation, heterocyclyl includes polycyclic non-aromatic rings having from 1 to 12 annular carbon atoms and 1 to 6 annular heteroatoms independently selected from nitrogen, oxygen and sulfur.

“Halo” or “halogen” refers to elements of the Group 17 series having atomic number 9 to 85. Preferred halo groups include fluoro, chloro, bromo and iodo. Where a residue is substituted with more than one halogen, it may be referred to by using a prefix corresponding to the number of halogen moieties attached, e.g., dihaloaryl, dihaloalkyl, trihaloaryl etc. refer to aryl and alkyl substituted with two (“di”) or three (“tri”) halo groups, which may be but are not necessarily the same halo; thus 4-chloro-3-fluorophenyl is within the scope of dihaloaryl. An alkyl group in which each hydrogen is replaced with a halo group is referred to as a “perhaloalkyl.” A preferred perhaloalkyl group is trifluoroalkyl (—CF₃). Similarly, “perhaloalkoxy” refers to an alkoxy group in which a halogen takes the place of each H in the hydrocarbon making up the alkyl moiety of the alkoxy group. An example of a perhaloalkoxy group is trifluoromethoxy (—OCF₃).

“Carbonyl” refers to the group C═O.

“Thiocarbonyl” refers to the group C═S.

“Oxo” refers to the moiety ═O.

“Optionally substituted” unless otherwise specified means that a group may be unsubstituted or substituted by one or more (e.g., 1, 2, 3, 4 or 5) of the substituents listed for that group in which the substituents may be the same of different. In one embodiment, an optionally substituted group has one substituent. In another embodiment, an optionally substituted group has two substituents. In another embodiment, an optionally substituted group has three substituents. In another embodiment, an optionally substituted group has four substituents. In some embodiments, an optionally substituted group has 1 to 2, 1 to 3, 1 to 4 or 1 to 5 substituents.

Unless clearly indicated otherwise, an “individual” as used herein intends a mammal, including but not limited to a primate, human, bovine, horse, feline, canine, rabbit, or rodent.

As used herein a receptor “modulator,” such as a FLT3 or Trk receptor modulator, encompasses both a receptor antagonist and a receptor agonist (e.g., a “Trk receptor modulator” encompasses both a Trk receptor antagonist and a Trk receptor agonist). In some aspects, the receptor modulator binds to or inhibits binding of a ligand to the receptor and/or reduces or eliminates or increases or enhances or mimics an activity of the receptor in a reversible or irreversible manner. In some aspects, the receptor modulator inhibits binding of a ligand to the receptor by at least about or by about any one of 10%, 20%, 30%, 40%, 50%, 60%, 70%, 80%, 90%, 95% or 100% as determined by an assay described herein. In some aspects, the receptor modulator reduces an activity of the receptor by at least about or about any of 10%, 20%, 30%, 40%, 50%, 60%, 70%, 80%, 90%, 95% or 100% as compared to the corresponding activity in the same subject prior to treatment with the receptor modulator or compared to the corresponding activity in other subjects not receiving the receptor modulator. In some aspects, a receptor modulator enhances an activity of the receptor by at least about or by about any of 10%, 20%, 30%, 40%, 50%, 60%, 70%, 80%, 90%, 95% or 100 or 200% or 300% or 400% or 500% or more as compared to the corresponding activity in the same subject prior to treatment with the receptor modulator or compared to the corresponding activity in other subjects not receiving the receptor modulator. In some aspects, the receptor modulator is capable of binding to the active site of the receptor (e.g., a binding site for a ligand). In some embodiments, the receptor modulator is capable of binding to an allosteric site of the receptor.

As used herein, “treatment” or “treating” is an approach for obtaining beneficial or desired results including clinical results. For purposes of this invention, beneficial or desired clinical results include, but are not limited to, one or more of the following: decreasing one more symptoms resulting from the disease, diminishing the extent of the disease, stabilizing the disease (e.g., preventing or delaying the worsening of the disease), preventing or delaying the spread (e.g., metastasis) of the disease, delay or slowing the progression of the disease, ameliorating the disease state, providing a remission (whether partial or total) of the disease, decreasing the dose of one or more other medications required to treat the disease, enhancing effect of another medication, delaying the progression of the disease, increasing the quality of life, and/or prolonging survival. Also encompassed by “treatment” is a reduction of pathological consequence of cancer. The methods of the invention contemplate any one or more of these aspects of treatment.

As used herein, “delaying” the development of cancer means to defer, hinder, slow, retard, stabilize, and/or postpone development of the disease. This delay can be of varying lengths of time, depending on the history of the disease and/or individual being treated. As is evident to one skilled in the art, a sufficient or significant delay can, in effect, encompass prevention, in that the individual does not develop the disease. A method that “delays” development of cancer is a method that reduces probability of disease development in a given time frame and/or reduces the extent of the disease in a given time frame, when compared to not using the method. Such comparisons are typically based on clinical studies, using a statistically significant number of subjects. Cancer development can be detectable using standard methods, such as routine physical exams, mammography, imaging, or biopsy. Development may also refer to disease progression that may be initially undetectable and includes occurrence, recurrence, and onset.

As used herein, an “at risk” individual is an individual who is at risk of developing cancer. An individual “at risk” may or may not have detectable disease, and may or may not have displayed detectable disease prior to the treatment methods described herein. “At risk” denotes that an individual has one or more so-called risk factors, which are measurable parameters that correlate with development of cancer, which are described herein. An individual having one or more of these risk factors has a higher probability of developing cancer than an individual without these risk factor(s).

As used herein, by “combination therapy” is meant a therapy that includes two or more different compounds. Thus, in one aspect, a combination therapy comprising a compound detailed herein and another compound is provided. In some variations, the combination therapy optionally includes one or more pharmaceutically acceptable carriers or excipients, non-pharmaceutically active compounds, and/or inert substances.

As used herein, the term “effective amount” intends such amount of a compound of the invention which in combination with its parameters of efficacy and toxicity, should be effective in a given therapeutic form. As is understood in the art, an effective amount may be in one or more doses, i.e., a single dose or multiple doses may be required to achieve the desired treatment endpoint. An effective amount may be considered in the context of administering one or more therapeutic agents, and a single agent may be considered to be given in an effective amount if, in conjunction with one or more other agents, a desirable or beneficial result may be or is achieved. Suitable doses of any of the co-administered compounds may optionally be lowered due to the combined action (e.g., additive or synergistic effects) of the compounds. In various embodiments, an effective amount of the composition or therapy may: (i) reduce the number of cancer cells; (ii) reduce tumor size; (iii) inhibit, retard, slow to some extent, and preferably stop cancer cell infiltration into peripheral organs; (iv) inhibit (e.g., slow to some extent and preferably stop) tumor metastasis; (v) inhibit tumor growth; (vi) prevent or delay occurrence and/or recurrence of a tumor; and/or (vii) relieve to some extent one or more of the symptoms associated with the cancer. In various embodiments, the amount is sufficient to ameliorate, palliate, lessen, and/or delay one or more of symptoms of cancer.

As is understood in the art, an “effective amount” may be in one or more doses, i.e., a single dose or multiple doses may be required to achieve the desired treatment endpoint. An effective amount may be considered in the context of administering one or more therapeutic agents, and a compound, or pharmaceutically acceptable salt thereof, may be considered to be given in an effective amount if, in conjunction with one or more other agents, a desirable or beneficial result may be or is achieved.

A “therapeutically effective amount” refers to an amount of a compound or salt thereof sufficient to produce a desired therapeutic outcome (e.g., reducing the severity or duration of, stabilizing the severity of, or eliminating one or more symptoms of cancer). For therapeutic use, beneficial or desired results include, e.g., decreasing one or more symptoms resulting from the disease (biochemical, histologic and/or behavioral), including its complications and intermediate pathological phenotypes presenting during development of the disease, increasing the quality of life of those suffering from the disease, decreasing the dose of other medications required to treat the disease, enhancing effect of another medication, delaying the progression of the disease, and/or prolonging survival of patients.

A “prophylactically effective amount” refers to an amount of a compound, or pharmaceutically acceptable salt thereof, sufficient to prevent or reduce the severity of one or more future symptoms of cancer when administered to an individual who is susceptible and/or who may develop cancer. For prophylactic use, beneficial or desired results include, e.g., results such as eliminating or reducing the risk, lessening the severity of future disease, or delaying the onset of the disease (e.g., delaying biochemical, histologic and/or behavioral symptoms of the disease, its complications, and intermediate pathological phenotypes presenting during future development of the disease).

It is understood that an effective amount of a compound or pharmaceutically acceptable salt thereof, including a prophylactically effective amount, may be given to an individual in the adjuvant setting, which refers to a clinical setting in which an individual has had a history of cancer, and generally (but not necessarily) has been responsive to therapy, which includes, but is not limited to, surgery (e.g., surgical resection), radiotherapy, and chemotherapy. However, because of their history of the cancer, these individuals are considered at risk of developing cancer. Treatment or administration in the “adjuvant setting” refers to a subsequent mode of treatment.

As used herein, “unit dosage form” refers to physically discrete units, suitable as unit dosages, each unit containing a predetermined quantity of active ingredient calculated to produce the desired therapeutic effect in association with the required pharmaceutical carrier. Unit dosage forms may contain a single or a combination therapy.

As used herein, the term “controlled release” refers to a drug-containing formulation or fraction thereof in which release of the drug is not immediate, i.e., with a “controlled release” formulation, administration does not result in immediate release of the drug into an absorption pool. The term encompasses depot formulations designed to gradually release the drug compound over an extended period of time. Controlled release formulations can include a wide variety of drug delivery systems, generally involving mixing the drug compound with carriers, polymers or other compounds having the desired release characteristics (e.g., pH-dependent or non-pH-dependent solubility, different degrees of water solubility, and the like) and formulating the mixture according to the desired route of delivery (e.g., coated capsules, implantable reservoirs, injectable solutions containing biodegradable capsules, and the like).

As used herein, by “pharmaceutically acceptable” or “pharmacologically acceptable” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug Administration.

In some embodiments, the salts of the compounds of the invention are pharmaceutically acceptable salts. “Pharmaceutically acceptable salts” are those salts which retain at least some of the biological activity of the free (non-salt) compound and which can be administered as drugs or pharmaceuticals to an individual. Such salts, for example, include: (1) acid addition salts, formed with inorganic acids such as hydrochloric acid, hydrobromic acid, sulfuric acid, nitric acid, phosphoric acid, and the like; or formed with organic acids such as acetic acid, oxalic acid, propionic acid, succinic acid, maleic acid, tartaric acid and the like; (2) salts formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine and the like. Acceptable inorganic bases include aluminum hydroxide, calcium hydroxide, potassium hydroxide, sodium carbonate, sodium hydroxide, and the like. Pharmaceutically acceptable salts can be prepared in situ in the manufacturing process, or by separately reacting a purified compound of the invention in its free acid or base form with a suitable organic or inorganic base or acid, respectively, and isolating the salt thus formed during subsequent purification.

The term “excipient” as used herein means an inert or inactive substance that may be used in the production of a drug or pharmaceutical, such as a tablet containing a compound of the invention as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include, e.g., calcium carbonate, dextrose, fructose dc (dc=“directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

### Compounds of the Invention

Compounds according to the invention are detailed herein, including in the Brief Summary of the Invention and the appended claims. The invention includes the use of all of the compounds described herein, including any and all stereoisomers, including geometric isomers (cis/trans), salts (including pharmaceutically acceptable salts) and solvates of the compounds described herein, as well as methods of making such compounds.

In some embodiments of Formula (I), X is N, Y is NH, and Z is CH; such selections form a pyrazole ring. In other embodiments, X is N, Y is NH, and Z is N; such selections form a triazole ring. In still other embodiments, X is N, Y is CH, and Z is NH or N(C₁₋₄alkyl); such selections form an imidazole ring.

In some embodiments, R¹ is propyl, isopropyl, butyl, isobutyl, tert-butyl, pentyl, isopentyl, hexyl, isohexyl, heptyl, or octyl, each optionally substituted as described for Formula (I). In other embodiments, R¹ is cyclopropyl, cyclobutyl, cyclopentyl, cyclohexyl, cycloheptyl, or cyclooctyl, each optionally substituted as described for Formula (I). In still other embodiments, R¹ is C₅₋₈alkyl or C₅₋₈cycloalkyl, each optionally substituted as described for Formula (I). In still other embodiments, R¹ is cyclopentyl or cyclohexyl, each optionally substituted as described for Formula (I). In still other embodiments, R¹ is cyclopentyl or cyclohexyl. In still other embodiments, R¹ is bicyclic C₅₋₈cycloalkyl (e.g., bicyclo[3.1.0]hexan-3-yl). In some embodiments, R¹ is 3- to 8-membered heterocyclyl, optionally substituted as described for Formula (I). In other embodiments, R¹ is a 5- to 7-membered heterocyclyl, optionally substituted as described for Formula (I). In some embodiments, R¹ is C₃₋₈alkyl substituted with 1, 2, or 3 substituents independently selected from —OH, methoxy, ethoxy, propyloxy, isopropoxy, —F, —CN, amino, methylamino, dimethylamino, and C₃₋₈cycloalkyl. In other embodiments, R¹ is C₃₋₈cycloalkyl or 3- to 8-membered heterocyclyl, optionally substituted with 1 to 4 substituents independently selected from methyl, ethyl, propyl, isopropyl, —OH, methoxy, —F, and —CF₃. In other embodiments, R¹ is cyclopentyl optionally substituted with 1 to 4 substituents independently selected from methyl and —F (e.g., 3,3-dimethylcyclopentyl, 3,3-difluorocyclopentyl and 3,3,4,4-tetrafluorocyclopentyl). In other embodiments, R¹ is 5- or 6-membered heterocyclyl (e.g., tetrahydro-2H-pyran-4-yl).

In some embodiments of Formula (I), R² is H or C₁₋₄alkyl. In still other embodiments, R² is C₂₋₄alkyl substituted as described for Formula (I). In still other embodiments, R² is H, methyl, or ethyl. In still other embodiments, R² is H. In still other embodiments, R² is C₁₋₄alkyl. In still other embodiments, R² is methyl.

In some embodiments of Formula (I), R³ is C₁₋₄alkyl optionally substituted as described for Formula (I). In other embodiments, R³ is methyl or ethyl. In other embodiments, R³ is a 5-membered heteroaryl optionally substituted as described for Formula (I). In other embodiments, the 5-membered heteroaryl is selected from the group consisting of thiophenyl, thiazolyl, oxazolyl, pyrazolyl, imidazolyl, oxadiazolyl, thiadizaolyl, and triazolyl. In some embodiments, R³ is unsubstituted oxazolyl, pyrazolyl, imidazolyl, oxadiazolyl, thiadiazolyl, or triazolyl. In some embodiments, the 5-membered heteroaryl is thiophenyl or thiazolyl. In some embodiments, R³ is thiophenyl or thiazolyl, each substituted with C₁₋₄alkyl, —OH, —OC₁₋₄alkyl, —CF₃, —CN, —CO₂H, —CO₂C₁₋₄alkyl, —C(O)NR^{a}R^{b}, or —NR^{e}R^{f}. In still other embodiments, R³ is a 6-membered heteroaryl optionally substituted as described for Formula (I). In still other embodiments, R³ is pyridinyl, optionally substituted as described for Formula (I). In still other embodiments, R³ is pyridinyl substituted with —OH (which includes the tautomeric form) or —F.

In other embodiments of Formula (I), R² and R³ taken together with the nitrogen to which they are attached form a 5- to 8-membered heterocyclyl ring optionally substituted with C₁₋₄alkyl, —OH, —OC₁₋₄alkyl, oxo, halo, —CF₃, —CN, —C(O)C₁₋₄alkyl, —CO₂H, —CO₂C₁₋₄alkyl, —C(O)NR^{g}R^{h}, or —NR^{g}R^{h}. In other embodiments, R² and R³ taken together with the nitrogen to which they are attached form a pyrrolidinyl or piperidinyl ring substituted with one or two substituents independently selected from the group consisting of —OH, —OC₁₋₄alkyl, oxo, halo, —CF₃, —CN, —CO₂H, —CO₂C₁₋₄alkyl, —C(O)NR^{g}R^{h}, and —NR^{g}R^{h}. In other embodiments, the piperidinyl ring is substituted with one or two substituents independently selected from the group consisting of —OH, —OC₁₋₄alkyl, oxo, —CF₃, —CN, —CO₂H, —CO₂C₁₋₄alkyl, —C(O)NR^{g}R^{h}, and —NR^{g}R^{h}. In other embodiments, R² and R³ taken together with the nitrogen to which they are attached form a morpholinyl, thiomorpholinyl, 1,1-dioxo-thiomorpholinyl, or piperazinyl ring, each independently optionally substituted as described for Formula (I). In other embodiments, R² and R³ taken together with the nitrogen to which they are attached form a piperazinyl ring, optionally substituted as described for Formula (I). In other embodiments, R² and R³ taken together with the nitrogen to which they are attached form a 5-azaspiro[2.4]heptan-5-yl or 6-azaspiro[2.5]octan-6-yl ring, each independently optionally substituted as described for Formula (I).

In some embodiments, a is 1. In some embodiments, a is 2. In some embodiments, b is 1. In some embodiments, b is 2.

In some embodiments, r is 0. In some embodiments, r is 1. In some embodiments, r is 2. In some embodiments, r is 3 or 4. In some embodiments, s is 0. In some embodiments, s is 1. In some embodiments, s is 2. In some embodiments, s is 3 or 4.

In some embodiments, each of R⁴ and R⁵ is independently methyl, —OH, methoxy, —F, —CF₃, —CN, amino, methylamino, dimethylamino, or oxo.

In some embodiments, R¹ is (a) C₅₋₈cycloalkyl optionally substituted with 1, 2, 3, or 4 substituents selected from the group consisting of C₁₋₄alkyl, —OH, —OC₁₋₄alkyl, —F, —CF₃, —CN, —CO₂H, —CO₂C₁₋₄alkyl, —C(O)NR^{a}R^{b}, and —NR^{a}R^{b}; wherein R^{a} and R^{b} are each independently H or C₁₋₄alkyl; or (b) 5- to 8-membered heterocyclyl optionally substituted with 1, 2, 3, or 4 substituents selected from the group consisting of C₁₋₄alkyl, —OH, —OC₁₋₄alkyl, —F, —CF₃, —CN, —CO₂H, —CO₂C₁₋₄alkyl, —C(O)NR^{a}R^{b}, and —NR^{a}R^{b}; wherein R^{a} and R^{b} are each independently H or C₁₋₄alkyl; R² is C₁₋₄alkyl, or R² and R³ taken together with the nitrogen to which they are attached form a 5- to 7-membered heterocyclyl ring optionally substituted with one or two substituents independently selected from the group consisting of C₁₋₄alkyl, —OH, —OC₁₋₄alkyl, oxo, halo, —CF₃, —CN, —CO₂H, —CO₂C₁₋₄alkyl, —C(O)NR^{g}R^{h}, and —NR^{g}R^{h}; wherein R^{g} and R^{h} are each independently H or C₁₋₄alkyl; and the remaining variables are as described for Formula (I). In such embodiments, the compound does not include a compound from Table X or a pharmaceutically acceptable salt thereof. In certain such embodiments, the compound does not include a compound selected from the group consisting of Compound Nos. 108X, 110X, 111X, 112X, 113X, 114X, 115X, 116X, 123X, 125X, 126X, 127X, 128X, 130X, 131X, 133X, 134X, 136X, 137X, 155X, 156X, 158X, 159X, 168X, 169X, 170X, 171X, 174X, 175X, 176X, 177X, 178X, 179X, 180X, 181X, 182X, 183X, 184X and 185X in Table X, and pharmaceutically acceptable salts thereof.

It is intended and understood that each and every variation of X, Y, Z, R¹, R², R³, R⁴, R⁵, a, b, r, and s described herein, where applicable, may be combined with each and every variation of these variables as described for Formula (I) as if each and every combination is individually described. For example, in some embodiments, the compound is of the formula (I), or a pharmaceutically acceptable salt thereof, wherein X, Y, Z, R⁴, R⁵, a, b, r, and s are as described for formula (I) or variations thereof; R¹ is C₅₋₈cycloalkyl (e.g., cyclopentyl, cyclohexyl or bicyclo[3.1.0]hexan-3-yl) or 5- to 7-membered heterocyclyl (e.g., tetrahydro-2H-pyran-4-yl), each optionally substituted as described for Formula (I); and R² and R³ taken together with the nitrogen to which they are attached form a 5- to 8-membered heterocyclyl (e.g., pyrrolidinyl, piperidinyl, morpholinyl, thiomorpholinyl, piperazinyl, 5-azaspiro[2.4]heptan-5-yl or 6-azaspiro[2.5]octan-6-yl) optionally substituted with C₁₋₄alkyl, —OH, —OC₁₋₄alkyl, oxo, halo, —CF₃, —CN, —C(O)C₁₋₄alkyl, —CO₂H, —CO₂C₁₋₄alkyl, —C(O)NR^{g}R^{h}, or —NR^{g}R^{h}. In some of these embodiments, R¹ is C₅₋₈cycloalkyl optionally substituted as described for Formula (I). In some of these embodiments, R¹ is 5- to 7-membered heterocyclyl optionally substituted as described for Formula (I). In some of these embodiments, a is 1. In some of these embodiments, a is 1 and b is 1. In some of these embodiments, r is 0. In some of these embodiments, s is 0. In some of these embodiments, r is 0 and s is 0. In some of these embodiments, X is N, Y is NH, and Z is CH. In some of these embodiments, X is N, Y is NH, and Z is N. In some of these embodiments, X is N, Y is CH, and Z is NH or N(C₁₋₄alkyl). In some of these embodiments, X is N, Y is NH, Z is CH, a is 1 and b is 1. In some of these embodiments, X is N, Y is NH, Z is N, a is 1 and b is 1. In some of these embodiments, X is N, Y is CH, Z is NH or N(C₁₋₄alkyl), a is 1 and b is 1. In some of these embodiments, the compound does not include a compound from Table X or a pharmaceutically acceptable salt thereof. In some of these embodiments, the compound does not include a compound selected from the group consisting of Compound Nos. 112X, 113X, 125X, 130X, 131X, 133X, 134X, 155X, 156X, 158X, 159X, 174X, 175X, 176X, 180X, 181X, 183X, 184X and 185X in Table X, and pharmaceutically acceptable salts thereof. In some of these embodiments, the compound or salt thereof inhibits one or two or three of Trk A, Trk B and Trk C. In some of these embodiments, the compound or salt thereof inhibits one or two or three of Trk A, Trk B and Trk C to a greater extent than it inhibits IGF-1R. In some of these embodiments, the compounds do not inhibit appreciably the activity of IGF-1R and/or IR receptors. In some of these embodiments, the compound or salt thereof inhibits one or more FMS-like tyrosine kinases (e.g., FLT3, FLT3(D835Y) and FLT3(ITD)). In some of these embodiments, the compound or salt thereof inhibits one or more FMS-like tyrosine kinases (e.g., FLT3, FLT3(D835Y) and FLT3(ITD)) to a greater extent than it inhibits IGF-1R. In some of these embodiments, the compounds do not inhibit appreciably the activity of IGF-1R and/or IR receptors.

In some embodiments, the compound is of the formula (I), or a pharmaceutically acceptable salt thereof, wherein X, Y, Z, R⁴, R⁵, a, b, r, and s are as described for formula (I) or variations thereof; R¹ is C₅₋₈cycloalkyl (e.g., cyclopentyl, cyclohexyl or bicyclo[3.1.0]hexan-3-yl) or 5- to 7-membered heterocyclyl (e.g., tetrahydro-2H-pyran-4-yl), each optionally substituted as described for Formula (I); R² is H or C₁₋₄alkyl (e.g., methyl or ethyl); and R³ is a 5-membered heteroaryl (e.g., thiophenyl, thiazolyl, oxazolyl, pyrazolyl, imidazolyl, oxadiazolyl, thiadizaolyl, or triazolyl) optionally substituted as described for Formula (I). In some of these embodiments, R² is C₁₋₄alkyl. In some of these embodiments, R² is H. In some of these embodiments, R¹ is C₅₋₈cycloalkyl optionally substituted as described for Formula (I). In some of these embodiments, R¹ is 5- to 7-membered heterocyclyl optionally substituted as described for Formula (I). In some of these embodiments, a is 1. In some of these embodiments, a is 1 and b is 1. In some of these embodiments, r is 0. In some of these embodiments, s is 0. In some of these embodiments, r is 0 and s is 0. In some of these embodiments, X is N, Y is NH, and Z is CH. In some of these embodiments, X is N, Y is NH, and Z is N. In some of these embodiments, X is N, Y is CH, and Z is NH or N(C₁₋₄alkyl). In some of these embodiments, X is N, Y is NH, Z is CH, a is 1 and b is 1. In some of these embodiments, X is N, Y is NH, Z is N, a is 1 and b is 1. In some of these embodiments, X is N, Y is CH, Z is NH or N(C₁₋₄alkyl), a is 1 and b is 1. In some of these embodiments, the compound does not include a compound from Table X or a pharmaceutically acceptable salt thereof. In some of these embodiments, the compound or salt thereof inhibits one or two or three of Trk A, Trk B and Trk C. In some of these embodiments, the compound or salt thereof inhibits one or two or three of Trk A, Trk B and Trk C to a greater extent than it inhibits IGF-1R. In some of these embodiments, the compounds do not inhibit appreciably the activity of IGF-1R and/or IR receptors. In some of these embodiments, the compound or salt thereof inhibits one or more FMS-like tyrosine kinases (e.g., FLT3, FLT3(D835Y) and FLT3(ITD)). In some of these embodiments, the compound or salt thereof inhibits one or more FMS-like tyrosine kinases (e.g., FLT3, FLT3(D835Y) and FLT3(ITD)) to a greater extent than it inhibits IGF-1R. In some of these embodiments, the compounds do not inhibit appreciably the activity of IGF-1R and/or IR receptors.

In some embodiments of Formula (II), X is N, Y is NH, and Z is CH; such selections form a pyrazole ring. In other embodiments, X is N, Y is NH, and Z is N; such selections form a triazole ring. In still other embodiments, X is N, Y is CH, and Z is NH or N(C₁₋₄alkyl); such selections form an imidazole ring.

In other embodiments of Formula (II), R¹ is propyl, isopropyl, butyl, isobutyl, tert-butyl, pentyl, isopentyl, hexyl, isohexyl, heptyl, or octyl, each optionally substituted as described for Formula (II). In other embodiments, R¹ is cyclopropyl, cyclobutyl, cyclopentyl, cyclohexyl, cycloheptyl, or cyclooctyl, each optionally substituted as described for Formula (II). In still other embodiments, R¹ is C₅₋₈alkyl or C₅₋₈cycloalkyl, each optionally substituted as described for Formula (II). In still other embodiments, R¹ is cyclopentyl or cyclohexyl, each optionally substituted as described for Formula (II). In some embodiments, R¹ is 3- to 8-membered heterocyclyl, optionally substituted as described for Formula (II). In other embodiments, R¹ is a 5- to 7-membered heterocyclyl, optionally substituted as described for Formula (II). In some embodiments, R¹ is C₃₋₈alkyl substituted with 1, 2, or 3 substituents independently selected from —OH, methoxy, ethoxy, propyloxy, isopropoxy, —F, —CN, amino, methylamino, dimethylamino, and C₃₋₈cycloalkyl. In other embodiments, R¹ is C₃₋₈cycloalkyl or 3- to 8-membered heterocyclyl, optionally substituted with 1 to 4 substituents independently selected from methyl, ethyl, propyl, isopropyl, —OH, methoxy, —F, and —CF₃. In still other embodiments, R¹ is monocyclic C₅₋₈cycloalkyl (e.g., cyclopentyl or cyclohexyl). In other embodiments, R¹ is bicyclic C₅₋₈cycloalkyl (e.g., bicyclo[3.1.0]hexan-3-yl). In other embodiments, R¹ is cyclopentyl optionally substituted with 1 to 4 substituents independently selected from methyl and —F (e.g., 3,3-dimethylcyclopentyl, 3,3-difluorocyclopentyl and 3,3,4,4-tetrafluorocyclopentyl). In other embodiments, R¹ is 5- or 6-membered heterocyclyl (e.g., tetrahydro-2H-pyran-4-yl).

In some embodiments of Formula (II), R² is H or C₁₋₄alkyl. In still other embodiments, R² is C₂₋₄alkyl substituted as described for Formula (II). In still other embodiments, R² is H, methyl, or ethyl. In still other embodiments, R² is H. In still other embodiments, R² is C₁₋₄alkyl. In still other embodiments, R² is methyl.

In some embodiments of Formula (II), R³ is C₁₋₄alkyl optionally substituted as described for Formula (II). In other embodiments, R³ is methyl or ethyl. In other embodiments, R³ is a 5-membered heteroaryl optionally substituted as described for Formula (II). In other embodiments, the 5-membered heteroaryl is selected from the group consisting of thiophenyl, thiazolyl, oxazolyl, pyrazolyl, imidazolyl, oxadiazolyl, thiadiazolyl, and triazolyl. In some embodiments, R³ is unsubstituted oxazolyl, pyrazolyl, imidazolyl, oxadiazolyl, thiadiazolyl, or triazolyl. In some embodiments, the 5-membered heteroaryl is thiophenyl or thiazolyl. In some embodiments, R³ is thiophenyl or thiazolyl, each substituted with C₁₋₄alkyl, —OH, —OC₁₋₄alkyl, —CF₃, —CN, —CO₂H, —CO₂C₁₋₄alkyl, —C(O)NR^{e}R^{f}, or —NR^{e}R^{f}. In still other embodiments, R³ is a 6-membered heteroaryl optionally substituted as described for Formula (II). In still other embodiments, R³ is pyridinyl, optionally substituted as described for Formula (II). In still other embodiments, R³ is pyridinyl substituted with —OH (which includes the tautomeric form) or —F.

In other embodiments of Formula (II), R² and R³ taken together with the nitrogen to which they are attached form a 5- to 8-membered heterocyclyl ring optionally substituted with C₁₋₄alkyl, —OH, —OC₁₋₄alkyl, oxo, halo, —CF₃, —CN, —C(O)C₁₋₄alkyl, —CO₂H, —CO₂C₁₋₄alkyl, —C(O)NR^{g}R^{h}, or —NR^{g}R^{h}. In other embodiments, R² and R³ taken together with the nitrogen to which they are attached form a pyrrolidinyl or piperidinyl ring substituted with one or two substituents independently selected from the group consisting of —OH, —OC₁₋₄alkyl, oxo, halo, —CF₃, —CN, —CO₂H, —CO₂C₁₋₄alkyl, —C(O)NR^{g}R^{h}, and —NR^{g}R^{h}. In other embodiments, the piperidinyl ring is substituted with one or two substituents selected from the group consisting of —OH, —OC₁₋₄alkyl, oxo, —CF₃, —CN, —CO₂H, —CO₂C₁₋₄alkyl, —C(O)NR^{g}R^{h}, and —NR^{g}R^{h}. In other embodiments, R² and R³ taken together with the nitrogen to which they are attached form a morpholinyl, thiomorpholinyl, 1,1-dioxo-thiomorpholinyl, or piperazinyl ring, each optionally substituted as described for Formula (II). In other embodiments, R² and R³ taken together with the nitrogen to which they are attached form a piperazinyl ring, optionally substituted as described for Formula (II). In other embodiments, R² and R³ taken together with the nitrogen to which they are attached form a 5-azaspiro[2.4]heptan-5-yl or 6-azaspiro[2.5]octan-6-yl ring, each independently optionally substituted as described for Formula (II).

In some embodiments of Formula (II), b is 1. In some embodiments, b is 2.

In some embodiments of Formula (I) is a compound of Formula (III):

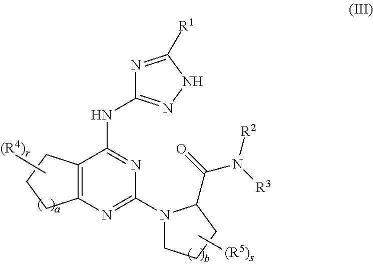

wherein R¹, R², R³, R⁴, R⁵, a, b, r, and s are each defined as for Formula (I);
or a pharmaceutically acceptable salt thereof.

In some embodiments of Formula (III), r and s are each 0. In some embodiments, a is 1. In other embodiments, b is 1 or 2.

In other embodiments of Formula (III), R¹ is propyl, isopropyl, butyl, isobutyl, tert-butyl, pentyl, isopentyl, hexyl, isohexyl, heptyl, or octyl, each optionally substituted as described for Formula (I). In other embodiments, R¹ is cyclopropyl, cyclobutyl, cyclopentyl, cyclohexyl, cycloheptyl, or cyclooctyl, each optionally substituted as described for Formula (I). In still other embodiments, R¹ is C₅₋₈alkyl or C₅₋₈cycloalkyl, each optionally substituted as described for Formula (I). In still other embodiments, R¹ is cyclopentyl or cyclohexyl, each optionally substituted as described for Formula (I). In still other embodiments, R¹ is cyclopentyl or cyclohexyl. In some embodiments, R¹ is 3- to 8-membered heterocyclyl, optionally substituted as described for Formula (I). In other embodiments, R¹ is a 5- to 7-membered heterocyclyl, optionally substituted as described for Formula (I). In some embodiments, R¹ is C₃₋₈alkyl substituted with 1, 2, or 3 substituents independently selected from —OH, methoxy, ethoxy, propyloxy, isopropoxy, —F, —CN, amino, methylamino, dimethylamino, and C₃₋₈cycloalkyl. In other embodiments, R¹ is C₃₋₈cycloalkyl or 3- to 8-membered heterocyclyl, optionally substituted with 1 to 4 methyl, ethyl, propyl, isopropyl, —OH, methoxy, —F, or —CF₃ groups. In other embodiments, R¹ is monocyclic C₅₋₈cycloalkyl (e.g., cyclopentyl or cyclohexyl). In other embodiments, R¹ is bicyclic C₅₋₈cycloalkyl (e.g., bicyclo[3.1.0]hexan-3-yl). In other embodiments, R¹ is cyclopentyl optionally substituted with 1 to 4 substituents independently selected from methyl and —F (e.g., 3,3-dimethylcyclopentyl, 3,3-difluorocyclopentyl and 3,3,4,4-tetrafluorocyclopentyl). In other embodiments, R¹ is 5- or 6-membered heterocyclyl (e.g., tetrahydro-2H-pyran-4-yl).

In some embodiments of Formula (III), R² is H or C₁₋₄alkyl. In still other embodiments, R² is C₂₋₄alkyl substituted as described for Formula (I). In still other embodiments, R² is H, methyl, or ethyl. In still other embodiments, R² is H. In still other embodiments, R² is C₁₋₄alkyl. In still other embodiments, R² is methyl.

In some embodiments of Formula (III), R³ is C₁₋₄alkyl optionally substituted as described for Formula (II). In other embodiments, R³ is methyl or ethyl. In other embodiments, R³ is a 5-membered heteroaryl optionally substituted as described for Formula (I). In other embodiments, the 5-membered heteroaryl is selected from the group consisting of thiophenyl, thiazolyl, oxazolyl, pyrazolyl, imidazolyl, oxadiazolyl, thiadiazolyl, and triazolyl. In some embodiments, R³ is unsubstituted oxazolyl, pyrazolyl, imidazolyl, oxadiazolyl, thiadiazolyl, or triazolyl. In some embodiments, the 5-membered heteroaryl is thiophenyl or thiazolyl. In some embodiments, R³ is thiophenyl or thiazolyl, each substituted with C₁₋₄alkyl, —OH, —OC₁₋₄alkyl,

—CF₃, —CN, —CO₂H, —CO₂C₁₋₄alkyl, —C(O)NR^{e}R^{f}, or —NR^{e}R^{f}. In still other embodiments, R³ is a 6-membered heteroaryl optionally substituted as described for Formula (I). In still other embodiments, R³ is pyridinyl, optionally substituted as described for Formula (I). In still other embodiments, R³ is pyridinyl substituted with —OH (which includes the tautomeric form) or —F.

In other embodiments of Formula (III), R² and R³ taken together with the nitrogen to which they are attached form a 5- to 8-membered heterocyclyl ring optionally substituted with C₁₋₄alkyl, —OH, —OC₁₋₄alkyl, oxo, halo, —CF₃, —CN, —C(O)C₁₋₄alkyl, —CO₂H, —CO₂C₁₋₄alkyl, —C(O)NR^{g}R^{h}, or —NR^{g}R^{h}. In other embodiments, R² and R³ taken together with the nitrogen to which they are attached form a pyrrolidinyl or piperidinyl ring substituted with one or two substituents selected from the group consisting of —OH, —OC₁₋₄alkyl, oxo, halo, —CF₃, —CN, —CO₂H, —CO₂C₁₋₄alkyl, —C(O)NR^{g}R^{h}, and —NR^{g}R^{h}. In other embodiments, the piperidinyl ring is substituted with one or two substituents selected from the group consisting of —OH, —OC₁₋₄alkyl, oxo, —CF₃, —CN, —CO₂H, —CO₂C₁₋₄alkyl, —C(O)NR^{g}R^{h}, and —NR^{g}R^{h}. In other embodiments, R² and R³ taken together with the nitrogen to which they are attached form a morpholinyl, thiomorpholinyl, 1,1-dioxo-thiomorpholinyl, or piperazinyl ring, each optionally substituted as described for Formula (I). In other embodiments, R² and R³ taken together with the nitrogen to which they are attached form a piperazinyl ring, optionally substituted as described for Formula (I).

In some embodiments of Formula (III), the compound is not a compound in Table X or a pharmaceutically acceptable salt thereof.

In some embodiments of Formula (I) is a compound of Formula (IV):

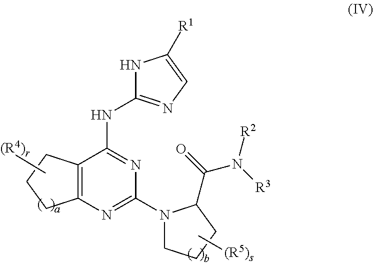

wherein R¹, R², R³, R⁴, R⁵, a, b, r, and s are each defined as for Formula (I);
or a pharmaceutically acceptable salt thereof.

In some embodiments of Formula (IV), r and s are each 0. In some embodiments, a is 1. In other embodiments, b is 1 or 2.

In other embodiments of Formula (IV), R¹ is propyl, isopropyl, butyl, isobutyl, tert-butyl, pentyl, isopentyl, hexyl, isohexyl, heptyl, or octyl, each optionally substituted as described for Formula (I). In other embodiments, R¹ is cyclopropyl, cyclobutyl, cyclopentyl, cyclohexyl, cycloheptyl, or cyclooctyl, each optionally substituted as described for Formula (I). In still other embodiments, R¹ is C₅₋₈alkyl or C₅₋₈cycloalkyl, each optionally substituted as described for Formula (I). In still other embodiments, R¹ is cyclopentyl or cyclohexyl, each optionally substituted as described for Formula (I). In some embodiments, R¹ is 3- to 8-membered heterocyclyl, optionally substituted as described for Formula (I). In other embodiments, R¹ is a 5- to 7-membered heterocyclyl, optionally substituted as described for Formula (II). In some embodiments, R¹ is C₃₋₈alkyl substituted with 1, 2, or 3 substituents independently selected from —OH, methoxy, ethoxy, propyloxy, isopropoxy, —F, —CN, amino, methylamino, dimethylamino, and C₃₋₈cycloalkyl. In other embodiments, R¹ is C₃₋₈cycloalkyl or 3- to 8-membered heterocyclyl, optionally substituted with 1 to 4 methyl, ethyl, propyl, isopropyl, —OH, methoxy, —F, or —CF₃ groups. In other embodiments, R¹ is monocyclic C₅₋₈cycloalkyl (e.g., cyclopentyl or cyclohexyl). In other embodiments, R¹ is bicyclic C₅₋₈cycloalkyl (e.g., bicyclo[3.1.0]hexan-3-yl). In other embodiments, R¹ is cyclopentyl optionally substituted with 1 to 4 substituents independently selected from methyl and —F (e.g., 3,3-dimethylcyclopentyl, 3,3-difluorocyclopentyl and 3,3,4,4-tetrafluorocyclopentyl). In other embodiments, R¹ is 5- or 6-membered heterocyclyl (e.g., tetrahydro-2H-pyran-4-yl).

In some embodiments of Formula (IV), R² is H or C₁₋₄alkyl. In still other embodiments, R² is C₂₋₄alkyl substituted as described for Formula (I). In still other embodiments, R² is H. methyl, or ethyl. In still other embodiments, R² is H. In still other embodiments, R² is C₁₋₄alkyl. In still other embodiments, R² is methyl.

In some embodiments of Formula (IV), R³ is C₁₋₄alkyl optionally substituted as described for Formula (II). In other embodiments, R³ is methyl or ethyl. In other embodiments, R³ is a 5-membered heteroaryl optionally substituted as described for Formula (I). In other embodiments, the 5-membered heteroaryl is selected from the group consisting of thiophenyl, thiazolyl, oxazolyl, pyrazolyl, imidazolyl, oxadiazolyl, thiadiazolyl, and triazolyl. In some embodiments, R³ is unsubstituted oxazolyl, pyrazolyl, imidazolyl, oxadiazolyl, thiadiazolyl, or triazolyl. In some embodiments, the 5-membered heteroaryl is thiophenyl or thiazolyl. In some embodiments, R³ is thiophenyl or thiazolyl, each substituted with C₁₋₄alkyl, —OH, —OC₁₋₄alkyl, —CF₃, —CN, —CO₂H, —CO₂C₁₋₄alkyl, —C(O)NR^{e}R^{f}, or —NR^{e}R^{f}. In still other embodiments, R³ is a 6-membered heteroaryl optionally substituted as described for Formula (I). In still other embodiments, R³ is pyridinyl, optionally substituted as described for Formula (I). In still other embodiments, R³ is pyridinyl substituted with —OH (which includes the tautomeric form) or —F.

In other embodiments of Formula (IV), R² and R³ taken together with the nitrogen to which they are attached form a 5- to 8-membered heterocyclyl ring optionally substituted with C₁₋₄alkyl, —OH, —OC₁₋₄alkyl, oxo, halo, —CF₃, —CN, —C(O)C₁₋₄alkyl, —CO₂H, —CO₂C₁₋₄alkyl, —C(O)NR^{g}R^{h}, or —NR^{g}R^{h}. In other embodiments, R² and R³ taken together with the nitrogen to which they are attached form a pyrrolidinyl or piperidinyl ring substituted with one or two substituents selected from the group consisting of —OH, —OC₁₋₄alkyl, oxo, halo, —CF₃, —CN, —CO₂H, —CO₂C₁₋₄alkyl, —C(O)NR^{g}R^{h}, and —NR^{g}R^{h}. In other embodiments, the piperidinyl ring is substituted with one or two substituents selected from the group consisting of —OH, —OC₁₋₄alkyl, oxo, —CF₃, —CN, —CO₂H, —CO₂C₁₋₄alkyl, —C(O)NR^{g}R^{h}, and —NR^{g}R^{h}. In other embodiments, R² and R³ taken together with the nitrogen to which they are attached form a morpholinyl, thiomorpholinyl, 1,1-dioxo-thiomorpholinyl, or piperazinyl ring, each optionally substituted as described for Formula (I). In other embodiments, R² and R³ taken together with the nitrogen to which they are attached form a piperazinyl ring, optionally substituted as described for Formula (I).

In some embodiments of Formula (IV), the compound is not a compound in Table X or a pharmaceutically acceptable salt thereof. In other embodiments of Formula (IV), the compound is not a compound selected from the group consisting of Compound Nos. 154X, 155X, 156X, 157X, 158X and 159X in Table X, and pharmaceutically acceptable salts thereof.

In other embodiments of Formula (IV):
R² is H or C₁₋₄alkyl optionally substituted with —OH, —OC₁₋₄alkyl, —F, —CF₃, —CN, or —NR^{c}R^{d}; wherein R^{c} and R^{d} are each independently H or C₁₋₄alkyl;
R³ is a 5- or 6-membered heteroaryl optionally substituted with one or more substituents selected from the group consisting of C₁₋₄alkyl, —OH, —OC₁₋₄alkyl, halo, —CF₃, —CN, —CO₂H,
   —CO₂C₁₋₄alkyl, —C(O)NR^{e}R^{f}, and —NR^{e}R^{f};
or R² and R³ taken together with the nitrogen to which they are attached form a piperidinyl ring substituted with one or two substituents selected from the group consisting of C₁₋₄alkyl, —OH, —OC₁₋₄alkyl, oxo, halo, —CF₃, —CN, —CO₂H, —CO₂C₁₋₄alkyl, —C(O)NR^{g}R^{h} and —NR^{g}R^{h}; or 5- to 7-membered heterocyclyl ring other than piperidinyl, wherein the heterocyclyl ring is optionally substituted with one or two substituents selected from the group consisting of C₁₋₄alkyl, —OH, —OC₁₋₄alkyl, oxo, halo, —CF₃, —CN, —CO₂H, —CO₂C₁₋₄alkyl, —C(O)NR^{g}R^{h}, and —NR^{g}R^{h};
   wherein R^{g} and R^{h} are each independently H or C₁₋₄alkyl.

In some embodiments of Formula (I) is a compound of Formula (V):

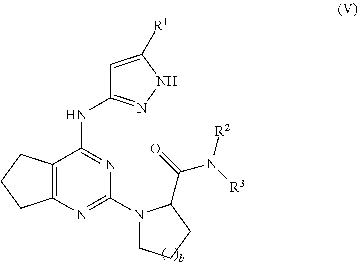

wherein R¹, R², R³, R⁴, R⁵, and b are each defined as for Formula (I);
or a pharmaceutically acceptable salt thereof.

In some embodiments of Formula (V), R¹ is C₃₋₇alkyl or C₃₋₇cycloalkyl. In other embodiments, R¹ is C₅₋₇cycloalkyl. In other embodiments, R¹ is monocyclic C₅₋₇cycloalkyl (e.g., cyclopentyl or cyclohexyl). In other embodiments, R¹ is bicyclic C₅₋₈cycloalkyl (e.g., bicyclo[3.1.0]hexan-3-yl). In other embodiments, R¹ is cyclopentyl optionally substituted with 1 to 4 substituents independently selected from methyl and —F (e.g., 3,3-dimethylcyclopentyl, 3,3-difluorocyclopentyl and 3,3,4,4-tetrafluorocyclopentyl). In other embodiments, R¹ is 5- or 6-membered heterocyclyl (e.g., tetrahydro-2H-pyran-4-yl).

In some embodiments of Formula (V), R² is H or C₁₋₄alkyl. In other embodiments, R² is methyl or ethyl. In some embodiments, R³ is a 5-membered heteroaryl optionally substituted as described for Formula (I). In other embodiments, the 5-membered heteroaryl is selected from the group consisting of thiophenyl, thiazolyl, oxazolyl, pyrazolyl, imidazolyl, oxadiazolyl, thiadiazolyl, and triazolyl. In some embodiments, R³ is unsubstituted oxazolyl, pyrazolyl, imidazolyl, oxadiazolyl, thiadiazolyl, or triazolyl. In some embodiments, the 5-membered heteroaryl is thiophenyl or thiazolyl. In some embodiments, R³ is thiophenyl or thiazolyl, each substituted with C₁₋₄alkyl, —OH, —OC₁₋₄alkyl, —CF₃, —CN, —CO₂H, —CO₂C₁₋₄alkyl, —C(O)NR^{e}R^{f}, or —NR^{e}R^{f}. In still other embodiments, R³ is a 6-membered heteroaryl optionally substituted as described for Formula (I). In still other embodiments, R³ is pyridinyl, optionally substituted as described for Formula (I). In still other embodiments, R³ is pyridinyl substituted with —OH (which includes the tautomeric form) or —F. In still other embodiments, R³ is a 5- or 6-membered heteroaryl optionally substituted with —OH or halo. In other embodiments, R³ is 2-hydroxypyridin-5-yl, 2-chlorothiophen-5-yl, thiophenyl, 5-chloro-thiazol-2-yl, thiazolyl, oxazolyl, imidazolyl, triazolyl, pyrazolyl, or 2-fluoropyridin-5-yl. In some embodiments, R³ is 2-chlorothiophen-5-yl, oxazolyl, imidazolyl, triazolyl, or pyrazolyl. In some embodiments, R² and R³ taken together with the nitrogen to which they are attached form R² and R³ taken together with the nitrogen to which they are attached form a 5- to 8-membered heterocyclyl ring optionally substituted with C₁₋₄alkyl, —OH, —OC₁₋₄alkyl, oxo, halo, —CF₃, —CN, —C(O)C₁₋₄alkyl, —CO₂H, —CO₂C₁₋₄alkyl, —C(O)NR^{g}R^{h}, or —NR^{g}R^{h}. In other embodiments, R² and R³ taken together with the nitrogen to which they are attached form a pyrrolidinyl or piperidinyl ring substituted with one or two substituents selected from the group consisting of —OH, —OC₁₋₄alkyl, oxo, halo, —CF₃, —CN, —CO₂H, —CO₂C₁₋₄alkyl, —C(O)NR^{g}R^{h}, and —NR^{g}R^{h}. In other embodiments, the piperidinyl ring is substituted with one or two substituents selected from the group consisting of —OH, —OC₁₋₄alkyl, oxo, —CF₃, —CN, —CO₂H, —CO₂C₁₋₄alkyl, —C(O)NR^{g}R^{h}, and —NR^{g}R^{h}. In other embodiments, R² and R³ taken together with the nitrogen to which they are attached form a morpholinyl, thiomorpholinyl, 1,1-dioxo-thiomorpholinyl, or piperazinyl ring, each optionally substituted as described for Formula (I). In other embodiments, R² and R³ taken together with the nitrogen to which they are attached form a piperazinyl ring, optionally substituted as described for Formula (I). In other embodiments, R² and R³ taken together with the nitrogen to which they are attached form morpholinyl, 4-methylpiperazinyl, 1,1-dioxothiomorpholin-1-yl, 4,4-dimethylpiperidinyl, 4,4-difluoropiperidinyl, or 4-methyl-3-oxo-piperazin-1-yl. In still other embodiments, R² and R³ taken together with the nitrogen to which they are attached form 4-methylpiperazinyl, 4,4-dimethylpiperidinyl, or 4-methyl-3-oxo-piperazin-1-yl. In other embodiments, R² and R³ taken together with the nitrogen to which they are attached form a 5-azaspiro[2.4]heptan-5-yl or 6-azaspiro[2.5]octan-6-yl ring, each independently optionally substituted as described for Formula (I).

In some embodiments of Formula (V), b is 1.

In some embodiments of Formula (V), the compound is not a compound from Table X or a pharmaceutically acceptable salt thereof. In other embodiments of Formula (V), the compound is not selected from the group consisting of Compound Nos. 2X, 3X, 4X, 6X, 7X, 8X, 9X, 16X, 17X, 19X, 20X, 24X, 29X, 32X, 42X, 55X, 60X, 61X, 62X, 63X, 64X, 68X, 69X, 72X, 73X, 74X, 75X, 79X, 82X, 84X, 90X, 91X, 95X, 97X, 98X, 100X, 111X, 112X, 113X, 114X, 115X, 116X, 119X, 120X, 121X, 122X, 123X, 124X, 125X, 140X, 141X, 154X, 168X, 169X, 170X, 171X, 180X, 183X and 184X in Table X, and pharmaceutically acceptable salts thereof. In some of these embodiments, the compound or salt thereof inhibits one or two or three of Trk A, Trk B and Trk C. In some of these embodiments, the compound or salt thereof inhibits one or two or three of Trk A, Trk B and Trk C to a greater extent than it inhibits IGF-1R. In some of these embodiments, the compounds do not inhibit appreciably the activity of IGF-1R and/or IR receptors. In some of these embodiments, the compound or salt thereof inhibits one or more FMS-like tyrosine kinases (e.g., FLT3, FLT3(D835Y) and FLT3(ITD)). In some of these embodiments, the compound or salt thereof inhibits one or more FMS-like tyrosine kinases (e.g., FLT3, FLT3(D835Y) and FLT3(ITD)) to a greater extent than it inhibits IGF-1R. In some of these embodiments, the compounds do not inhibit appreciably the activity of IGF-1R and/or IR receptors.

In some embodiments of Formula (I) is a compound of Formula (VI):

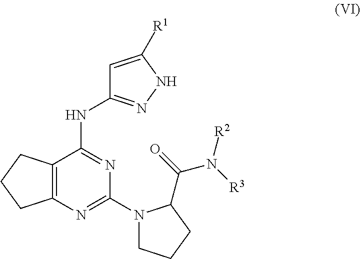

wherein R¹ is C₃₋₈cycloalkyl;
R² is H or C₁₋₄alkyl;
R³ is a 5-membered heteroaryl optionally substituted with C₁₋₄alkyl, —OH, —OC₁₋₄alkyl, halo, —CF₃, —CN, —CO₂H, —CO₂C₁₋₄alkyl, —C(O)NR^{e}R^{f}, or —NR^{e}R^{f};
or R² and R³ taken together with the nitrogen to which they are attached form a 5- to 7-membered heterocyclyl ring optionally substituted with 1 or 2 substituents selected from the group consisting of C₁₋₄alkyl, —OH, —OC₁₋₄alkyl, oxo, halo, —CF₃, —CN, —C(O)C₁₋₄alkyl, —CO₂H, —CO₂C₁₋₄alkyl, —C(O)NR^{g}R^{h}, and —NR^{g}R^{h};
where R^{a}, R^{b}, R^{e}, R^{f}, R^{g}, and R^{h} are defined as for Formula (I);
   or a pharmaceutically acceptable salt thereof.

In some embodiments of Formula (VI), R¹ is cyclopentyl. In other embodiments, R¹ is cyclohexyl.

In some embodiments of Formula (VI), R² is H. In other embodiments, R² is methyl, ethyl, propyl, or isopropyl. In other embodiments, R² is methyl.

In some embodiments of Formula (VI), In other embodiments, R³ is a 5-membered heteroaryl substituted with methyl, ethyl, isopropyl, methoxy, acetyl, or amino. In other embodiments, R³ is an unsubstituted 5-membered heteroaryl. In other embodiments, the 5-membered heteroaryl is thiophenyl, thiazolyl, oxazolyl, imidazolyl, pyrazolyl, triazolyl, oxadiazolyl, or thiadiazolyl. In other embodiments, the 5-membered heteroaryl is thiazolyl.

In some embodiments of Formula (VI), R² and R³ taken together with the nitrogen to which they are attached form a 6-membered heterocyclyl ring, optionally substituted as described for Formula (VI). In other embodiments, R² and R³ taken together with the nitrogen to which they are attached form piperidine or piperazine, optionally substituted with one or two substituents selected from the group consisting of C₁₋₄alkyl, oxo, and —C(O)C₁₋₄alkyl. In other embodiments, the substituents are selected from methyl, oxo, and acetyl.

In some embodiments of Formula (VI), the compound is not a compound from Table X or a pharmaceutically acceptable salt thereof. In other embodiments of Formula (VI), the compound is not a compound selected from the group consisting of Compound Nos. 24X, 111X, 112X, 113X, 125X, 180X, 183X and 184X in Table X, and pharmaceutically acceptable salts thereof. In some of these embodiments, the compound or salt thereof inhibits one or two or three of Trk A, Trk B and Trk C. In some of these embodiments, the compound or salt thereof inhibits one or two or three of Trk A, Trk B and Trk C to a greater extent than it inhibits IGF-1R. In some of these embodiments, the compounds do not inhibit appreciably the activity of IGF-1R and/or IR receptors. In some of these embodiments, the compound or salt thereof inhibits one or more FMS-like tyrosine kinases (e.g., FLT3, FLT3(D835Y) and FLT3(ITD)). In some of these embodiments, the compound or salt thereof inhibits one or more FMS-like tyrosine kinases (e.g., FLT3, FLT3(D835Y) and FLT3(ITD)) to a greater extent than it inhibits IGF-1R. In some of these embodiments, the compounds do not inhibit appreciably the activity of IGF-1R and/or IR receptors.

Representative compounds of the invention, and their stereoisomers, are listed in Table 1. For compounds bearing one or more chiral centers, the racemate is assigned an example number, and each unique stereoisomer is assigned a suffix “a”, “b”, etc. For example, racemic compound 1, bearing one chiral center, may be resolved into its individual enantiomers 1a and 1b. Similarly, racemic compound 100, bearing two chiral centers, can be resolved into its individual stereoisomers 100a, 100b, 100c and 100d.

In some embodiments, a compound as described herein is selected from the group consisting of those in Table 1 and pharmaceutically acceptable salts thereof. (“Compound” is abbreviated as “Cpd”.)

TABLE 1

| Cpd No. | Structure | Compound Name |
|---|---|---|
| 1 | 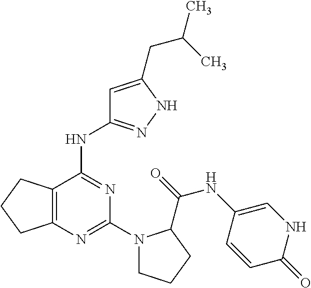 [SMILES: CC(C)Cc1cc(Nc2nc(N3CCCC3C(=O)Nc3ccc(=O)[nH]c3)nc3c2CCC3)n[nH]1] | 1-(4-(5-isobutyl-1H-pyrazol-3-ylamino)-6,7- dihydro-5H-cyclopenta[d]pyrimidin-2-yl)-N-(6- oxo-1,6-dihydropyridin-3-yl)pyrrolidine-2- carboxamide |
| 1a | | (R)-1-(4-(5-isobutyl-1H-pyrazol-3-ylamino)-6,7- |
| | | dihydro-5H-cyclopenta[d]pyrimidin-2-yl)-N-(6- |
| | | oxo-1,6-dihydropyridin-3-yl)pyrrolidine-2- |
| | | carboxamide |
| 1b | | (S)-1-(4-(5-isobutyl-1H-pyrazol-3-ylamino)-6,7- |
| | | dihydro-5H-cyclopenta[d]pyrimidin-2-yl)-N-(6- |
| | | oxo-1,6-dihydropyridin-3-yl)pyrrolidine-2- |
| | | carboxamide |
| 2 | 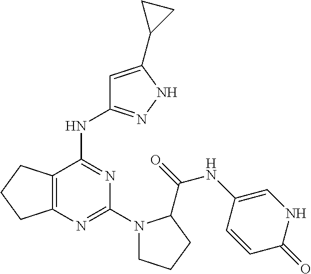 [SMILES: O=C(Nc1ccc(=O)[nH]c1)C1CCCN1c1nc2c(c(Nc3cc(C4CC4)[nH]n3)n1)CCC2] | 1-(4-(5-cyclopropyl-1H-pyrazol-3-ylamino)-6,7- dihydro-5H-cyclopenta[d]pyrimidin-2-yl)-N-(6- oxo-1,6-dihydropyridin-3-yl)pyrrolidine-2- carboxamide |
| 2a | | (R)-1-(4-(5-cyclopropyl-1H-pyrazol-3-ylamino)- |
| | | 6,7-dihydro-5H-cyclopenta[d]pyrimidin-2-yl)- |
| | | N-(6-oxo-1,6-dihydropyridin-3-yl)pyrrolidine-2- |
| | | carboxamide |
| 2b | | (S)-1-(4-(5-cyclopropyl-1H-pyrazol-3-ylamino)- |
| | | 6,7-dihydro-5H-cyclopenta[d]pyrimidin-2-yl)- |
| | | N-(6-oxo-1,6-dihydropyridin-3-yl)pyrrolidine-2- |
| | | carboxamide |
| 3 | 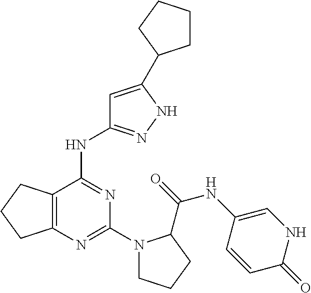 [SMILES: O=C(Nc1ccc(=O)[nH]c1)C1CCCN1c1nc2c(c(Nc3cc(C4CCCC4)[nH]n3)n1)CCC2] | 1-(4-(5-cyclopentyl-1H-pyrazol-3-ylamino)-6,7- dihydro-5H-cyclopenta[d]pyrimidin-2-yl)-N-(6- oxo-1,6-dihydropyridin-3-yl)pyrrolidine-2- carboxamide |
| 3a | | (R)-1-(4-(5-cyclopentyl-1H-pyrazol-3-ylamino)- |
| | | 6,7-dihydro-5H-cyclopenta[d]pyrimidin-2-yl)- |
| | | N-(6-oxo-1,6-dihydropyridin-3-yl)pyrrolidine-2- |
| | | carboxamide |
| 3b | | (S)-1-(4-(5-cyclopentyl-1H-pyrazol-3-ylamino)- |
| | | 6,7-dihydro-5H-cyclopenta[d]pyrimidin-2-yl)- |
| | | N-(6-oxo-1,6-dihydropyridin-3-yl)pyrrolidine-2- |
| | | carboxamide |
| 4 | 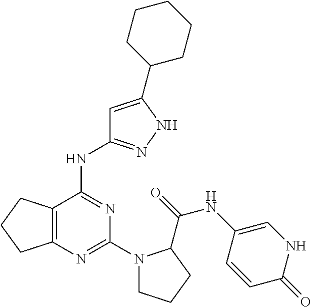 [SMILES: O=C(Nc1ccc(=O)[nH]c1)C1CCCN1c1nc2c(c(Nc3cc(C4CCCCC4)[nH]n3)n1)CCC2] | 1-(4-(5-cyclohexyl-1H-pyrazol-3-ylamino)-6,7- dihydro-5H-cyclopenta[d]pyrimidin-2-yl)-N-(6- oxo-1,6-dihydropyridin-3-yl)pyrrolidine-2- carboxamide |
| 4a | | (R)-1-(4-(5-cyclohexyl-1H-pyrazol-3-ylamino)- |
| | | 6,7-dihydro-5H-cyclopenta[d]pyrimidin-2-yl)- |
| | | N-(6-oxo-1,6-dihydropyridin-3-yl)pyrrolidine-2- |
| | | carboxamide |
| 4b | | (S)-1-(4-(5-cyclohexyl-1H-pyrazol-3-ylamino)- |
| | | 6,7-dihydro-5H-cyclopenta[d]pyrimidin-2-yl)- |
| | | N-(6-oxo-1,6-dihydropyridin-3-yl)pyrrolidine-2- |
| | | carboxamide |
| 5 | 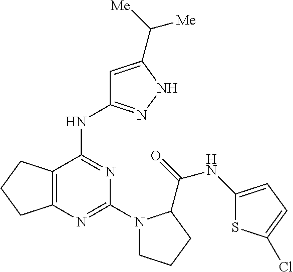 [SMILES: CC(C)c1cc(Nc2nc(N3CCCC3C(=O)Nc3ccc(Cl)s3)nc3c2CCC3)n[nH]1] | N-(5-chlorothiophen-2-yl)-1-(4-(5-isopropyl- 1H-pyrazol-3-ylamino)-6,7-dihydro-5H- cyclopenta[d]pyrimidin-2-yl)pyrrolidine-2- carboxamide |
| 5a | | (R)-N-(5-chlorothiophen-2-yl)-1-(4-(5- |
| | | isopropyl-1H-pyrazol-3-ylamino)-6,7-dihydro- |
| | | 5H-cyclopenta[d]pyrimidin-2-yl)pyrrolidine-2- |
| | | carboxamide |
| 5b | | (S)-N-(5-chlorothiophen-2-yl)-1-(4-(5-isopropyl- |
| | | 1H-pyrazol-3-ylamino)-6,7-dihydro-5H- |
| | | cyclopenta[d]pyrimidin-2-yl)pyrrolidine-2- |
| | | carboxamide |
| 6 | 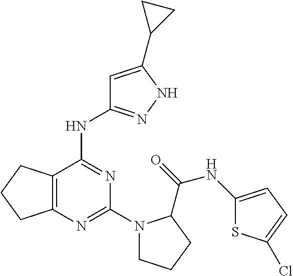 [SMILES: O=C(Nc1ccc(Cl)s1)C1CCCN1c1nc2c(c(Nc3cc(C4CC4)[nH]n3)n1)CCC2] | N-(5-chlorothiophen-2-yl)-1-(4-(5-cyclopropyl- 1H-pyrazol-3-ylamino)-6,7-dihydro-5H- cyclopenta[d]pyrimidin-2-yl)pyrrolidine-2- carboxamide |
| 6a | | (R)-N-(5-chlorothiophen-2-yl)-1-(4-(5- |
| | | cyclopropyl-1H-pyrazol-3-ylamino)-6,7- |
| | | dihydro-5H-cyclopenta[d]pyrimidin-2- |
| | | yl)pyrrolidine-2-carboxamide |
| 6b | | (S)-N-(5-chlorothiophen-2-yl)-1-(4-(5- |
| | | cyclopropyl-1H-pyrazol-3-ylamino)-6,7- |
| | | dihydro-5H-cyclopenta[d]pyrimidin-2- |
| | | yl)pyrrolidine-2-carboxamide |
| 7 | 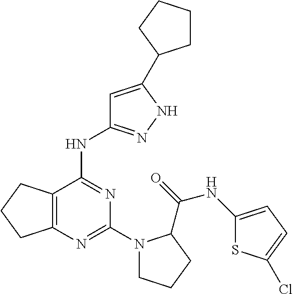 [SMILES: O=C(Nc1ccc(Cl)s1)C1CCCN1c1nc2c(c(Nc3cc(C4CCCC4)[nH]n3)n1)CCC2] | N-(5-chlorothiophen-2-yl)-1-(4-(5-cyclopentyl- 1H-pyrazol-3-ylamino)-6,7-dihydro-5H- cyclopenta[d]pyrimidin-2-yl)pyrrolidine-2- carboxamide |
| 7a | | (R)-N-(5-chlorothiophen-2-yl)-1-(4-(5- |
| | | cyclopentyl-1H-pyrazol-3-ylamino)-6,7- |
| | | dihydro-5H-cyclopenta[d]pyrimidin-2- |
| | | yl)pyrrolidine-2-carboxamide |
| 7b | | (S)-N-(5-chlorothiophen-2-yl)-1-(4-(5- |
| | | cyclopentyl-1H-pyrazol-3-ylamino)-6,7- |
| | | dihydro-5H-cyclopenta[d]pyrimidin-2- |
| | | yl)pyrrolidine-2-carboxamide |
| 8 | 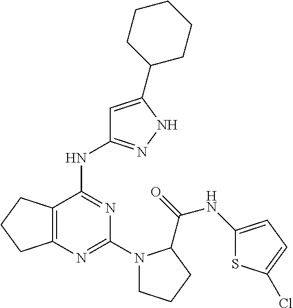 [SMILES: O=C(Nc1ccc(Cl)s1)C1CCCN1c1nc2c(c(Nc3cc(C4CCCCC4)[nH]n3)n1)CCC2] | N-(5-chlorothiophen-2-yl)-1-(4-(5-cyclohexyl- 1H-pyrazol-3-ylamino)-6,7-dihydro-5H- cyclopenta[d]pyrimidin-2-yl)pyrrolidine-2- carboxamide |
| 8a | | (R)-N-(5-chlorothiophen-2-yl)-1-(4-(5- |
| | | cyclohexyl-1H-pyrazol-3-ylamino)-6,7-dihydro- |
| | | 5H-cyclopenta[d]pyrimidin-2-yl)pyrrolidine-2- |
| | | carboxamide |
| 8b | | (S)-N-(5-chlorothiophen-2-yl)-1-(4-(5- |
| | | cyclohexyl-1H-pyrazol-3-ylamino)-6,7-dihydro- |
| | | 5H-cyclopenta[d]pyrimidin-2-yl)pyrrolidine-2- |
| | | carboxamide |
| 9 | 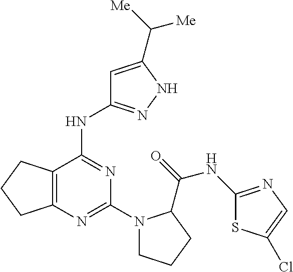 [SMILES: CC(C)c1cc(Nc2nc(N3CCCC3C(=O)Nc3ncc(Cl)s3)nc3c2CCC3)n[nH]1] | N-(5-chlorothiazol-2-yl)-1-(4-(5-isopropyl-1H- pyrazol-3-ylamino)-6,7-dihydro-5H- cyclopenta[d]pyrimidin-2-yl)pyrrolidine-2- carboxamide |
| 9a | | (R)-N-(5-chlorothiazol-2-yl)-1-(4-(5-isopropyl- |
| | | 1H-pyrazol-3-ylamino)-6,7-dihydro-5H- |
| | | cyclopenta[d]pyrimidin-2-yl)pyrrolidine-2- |
| | | carboxamide |
| 9b | | (S)-N-(5-chlorothiazol-2-yl)-1-(4-(5-isopropyl- |
| | | 1H-pyrazol-3-ylamino)-6,7-dihydro-5H- |
| | | cyclopenta[d]pyrimidin-2-yl)pyrrolidine-2- |
| | | carboxamide |
| 10 | 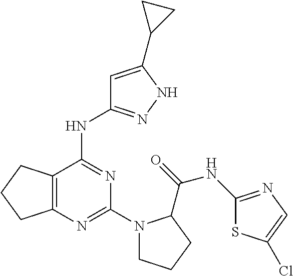 [SMILES: O=C(Nc1ncc(Cl)s1)C1CCCN1c1nc2c(c(Nc3cc(C4CC4)[nH]n3)n1)CCC2] | N-(5-chlorothiazol-2-yl)-1-(4-(5-cyclopropyl- 1H-pyrazol-3-ylamino)-6,7-dihydro-5H- cyclopenta[d]pyrimidin-2-yl)pyrrolidine-2- carboxamide |
| 10a | | (R)-N-(5-chlorothiazol-2-yl)-1-(4-(5- |
| | | cyclopropyl-1H-pyrazol-3-ylamino)-6,7- |
| | | dihydro-5H-cyclopenta[d]pyrimidin-2- |
| | | yl)pyrrolidine-2-carboxamide |
| 10b | | (S)-N-(5-chlorothiazol-2-yl)-1-(4-(5- |
| | | cyclopropyl-1H-pyrazol-3-ylamino)-6,7- |
| | | dihydro-5H-cyclopenta[d]pyrimidin-2- |
| | | yl)pyrrolidine-2-carboxamide |
| 11 | 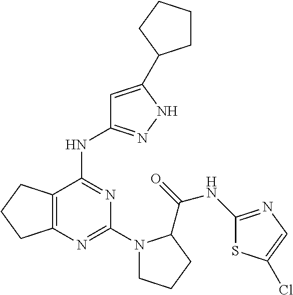 [SMILES: O=C(Nc1ncc(Cl)s1)C1CCCN1c1nc2c(c(Nc3cc(C4CCCC4)[nH]n3)n1)CCC2] | N-(5-chlorothiazol-2-yl)-1-(4-(5-cyclopentyl- 1H-pyrazol-3-ylamino)-6,7-dihydro-5H- cyclopenta[d]pyrimidin-2-yl)pyrrolidine-2- carboxamide |
| 11a | | (R)-N-(5-chlorothiazol-2-yl)-1-(4-(5- |
| | | cyclopentyl-1H-pyrazol-3-ylamino)-6,7- |
| | | dihydro-5H-cyclopenta[d]pyrimidin-2- |
| | | yl)pyrrolidine-2-carboxamide |
| 11b | | (S)-N-(5-chlorothiazol-2-yl)-1-(4-(5- |
| | | cyclopentyl-1H-pyrazol-3-ylamino)-6,7- |
| | | dihydro-5H-cyclopenta[d]pyrimidin-2- |
| | | yl)pyrrolidine-2-carboxamide |
| 12 | 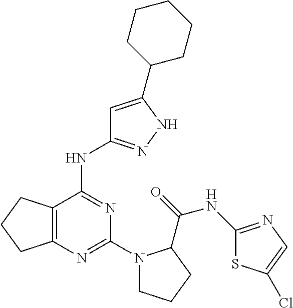 [SMILES: O=C(Nc1ncc(Cl)s1)C1CCCN1c1nc2c(c(Nc3cc(C4CCCCC4)[nH]n3)n1)CCC2] | N-(5-chlorothiazol-2-yl)-1-(4-(5-cyclohexyl-1H- pyrazol-3-ylamino)-6,7-dihydro-5H- cyclopenta[d]pyrimidin-2-yl)pyrrolidine-2- carboxamide |
| 12a | | (R)-N-(5-chlorothiazol-2-yl)-1-(4-(5-cyclohexyl- |
| | | 1H-pyrazol-3-ylamino)-6,7-dihydro-5H- |
| | | cyclopenta[d]pyrimidin-2-yl)pyrrolidine-2- |
| | | carboxamide |
| 12b | | (S)-N-(5-chlorothiazol-2-yl)-1-(4-(5-cyclohexyl- |
| | | 1H-pyrazol-3-ylamino)-6,7-dihydro-5H- |
| | | cyclopenta[d]pyrimidin-2-yl)pyrrolidine-2- |
| | | carboxamide |
| 13 | 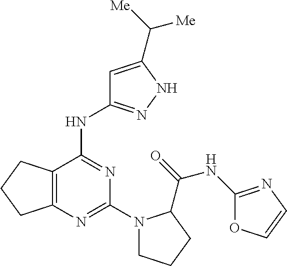 [SMILES: CC(C)c1cc(Nc2nc(N3CCCC3C(=O)Nc3ncco3)nc3c2CCC3)n[nH]1] | 1-(4-(5-isopropyl-1H-pyrazol-3-ylamino)-6,7- dihydro-5H-cyclopenta[d]pyrimidin-2-yl)-N- (oxazol-2-yl)pyrrolidine-2-carboxamide |
| 13a | | (R)-1-(4-(5-isopropyl-1H-pyrazol-3-ylamino)- |
| | | 6,7-dihydro-5H-cyclopenta[d]pyrimidin-2-yl)- |
| | | N-(oxazol-2-yl)pyrrolidine-2-carboxamide |
| 13b | | (S)-1-(4-(5-isopropyl-1H-pyrazol-3-ylamino)- |
| | | 6,7-dihydro-5H-cyclopenta[d]pyrimidin-2-yl)- |
| | | N-(oxazol-2-yl)pyrrolidine-2-carboxamide |
| 14 | 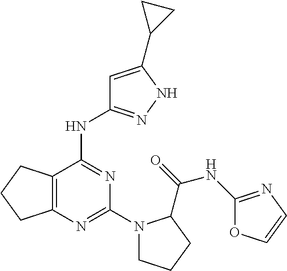 [SMILES: O=C(Nc1ncco1)C1CCCN1c1nc2c(c(Nc3cc(C4CC4)[nH]n3)n1)CCC2] | 1-(4-(5-cyclopropyl-1H-pyrazol-3-ylamino)-6,7- dihydro-5H-cyclopenta[d]pyrimidin-2-yl)-N- (oxazol-2-yl)pyrrolidine-2-carboxamide |
| 14a | | (R)-1-(4-(5-cyclopropyl-1H-pyrazol-3-ylamino)- |
| | | 6,7-dihydro-5H-cyclopenta[d]pyrimidin-2-yl)- |
| | | N-(oxazol-2-yl)pyrrolidine-2-carboxamide |
| 14b | | (S)-1-(4-(5-cyclopropyl-1H-pyrazol-3-ylamino)- |
| | | 6,7-dihydro-5H-cyclopenta[d]pyrimidin-2-yl)- |
| | | N-(oxazol-2-yl)pyrrolidine-2-carboxamide |
| 15 | 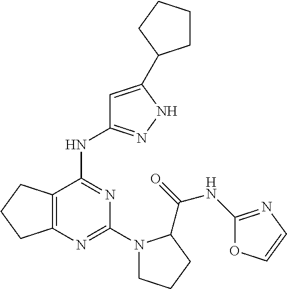 [SMILES: O=C(Nc1ncco1)C1CCCN1c1nc2c(c(Nc3cc(C4CCCC4)[nH]n3)n1)CCC2] | 1-(4-(5-cyclopentyl-1H-pyrazol-3-ylamino)-6,7- dihydro-5H-cyclopenta[d]pyrimidin-2-yl)-N- (oxazol-2-yl)pyrrolidine-2-carboxamide |
| 15a | | (R)-1-(4-(5-cyclopentyl-1H-pyrazol-3-ylamino)- |
| | | 6,7-dihydro-5H-cyclopenta[d]pyrimidin-2-yl)- |
| | | N-(oxazol-2-yl)pyrrolidine-2-carboxamide |
| 15b | | (S)-1-(4-(5-cyclopentyl-1H-pyrazol-3-ylamino)- |
| | | 6,7-dihydro-5H-cyclopenta[d]pyrimidin-2-yl)- |
| | | N-(oxazol-2-yl)pyrrolidine-2-carboxamide |
| 16 | 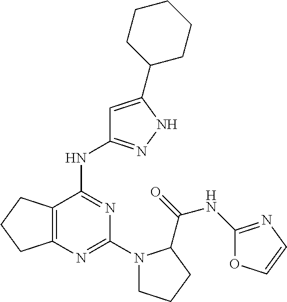 [SMILES: O=C(Nc1ncco1)C1CCCN1c1nc2c(c(Nc3cc(C4CCCCC4)[nH]n3)n1)CCC2] | 1-(4-(5-cyclohexyl-1H-pyrazol-3-ylamino)-6,7- dihydro-5H-cyclopenta[d]pyrimidin-2-yl)-N- (oxazol-2-yl)pyrrolidine-2-carboxamide |
| 16a | | (R)-1-(4-(5-cyclohexyl-1H-pyrazol-3-ylamino)- |
| | | 6,7-dihydro-5H-cyclopenta[d]pyrimidin-2-yl)- |
| | | N-(oxazol-2-yl)pyrrolidine-2-carboxamide |
| 16b | | (S)-1-(4-(5-cyclohexyl-1H-pyrazol-3-ylamino)- |
| | | 6,7-dihydro-5H-cyclopenta[d]pyrimidin-2-yl)- |
| | | N-(oxazol-2-yl)pyrrolidine-2-carboxamide |
| 17 | 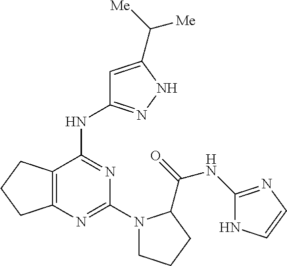 [SMILES: CC(C)c1cc(Nc2nc(N3CCCC3C(=O)Nc3ncc[nH]3)nc3c2CCC3)n[nH]1] | N-(1H-imidazol-2-yl)-1-(4-(5-isopropyl-1H- pyrazol-3-ylamino)-6,7-dihydro-5H- cyclopenta[d]pyrimidin-2-yl)pyrrolidine-2- carboxamide |
| 17a | | (R)-N-(1H-imidazol-2-yl)-1-(4-(5-isopropyl-1H- |
| | | pyrazol-3-ylamino)-6,7-dihydro-5H- |
| | | cyclopenta[d]pyrimidin-2-yl)pyrrolidine-2- |
| | | carboxamide |
| 17b | | (S)-N-(1H-imidazol-2-yl)-1-(4-(5-isopropyl-1H- |
| | | pyrazol-3-ylamino)-6,7-dihydro-5H- |
| | | cyclopenta[d]pyrimidin-2-yl)pyrrolidine-2- |
| | | carboxamide |
| 18 | 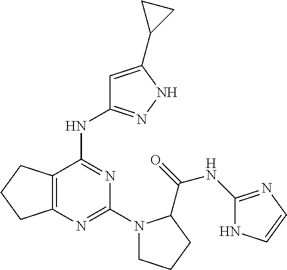 [SMILES: O=C(Nc1ncc[nH]1)C1CCCN1c1nc2c(c(Nc3cc(C4CC4)[nH]n3)n1)CCC2] | 1-(4-(5-cyclopropyl-1H-pyrazol-3-ylamino)-6,7- dihydro-5H-cyclopenta[d]pyrimidin-2-yl)-N- (1H-imidazol-2-yl)pyrrolidine-2-carboxamide |
| 18a | | (R)-1-(4-(5-cyclopropyl-1H-pyrazol-3-ylamino)- |
| | | 6,7-dihydro-5H-cyclopenta[d]pyrimidin-2-yl)- |
| | | N-(1H-imidazol-2-yl)pyrrolidine-2-carboxamide |
| 18b | | (S)-1-(4-(5-cyclopropyl-1H-pyrazol-3-ylamino)- |
| | | 6,7-dihydro-5H-cyclopenta[d]pyrimidin-2-yl)- |
| | | N-(1H-imidazol-2-yl)pyrrolidine-2-carboxamide |
| 19 | 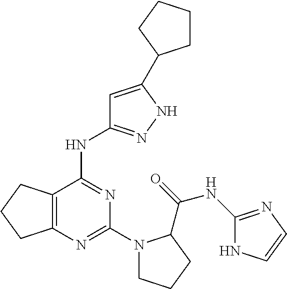 [SMILES: O=C(Nc1ncc[nH]1)C1CCCN1c1nc2c(c(Nc3cc(C4CCCC4)[nH]n3)n1)CCC2] | 1-(4-(5-cyclopentyl-1H-pyrazol-3-ylamino)-6,7- dihydro-5H-cyclopenta[d]pyrimidin-2-yl)-N- (1H-imidazol-2-yl)pyrrolidine-2-carboxamide |
| 19a | | (R)-1-(4-(5-cyclopentyl-1H-pyrazol-3-ylamino)- |
| | | 6,7-dihydro-5H-cyclopenta[d]pyrimidin-2-yl)- |
| | | N-(1H-imidazol-2-yl)pyrrolidine-2-carboxamide |
| 19b | | (S)-1-(4-(5-cyclopentyl-1H-pyrazol-3-ylamino)- |
| | | 6,7-dihydro-5H-cyclopenta[d]pyrimidin-2-yl)- |
| | | N-(1H-imidazol-2-yl)pyrrolidine-2-carboxamide |
| 20 | 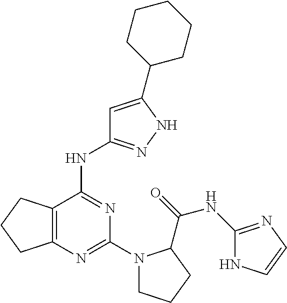 [SMILES: O=C(Nc1ncc[nH]1)C1CCCN1c1nc2c(c(Nc3cc(C4CCCCC4)[nH]n3)n1)CCC2] | 1-(4-(5-cyclohexyl-1H-pyrazol-3-ylamino)-6,7- dihydro-5H-cyclopenta[d]pyrimidin-2-yl)-N- (1H-imidazol-2-yl)pyrrolidine-2-carboxamide |
| 20a | | (R)-1-(4-(5-cyclohexyl-1H-pyrazol-3-ylamino)- |
| | | 6,7-dihydro-5H-cyclopenta[d]pyrimidin-2-yl)- |
| | | N-(1H-imidazol-2-yl)pyrrolidine-2-carboxamide |
| 20b | | (S)-1-(4-(5-cyclohexyl-1H-pyrazol-3-ylamino)- |
| | | 6,7-dihydro-5H-cyclopenta[d]pyrimidin-2-yl)- |
| | | N-(1H-imidazol-2-yl)pyrrolidine-2-carboxamide |
| 21 | 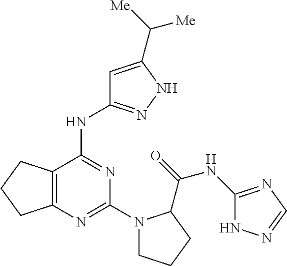 [SMILES: CC(C)c1cc(Nc2nc(N3CCCC3C(=O)Nc3ncn[nH]3)nc3c2CCC3)n[nH]1] | 1-(4-(5-isopropyl-1H-pyrazol-3-ylamino)-6,7- dihydro-5H-cyclopenta[d]pyrimidin-2-yl)-N- (1H-1,2,4-triazol-5-yl)pyrrolidine-2- carboxamide |
| 21a | | (R)-1-(4-(5-isopropyl-1H-pyrazol-3-ylamino)- |
| | | 6,7-dihydro-5H-cyclopenta[d]pyrimidin-2-yl)- |
| | | N-(1H-1,2,4-triazol-5-yl)pyrrolidine-2- |
| | | carboxamide |
| 21b | | (S)-1-(4-(5-isopropyl-1H-pyrazol-3-ylamino)- |
| | | 6,7-dihydro-5H-cyclopenta[d]pyrimidin-2-yl)- |
| | | N-(1H-1,2,4-triazol-5-yl)pyrrolidine-2- |
| | | carboxamide |
| 22 | 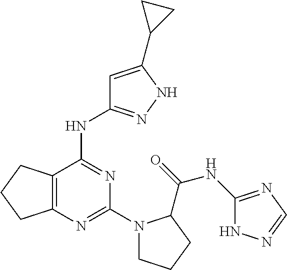 [SMILES: O=C(Nc1ncn[nH]1)C1CCCN1c1nc2c(c(Nc3cc(C4CC4)[nH]n3)n1)CCC2] | 1-(4-(5-cyclopropyl-1H-pyrazol-3-ylamino)-6,7- dihydro-5H-cyclopenta[d]pyrimidin-2-yl)-N- (1H-1,2,4-triazol-5-yl)pyrrolidine-2- carboxamide |
| 22a | | (R)-1-(4-(5-cyclopropyl-1H-pyrazol-3-ylamino)- |
| | | 6,7-dihydro-5H-cyclopenta[d]pyrimidin-2-yl)- |
| | | N-(1H-1,2,4-triazol-5-yl)pyrrolidine-2- |
| | | carboxamide |
| 22b | | (S)-1-(4-(5-cyclopropyl-1H-pyrazol-3-ylamino)- |
| | | 6,7-dihydro-5H-cyclopenta[d]pyrimidin-2-yl)- |
| | | N-(1H-1,2,4-triazol-5-yl)pyrrolidine-2- |
| | | carboxamide |
| 23 | 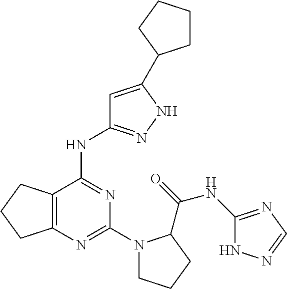 [SMILES: O=C(Nc1ncn[nH]1)C1CCCN1c1nc2c(c(Nc3cc(C4CCCC4)[nH]n3)n1)CCC2] | 1-(4-(5-cyclopentyl-1H-pyrazol-3-ylamino)-6,7- dihydro-5H-cyclopenta[d]pyrimidin-2-yl)-N- (1H-1,2,4-triazol-5-yl)pyrrolidine-2- carboxamide |
| 23a | | (R)-1-(4-(5-cyclopentyl-1H-pyrazol-3-ylamino)- |
| | | 6,7-dihydro-5H-cyclopenta[d]pyrimidin-2-yl)- |
| | | N-(1H-1,2,4-triazol-5-yl)pyrrolidine-2- |
| | | carboxamide |
| 23b | | (S)-1-(4-(5-cyclopentyl-1H-pyrazol-3-ylamino)- |
| | | 6,7-dihydro-5H-cyclopenta[d]pyrimidin-2-yl)- |
| | | N-(1H-1,2,4-triazol-5-yl)pyrrolidine-2- |
| | | carboxamide |
| 24 | 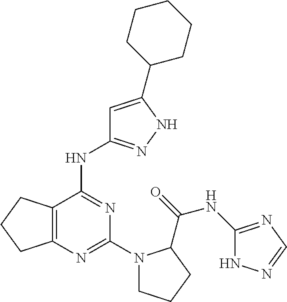 [SMILES: O=C(Nc1ncn[nH]1)C1CCCN1c1nc2c(c(Nc3cc(C4CCCCC4)[nH]n3)n1)CCC2] | 1-(4-(5-cyclohexyl-1H-pyrazol-3-ylamino)-6,7- dihydro-5H-cyclopenta[d]pyrimidin-2-yl)-N- (1H-1,2,4-triazol-5-yl)pyrrolidine-2- carboxamide |
| 24a | | (R)-1-(4-(5-cyclohexyl-1H-pyrazol-3-ylamino)- |
| | | 6,7-dihydro-5H-cyclopenta[d]pyrimidin-2-yl)- |
| | | N-(1H-1,2,4-triazol-5-yl)pyrrolidine-2- |
| | | carboxamide |
| 24b | | (S)-1-(4-(5-cyclohexyl-1H-pyrazol-3-ylamino)- |
| | | 6,7-dihydro-5H-cyclopenta[d]pyrimidin-2-yl)- |
| | | N-(1H-1,2,4-triazol-5-yl)pyrrolidine-2- |
| | | carboxamide |
| 25 | 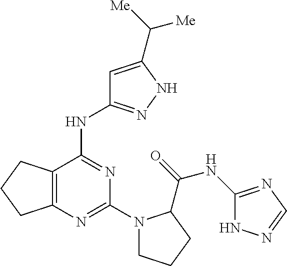 [SMILES: CC(C)c1cc(Nc2nc(N3CCCC3C(=O)Nc3ncn[nH]3)nc3c2CCC3)n[nH]1] | 1-(4-(5-isopropyl-1H-pyrazol-3-ylamino)-6,7- dihydro-5H-cyclopenta[d]pyrimidin-2-yl)-N- (1H-pyrazol-5-yl)pyrrolidine-2-carboxamide |
| 25a | | (R)-1-(4-(5-isopropyl-1H-pyrazol-3-ylamino)- |
| | | 6,7-dihydro-5H-cyclopenta[d]pyrimidin-2-yl)- |
| | | N-(1H-pyrazol-5-yl)pyrrolidine-2-carboxamide |
| 25b | | (S)-1-(4-(5-isopropyl-1H-pyrazol-3-ylamino)- |
| | | 6,7-dihydro-5H-cyclopenta[d]pyrimidin-2-yl)- |
| | | N-(1H-pyrazol-5-yl)pyrrolidine-2-carboxamide |
| 26 | 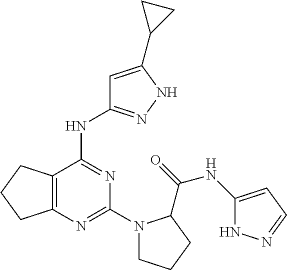 [SMILES: O=C(Nc1ccn[nH]1)C1CCCN1c1nc2c(c(Nc3cc(C4CC4)[nH]n3)n1)CCC2] | 1-(4-(5-cyclopropyl-1H-pyrazol-3-ylamino)-6,7- dihydro-5H-cyclopenta[d]pyrimidin-2-yl)-N- (1H-pyrazol-5-yl)pyrrolidine-2-carboxamide |
| 26a | | (R)-1-(4-(5-cyclopropyl-1H-pyrazol-3-ylamino)- |
| | | 6,7-dihydro-5H-cyclopenta[d]pyrimidin-2-yl)- |
| | | N-(1H-pyrazol-5-yl)pyrrolidine-2-carboxamide |
| 26b | | (S)-1-(4-(5-cyclopropyl-1H-pyrazol-3-ylamino)- |
| | | 6,7-dihydro-5H-cyclopenta[d]pyrimidin-2-yl)- |
| | | N-(1H-pyrazol-5-yl)pyrrolidine-2-carboxamide |
| 27 | 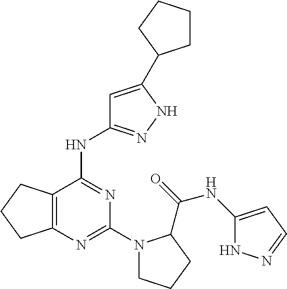 [SMILES: O=C(Nc1ccn[nH]1)C1CCCN1c1nc2c(c(Nc3cc(C4CCCC4)[nH]n3)n1)CCC2] | 1-(4-(5-cyclopentyl-1H-pyrazol-3-ylamino)-6,7- dihydro-5H-cyclopenta[d]pyrimidin-2-yl)-N- (1H-pyrazol-5-yl)pyrrolidine-2-carboxamide |
| 27b | | (R)-1-(4-(5-cyclopentyl-1H-pyrazol-3-ylamino)- |
| | | 6,7-dihydro-5H-cyclopenta[d]pyrimidin-2-yl)- |
| | | N-(1H-pyrazol-5-yl)pyrrolidine-2-carboxamide |
| 27b | | (S)-1-(4-(5-cyclopentyl-1H-pyrazol-3-ylamino)- |
| | | 6,7-dihydro-5H-cyclopenta[d]pyrimidin-2-yl)- |
| | | N-(1H-pyrazol-5-yl)pyrrolidine-2-carboxamide |
| 28 | 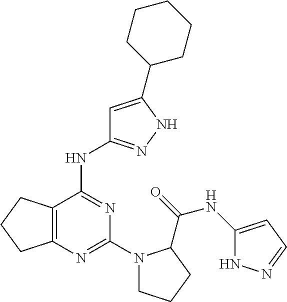 [SMILES: O=C(Nc1ccn[nH]1)C1CCCN1c1nc2c(c(Nc3cc(C4CCCCC4)[nH]n3)n1)CCC2] | 1-(4-(5-cyclohexyl-1H-pyrazol-3-ylamino)-6,7- dihydro-5H-cyclopenta[d]pyrimidin-2-yl)-N- (1H-pyrazol-5-yl)pyrrolidine-2-carboxamide |
| 28a | | (R)-1-(4-(5-cyclohexyl-1H-pyrazol-3-ylamino)- |
| | | 6,7-dihydro-5H-cyclopenta[d]pyrimidin-2-yl)- |
| | | N-(1H-pyrazol-5-yl)pyrrolidine-2-carboxamide |
| 28b | | (S)-1-(4-(5-cyclohexyl-1H-pyrazol-3-ylamino)- |
| | | 6,7-dihydro-5H-cyclopenta[d]pyrimidin-2-yl)- |
| | | N-(1H-pyrazol-5-yl)pyrrolidine-2-carboxamide |
| 29 | 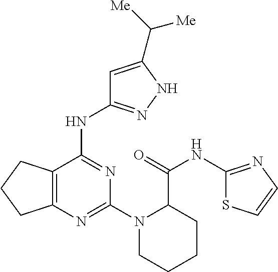 [SMILES: CC(C)c1cc(Nc2nc(N3CCCCC3C(=O)Nc3nccs3)nc3c2CCC3)n[nH]1] | 1-(4-(5-isopropyl-1H-pyrazol-3-ylamino)-6,7- dihydro-5H-cyclopenta[d]pyrimidin-2-yl)-N- (thiazol-2-yl)piperidine-2-carboxamide |
| 29a | | (R)-1-(4-(5-isopropyl-1H-pyrazol-3-ylamino)- |
| | | 6,7-dihydro-5H-cyclopenta[d]pyrimidin-2-yl)- |
| | | N-(thiazol-2-yl)piperidine-2-carboxamide |
| 29b | | (S)-1-(4-(5-isopropyl-1H-pyrazol-3-ylamino)- |
| | | 6,7-dihydro-5H-cyclopenta[d]pyrimidin-2-yl)- |
| | | N-(thiazol-2-yl)piperidine-2-carboxamide |
| 30 | 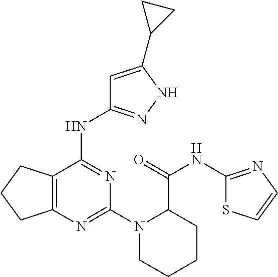 [SMILES: O=C(Nc1nccs1)C1CCCCN1c1nc2c(c(Nc3cc(C4CC4)[nH]n3)n1)CCC2] | 1-(4-(5-cyclopropyl-1H-pyrazol-3-ylamino)-6,7- dihydro-5H-cyclopenta[d]pyrimidin-2-yl)-N- (thiazol-2-yl)piperidine-2-carboxamide |
| 30a | | (R)-1-(4-(5-cyclopropyl-1H-pyrazol-3-ylamino)- |
| | | 6,7-dihydro-5H-cyclopenta[d]pyrimidin-2-yl)- |
| | | N-(thiazol-2-yl)piperidine-2-carboxamide |
| 30b | | (S)-1-(4-(5-cyclopropyl-1H-pyrazol-3-ylamino)- |
| | | 6,7-dihydro-5H-cyclopenta[d]pyrimidin-2-yl)- |
| | | N-(thiazol-2-yl)piperidine-2-carboxamide |
| 31 | 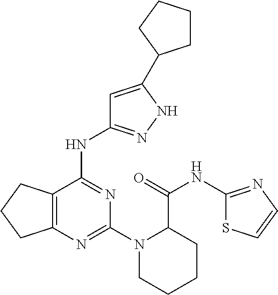 [SMILES: O=C(Nc1nccs1)C1CCCCN1c1nc2c(c(Nc3cc(C4CCCC4)[nH]n3)n1)CCC2] | 1-(4-(5-cyclopentyl-1H-pyrazol-3-ylamino)-6,7- dihydro-5H-cyclopenta[d]pyrimidin-2-yl)-N- (thiazol-2-yl)piperidine-2-carboxamide |
| 31a | | (R)-1-(4-(5-cyclopentyl-1H-pyrazol-3-ylamino)- |
| | | 6,7-dihydro-5H-cyclopenta[d]pyrimidin-2-yl)- |
| | | N-(thiazol-2-yl)piperidine-2-carboxamide |
| 31b | | (S)-1-(4-(5-cyclopentyl-1H-pyrazol-3-ylamino)- |
| | | 6,7-dihydro-5H-cyclopenta[d]pyrimidin-2-yl)- |
| | | N-(thiazol-2-yl)piperidine-2-carboxamide |
| 32 | 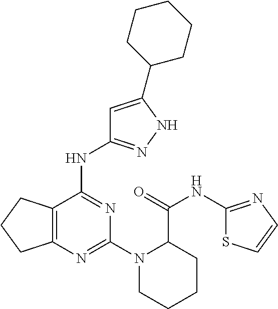 [SMILES: O=C(Nc1nccs1)C1CCCCN1c1nc2c(c(Nc3cc(C4CCCCC4)[nH]n3)n1)CCC2] | 1-(4-(5-cyclohexyl-1H-pyrazol-3-ylamino)-6,7- dihydro-5H-cyclopenta[d]pyrimidin-2-yl)-N- (thiazol-2-yl)piperidine-2-carboxamide |
| 32a | | (R)-1-(4-(5-cyclohexyl-1H-pyrazol-3-ylamino)- |
| | | 6,7-dihydro-5H-cyclopenta[d]pyrimidin-2-yl)- |
| | | N-(thiazol-2-yl)piperidine-2-carboxamide |
| 32b | | (S)-1-(4-(5-cyclohexyl-1H-pyrazol-3-ylamino)- |
| | | 6,7-dihydro-5H-cyclopenta[d]pyrimidin-2-yl)- |
| | | N-(thiazol-2-yl)piperidine-2-carboxamide |
| 33 | 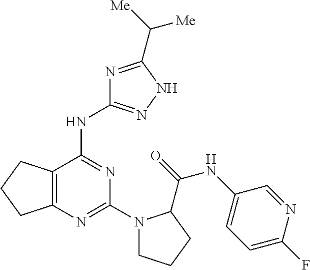 [SMILES: CC(C)c1nc(Nc2nc(N3CCCC3C(=O)Nc3ccc(F)nc3)nc3c2CCC3)n[nH]1] | N-(6-fluoropyridin-3-yl)-1-(4-(5-isopropyl-1H- 1,2,4-triazol-3-ylamino)-6,7-dihydro-5H- cyclopenta[d]pyrimidin-2-yl)pyrrolidine-2- carboxamide |
| 33a | | (R)-N-(6-fluoropyridin-3-yl)-1-(4-(5-isopropyl- |
| | | 1H-1,2,4-triazol-3-ylamino)-6,7-dihydro-5H- |
| | | cyclopenta[d]pyrimidin-2-yl)pyrrolidine-2- |
| | | carboxamide |
| 33b | | (S)-N-(6-fluoropyridin-3-yl)-1-(4-(5-isopropyl- |
| | | 1H-1,2,4-triazol-3-ylamino)-6,7-dihydro-5H- |
| | | cyclopenta[d]pyrimidin-2-yl)pyrrolidine-2- |
| | | carboxamide |
| 34 | 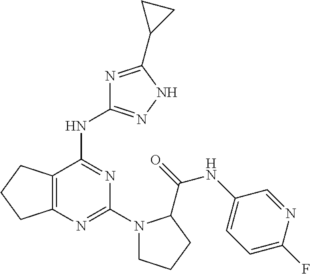 [SMILES: O=C(Nc1ccc(F)nc1)C1CCCN1c1nc2c(c(Nc3n[nH]c(C4CC4)n3)n1)CCC2] | 1-(4-(5-cyclopropyl-1H-1,2,4-triazol-3- ylamino)-6,7-dihydro-5H- cyclopenta[d]pyrimidin-2-yl)-N-(6- fluoropyridin-3-yl)pyrrolidine-2-carboxamide |
| 34a | | (R)-1-(4-(5-cyclopropyl-1H-1,2,4-triazol-3- |
| | | ylamino)-6,7-dihydro-5H- |
| | | cyclopenta[d]pyrimidin-2-yl)-N-(6- |
| | | fluoropyridin-3-yl)pyrrolidine-2-carboxamide |
| 34b | | (S)-1-(4-(5-cyclopropyl-1H-1,2,4-triazol-3- |
| | | ylamino)-6,7-dihydro-5H- |
| | | cyclopenta[d]pyrimidin-2-yl)-N-(6- |
| | | fluoropyridin-3-yl)pyrrolidine-2-carboxamide |
| 35 | 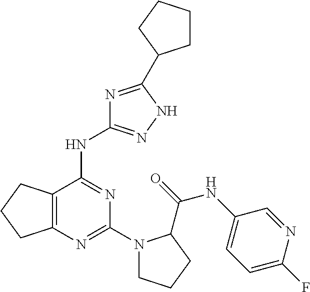 [SMILES: O=C(Nc1ccc(F)nc1)C1CCCN1c1nc2c(c(Nc3n[nH]c(C4CCCC4)n3)n1)CCC2] | 1-(4-(5-cyclopentyl-1H-1,2,4-triazol-3- ylamino)-6,7-dihydro-5H- cyclopenta[d]pyrimidin-2-yl)-N-(6- fluoropyridin-3-yl)pyrrolidine-2-carboxamide |
| 35a | | (R)-1-(4-(5-cyclopentyl-1H-1,2,4-triazol-3- |
| | | ylamino)-6,7-dihydro-5H- |
| | | cyclopenta[d]pyrimidin-2-yl)-N-(6- |
| | | fluoropyridin-3-yl)pyrrolidine-2-carboxamide |
| 35b | | (S)-1-(4-(5-cyclopentyl-1H-1,2,4-triazol-3- |
| | | ylamino)-6,7-dihydro-5H- |
| | | cyclopenta[d]pyrimidin-2-yl)-N-(6- |
| | | fluoropyridin-3-yl)pyrrolidine-2-carboxamide |
| 36 | 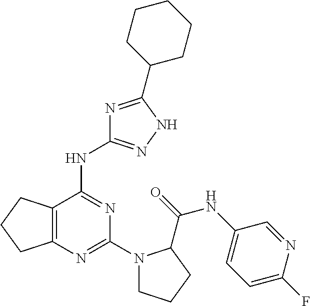 [SMILES: O=C(Nc1ccc(F)nc1)C1CCCN1c1nc2c(c(Nc3n[nH]c(C4CCCCC4)n3)n1)CCC2] | 1-(4-(5-cyclohexyl-1H-1,2,4-triazol-3-ylamino)- 6,7-dihydro-5H-cyclopenta[d]pyrimidin-2-yl)- N-(6-fluoropyridin-3-yl)pyrrolidine-2- carboxamide |
| 36a | | (R)-1-(4-(5-cyclohexyl-1H-1,2,4-triazol-3- |
| | | ylamino)-6,7-dihydro-5H- |
| | | cyclopenta[d]pyrimidin-2-yl)-N-(6- |
| | | fluoropyridin-3-yl)pyrrolidine-2-carboxamide |
| 36b | | (S)-1-(4-(5-cyclohexyl-1H-1,2,4-triazol-3- |
| | | ylamino)-6,7-dihydro-5H- |
| | | cyclopenta[d]pyrimidin-2-yl)-N-(6- |
| | | fluoropyridin-3-yl)pyrrolidine-2-carboxamide |
| 37 | 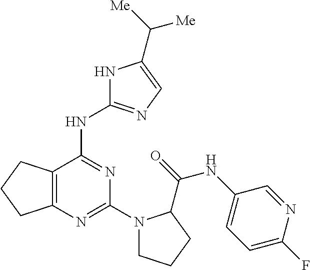 [SMILES: CC(C)c1cnc(Nc2nc(N3CCCC3C(=O)Nc3ccc(F)nc3)nc3c2CCC3)[nH]1] | N-(6-fluoropyridin-3-yl)-1-(4-(5-isopropyl-1H- imidazol-2-ylamino)-6,7-dihydro-5H- cyclopenta[d]pyrimidin-2-yl)pyrrolidine-2- carboxamide |
| 37a | | (R)-N-(6-fluoropyridin-3-yl)-1-(4-(5-isopropyl- |
| | | 1H-imidazol-2-ylamino)-6,7-dihydro-5H- |
| | | cyclopenta[d]pyrimidin-2-yl)pyrrolidine-2- |
| | | carboxamide |
| 37b | | (S)-N-(6-fluoropyridin-3-yl)-1-(4-(5-isopropyl- |
| | | 1H-imidazol-2-ylamino)-6,7-dihydro-5H- |
| | | cyclopenta[d]pyrimidin-2-yl)pyrrolidine-2- |
| | | carboxamide |
| 38 | 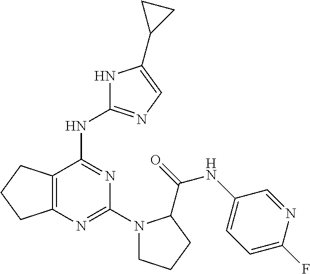 [SMILES: O=C(Nc1ccc(F)nc1)C1CCCN1c1nc2c(c(Nc3ncc(C4CC4)[nH]3)n1)CCC2] | 1-(4-(5-cyclopropyl-1H-imidazol-2-ylamino)- 6,7-dihydro-5H-cyclopenta[d]pyrimidin-2-yl)- N-(6-fluoropyridin-3-yl)pyrrolidine-2- carboxamide |
| 38a | | (R)-1-(4-(5-cyclopropyl-1H-imidazol-2- |
| | | ylamino)-6,7-dihydro-5H- |
| | | cyclopenta[d]pyrimidin-2-yl)-N-(6- |
| | | fluoropyridin-3-yl)pyrrolidine-2-carboxamide |
| 38b | | (S)-1-(4-(5-cyclopropyl-1H-imidazol-2- |
| | | ylamino)-6,7-dihydro-5H- |
| | | cyclopenta[d]pyrimidin-2-yl)-N-(6- |
| | | fluoropyridin-3-yl)pyrrolidine-2-carboxamide |
| 39 | 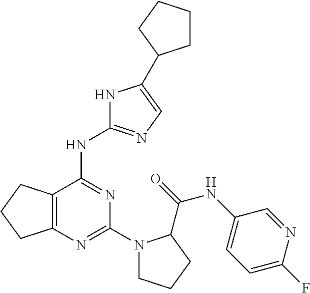 [SMILES: O=C(Nc1ccc(F)nc1)C1CCCN1c1nc2c(c(Nc3ncc(C4CCCC4)[nH]3)n1)CCC2] | 1-(4-(5-cyclopentyl-1H-imidazol-2-ylamino)- 6,7-dihydro-5H-cyclopenta[d]pyrimidin-2-yl)- N-(6-fluoropyridin-3-yl)pyrrolidine-2- carboxamide |
| 39a | | (R)-1-(4-(5-cyclopentyl-1H-imidazol-2- |
| | | ylamino)-6,7-dihydro-5H- |
| | | cyclopenta[d]pyrimidin-2-yl)-N-(6- |
| | | fluoropyridin-3-yl)pyrrolidine-2-carboxamide |
| 39b | | (S)-1-(4-(5-cyclopentyl-1H-imidazol-2- |
| | | ylamino)-6,7-dihydro-5H- |
| | | cyclopenta[d]pyrimidin-2-yl)-N-(6- |
| | | fluoropyridin-3-yl)pyrrolidine-2-carboxamide |
| 40 | 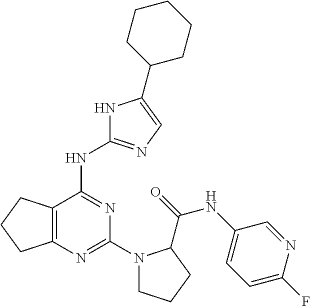 [SMILES: O=C(Nc1ccc(F)nc1)C1CCCN1c1nc2c(c(Nc3ncc(C4CCCCC4)[nH]3)n1)CCC2] | 1-(4-(5-cyclohexyl-1H-imidazol-2-ylamino)-6,7- dihydro-5H-cyclopenta[d]pyrimidin-2-yl)-N-(6- fluoropyridin-3-yl)pyrrolidine-2-carboxamide |
| 40a | | (R)-1-(4-(5-cyclohexyl-1H-imidazol-2-ylamino)- |
| | | 6,7-dihydro-5H-cyclopenta[d]pyrimidin-2-yl)- |
| | | N-(6-fluoropyridin-3-yl)pyrrolidine-2- |
| | | carboxamide |
| 40b | | (S)-1-(4-(5-cyclohexyl-1H-imidazol-2-ylamino)- |
| | | 6,7-dihydro-5H-cyclopenta[d]pyrimidin-2-yl)- |
| | | N-(6-fluoropyridin-3-yl)pyrrolidine-2- |
| | | carboxamide |
| 41 | 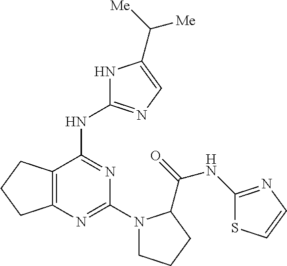 [SMILES: CC(C)c1cnc(Nc2nc(N3CCCC3C(=O)Nc3nccs3)nc3c2CCC3)[nH]1] | 1-(4-(5-isopropyl-1H-imidazol-2-ylamino)-6,7- dihydro-5H-cyclopenta[d]pyrimidin-2-yl)-N- (thiazol-2-yl)pyrrolidine-2-carboxamide |
| 41a | | (R)-1-(4-(5-isopropyl-1H-imidazol-2-ylamino)- |
| | | 6,7-dihydro-5H-cyclopenta[d]pyrimidin-2-yl)- |
| | | N-(thiazol-2-yl)pyrrolidine-2-carboxamide |
| 41b | | (S)-1-(4-(5-isopropyl-1H-imidazol-2-ylamino)- |
| | | 6,7-dihydro-5H-cyclopenta[d]pyrimidin-2-yl)- |
| | | N-(thiazol-2-yl)pyrrolidine-2-carboxamide |
| 42 | 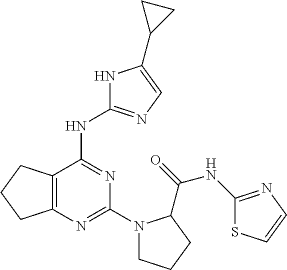 [SMILES: O=C(Nc1nccs1)C1CCCN1c1nc2c(c(Nc3ncc(C4CC4)[nH]3)n1)CCC2] | 1-(4-(5-cyclopropyl-1H-imidazol-2-ylamino)- 6,7-dihydro-5H-cyclopenta[d]pyrimidin-2-yl)- N-(thiazol-2-yl)pyrrolidine-2-carboxamide |
| 42a | | (R)-1-(4-(5-cyclopropyl-1H-imidazol-2- |
| | | ylamino)-6,7-dihydro-5H- |
| | | cyclopenta[d]pyrimidin-2-yl)-N-(thiazol-2- |
| | | yl)pyrrolidine-2-carboxamide |
| 42b | | (S)-1-(4-(5-cyclopropyl-1H-imidazol-2- |
| | | ylamino)-6,7-dihydro-5H- |
| | | cyclopenta[d]pyrimidin-2-yl)-N-(thiazol-2- |
| | | yl)pyrrolidine-2-carboxamide |
| 43 | 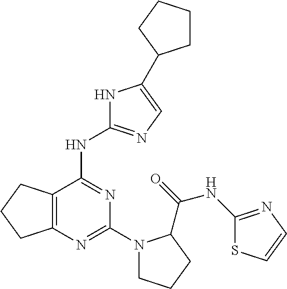 [SMILES: O=C(Nc1nccs1)C1CCCN1c1nc2c(c(Nc3ncc(C4CCCC4)[nH]3)n1)CCC2] | 1-(4-(5-cyclopentyl-1H-imidazol-2-ylamino)- 6,7-dihydro-5H-cyclopenta[d]pyrimidin-2-yl)- N-(thiazol-2-yl)pyrrolidine-2-carboxamide |
| 43a | | (R)-1-(4-(5-cyclopentyl-1H-imidazol-2- |
| | | ylamino)-6,7-dihydro-5H- |
| | | cyclopenta[d]pyrimidin-2-yl)-N-(thiazol-2- |
| | | yl)pyrrolidine-2-carboxamide |
| 43b | | (S)-1-(4-(5-cyclopentyl-1H-imidazol-2- |
| | | ylamino)-6,7-dihydro-5H- |
| | | cyclopenta[d]pyrimidin-2-yl)-N-(thiazol-2- |
| | | yl)pyrrolidine-2-carboxamide |
| 44 | 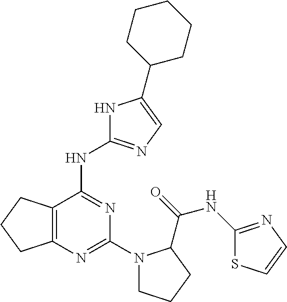 [SMILES: O=C(Nc1nccs1)C1CCCN1c1nc2c(c(Nc3ncc(C4CCCCC4)[nH]3)n1)CCC2] | 1-(4-(5-cyclohexyl-1H-imidazol-2-ylamino)-6,7- dihydro-5H-cyclopenta[d]pyrimidin-2-yl)-N- (thiazol-2-yl)pyrrolidine-2-carboxamide |
| 44a | | (R)-1-(4-(5-cyclohexyl-1H-imidazol-2-ylamino)- |
| | | 6,7-dihydro-5H-cyclopenta[d]pyrimidin-2-yl)- |
| | | N-(thiazol-2-yl)pyrrolidine-2-carboxamide |
| 44b | | (S)-1-(4-(5-cyclohexyl-1H-imidazol-2-ylamino)- |
| | | 6,7-dihydro-5H-cyclopenta[d]pyrimidin-2-yl)- |
| | | N-(thiazol-2-yl)pyrrolidine-2-carboxamide |
| 45 | 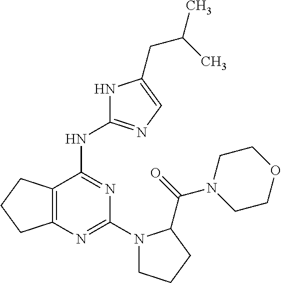 [SMILES: CC(C)Cc1cnc(Nc2nc(N3CCCC3C(=O)N3CCOCC3)nc3c2CCC3)[nH]1] | (1-(4-(5-isobutyl-1H-imidazol-2-ylamino)-6,7- dihydro-5H-cyclopenta[d]pyrimidin-2- yl)pyrrolidin-2-yl)(morpholino)methanone |
| 45a | | (R)-(1-(4-(5-isobutyl-1H-imidazol-2-ylamino)- |
| | | 6,7-dihydro-5H-cyclopenta[d]pyrimidin-2- |
| | | yl)pyrrolidin-2-yl)(morpholino)methanone |
| 45b | | (S)-(1-(4-(5-isobutyl-1H-imidazol-2-ylamino)- |
| | | 6,7-dihydro-5H-cyclopenta[d]pyrimidin-2- |
| | | yl)pyrrolidin-2-yl)(morpholino)methanone |
| 46 | 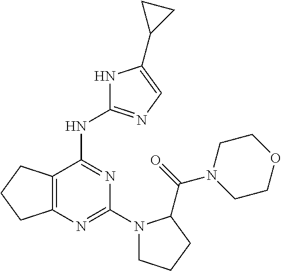 [SMILES: O=C(C1CCCN1c1nc2c(c(Nc3ncc(C4CC4)[nH]3)n1)CCC2)N1CCOCC1] | (1-(4-(5-cyclopropyl-1H-imidazol-2-ylamino)- 6,7-dihydro-5H-cyclopenta[d]pyrimidin-2- yl)pyrrolidin-2-yl)(morpholino)methanone |
| 46a | | (R)-(1-(4-(5-cyclopropyl-1H-imidazol-2- |
| | | ylamino)-6,7-dihydro-5H- |
| | | cyclopenta[d]pyrimidin-2-yl)pyrrolidin-2- |
| | | yl)(morpholino)methanone |
| 46b | | (S)-(1-(4-(5-cyclopropyl-1H-imidazol-2- |
| | | ylamino)-6,7-dihydro-5H- |
| | | cyclopenta[d]pyrimidin-2-yl)pyrrolidin-2- |
| | | yl)(morpholino)methanone |
| 47 | 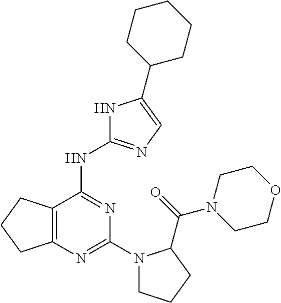 [SMILES: O=C(C1CCCN1c1nc2c(c(Nc3ncc(C4CCCCC4)[nH]3)n1)CCC2)N1CCOCC1] | (1-(4-(5-cyclohexyl-1H-imidazol-2-ylamino)- 6,7-dihydro-5H-cyclopenta[d]pyrimidin-2- yl)pyrrolidin-2-yl)(morpholino)methanone |
| 47a | | (R)-(1-(4-(5-cyclohexyl-1H-imidazol-2- |
| | | ylamino)-6,7-dihydro-5H- |
| | | cyclopenta[d]pyrimidin-2-yl)pyrrolidin-2- |
| | | yl)(morpholino)methanone |
| 47b | | (S)-(1-(4-(5-cyclohexyl-1H-imidazol-2- |
| | | ylamino)-6,7-dihydro-5H- |
| | | cyclopenta[d]pyrimidin-2-yl)pyrrolidin-2- |
| | | yl)(morpholino)methanone |
| 48 | 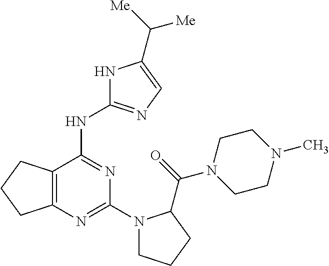 [SMILES: CC(C)c1cnc(Nc2nc(N3CCCC3C(=O)N3CCN(C)CC3)nc3c2CCC3)[nH]1] | (1-(4-(5-isopropyl-1H-imidazol-2-ylamino)-6,7- dihydro-5H-cyclopenta[d]pyrimidin-2- yl)pyrrolidin-2-yl)(4-methylpiperazin-1- yl)methanone |
| 48a | | (R)-(1-(4-(5-isopropyl-1H-imidazol-2-ylamino)- |
| | | 6,7-dihydro-5H-cyclopenta[d]pyrimidin-2- |
| | | yl)pyrrolidin-2-yl)(4-methylpiperazin-1- |
| | | yl)methanone |
| 48b | | (S)-(1-(4-(5-isopropyl-1H-imidazol-2-ylamino)- |
| | | 6,7-dihydro-5H-cyclopenta[d]pyrimidin-2- |
| | | yl)pyrrolidin-2-yl)(4-methylpiperazin-1- |
| | | yl)methanone |
| 49 | 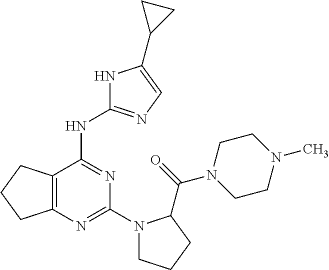 [SMILES: CN1CCN(C(=O)C2CCCN2c2nc3c(c(Nc4ncc(C5CC5)[nH]4)n2)CCC3)CC1] | (1-(4-(5-cyclopropyl-1H-imidazol-2-ylamino)- 6,7-dihydro-5H-cyclopenta[d]pyrimidin-2- yl)pyrrolidin-2-yl)(4-methylpiperazin-1- yl)methanone |
| 49a | | (R)-(1-(4-(5-cyclopropyl-1H-imidazol-2- |
| | | ylamino)-6,7-dihydro-5H- |
| | | cyclopenta[d]pyrimidin-2-yl)pyrrolidin-2-yl)(4- |
| | | methylpiperazin-1-yl)methanone |
| 49b | | (S)-(1-(4-(5-cyclopropyl-1H-imidazol-2- |
| | | ylamino)-6,7-dihydro-5H- |
| | | cyclopenta[d]pyrimidin-2-yl)pyrrolidin-2-yl)(4- |
| | | methylpiperazin-1-yl)methanone |
| 50 | 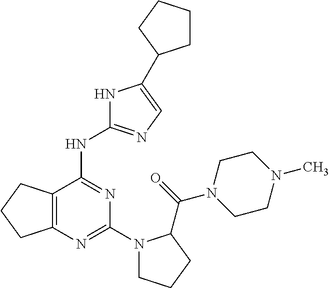 [SMILES: CN1CCN(C(=O)C2CCCN2c2nc3c(c(Nc4ncc(C5CCCC5)[nH]4)n2)CCC3)CC1] | (1-(4-(5-cyclopentyl-1H-imidazol-2-ylamino)- 6,7-dihydro-5H-cyclopenta[d]pyrimidin-2- yl)pyrrolidin-2-yl)(4-methylpiperazin-1- yl)methanone |
| 50a | | (R)-(1-(4-(5-cyclopentyl-1H-imidazol-2- |
| | | ylamino)-6,7-dihydro-5H- |
| | | cyclopenta[d]pyrimidin-2-yl)pyrrolidin-2-yl)(4- |
| | | methylpiperazin-1-yl)methanone |
| 50b | | (S)-(1-(4-(5-cyclopentyl-1H-imidazol-2- |
| | | ylamino)-6,7-dihydro-5H- |
| | | cyclopenta[d]pyrimidin-2-yl)pyrrolidin-2-yl)(4- |
| | | methylpiperazin-1-yl)methanone |
| 51 | 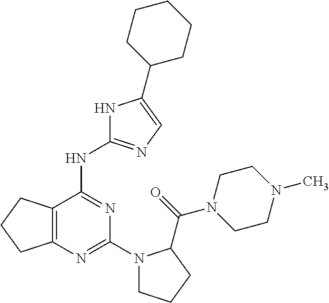 [SMILES: CN1CCN(C(=O)C2CCCN2c2nc3c(c(Nc4ncc(C5CCCCC5)[nH]4)n2)CCC3)CC1] | (1-(4-(5-cyclohexyl-1H-imidazol-2-ylamino)- 6,7-dihydro-5H-cyclopenta[d]pyrimidin-2- yl)pyrrolidin-2-yl)(4-methylpiperazin-1- yl)methanone |
| 51a | | (R)-(1-(4-(5-cyclohexyl-1H-imidazol-2- |
| | | ylamino)-6,7-dihydro-5H- |
| | | cyclopenta[d]pyrimidin-2-yl)pyrrolidin-2-yl)(4- |
| | | methylpiperazin-1-yl)methanone |
| 51b | | (S)-(1-(4-(5-cyclohexyl-1H-imidazol-2- |
| | | ylamino)-6,7-dihydro-5H- |
| | | cyclopenta[d]pyrimidin-2-yl)pyrrolidin-2-yl)(4- |
| | | methylpiperazin-1-yl)methanone |
| 52 | 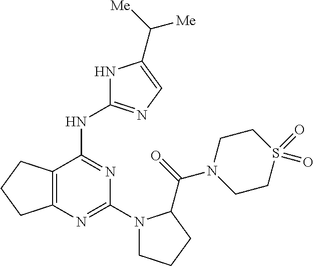 [SMILES: CC(C)c1cnc(Nc2nc(N3CCCC3C(=O)N3CCS(=O)(=O)CC3)nc3c2CCC3)[nH]1] | 4-(1-{4-[(4-isopropyl-3H-imidazol-2-yl)- amino]-5H,6H,7H-cyclopenta[d]-pyrimidin-2- yl}pyrrolidine-2-carbonyl)-1λ⁶-thiomorpholine- 1,1-dione |
| 52a | | (R)-4-(1-{4-[(4-isopropyl-3H-imidazol-2-yl)- |
| | | amino]-5H,6H,7H-cyclopenta[d]-pyrimidin-2- |
| | | yl}pyrrolidine-2-carbonyl)-1λ⁶-thiomorpholine- |
| | | 1,1-dione |
| 52b | | (S)-4-(1-{4-[(4-isopropyl-3H-imidazol-2-yl)- |
| | | amino]-5H,6H,7H-cyclopenta[d]-pyrimidin-2- |
| | | yl}pyrrolidine-2-carbonyl)-1λ⁶-thiomorpholine- |
| | | 1,1-dione |
| 53 | 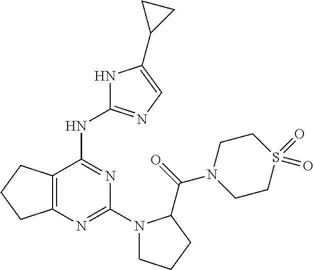 [SMILES: O=C(C1CCCN1c1nc2c(c(Nc3ncc(C4CC4)[nH]3)n1)CCC2)N1CCS(=O)(=O)CC1] | 4-(1-{4-[(4-cyclopropyl-3H-imidazol-2- yl)amino]-5H,6H,7H-cyclopenta[d]-pyrimidin- 2-yl}pyrrolidine-2-carbonyl)-1λ⁶- thiomorpholine-1,1-dione |
| 53a | | (R)-4-(1-{4-[(4-cyclopropyl-3H-imidazol-2- |
| | | yl)amino]-5H,6H,7H-cyclopenta[d]-pyrimidin- |
| | | 2-yl}pyrrolidine-2-carbonyl)-1λ⁶- |
| | | thiomorpholine-1,1-dione |
| 53b | | (S)-4-(1-{4-[(4-cyclopropyl-3H-imidazol-2- |
| | | yl)amino]-5H,6H,7H-cyclopenta[d]-pyrimidin- |
| | | 2-yl}pyrrolidine-2-carbonyl)-1λ⁶- |
| | | thiomorpholine-1,1-dione |
| 54 | 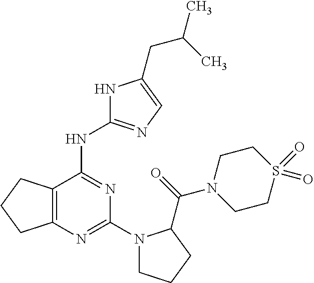 [SMILES: CC(C)Cc1cnc(Nc2nc(N3CCCC3C(=O)N3CCS(=O)(=O)CC3)nc3c2CCC3)[nH]1] | 4-[1-(4-{[4-(2-methylpropyl)-3H-imidazol-2- yl]amino}-5H,6H,7H-cyclopenta[d]-pyrimidin- 2-yl)pyrrolidine-2-carbonyl]- 1λ⁶-thiomorpholine-1,1-dione |
| 54a | | (R)-4-[1-(4-{[4-(2-methylpropyl)-3H-imidazol- |
| | | 2-yl]amino}-5H,6H,7H-cyclopenta[d]- |
| | | pyrimidin-2-yl)pyrrolidine-2-carbonyl]- |
| | | 1λ⁶-thiomorpholine-1,1-dione |
| 54b | | (S)-4-[1-(4-{[4-(2-methylpropyl)-3H-imidazol- |
| | | 2-yl]amino}-5H,6H,7H-cyclopenta[d]- |
| | | pyrimidin-2-yl)pyrrolidine-2-carbonyl]- |
| | | 1λ⁶-thiomorpholine-1,1-dione |
| 55 | 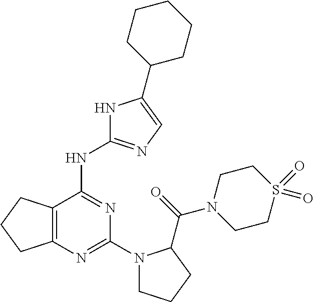 [SMILES: O=C(C1CCCN1c1nc2c(c(Nc3ncc(C4CCCCC4)[nH]3)n1)CCC2)N1CCS(=O)(=O)CC1] | 4-(1-{4-[(4-cyclohexyl-3H-imidazol-2- yl)amino]-5H,6H,7H-cyclopenta[d]-pyrimidin- 2-yl}pyrrolidine-2-carbonyl)-1λ⁶- thiomorpholine-1,1-dione |
| 55a | | (R)-4-(1-{4-[(4-cyclohexyl-3H-imidazol-2- |
| | | yl)amino]-5H,6H,7H-cyclopenta[d]-pyrimidin- |
| | | 2-yl}pyrrolidine-2-carbonyl)-1λ⁶- |
| | | thiomorpholine-1,1-dione |
| 55b | | (S)-4-(1-{4-[(4-cyclohexyl-3H-imidazol-2- |
| | | yl)amino]-5H,6H,7H-cyclopenta[d]-pyrimidin- |
| | | 2-yl}pyrrolidine-2-carbonyl)-1λ⁶- |
| | | thiomorpholine-1,1-dione |
| 56 | 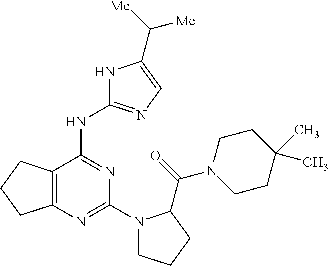 [SMILES: CC(C)c1cnc(Nc2nc(N3CCCC3C(=O)N3CCC(C)(C)CC3)nc3c2CCC3)[nH]1] | (4,4-dimethylpiperidin-1-yl)(1-(4-(5-isopropyl- 1H-imidazol-2-ylamino)-6,7-dihydro-5H- cyclopenta[d]pyrimidin-2-yl)pyrrolidin-2- yl)methanone |
| 56a | | (R)-(4,4-dimethylpiperidin-1-yl)(1-(4-(5- |
| | | isopropyl-1H-imidazol-2-ylamino)-6,7-dihydro- |
| | | 5H-cyclopenta[d]pyrimidin-2-yl)pyrrolidin-2- |
| | | yl)methanone |
| 56b | | (S)-(4,4-dimethylpiperidin-1-yl)(1-(4-(5- |
| | | isopropyl-1H-imidazol-2-ylamino)-6,7-dihydro- |
| | | 5H-cyclopenta[d]pyrimidin-2-yl)pyrrolidin-2- |
| | | yl)methanone |
| 57 | 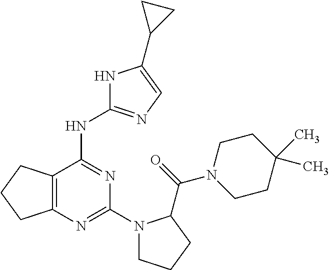 [SMILES: CC1(C)CCN(C(=O)C2CCCN2c2nc3c(c(Nc4ncc(C5CC5)[nH]4)n2)CCC3)CC1] | (1-(4-(5-cyclopropyl-1H-imidazol-2-ylamino)- 6,7-dihydro-5H-cyclopenta[d]pyrimidin-2- yl)pyrrolidin-2-yl)(4,4-dimethylpiperidin-1- yl)methanone |
| 57a | | (R)-(1-(4-(5-cyclopropyl-1H-imidazol-2- |
| | | ylamino)-6,7-dihydro-5H- |
| | | cyclopenta[d]pyrimidin-2-yl)pyrrolidin-2- |
| | | yl)(4,4-dimethylpiperidin-1-yl)methanone |
| 57b | | (S)-(1-(4-(5-cyclopropyl-1H-imidazol-2- |
| | | ylamino)-6,7-dihydro-5H- |
| | | cyclopenta[d]pyrimidin-2-yl)pyrrolidin-2- |
| | | yl)(4,4-dimethylpiperidin-1-yl)methanone |
| 58 | 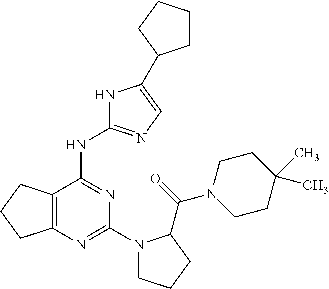 [SMILES: CC1(C)CCN(C(=O)C2CCCN2c2nc3c(c(Nc4ncc(C5CCCC5)[nH]4)n2)CCC3)CC1] | (1-(4-(5-cyclopentyl-1H-imidazol-2-ylamino)- 6,7-dihydro-5H-cyclopenta[d]pyrimidin-2- yl)pyrrolidin-2-yl)(4,4-dimethylpiperidin-1- yl)methanone |
| 58a | | (R)-(1-(4-(5-cyclopentyl-1H-imidazol-2- |
| | | ylamino)-6,7-dihydro-5H- |
| | | cyclopenta[d]pyrimidin-2-yl)pyrrolidin-2- |
| | | yl)(4,4-dimethylpiperidin-1-yl)methanone |
| 58b | | (S)-(1-(4-(5-cyclopentyl-1H-imidazol-2- |
| | | ylamino)-6,7-dihydro-5H- |
| | | cyclopenta[d]pyrimidin-2-yl)pyrrolidin-2- |
| | | yl)(4,4-dimethylpiperidin-1-yl)methanone |
| 59 | 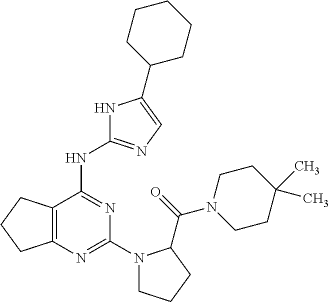 [SMILES: CC1(C)CCN(C(=O)C2CCCN2c2nc3c(c(Nc4ncc(C5CCCCC5)[nH]4)n2)CCC3)CC1] | (1-(4-(5-cyclohexyl-1H-imidazol-2-ylamino)- 6,7-dihydro-5H-cyclopenta[d]pyrimidin-2- yl)pyrrolidin-2-yl)(4,4-dimethylpiperidin-1- yl)methanone |
| 59a | | (R)-(1-(4-(5-cyclohexyl-1H-imidazol-2- |
| | | ylamino)-6,7-dihydro-5H- |
| | | cyclopenta[d]pyrimidin-2-yl)pyrrolidin-2- |
| | | yl)(4,4-dimethylpiperidin-1-yl)methanone |
| 59b | | (S)-(1-(4-(5-cyclohexyl-1H-imidazol-2- |
| | | ylamino)-6,7-dihydro-5H- |
| | | cyclopenta[d]pyrimidin-2-yl)pyrrolidin-2- |
| | | yl)(4,4-dimethylpiperidin-1-yl)methanone |
| 60 | 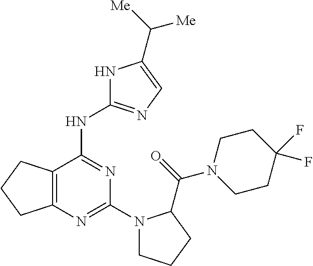 [SMILES: CC(C)c1cnc(Nc2nc(N3CCCC3C(=O)N3CCC(F)(F)CC3)nc3c2CCC3)[nH]1] | (4,4-difluoropiperidin-1-yl)(1-(4-(5-isopropyl- 1H-imidazol-2-ylamino)-6,7-dihydro-5H- cyclopenta[d]pyrimidin-2-yl)pyrrolidin-2- yl)methanone |
| 60a | | (R)-(4,4-difluoropiperidin-1-yl)(1-(4-(5- |
| | | isopropyl-1H-imidazol-2-ylamino)-6,7-dihydro- |
| | | 5H-cyclopenta[d]pyrimidin-2-yl)pyrrolidin-2- |
| | | yl)methanone |
| 60b | | (S)-(4,4-difluoropiperidin-1-yl)(1-(4-(5- |
| | | isopropyl-1H-imidazol-2-ylamino)-6,7-dihydro- |
| | | 5H-cyclopenta[d]pyrimidin-2-yl)pyrrolidin-2- |
| | | yl)methanone |
| 61 | 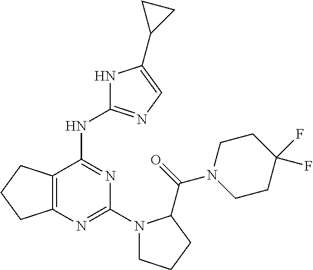 [SMILES: O=C(C1CCCN1c1nc2c(c(Nc3ncc(C4CC4)[nH]3)n1)CCC2)N1CCC(F)(F)CC1] | (1-(4-(5-cyclopropyl-1H-imidazol-2-ylamino)- 6,7-dihydro-5H-cyclopenta[d]pyrimidin-2- yl)pyrrolidin-2-yl)(4,4-difluoropiperidin-1- yl)methanone |
| 61a | | (R)-(1-(4-(5-cyclopropyl-1H-imidazol-2- |
| | | ylamino)-6,7-dihydro-5H- |
| | | cyclopenta[d]pyrimidin-2-yl)pyrrolidin-2- |
| | | yl)(4,4-difluoropiperidin-1-yl)methanone |
| 61b | | (S)-(1-(4-(5-cyclopropyl-1H-imidazol-2- |
| | | ylamino)-6,7-dihydro-5H- |
| | | cyclopenta[d]pyrimidin-2-yl)pyrrolidin-2- |
| | | yl)(4,4-difluoropiperidin-1-yl)methanone |
| 62 | 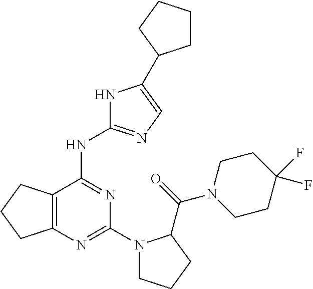 [SMILES: O=C(C1CCCN1c1nc2c(c(Nc3ncc(C4CCCC4)[nH]3)n1)CCC2)N1CCC(F)(F)CC1] | (1-(4-(5-cyclopentyl-1H-imidazol-2-ylamino)- 6,7-dihydro-5H-cyclopenta[d]pyrimidin-2- yl)pyrrolidin-2-yl)(4,4-difluoropiperidin-1- yl)methanone |
| 62a | | (R)-(1-(4-(5-cyclopentyl-1H-imidazol-2- |
| | | ylamino)-6,7-dihydro-5H- |
| | | cyclopenta[d]pyrimidin-2-yl)pyrrolidin-2- |
| | | yl)(4,4-difluoropiperidin-1-yl)methanone |
| 62b | | (S)-(1-(4-(5-cyclopentyl-1H-imidazol-2- |
| | | ylamino)-6,7-dihydro-5H- |
| | | cyclopenta[d]pyrimidin-2-yl)pyrrolidin-2- |
| | | yl)(4,4-difluoropiperidin-1-yl)methanone |
| 63 | 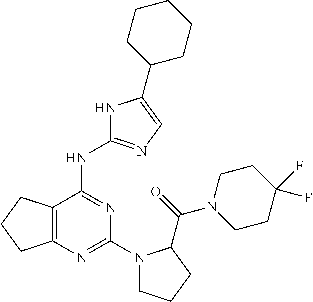 [SMILES: O=C(C1CCCN1c1nc2c(c(Nc3ncc(C4CCCCC4)[nH]3)n1)CCC2)N1CCC(F)(F)CC1] | (1-(4-(5-cyclohexyl-1H-imidazol-2-ylamino)- 6,7-dihydro-5H-cyclopenta[d]pyrimidin-2- yl)pyrrolidin-2-yl)(4,4-difluoropiperidin-1- yl)methanone |
| 63a | | (R)-(1-(4-(5-cyclohexyl-1H-imidazol-2- |
| | | ylamino)-6,7-dihydro-5H- |
| | | cyclopenta[d]pyrimidin-2-yl)pyrrolidin-2- |
| | | yl)(4,4-difluoropiperidin-1-yl)methanone |
| 63b | | (S)-(1-(4-(5-cyclohexyl-1H-imidazol-2- |
| | | ylamino)-6,7-dihydro-5H- |
| | | cyclopenta[d]pyrimidin-2-yl)pyrrolidin-2- |
| | | yl)(4,4-difluoropiperidin-1-yl)methanone |
| 64 | 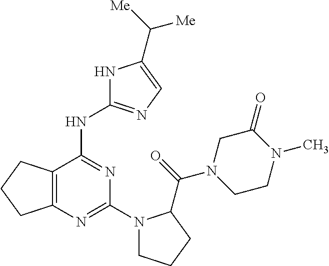 [SMILES: CC(C)c1cnc(Nc2nc(N3CCCC3C(=O)N3CCN(C)C(=O)C3)nc3c2CCC3)[nH]1] | 4-(1-(4-(5-isopropyl-1H-imidazol-2-ylamino)- 6,7-dihydro-5H-cyclopenta[d]pyrimidin-2- yl)pyrrolidine-2-carbonyl)-1-methylpiperazin-2- one |
| 64a | | (R)-4-(1-(4-(5-isopropyl-1H-imidazol-2- |
| | | ylamino)-6,7-dihydro-5H- |
| | | cyclopenta[d]pyrimidin-2-yl)pyrrolidine-2- |
| | | carbonyl)-1-methylpiperazin-2-one |
| 64b | | (S)-4-(1-(4-(5-isopropyl-1H-imidazol-2- |
| | | ylamino)-6,7-dihydro-5H- |
| | | cyclopenta[d]pyrimidin-2-yl)pyrrolidine-2- |
| | | carbonyl)-1-methylpiperazin-2-one |
| 65 | 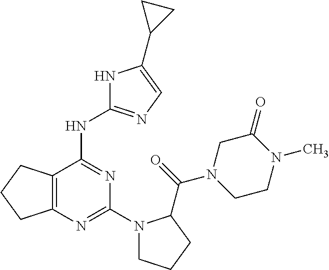 [SMILES: CN1CCN(C(=O)C2CCCN2c2nc3c(c(Nc4ncc(C5CC5)[nH]4)n2)CCC3)CC1=O] | 4-(1-(4-(5-cyclopropyl-1H-imidazol-2-ylamino)- 6,7-dihydro-5H-cyclopenta[d]pyrimidin-2- yl)pyrrolidine-2-carbonyl)-1-methylpiperazin-2- one |
| 65a | | (R)-4-(1-(4-(5-cyclopropyl-1H-imidazol-2- |
| | | ylamino)-6,7-dihydro-5H- |
| | | cyclopenta[d]pyrimidin-2-yl)pyrrolidine-2- |
| | | carbonyl)-1-methylpiperazin-2-one |
| 65b | | (S)-4-(1-(4-(5-cyclopropyl-1H-imidazol-2- |
| | | ylamino)-6,7-dihydro-5H- |
| | | cyclopenta[d]pyrimidin-2-yl)pyrrolidine-2- |
| | | carbonyl)-1-methylpiperazin-2-one |
| 66 | 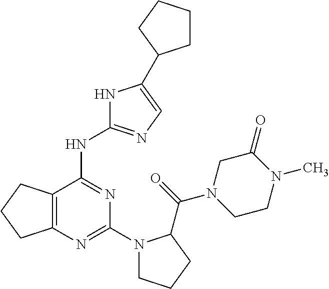 [SMILES: CN1CCN(C(=O)C2CCCN2c2nc3c(c(Nc4ncc(C5CCCC5)[nH]4)n2)CCC3)CC1=O] | 4-(1-(4-(5-cyclopentyl-1H-imidazol-2-ylamino)- 6,7-dihydro-5H-cyclopenta[d]pyrimidin-2- yl)pyrrolidine-2-carbonyl)-1-methylpiperazin-2- one |
| 66a | | (R)-4-(1-(4-(5-cyclopentyl-1H-imidazol-2- |
| | | ylamino)-6,7-dihydro-5H- |
| | | cyclopenta[d]pyrimidin-2-yl)pyrrolidine-2- |
| | | carbonyl)-1-methylpiperazin-2-one |
| 66b | | (S)-4-(1-(4-(5-cyclopentyl-1H-imidazol-2- |
| | | ylamino)-6,7-dihydro-5H- |
| | | cyclopenta[d]pyrimidin-2-yl)pyrrolidine-2- |
| | | carbonyl)-1-methylpiperazin-2-one |
| 67 | 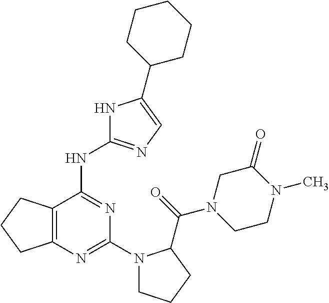 [SMILES: CN1CCN(C(=O)C2CCCN2c2nc3c(c(Nc4ncc(C5CCCCC5)[nH]4)n2)CCC3)CC1=O] | 4-(1-(4-(5-cyclohexyl-1H-imidazol-2-ylamino)- 6,7-dihydro-5H-cyclopenta[d]pyrimidin-2- yl)pyrrolidine-2-carbonyl)-1-methylpiperazin-2- one |
| 67a | | (R)-4-(1-(4-(5-cyclohexyl-1H-imidazol-2- |
| | | ylamino)-6,7-dihydro-5H- |
| | | cyclopenta[d]pyrimidin-2-yl)pyrrolidine-2- |
| | | carbonyl)-1-methylpiperazin-2-one |
| 67b | | (S)-4-(1-(4-(5-cyclohexyl-1H-imidazol-2- |
| | | ylamino)-6,7-dihydro-5H- |
| | | cyclopenta[d]pyrimidin-2-yl)pyrrolidine-2- |
| | | carbonyl)-1-methylpiperazin-2-one |
| 68 | 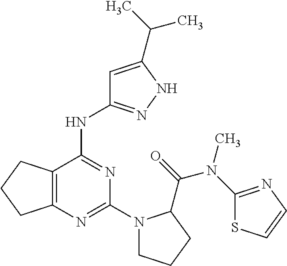 [SMILES: CC(C)c1cc(Nc2nc(N3CCCC3C(=O)N(C)c3nccs3)nc3c2CCC3)n[nH]1] | 1-(4-(5-isopropyl-1H-pyrazol-3-ylamino)-6,7- dihydro-5H-cyclopenta[d]pyrimidin-2-yl)-N- methyl-N-(thiazol-2-yl)pyrrolidine-2- carboxamide |
| 68a | | (R)-1-(4-(5-isopropyl-1H-pyrazol-3-ylamino)- |
| | | 6,7-dihydro-5H-cyclopenta[d]pyrimidin-2-yl)- |
| | | N-methyl-N-(thiazol-2-yl)pyrrolidine-2- |
| | | carboxamide |
| 68b | | (S)-1-(4-(5-isopropyl-1H-pyrazol-3-ylamino)- |
| | | 6,7-dihydro-5H-cyclopenta[d]pyrimidin-2-yl)- |
| | | N-methyl-N-(thiazol-2-yl)pyrrolidine-2- |
| | | carboxamide |
| 69 | 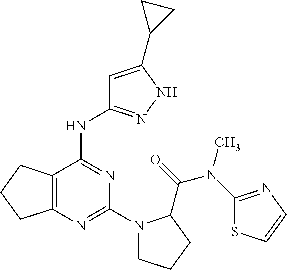 [SMILES: CN(C(=O)C1CCCN1c1nc2c(c(Nc3cc(C4CC4)[nH]n3)n1)CCC2)c1nccs1] | 1-(4-(5-cyclopropyl-1H-pyrazol-3-ylamino)-6,7- dihydro-5H-cyclopenta[d]pyrimidin-2-yl)-N- methyl-N-(thiazol-2-yl)pyrrolidine-2- carboxamide |
| 69a | | (R)-1-(4-(5-cyclopropyl-1H-pyrazol-3-ylamino)- |
| | | 6,7-dihydro-5H-cyclopenta[d]pyrimidin-2-yl)- |
| | | N-methyl-N-(thiazol-2-yl)pyrrolidine-2- |
| | | carboxamide |
| 69b | | (S)-1-(4-(5-cyclopropyl-1H-pyrazol-3-ylamino)- |
| | | 6,7-dihydro-5H-cyclopenta[d]pyrimidin-2-yl)- |
| | | N-methyl-N-(thiazol-2-yl)pyrrolidine-2- |
| | | carboxamide |
| 70 | 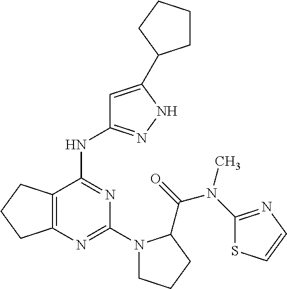 [SMILES: CN(C(=O)C1CCCN1c1nc2c(c(Nc3cc(C4CCCC4)[nH]n3)n1)CCC2)c1nccs1] | 1-(4-(5-cyclopentyl-1H-pyrazol-3-ylamino)-6,7- dihydro-5H-cyclopenta[d]pyrimidin-2-yl)-N- methyl-N-(thiazol-2-yl)pyrrolidine-2- carboxamide |
| 70a | | (R)-1-(4-(5-cyclopentyl-1H-pyrazol-3-ylamino)- |
| | | 6,7-dihydro-5H-cyclopenta[d]pyrimidin-2-yl)- |
| | | N-methyl-N-(thiazol-2-yl)pyrrolidine-2- |
| | | carboxamide |
| 70b | | (S)-1-(4-(5-cyclopentyl-1H-pyrazol-3-ylamino)- |
| | | 6,7-dihydro-5H-cyclopenta[d]pyrimidin-2-yl)- |
| | | N-methyl-N-(thiazol-2-yl)pyrrolidine-2- |
| | | carboxamide |
| 71 | 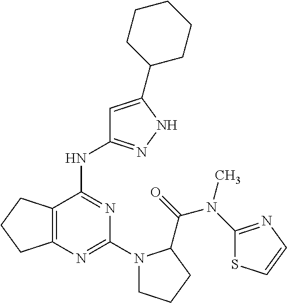 [SMILES: CN(C(=O)C1CCCN1c1nc2c(c(Nc3cc(C4CCCCC4)[nH]n3)n1)CCC2)c1nccs1] | 1-(4-(5-cyclohexyl-1H-pyrazol-3-ylamino)-6,7- dihydro-5H-cyclopenta[d]pyrimidin-2-yl)-N- methyl-N-(thiazol-2-yl)pyrrolidine-2- carboxamide |
| 71a | | (R)-1-(4-(5-cyclohexyl-1H-pyrazol-3-ylamino)- |
| | | 6,7-dihydro-5H-cyclopenta[d]pyrimidin-2-yl)- |
| | | N-methyl-N-(thiazol-2-yl)pyrrolidine-2- |
| | | carboxamide |
| 71b | | (S)-1-(4-(5-cyclohexyl-1H-pyrazol-3-ylamino)- |
| | | 6,7-dihydro-5H-cyclopenta[d]pyrimidin-2-yl)- |
| | | N-methyl-N-(thiazol-2-yl)pyrrolidine-2- |
| | | carboxamide |
| 72 | 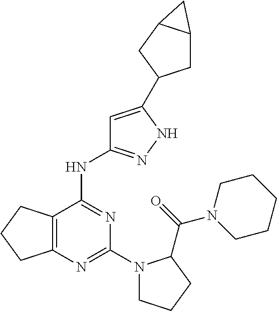 [SMILES: O=C(C1CCCN1c1nc2c(c(Nc3cc(C4CC5CC5C4)[nH]n3)n1)CCC2)N1CCCCC1] | (1-(4-(5-(bicyclo[3.1.0]hexan-3-yl)-1H-pyrazol- 3-ylamino)-6,7-dihydro-5H- cyclopenta[d]pyrimidin-2-yl)pyrrolidin-2- yl)(piperidin-1-yl)methanone |
| 72a | | (R)-(1-(4-(5-(bicyclo[3.1.0]hexan-3-yl)-1H- |
| | | pyrazol-3-ylamino)-6,7-dihydro-5H- |
| | | cyclopenta[d]pyrimidin-2-yl)pyrrolidin-2- |
| | | yl)(piperidin-1-yl)methanone |
| 72b | | (S)-(1-(4-(5-(bicyclo[3.1.0]hexan-3-yl)-1H- |
| | | pyrazol-3-ylamino)-6,7-dihydro-5H- |
| | | cyclopenta[d]pyrimidin-2-yl)pyrrolidin-2- |
| | | yl)(piperidin-1-yl)methanone |
| 73 | 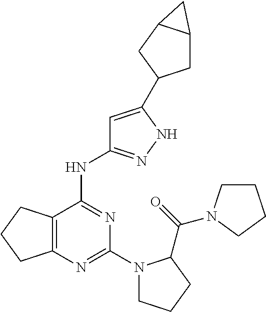 [SMILES: O=C(C1CCCN1c1nc2c(c(Nc3cc(C4CC5CC5C4)[nH]n3)n1)CCC2)N1CCCC1] | (1-(4-(5-(bicyclo[3.1.0]hexan-3-yl)-1H-pyrazol- 3-ylamino)-6,7-dihydro-5H- cyclopenta[d]pyrimidin-2-yl)pyrrolidin-2- yl)(pyrrolidin-1-yl)methanone |
| 73a | | (R)-(1-(4-(5-(bicyclo[3.1.0]hexan-3-yl)-1H- |
| | | pyrazol-3-ylamino)-6,7-dihydro-5H- |
| | | cyclopenta[d]pyrimidin-2-yl)pyrrolidin-2- |
| | | yl)(pyrrolidin-1-yl)methanone |
| 73b | | (S)-(1-(4-(5-(bicyclo[3.1.0]hexan-3-yl)-1H- |
| | | pyrazol-3-ylamino)-6,7-dihydro-5H- |
| | | cyclopenta[d]pyrimidin-2-yl)pyrrolidin-2- |
| | | yl)(pyrrolidin-1-yl)methanone |
| 74 | 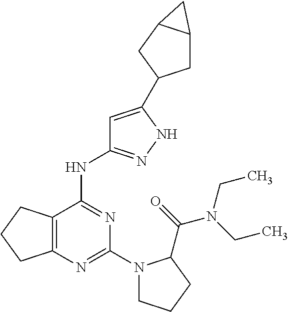 [SMILES: CCN(CC)C(=O)C1CCCN1c1nc2c(c(Nc3cc(C4CC5CC5C4)[nH]n3)n1)CCC2] | 1-(4-(5-(bicyclo[3.1.0]hexan-3-yl)-1H-pyrazol- 3-ylamino)-6,7-dihydro-5H- cyclopenta[d]pyrimidin-2-yl)-N,N- diethylpyrrolidine-2-carboxamide |
| 74a | | (R)-1-(4-(5-(bicyclo[3.1.0]hexan-3-yl)-1H- |
| | | pyrazol-3-ylamino)-6,7-dihydro-5H- |
| | | cyclopenta[d]pyrimidin-2-yl)-N,N- |
| | | diethylpyrrolidine-2-carboxamide |
| 74b | | (S)-1-(4-(5-(bicyclo[3.1.0]hexan-3-yl)-1H- |
| | | pyrazol-3-ylamino)-6,7-dihydro-5H- |
| | | cyclopenta[d]pyrimidin-2-yl)-N,N- |
| | | diethylpyrrolidine-2-carboxamide |
| 75 | 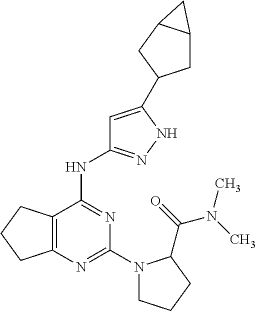 [SMILES: CN(C)C(=O)C1CCCN1c1nc2c(c(Nc3cc(C4CC5CC5C4)[nH]n3)n1)CCC2] | 1-(4-(5-(bicyclo[3.1.0]hexan-3-yl)-1H-pyrazol- 3-ylamino)-6,7-dihydro-5H- cyclopenta[d]pyrimidin-2-yl)-N,N- dimethylpyrrolidine-2-carboxamide |
| 75a | | (R)-1-(4-(5-(bicyclo[3.1.0]hexan-3-yl)-1H- |
| | | pyrazol-3-ylamino)-6,7-dihydro-5H- |
| | | cyclopenta[d]pyrimidin-2-yl)-N,N- |
| | | dimethylpyrrolidine-2-carboxamide |
| 75b | | (S)-1-(4-(5-(bicyclo[3.1.0]hexan-3-yl)-1H- |
| | | pyrazol-3-ylamino)-6,7-dihydro-5H- |
| | | cyclopenta[d]pyrimidin-2-yl)-N,N- |
| | | dimethylpyrrolidine-2-carboxamide |
| 76 | 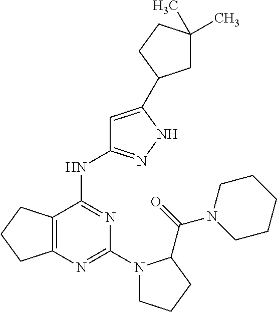 [SMILES: CC1(C)CCC(c2cc(Nc3nc(N4CCCC4C(=O)N4CCCCC4)nc4c3CCC4)n[nH]2)C1] | (1-(4-(5-(3,3-dimethylcyclopentyl)-1H-pyrazol- 3-ylamino)-6,7-dihydro-5H- cyclopenta[d]pyrimidin-2-yl)pyrrolidin-2- yl)(piperidin-1-yl)methanone |
| 76a | | (R,R)-(1-(4-(5-(3,3-dimethylcyclopentyl)-1H- |
| | | pyrazol-3-ylamino)-6,7-dihydro-5H- |
| | | cyclopenta[d]pyrimidin-2-yl)pyrrolidin-2- |
| | | yl)(piperidin-1-yl)methanone |
| 76b | | (R,S)-(1-(4-(5-(3,3-dimethylcyclopentyl)-1H- |
| | | pyrazol-3-ylamino)-6,7-dihydro-5H- |
| | | cyclopenta[d]pyrimidin-2-yl)pyrrolidin-2- |
| | | yl)(piperidin-1-yl)methanone |
| 76c | | (S,R)-(1-(4-(5-(3,3-dimethylcyclopentyl)-1H- |
| | | pyrazol-3-ylamino)-6,7-dihydro-5H- |
| | | cyclopenta[d]pyrimidin-2-yl)pyrrolidin-2- |
| | | yl)(piperidin-1-yl)methanone |
| 76d | | (S,S)-(1-(4-(5-(3,3-dimethylcyclopentyl)-1H- |
| | | pyrazol-3-ylamino)-6,7-dihydro-5H- |
| | | cyclopenta[d]pyrimidin-2-yl)pyrrolidin-2- |
| | | yl)(piperidin-1-yl)methanone |
| 77 | 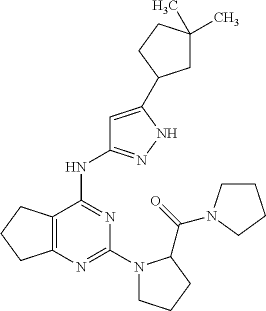 [SMILES: CC1(C)CCC(c2cc(Nc3nc(N4CCCC4C(=O)N4CCCC4)nc4c3CCC4)n[nH]2)C1] | (1-(4-(5-(3,3-dimethylcyclopentyl)-1H-pyrazol- 3-ylamino)-6,7-dihydro-5H- cyclopenta[d]pyrimidin-2-yl)pyrrolidin-2- yl)(pyrrolidin-1-yl)methanone |
| 77a | | (R,R)-(1-(4-(5-(3,3-dimethylcyclopentyl)-1H- |
| | | pyrazol-3-ylamino)-6,7-dihydro-5H- |
| | | cyclopenta[d]pyrimidin-2-yl)pyrrolidin-2- |
| | | yl)(pyrrolidin-1-yl)methanone |
| 77b | | (R,S)-(1-(4-(5-(3,3-dimethylcyclopentyl)-1H- |
| | | pyrazol-3-ylamino)-6,7-dihydro-5H- |
| | | cyclopenta[d]pyrimidin-2-yl)pyrrolidin-2- |
| | | yl)(pyrrolidin-1-yl)methanone |
| 77c | | (S,R)-(1-(4-(5-(3,3-dimethylcyclopentyl)-1H- |
| | | pyrazol-3-ylamino)-6,7-dihydro-5H- |
| | | cyclopenta[d]pyrimidin-2-yl)pyrrolidin-2- |
| | | yl)(pyrrolidin-1-yl)methanone |
| 77d | | (S,S)-(1-(4-(5-(3,3-dimethylcyclopentyl)-1H- |
| | | pyrazol-3-ylamino)-6,7-dihydro-5H- |
| | | cyclopenta[d]pyrimidin-2-yl)pyrrolidin-2- |
| | | yl)(pyrrolidin-1-yl)methanone |
| 78 | 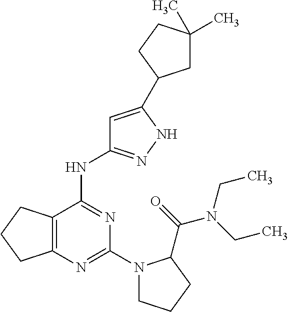 [SMILES: CCN(CC)C(=O)C1CCCN1c1nc2c(c(Nc3cc(C4CCC(C)(C)C4)[nH]n3)n1)CCC2] | 1-(4-(5-(3,3-dimethylcyclopentyl)-1H-pyrazol- 3-ylamino)-6,7-dihydro-5H- cyclopenta[d]pyrimidin-2-yl)-N,N- diethylpyrrolidine-2-carboxamide |
| 78a | | (R,R)-1-(4-(5-(3,3-dimethylcyclopentyl)-1H- |
| | | pyrazol-3-ylamino)-6,7-dihydro-5H- |
| | | cyclopenta[d]pyrimidin-2-yl)-N,N- |
| | | diethylpyrrolidine-2-carboxamide |
| 78b | | (R,S)-1-(4-(5-(3,3-dimethylcyclopentyl)-1H- |
| | | pyrazol-3-ylamino)-6,7-dihydro-5H- |
| | | cyclopenta[d]pyrimidin-2-yl)-N,N- |
| | | diethylpyrrolidine-2-carboxamide |
| 78c | | (S,R)-1-(4-(5-(3,3-dimethylcyclopentyl)-1H- |
| | | pyrazol-3-ylamino)-6,7-dihydro-5H- |
| | | cyclopenta[d]pyrimidin-2-yl)-N,N- |
| | | diethylpyrrolidine-2-carboxamide |
| 78d | | (S,S)-1-(4-(5-(3,3-dimethylcyclopentyl)-1H- |
| | | pyrazol-3-ylamino)-6,7-dihydro-5H- |
| | | cyclopenta[d]pyrimidin-2-yl)-N,N- |
| | | diethylpyrrolidine-2-carboxamide |
| 79 | 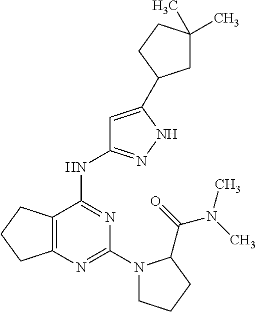 [SMILES: CN(C)C(=O)C1CCCN1c1nc2c(c(Nc3cc(C4CCC(C)(C)C4)[nH]n3)n1)CCC2] | 1-(4-(5-(3,3-dimethylcyclopentyl)-1H-pyrazol- 3-ylamino)-6,7-dihydro-5H- cyclopenta[d]pyrimidin-2-yl)-N,N- dimethylpyrrolidine-2-carboxamide |
| 79a | | (R,R)-1-(4-(5-(3,3-dimethylcyclopentyl)-1H- |
| | | pyrazol-3-ylamino)-6,7-dihydro-5H- |
| | | cyclopenta[d]pyrimidin-2-yl)-N,N- |
| | | dimethylpyrrolidine-2-carboxamide |
| 79b | | (R,S)-1-(4-(5-(3,3-dimethylcyclopentyl)-1H- |
| | | pyrazol-3-ylamino)-6,7-dihydro-5H- |
| | | cyclopenta[d]pyrimidin-2-yl)-N,N- |
| | | dimethylpyrrolidine-2-carboxamide |
| 79c | | (S,R)-1-(4-(5-(3,3-dimethylcyclopentyl)-1H- |
| | | pyrazol-3-ylamino)-6,7-dihydro-5H- |
| | | cyclopenta[d]pyrimidin-2-yl)-N,N- |
| | | dimethylpyrrolidine-2-carboxamide |
| 79d | | (S,S)-1-(4-(5-(3,3-dimethylcyclopentyl)-1H- |
| | | pyrazol-3-ylamino)-6,7-dihydro-5H- |
| | | cyclopenta[d]pyrimidin-2-yl)-N,N- |
| | | dimethylpyrrolidine-2-carboxamide |
| 80 | 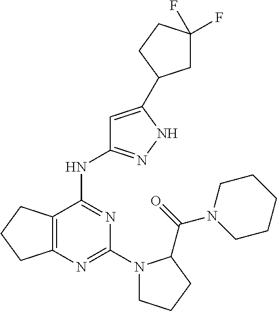 [SMILES: O=C(C1CCCN1c1nc2c(c(Nc3cc(C4CCC(F)(F)C4)[nH]n3)n1)CCC2)N1CCCCC1] | (1-(4-(5-(3,3-difluorocyclopentyl)-1H-pyrazol- 3-ylamino)-6,7-dihydro-5H- cyclopenta[d]pyrimidin-2-yl)pyrrolidin-2- yl)(piperidin-1-yl)methanone |
| 80a | | (R,R)-(1-(4-(5-(3,3-difluorocyclopentyl)-1H- |
| | | pyrazol-3-ylamino)-6,7-dihydro-5H- |
| | | cyclopenta[d]pyrimidin-2-yl)pyrrolidin-2- |
| | | yl)(piperidin-1-yl)methanone |
| 80b | | (R,S)-(1-(4-(5-(3,3-difluorocyclopentyl)-1H- |
| | | pyrazol-3-ylamino)-6,7-dihydro-5H- |
| | | cyclopenta[d]pyrimidin-2-yl)pyrrolidin-2- |
| | | yl)(piperidin-1-yl)methanone |
| 80c | | (S,R)-(1-(4-(5-(3,3-difluorocyclopentyl)-1H- |
| | | pyrazol-3-ylamino)-6,7-dihydro-5H- |
| | | cyclopenta[d]pyrimidin-2-yl)pyrrolidin-2- |
| | | yl)(piperidin-1-yl)methanone |
| 80d | | (S,S)-(1-(4-(5-(3,3-difluorocyclopentyl)-1H- |
| | | pyrazol-3-ylamino)-6,7-dihydro-5H- |
| | | cyclopenta[d]pyrimidin-2-yl)pyrrolidin-2- |
| | | yl)(piperidin-1-yl)methanone |
| 81 | 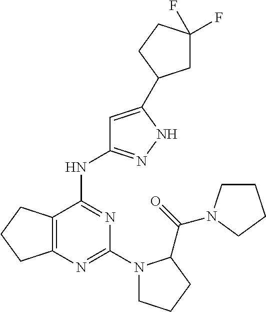 [SMILES: O=C(C1CCCN1c1nc2c(c(Nc3cc(C4CCC(F)(F)C4)[nH]n3)n1)CCC2)N1CCCC1] | (1-(4-(5-(3,3-difluorocyclopentyl)-1H-pyrazol- 3-ylamino)-6,7-dihydro-5H- cyclopenta[d]pyrimidin-2-yl)pyrrolidin-2- yl)(pyrrolidin-1-yl)methanone |
| 81a | | (R,R)-(1-(4-(5-(3,3-difluorocyclopentyl)-1H- |
| | | pyrazol-3-ylamino)-6,7-dihydro-5H- |
| | | cyclopenta[d]pyrimidin-2-yl)pyrrolidin-2- |
| | | yl)(pyrrolidin-1-yl)methanone |
| 81b | | (R,S)-(1-(4-(5-(3,3-difluorocyclopentyl)-1H- |
| | | pyrazol-3-ylamino)-6,7-dihydro-5H- |
| | | cyclopenta[d]pyrimidin-2-yl)pyrrolidin-2- |
| | | yl)(pyrrolidin-1-yl)methanone |
| 81c | | (S,R)-(1-(4-(5-(3,3-difluorocyclopentyl)-1H- |
| | | pyrazol-3-ylamino)-6,7-dihydro-5H- |
| | | cyclopenta[d]pyrimidin-2-yl)pyrrolidin-2- |
| | | yl)(pyrrolidin-1-yl)methanone |
| 81d | | (S,S)-(1-(4-(5-(3,3-difluorocyclopentyl)-1H- |
| | | pyrazol-3-ylamino)-6,7-dihydro-5H- |
| | | cyclopenta[d]pyrimidin-2-yl)pyrrolidin-2- |
| | | yl)(pyrrolidin-1-yl)methanone |
| 82 | 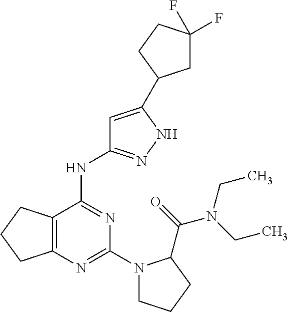 [SMILES: CCN(CC)C(=O)C1CCCN1c1nc2c(c(Nc3cc(C4CCC(F)(F)C4)[nH]n3)n1)CCC2] | 1-(4-(5-(3,3-difluorocyclopentyl)-1H-pyrazol-3- ylamino)-6,7-dihydro-5H- cyclopenta[d]pyrimidin-2-yl)-N,N- diethylpyrrolidine-2-carboxamide |
| 82a | | (R,R)-1-(4-(5-(3,3-difluorocyclopentyl)-1H- |
| | | pyrazol-3-ylamino)-6,7-dihydro-5H- |
| | | cyclopenta[d]pyrimidin-2-yl)-N,N- |
| | | diethylpyrrolidine-2-carboxamide |
| 82b | | (R,S)-1-(4-(5-(3,3-difluorocyclopentyl)-1H- |
| | | pyrazol-3-ylamino)-6,7-dihydro-5H- |
| | | cyclopenta[d]pyrimidin-2-yl)-N,N- |
| | | diethylpyrrolidine-2-carboxamide |
| 82c | | (S,R)-1-(4-(5-(3,3-difluorocyclopentyl)-1H- |
| | | pyrazol-3-ylamino)-6,7-dihydro-5H- |
| | | cyclopenta[d]pyrimidin-2-yl)-N,N- |
| | | diethylpyrrolidine-2-carboxamide |
| 82d | | (S,S)-1-(4-(5-(3,3-difluorocyclopentyl)-1H- |
| | | pyrazol-3-ylamino)-6,7-dihydro-5H- |
| | | cyclopenta[d]pyrimidin-2-yl)-N,N- |
| | | diethylpyrrolidine-2-carboxamide |
| 83 | 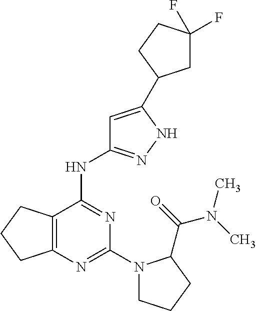 [SMILES: CN(C)C(=O)C1CCCN1c1nc2c(c(Nc3cc(C4CCC(F)(F)C4)[nH]n3)n1)CCC2] | 1-(4-(5-(3,3-difluorocyclopentyl)-1H-pyrazol-3- ylamino)-6,7-dihydro-5H- cyclopenta[d]pyrimidin-2-yl)-N,N- dimethylpyrrolidine-2-carboxamide |
| 83a | | (R,R)-1-(4-(5-(3,3-difluorocyclopentyl)-1H- |
| | | pyrazol-3-ylamino)-6,7-dihydro-5H- |
| | | cyclopenta[d]pyrimidin-2-yl)-N,N- |
| | | dimethylpyrrolidine-2-carboxamide |
| 83b | | (R,S)-1-(4-(5-(3,3-difluorocyclopentyl)-1H- |
| | | pyrazol-3-ylamino)-6,7-dihydro-5H- |
| | | cyclopenta[d]pyrimidin-2-yl)-N,N- |
| | | dimethylpyrrolidine-2-carboxamide |
| 83c | | (S,R)-1-(4-(5-(3,3-difluorocyclopentyl)-1H- |
| | | pyrazol-3-ylamino)-6,7-dihydro-5H- |
| | | cyclopenta[d]pyrimidin-2-yl)-N,N- |
| | | dimethylpyrrolidine-2-carboxamide |
| 83d | | (S,S)-1-(4-(5-(3,3-difluorocyclopentyl)-1H- |
| | | pyrazol-3-ylamino)-6,7-dihydro-5H- |
| | | cyclopenta[d]pyrimidin-2-yl)-N,N- |
| | | dimethylpyrrolidine-2-carboxamide |
| 84 | 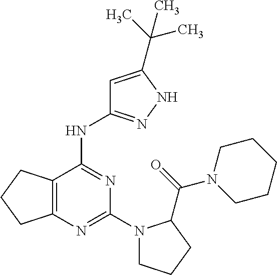 [SMILES: CC(C)(C)c1cc(Nc2nc(N3CCCC3C(=O)N3CCCCC3)nc3c2CCC3)n[nH]1] | (1-(4-(5-tert-butyl-1H-pyrazol-3-ylamino)-6,7- dihydro-5H-cyclopenta[d]pyrimidin-2- yl)pyrrolidin-2-yl)(piperidin-1-yl)methanone |
| 84a | | (R)-(1-(4-(5-tert-butyl-1H-pyrazol-3-ylamino)- |
| | | 6,7-dihydro-5H-cyclopenta[d]pyrimidin-2- |
| | | yl)pyrrolidin-2-yl)(piperidin-1-yl)methanone |
| 84b | | (S)-(1-(4-(5-tert-butyl-1H-pyrazol-3-ylamino)- |
| | | 6,7-dihydro-5H-cyclopenta[d]pyrimidin-2- |
| | | yl)pyrrolidin-2-yl)(piperidin-1-yl)methanone |
| 85 | 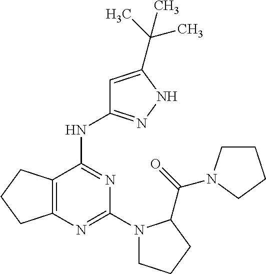 [SMILES: CC(C)(C)c1cc(Nc2nc(N3CCCC3C(=O)N3CCCC3)nc3c2CCC3)n[nH]1] | (1-(4-(5-tert-butyl-1H-pyrazol-3-ylamino)-6,7- dihydro-5H-cyclopenta[d]pyrimidin-2- yl)pyrrolidin-2-yl)(pyrrolidin-1-yl)methanone |
| 85a | | (R)-(1-(4-(5-tert-butyl-1H-pyrazol-3-ylamino)- |
| | | 6,7-dihydro-5H-cyclopenta[d]pyrimidin-2- |
| | | yl)pyrrolidin-2-yl)(pyrrolidin-1-yl)methanone |
| 85b | | (S)-(1-(4-(5-tert-butyl-1H-pyrazol-3-ylamino)- |
| | | 6,7-dihydro-5H-cyclopenta[d]pyrimidin-2- |
| | | yl)pyrrolidin-2-yl)(pyrrolidin-1-yl)methanone |
| 86 | 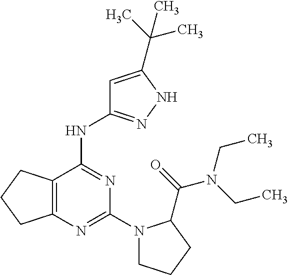 [SMILES: CCN(CC)C(=O)C1CCCN1c1nc2c(c(Nc3cc(C(C)(C)C)[nH]n3)n1)CCC2] | 1-(4-(5-tert-butyl-1H-pyrazol-3-ylamino)-6,7- dihydro-5H-cyclopenta[d]pyrimidin-2-yl)-N,N- diethylpyrrolidine-2-carboxamide |
| 86a | | (R)-1-(4-(5-tert-butyl-1H-pyrazol-3-ylamino)- |
| | | 6,7-dihydro-5H-cyclopenta[d]pyrimidin-2-yl)- |
| | | N,N-diethylpyrrolidine-2-carboxamide |
| 86b | | (S)-1-(4-(5-tert-butyl-1H-pyrazol-3-ylamino)- |
| | | 6,7-dihydro-5H-cyclopenta[d]pyrimidin-2-yl)- |
| | | N,N-diethylpyrrolidine-2-carboxamide |
| 87 | 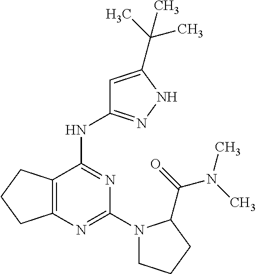 [SMILES: CN(C)C(=O)C1CCCN1c1nc2c(c(Nc3cc(C(C)(C)C)[nH]n3)n1)CCC2] | 1-(4-(5-tert-butyl-1H-pyrazol-3-ylamino)-6,7- dihydro-5H-cyclopenta[d]pyrimidin-2-yl)-N,N- dimethylpyrrolidine-2-carboxamide |
| 87a | | (R)-1-(4-(5-tert-butyl-1H-pyrazol-3-ylamino)- |
| | | 6,7-dihydro-5H-cyclopenta[d]pyrimidin-2-yl)- |
| | | N,N-dimethylpyrrolidine-2-carboxamide |
| 87b | | (S)-1-(4-(5-tert-butyl-1H-pyrazol-3-ylamino)- |
| | | 6,7-dihydro-5H-cyclopenta[d]pyrimidin-2-yl)- |
| | | N,N-dimethylpyrrolidine-2-carboxamide |
| 88 | 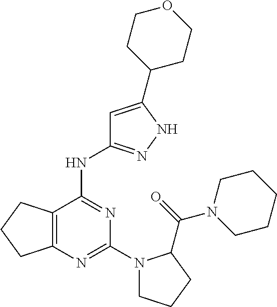 [SMILES: O=C(C1CCCN1c1nc2c(c(Nc3cc(C4CCOCC4)[nH]n3)n1)CCC2)N1CCCCC1] | piperidin-1-yl(1-(4-(5-(tetrahydro-2H-pyran-4- yl)-1H-pyrazol-3-ylamino)-6,7-dihydro-5H- cyclopenta[d]pyrimidin-2-yl)pyrrolidin-2- yl)methanone |
| 88a | | (R)-piperidin-1-yl(1-(4-(5-(tetrahydro-2H-pyran- |
| | | 4-yl)-1H-pyrazol-3-ylamino)-6,7-dihydro-5H- |
| | | cyclopenta[d]pyrimidin-2-yl)pyrrolidin-2- |
| | | yl)methanone |
| 88b | | (S)-piperidin-1-yl(1-(4-(5-(tetrahydro-2H-pyran- |
| | | 4-yl)-1H-pyrazol-3-ylamino)-6,7-dihydro-5H- |
| | | cyclopenta[d]pyrimidin-2-yl)pyrrolidin-2- |
| | | yl)methanone |
| 89 | 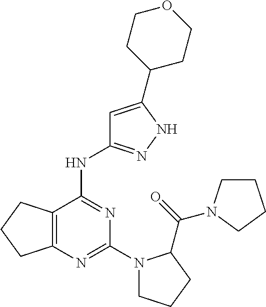 [SMILES: O=C(C1CCCN1c1nc2c(c(Nc3cc(C4CCOCC4)[nH]n3)n1)CCC2)N1CCCC1] | pyrrolidin-1-yl(1-(4-(5-(tetrahydro-2H-pyran-4- yl)-1H-pyrazol-3-ylamino)-6,7-dihydro-5H- cyclopenta[d]pyrimidin-2-yl)pyrrolidin-2- yl)methanone |
| 89a | | (R)-pyrrolidin-1-yl(1-(4-(5-(tetrahydro-2H- |
| | | pyran-4-yl)-1H-pyrazol-3-ylamino)-6,7-dihydro- |
| | | 5H-cyclopenta[d]pyrimidin-2-yl)pyrrolidin-2- |
| | | yl)methanone |
| 89b | | (S)-pyrrolidin-1-yl(1-(4-(5-(tetrahydro-2H- |
| | | pyran-4-yl)-1H-pyrazol-3-ylamino)-6,7-dihydro- |
| | | 5H-cyclopenta[d]pyrimidin-2-yl)pyrrolidin-2- |
| | | yl)methanone |
| 90 | 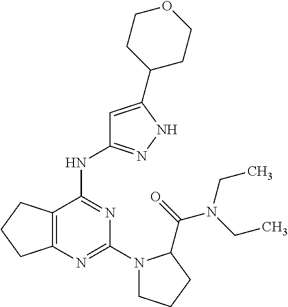 [SMILES: CCN(CC)C(=O)C1CCCN1c1nc2c(c(Nc3cc(C4CCOCC4)[nH]n3)n1)CCC2] | N,N-diethyl-1-(4-(5-(tetrahydro-2H-pyran-4-yl)- 1H-pyrazol-3-ylamino)-6,7-dihydro-5H- cyclopenta[d]pyrimidin-2-yl)pyrrolidine-2- carboxamide |
| 90a | | (R)-N,N-diethyl-1-(4-(5-(tetrahydro-2H-pyran- |
| | | 4-yl)-1H-pyrazol-3-ylamino)-6,7-dihydro-5H- |
| | | cyclopenta[d]pyrimidin-2-yl)pyrrolidine-2- |
| | | carboxamide |
| 90b | | (S)-N,N-diethyl-1-(4-(5-(tetrahydro-2H-pyran-4- |
| | | yl)-1H-pyrazol-3-ylamino)-6,7-dihydro-5H- |
| | | cyclopenta[d]pyrimidin-2-yl)pyrrolidine-2- |
| | | carboxamide |
| 91 | 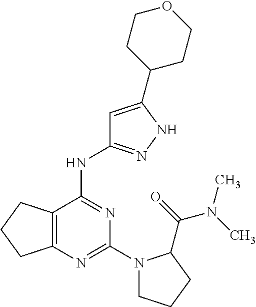 [SMILES: CN(C)C(=O)C1CCCN1c1nc2c(c(Nc3cc(C4CCOCC4)[nH]n3)n1)CCC2] | N,N-dimethyl-1-(4-(5-(tetrahydro-2H-pyran-4- yl)-1H-pyrazol-3-ylamino)-6,7-dihydro-5H- cyclopenta[d]pyrimidin-2-yl)pyrrolidine-2- carboxamide |
| 91a | | (R)-N,N-dimethyl-1-(4-(5-(tetrahydro-2H-pyran- |
| | | 4-yl)-1H-pyrazol-3-ylamino)-6,7-dihydro-5H- |
| | | cyclopenta[d]pyrimidin-2-yl)pyrrolidine-2- |
| | | carboxamide |
| 91b | | (S)-N,N-dimethyl-1-(4-(5-(tetrahydro-2H-pyran- |
| | | 4-yl)-1H-pyrazol-3-ylamino)-6,7-dihydro-5H- |
| | | cyclopenta[d]pyrimidin-2-yl)pyrrolidine-2- |
| | | carboxamide |
| 92 | 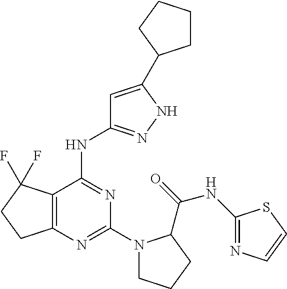 [SMILES: O=C(Nc1nccs1)C1CCCN1c1nc2c(c(Nc3cc(C4CCCC4)[nH]n3)n1)C(F)(F)CC2] | 1-(4-(5-cyclopentyl-1H-pyrazol-3-ylamino)-5,5- difluoro-6,7-dihydro-5H- cyclopenta[d]pyrimidin-2-yl)-N-(thiazol-2- yl)pyrrolidine-2-carboxamide |
| 92a | | (R)-1-(4-(5-cyclopentyl-1H-pyrazol-3-ylamino)- |
| | | 5,5-difluoro-6,7-dihydro-5H- |
| | | cyclopenta[d]pyrimidin-2-yl)-N-(thiazol-2- |
| | | yl)pyrrolidine-2-carboxamide |
| 92b | | (S)-1-(4-(5-cyclopentyl-1H-pyrazol-3-ylamino)- |
| | | 5,5-difluoro-6,7-dihydro-5H- |
| | | cyclopenta[d]pyrimidin-2-yl)-N-(thiazol-2- |
| | | yl)pyrrolidine-2-carboxamide |
| 93 | 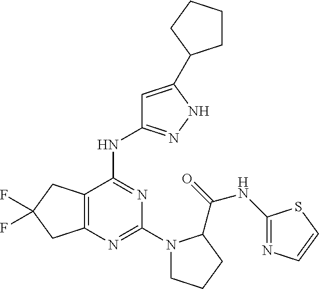 [SMILES: O=C(Nc1nccs1)C1CCCN1c1nc2c(c(Nc3cc(C4CCCC4)[nH]n3)n1)CC(F)(F)C2] | 1-(4-(5-cyclopentyl-1H-pyrazol-3-ylamino)-6,6- difluoro-6,7-dihydro-5H- cyclopenta[d]pyrimidin-2-yl)-N-(thiazol-2- yl)pyrrolidine-2-carboxamide |
| 93a | | (R)-1-(4-(5-cyclopentyl-1H-pyrazol-3-ylamino)- |
| | | 6,6-difluoro-6,7-dihydro-5H- |
| | | cyclopenta[d]pyrimidin-2-yl)-N-(thiazol-2- |
| | | yl)pyrrolidine-2-carboxamide |
| 93b | | (S)-1-(4-(5-cyclopentyl-1H-pyrazol-3-ylamino)- |
| | | 6,6-difluoro-6,7-dihydro-5H- |
| | | cyclopenta[d]pyrimidin-2-yl)-N-(thiazol-2- |
| | | yl)pyrrolidine-2-carboxamide |
| 94 | 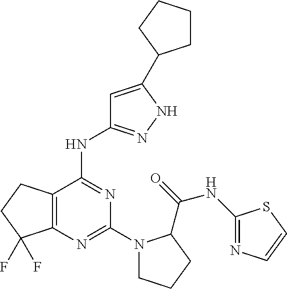 [SMILES: O=C(Nc1nccs1)C1CCCN1c1nc(Nc2cc(C3CCCC3)[nH]n2)c2c(n1)C(F)(F)CC2] | 1-(4-(5-cyclopentyl-1H-pyrazol-3-ylamino)-7,7- difluoro-6,7-dihydro-5H- cyclopenta[d]pyrimidin-2-yl)-N-(thiazol-2- yl)pyrrolidine-2-carboxamide |
| 94a | | (R)-1-(4-(5-cyclopentyl-1H-pyrazol-3-ylamino)- |
| | | 7,7-difluoro-6,7-dihydro-5H- |
| | | cyclopenta[d]pyrimidin-2-yl)-N-(thiazol-2- |
| | | yl)pyrrolidine-2-carboxamide |
| 94b | | (S)-1-(4-(5-cyclopentyl-1H-pyrazol-3-ylamino)- |
| | | 7,7-difluoro-6,7-dihydro-5H- |
| | | cyclopenta[d]pyrimidin-2-yl)-N-(thiazol-2- |
| | | yl)pyrrolidine-2-carboxamide |
| 95 | 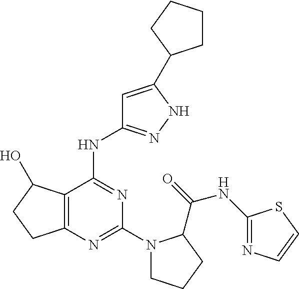 [SMILES: O=C(Nc1nccs1)C1CCCN1c1nc2c(c(Nc3cc(C4CCCC4)[nH]n3)n1)C(O)CC2] | 1-(4-(5-cyclopentyl-1H-pyrazol-3-ylamino)-5- hydroxy-6,7-dihydro-5H- cyclopenta[d]pyrimidin-2-yl)-N-(thiazol-2- yl)pyrrolidine-2-carboxamide |
| 95a | | (R,R)-1-(4-(5-cyclopentyl-1H-pyrazol-3- |
| | | ylamino)-5-hydroxy-6,7-dihydro-5H- |
| | | cyclopenta[d]pyrimidin-2-yl)-N-(thiazol-2- |
| | | yl)pyrrolidine-2-carboxamide |
| 95b | | (R,S)-1-(4-(5-cyclopentyl-1H-pyrazol-3- |
| | | ylamino)-5-hydroxy-6,7-dihydro-5H- |
| | | cyclopenta[d]pyrimidin-2-yl)-N-(thiazol-2- |
| | | yl)pyrrolidine-2-carboxamide |
| 95c | | (S,R)-1-(4-(5-cyclopentyl-1H-pyrazol-3- |
| | | ylamino)-5-hydroxy-6,7-dihydro-5H- |
| | | cyclopenta[d]pyrimidin-2-yl)-N-(thiazol-2- |
| | | yl)pyrrolidine-2-carboxamide |
| 95d | | (S,S)-1-(4-(5-cyclopentyl-1H-pyrazol-3- |
| | | ylamino)-5-hydroxy-6,7-dihydro-5H- |
| | | cyclopenta[d]pyrimidin-2-yl)-N-(thiazol-2- |
| | | yl)pyrrolidine-2-carboxamide |
| 96 | 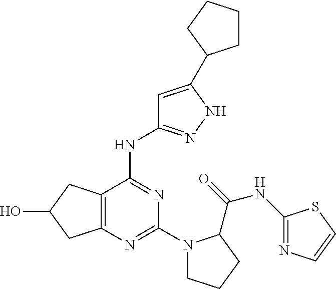 [SMILES: O=C(Nc1nccs1)C1CCCN1c1nc2c(c(Nc3cc(C4CCCC4)[nH]n3)n1)CC(O)C2] | 1-(4-(5-cyclopentyl-1H-pyrazol-3-ylamino)-6- hydroxy-6,7-dihydro-5H- cyclopenta[d]pyrimidin-2-yl)-N-(thiazol-2- yl)pyrrolidine-2-carboxamide |
| 96a | | (R,R)-1-(4-(5-cyclopentyl-1H-pyrazol-3- |
| | | ylamino)-6-hydroxy-6,7-dihydro-5H- |
| | | cyclopenta[d]pyrimidin-2-yl)-N-(thiazol-2- |
| | | yl)pyrrolidine-2-carboxamide |
| 96b | | (R,S)-1-(4-(5-cyclopentyl-1H-pyrazol-3- |
| | | ylamino)-6-hydroxy-6,7-dihydro-5H- |
| | | cyclopenta[d]pyrimidin-2-yl)-N-(thiazol-2- |
| | | yl)pyrrolidine-2-carboxamide |
| 96c | | (S,R)-1-(4-(5-cyclopentyl-1H-pyrazol-3- |
| | | ylamino)-6-hydroxy-6,7-dihydro-5H- |
| | | cyclopenta[d]pyrimidin-2-yl)-N-(thiazol-2- |
| | | yl)pyrrolidine-2-carboxamide |
| 96d | | (S,S)-1-(4-(5-cyclopentyl-1H-pyrazol-3- |
| | | ylamino)-6-hydroxy-6,7-dihydro-5H- |
| | | cyclopenta[d]pyrimidin-2-yl)-N-(thiazol-2- |
| | | yl)pyrrolidine-2-carboxamide |
| 97 | 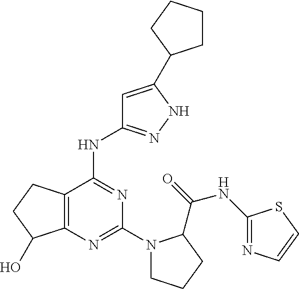 [SMILES: O=C(Nc1nccs1)C1CCCN1c1nc(Nc2cc(C3CCCC3)[nH]n2)c2c(n1)C(O)CC2] | 1-(4-(5-cyclopentyl-1H-pyrazol-3-ylamino)-7- hydroxy-6,7-dihydro-5H- cyclopenta[d]pyrimidin-2-yl)-N-(thiazol-2- yl)pyrrolidine-2-carboxamide |
| 97a | | (R,R)-1-(4-(5-cyclopentyl-1H-pyrazol-3- |
| | | ylamino)-7-hydroxy-6,7-dihydro-5H- |
| | | cyclopenta[d]pyrimidin-2-yl)-N-(thiazol-2- |
| | | yl)pyrrolidine-2-carboxamide |
| 97b | | (R,S)-1-(4-(5-cyclopentyl-1H-pyrazol-3- |
| | | ylamino)-7-hydroxy-6,7-dihydro-5H- |
| | | cyclopenta[d]pyrimidin-2-yl)-N-(thiazol-2- |
| | | yl)pyrrolidine-2-carboxamide |
| 97c | | (S,R)-1-(4-(5-cyclopentyl-1H-pyrazol-3- |
| | | ylamino)-7-hydroxy-6,7-dihydro-5H- |
| | | cyclopenta[d]pyrimidin-2-yl)-N-(thiazol-2- |
| | | yl)pyrrolidine-2-carboxamide |
| 97d | | (S,S)-1-(4-(5-cyclopentyl-1H-pyrazol-3- |
| | | ylamino)-7-hydroxy-6,7-dihydro-5H- |
| | | cyclopenta[d]pyrimidin-2-yl)-N-(thiazol-2- |
| | | yl)pyrrolidine-2-carboxamide |
| 98 | 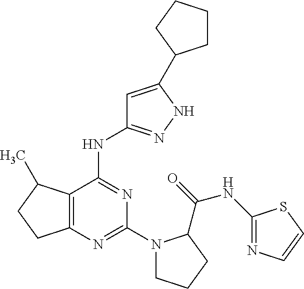 [SMILES: CC1CCc2nc(N3CCCC3C(=O)Nc3nccs3)nc(Nc3cc(C4CCCC4)[nH]n3)c21] | 1-(4-(5-cyclopentyl-1H-pyrazol-3-ylamino)-5- methyl-6,7-dihydro-5H-cyclopenta[d]pyrimidin- 2-yl)-N-(thiazol-2-yl)pyrrolidine-2-carboxamide |
| 98a | | (R,R)-1-(4-(5-cyclopentyl-1H-pyrazol-3- |
| | | ylamino)-5-methyl-6,7-dihydro-5H- |
| | | cyclopenta[d]pyrimidin-2-yl)-N-(thiazol-2- |
| | | yl)pyrrolidine-2-carboxamide |
| 98b | | (R,S)-1-(4-(5-cyclopentyl-1H-pyrazol-3- |
| | | ylamino)-5-methyl-6,7-dihydro-5H- |
| | | cyclopenta[d]pyrimidin-2-yl)-N-(thiazol-2- |
| | | yl)pyrrolidine-2-carboxamide |
| 98c | | (S,R)-1-(4-(5-cyclopentyl-1H-pyrazol-3- |
| | | ylamino)-5-methyl-6,7-dihydro-5H- |
| | | cyclopenta[d]pyrimidin-2-yl)-N-(thiazol-2- |
| | | yl)pyrrolidine-2-carboxamide |
| 98d | | (S,S)-1-(4-(5-cyclopentyl-1H-pyrazol-3- |
| | | ylamino)-5-methyl-6,7-dihydro-5H- |
| | | cyclopenta[d]pyrimidin-2-yl)-N-(thiazol-2- |
| | | yl)pyrrolidine-2-carboxamide |
| 99 | 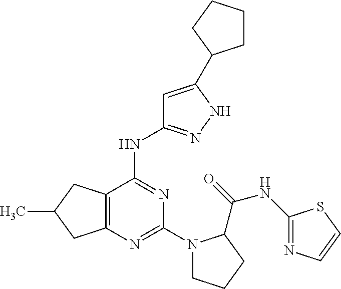 [SMILES: CC1Cc2nc(N3CCCC3C(=O)Nc3nccs3)nc(Nc3cc(C4CCCC4)[nH]n3)c2C1] | 1-(4-(5-cyclopentyl-1H-pyrazol-3-ylamino)-6- methyl-6,7-dihydro-5H-cyclopenta[d]pyrimidin- 2-yl)-N-(thiazol-2-yl)pyrrolidine-2-carboxamide |
| 99a | | (R,R)-1-(4-(5-cyclopentyl-1H-pyrazol-3- |
| | | ylamino)-6-methyl-6,7-dihydro-5H- |
| | | cyclopenta[d]pyrimidin-2-yl)-N-(thiazol-2- |
| | | yl)pyrrolidine-2-carboxamide |
| 99b | | (R,S)-1-(4-(5-cyclopentyl-1H-pyrazol-3- |
| | | ylamino)-6-methyl-6,7-dihydro-5H- |
| | | cyclopenta[d]pyrimidin-2-yl)-N-(thiazol-2- |
| | | yl)pyrrolidine-2-carboxamide |
| 99c | | (S,R)-1-(4-(5-cyclopentyl-1H-pyrazol-3- |
| | | ylamino)-6-methyl-6,7-dihydro-5H- |
| | | cyclopenta[d]pyrimidin-2-yl)-N-(thiazol-2- |
| | | yl)pyrrolidine-2-carboxamide |
| 99d | | (S,S)-1-(4-(5-cyclopentyl-1H-pyrazol-3- |
| | | ylamino)-6-methyl-6,7-dihydro-5H- |
| | | cyclopenta[d]pyrimidin-2-yl)-N-(thiazol-2- |
| | | yl)pyrrolidine-2-carboxamide |
| 100 | 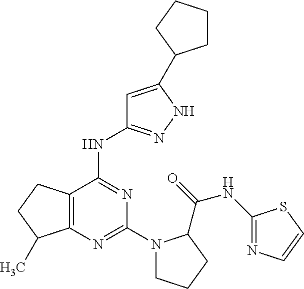 [SMILES: CC1CCc2c(Nc3cc(C4CCCC4)[nH]n3)nc(N3CCCC3C(=O)Nc3nccs3)nc21] | 1-(4-(5-cyclopentyl-1H-pyrazol-3-ylamino)-7- methyl-6,7-dihydro-5H-cyclopenta[d]pyrimidin- 2-yl)-N-(thiazol-2-yl)pyrrolidine-2-carboxamide |
| 100a | | (R,R)-1-(4-(5-cyclopentyl-1H-pyrazol-3- |
| | | ylamino)-7-methyl-6,7-dihydro-5H- |
| | | cyclopenta[d]pyrimidin-2-yl)-N-(thiazol-2- |
| | | yl)pyrrolidine-2-carboxamide |
| 100b | | (R,S)-1-(4-(5-cyclopentyl-1H-pyrazol-3- |
| | | ylamino)-7-methyl-6,7-dihydro-5H- |
| | | cyclopenta[d]pyrimidin-2-yl)-N-(thiazol-2- |
| | | yl)pyrrolidine-2-carboxamide |
| 100c | | (S,R)-1-(4-(5-cyclopentyl-1H-pyrazol-3- |
| | | ylamino)-7-methyl-6,7-dihydro-5H- |
| | | cyclopenta[d]pyrimidin-2-yl)-N-(thiazol-2- |
| | | yl)pyrrolidine-2-carboxamide |
| 100d | | (S,S)-1-(4-(5-cyclopentyl-1H-pyrazol-3- |
| | | ylamino)-7-methyl-6,7-dihydro-5H- |
| | | cyclopenta[d]pyrimidin-2-yl)-N-(thiazol-2- |
| | | yl)pyrrolidine-2-carboxamide |
| 101 | 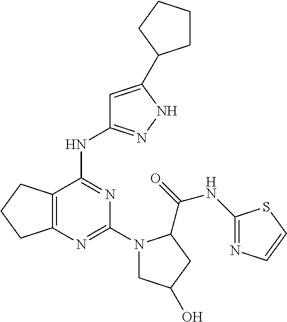 [SMILES: O=C(Nc1nccs1)C1CC(O)CN1c1nc2c(c(Nc3cc(C4CCCC4)[nH]n3)n1)CCC2] | 1-(4-(5-cyclopentyl-1H-pyrazol-3-ylamino)-6,7- dihydro-5H-cyclopenta[d]pyrimidin-2-yl)-4- hydroxy-N-(thiazol-2-yl)pyrrolidine-2- carboxamide |
| 101a | | (R,R)-1-(4-(5-cyclopentyl-1H-pyrazol-3- |
| | | ylamino)-6,7-dihydro-5H- |
| | | cyclopenta[d]pyrimidin-2-yl)-4-hydroxy-N- |
| | | (thiazol-2-yl)pyrrolidine-2-carboxamide |
| 101b | | (R,S)-1-(4-(5-cyclopentyl-1H-pyrazol-3- |
| | | ylamino)-6,7-dihydro-5H- |
| | | cyclopenta[d]pyrimidin-2-yl)-4-hydroxy-N- |
| | | (thiazol-2-yl)pyrrolidine-2-carboxamide |
| 101c | | (S,R)-1-(4-(5-cyclopentyl-1H-pyrazol-3- |
| | | ylamino)-6,7-dihydro-5H- |
| | | cyclopenta[d]pyrimidin-2-yl)-4-hydroxy-N- |
| | | (thiazol-2-yl)pyrrolidine-2-carboxamide |
| 101d | | (S,S)-1-(4-(5-cyclopentyl-1H-pyrazol-3- |
| | | ylamino)-6,7-dihydro-5H- |
| | | cyclopenta[d]pyrimidin-2-yl)-4-hydroxy-N- |
| | | (thiazol-2-yl)pyrrolidine-2-carboxamide |
| 102 | 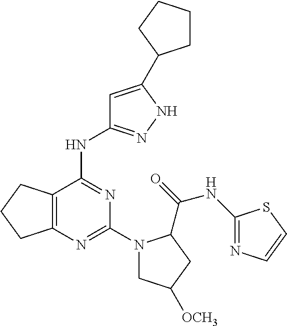 [SMILES: COC1CC(C(=O)Nc2nccs2)N(c2nc3c(c(Nc4cc(C5CCCC5)[nH]n4)n2)CCC3)C1] | 1-(4-(5-cyclopentyl-1H-pyrazol-3-ylamino)-6,7- dihydro-5H-cyclopenta[d]pyrimidin-2-yl)-4- methoxy-N-(thiazol-2-yl)pyrrolidine-2- carboxamide |
| 102a | | (R,R)-1-(4-(5-cyclopentyl-1H-pyrazol-3- |
| | | ylamino)-6,7-dihydro-5H- |
| | | cyclopenta[d]pyrimidin-2-yl)-4-methoxy-N- |
| | | (thiazol-2-yl)pyrrolidine-2-carboxamide |
| 102b | | (R,S)-1-(4-(5-cyclopentyl-1H-pyrazol-3- |
| | | ylamino)-6,7-dihydro-5H- |
| | | cyclopenta[d]pyrimidin-2-yl)-4-methoxy-N- |
| | | (thiazol-2-yl)pyrrolidine-2-carboxamide |
| 102c | | (S,R)-1-(4-(5-cyclopentyl-1H-pyrazol-3- |
| | | ylamino)-6,7-dihydro-5H- |
| | | cyclopenta[d]pyrimidin-2-yl)-4-methoxy-N- |
| | | (thiazol-2-yl)pyrrolidine-2-carboxamide |
| 102d | | (S,S)-1-(4-(5-cyclopentyl-1H-pyrazol-3- |
| | | ylamino)-6,7-dihydro-5H- |
| | | cyclopenta[d]pyrimidin-2-yl)-4-methoxy-N- |
| | | (thiazol-2-yl)pyrrolidine-2-carboxamide |
| 103 | 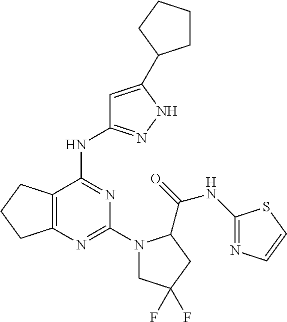 [SMILES: O=C(Nc1nccs1)C1CC(F)(F)CN1c1nc2c(c(Nc3cc(C4CCCC4)[nH]n3)n1)CCC2] | 1-(4-(5-cyclopentyl-1H-pyrazol-3-ylamino)-6,7- dihydro-5H-cyclopenta[d]pyrimidin-2-yl)-4,4- difluoro-N-(thiazol-2-yl)pyrrolidine-2- carboxamide |
| 103a | | (R)-1-(4-(5-cyclopentyl-1H-pyrazol-3-ylamino)- |
| | | 6,7-dihydro-5H-cyclopenta[d]pyrimidin-2-yl)- |
| | | 4,4-difluoro-N-(thiazol-2-yl)pyrrolidine-2- |
| | | carboxamide |
| 103b | | (S)-1-(4-(5-cyclopentyl-1H-pyrazol-3-ylamino)- |
| | | 6,7-dihydro-5H-cyclopenta[d]pyrimidin-2-yl)- |
| | | 4,4-difluoro-N-(thiazol-2-yl)pyrrolidine-2- |
| | | carboxamide |
| 104 | 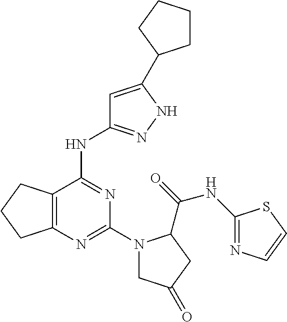 [SMILES: O=C1CC(C(=O)Nc2nccs2)N(c2nc3c(c(Nc4cc(C5CCCC5)[nH]n4)n2)CCC3)C1] | 1-(4-(5-cyclopentyl-1H-pyrazol-3-ylamino)-6,7- dihydro-5H-cyclopenta[d]pyrimidin-2-yl)-4- oxo-N-(thiazol-2-yl)pyrrolidine-2-carboxamide |
| 104a | | (R)-1-(4-(5-cyclopentyl-1H-pyrazol-3-ylamino)- |
| | | 6,7-dihydro-5H-cyclopenta[d]pyrimidin-2-yl)-4- |
| | | oxo-N-(thiazol-2-yl)pyrrolidine-2-carboxamide |
| 104b | | (S)-1-(4-(5-cyclopentyl-1H-pyrazol-3-ylamino)- |
| | | 6,7-dihydro-5H-cyclopenta[d]pyrimidin-2-yl)-4- |
| | | oxo-N-(thiazol-2-yl)pyrrolidine-2-carboxamide |
| 105 | 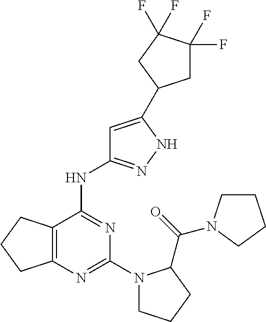 [SMILES: O=C(C1CCCN1c1nc2c(c(Nc3cc(C4CC(F)(F)C(F)(F)C4)[nH]n3)n1)CCC2)N1CCCC1] | pyrrolidin-1-yl(1-(4-(5-(3,3,4,4- tetrafluorocyclopentyl)-1H-pyrazol-3-ylamino)- 6,7-dihydro-5H-cyclopenta[d]pyrimidin-2- yl)pyrrolidin-2-yl)methanone |
| 105a | | (R)-pyrrolidin-1-yl(1-(4-(5-(3,3,4,4- |
| | | tetrafluorocyclopentyl)-1H-pyrazol-3-ylamino)- |
| | | 6,7-dihydro-5H-cyclopenta[d]pyrimidin-2- |
| | | yl)pyrrolidin-2-yl)methanone |
| 105b | | (S)-pyrrolidin-1-yl(1-(4-(5-(3,3,4,4- |
| | | tetrafluorocyclopentyl)-1H-pyrazol-3-ylamino)- |
| | | 6,7-dihydro-5H-cyclopenta[d]pyrimidin-2- |
| | | yl)pyrrolidin-2-yl)methanone |
| 106 | 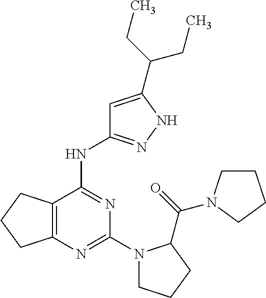 [SMILES: CCC(CC)c1cc(Nc2nc(N3CCCC3C(=O)N3CCCC3)nc3c2CCC3)n[nH]1] | (1-(4-(5-(pentan-3-yl)-1H-pyrazol-3-ylamino)- 6,7-dihydro-5H-cyclopenta[d]pyrimidin-2- yl)pyrrolidin-2-yl)(pyrrolidin-1-yl)methanone |
| 106a | | (R)-(1-(4-(5-(pentan-3-yl)-1H-pyrazol-3- |
| | | ylamino)-6,7-dihydro-5H- |
| | | cyclopenta[d]pyrimidin-2-yl)pyrrolidin-2- |
| | | yl)(pyrrolidin-1-yl)methanone |
| 106b | | (S)-(1-(4-(5-(pentan-3-yl)-1H-pyrazol-3- |
| | | ylamino)-6,7-dihydro-5H- |
| | | cyclopenta[d]pyrimidin-2-yl)pyrrolidin-2- |
| | | yl)(pyrrolidin-1-yl)methanone |
| 107 | 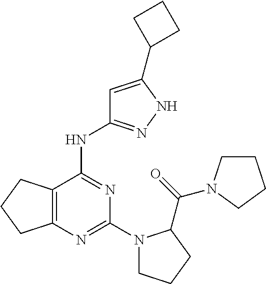 [SMILES: O=C(C1CCCN1c1nc2c(c(Nc3cc(C4CCC4)[nH]n3)n1)CCC2)N1CCCC1] | (1-(4-(5-cyclobutyl-1H-pyrazol-3-ylamino)-6,7- dihydro-5H-cyclopenta[d]pyrimidin-2- yl)pyrrolidin-2-yl)(pyrrolidin-1-yl)methanone |
| 107a | | (R)-(1-(4-(5-cyclobutyl-1H-pyrazol-3-ylamino)- |
| | | 6,7-dihydro-5H-cyclopenta[d]pyrimidin-2- |
| | | yl)pyrrolidin-2-yl)(pyrrolidin-1-yl)methanone |
| 107b | | (S)-(1-(4-(5-cyclobutyl-1H-pyrazol-3-ylamino)- |
| | | 6,7-dihydro-5H-cyclopenta[d]pyrimidin-2- |
| | | yl)pyrrolidin-2-yl)(pyrrolidin-1-yl)methanone |
| 108 | 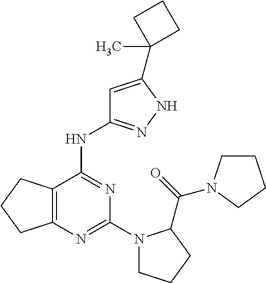 [SMILES: CC1(c2cc(Nc3nc(N4CCCC4C(=O)N4CCCC4)nc4c3CCC4)n[nH]2)CCC1] | (1-(4-(5-(1-methylcyclobutyl)-1H-pyrazol-3- ylamino)-6,7-dihydro-5H- cyclopenta[d]pyrimidin-2-yl)pyrrolidin-2- yl)(pyrrolidin-1-yl)methanone |
| 108a | | (R)-(1-(4-(5-(1-methylcyclobutyl)-1H-pyrazol-3- |
| | | ylamino)-6,7-dihydro-5H- |
| | | cyclopenta[d]pyrimidin-2-yl)pyrrolidin-2- |
| | | yl)(pyrrolidin-1-yl)methanone |
| 108b | | (S)-(1-(4-(5-(1-methylcyclobutyl)-1H-pyrazol-3- |
| | | ylamino)-6,7-dihydro-5H- |
| | | cyclopenta[d]pyrimidin-2-yl)pyrrolidin-2- |
| | | yl)(pyrrolidin-1-yl)methanone |
| 109 | 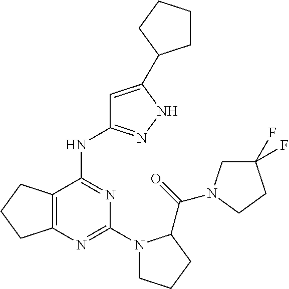 [SMILES: O=C(C1CCCN1c1nc2c(c(Nc3cc(C4CCCC4)[nH]n3)n1)CCC2)N1CCC(F)(F)C1] | (1-(4-(5-cyclopentyl-1H-pyrazol-3-ylamino)- 6,7-dihydro-5H-cyclopenta[d]pyrimidin-2- yl)pyrrolidin-2-yl)(3,3-difluoropyrrolidin-1- yl)methanone |
| 109a | | (R)-(1-(4-(5-cyclopentyl-1H-pyrazol-3- |
| | | ylamino)-6,7-dihydro-5H- |
| | | cyclopenta[d]pyrimidin-2-yl)pyrrolidin-2- |
| | | yl)(3,3-difluoropyrrolidin-1-yl)methanone |
| 109b | | (S)-(1-(4-(5-cyclopentyl-1H-pyrazol-3-ylamino)- |
| | | 6,7-dihydro-5H-cyclopenta[d]pyrimidin-2- |
| | | yl)pyrrolidin-2-yl)(3,3-difluoropyrrolidin-1- |
| | | yl)methanone |
| 110 | 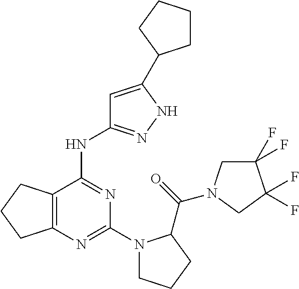 [SMILES: O=C(C1CCCN1c1nc2c(c(Nc3cc(C4CCCC4)[nH]n3)n1)CCC2)N1CC(F)(F)C(F)(F)C1] | (1-(4-(5-cyclopentyl-1H-pyrazol-3-ylamino)- 6,7-dihydro-5H-cyclopenta[d]pyrimidin-2- yl)pyrrolidin-2-yl)(3,3,4,4-tetrafluoropyrrolidin- 1-yl)methanone |
| 110a | | (R)-(1-(4-(5-cyclopentyl-1H-pyrazol-3- |
| | | ylamino)-6,7-dihydro-5H- |
| | | cyclopenta[d]pyrimidin-2-yl)pyrrolidin-2- |
| | | yl)(3,3,4,4-tetrafluoropyrrolidin-1-yl)methanone |
| 110b | | (S)-(1-(4-(5-cyclopentyl-1H-pyrazol-3-ylamino)- |
| | | 6,7-dihydro-5H-cyclopenta[d]pyrimidin-2- |
| | | yl)pyrrolidin-2-yl)(3,3,4,4-tetrafluoropyrrolidin- |
| | | 1-yl)methanone |
| 111 | 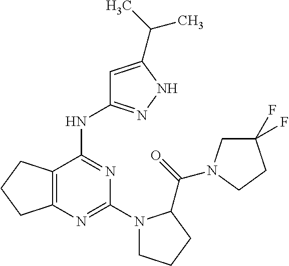 [SMILES: CC(C)c1cc(Nc2nc(N3CCCC3C(=O)N3CCC(F)(F)C3)nc3c2CCC3)n[nH]1] | (3,3-difluoropyrrolidin-1-yl)(1-(4-(5-isopropyl- 1H-pyrazol-3-ylamino)-6,7-dihydro-5H- cyclopenta[d]pyrimidin-2-yl)pyrrolidin-2- yl)methanone |
| 111a | | (R)-(3,3-difluoropyrrolidin-1-yl)(1-(4-(5- |
| | | isopropyl-1H-pyrazol-3-ylamino)-6,7-dihydro- |
| | | 5H-cyclopenta[d]pyrimidin-2-yl)pyrrolidin-2- |
| | | yl)methanone |
| 111b | | (S)-(3,3-difluoropyrrolidin-1-yl)(1-(4-(5- |
| | | isopropyl-1H-pyrazol-3-ylamino)-6,7-dihydro- |
| | | 5H-cyclopenta[d]pyrimidin-2-yl)pyrrolidin-2- |
| | | yl)methanone |
| 112 | 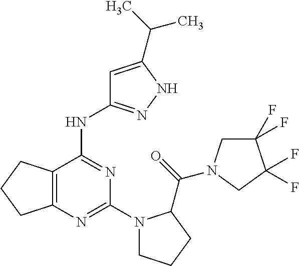 [SMILES: CC(C)c1cc(Nc2nc(N3CCCC3C(=O)N3CC(F)(F)C(F)(F)C3)nc3c2CCC3)n[nH]1] | (1-(4-(5-isopropyl-1H-pyrazol-3-ylamino)-6,7- dihydro-5H-cyclopenta[d]pyrimidin-2- yl)pyrrolidin-2-yl)(3,3,4,4-tetrafluoropyrrolidin- 1-yl)methanone |
| 112a | | (R)-(1-(4-(5-isopropyl-1H-pyrazol-3-ylamino)- |
| | | 6,7-dihydro-5H-cyclopenta[d]pyrimidin-2- |
| | | yl)pyrrolidin-2-yl)(3,3,4,4-tetrafluoropyrrolidin- |
| | | 1-yl)methanone |
| 112b | | (S)-(1-(4-(5-isopropyl-1H-pyrazol-3-ylamino)- |
| | | 6,7-dihydro-5H-cyclopenta[d]pyrimidin-2- |
| | | yl)pyrrolidin-2-yl)(3,3,4,4-tetrafluoropyrrolidin- |
| | | 1-yl)methanone |
| 113 | 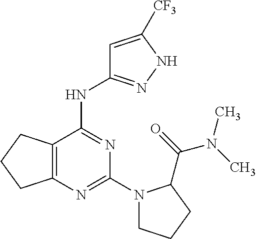 [SMILES: CN(C)C(=O)C1CCCN1c1nc2c(c(Nc3cc(C(F)(F)F)[nH]n3)n1)CCC2] | N,N-dimethyl-1-(4-(5-(trifluoromethyl)-1H- pyrazol-3-ylamino)-6,7-dihydro-5H- cyclopenta[d]pyrimidin-2-yl)pyrrolidine-2- carboxamide |
| 113a | | (R)-N,N-dimethyl-1-(4-(5-(trifluoromethyl)-1H- |
| | | pyrazol-3-ylamino)-6,7-dihydro-5H- |
| | | cyclopenta[d]pyrimidin-2-yl)pyrrolidine-2- |
| | | carboxamide |
| 113b | | (S)-N,N-dimethyl-1-(4-(5-(trifluoromethyl)-1H- |
| | | pyrazol-3-ylamino)-6,7-dihydro-5H- |
| | | cyclopenta[d]pyrimidin-2-yl)pyrrolidine-2- |
| | | carboxamide |
| 114 | 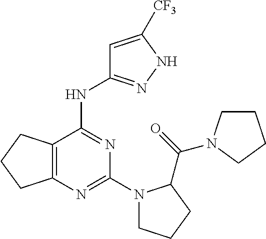 [SMILES: O=C(C1CCCN1c1nc2c(c(Nc3cc(C(F)(F)F)[nH]n3)n1)CCC2)N1CCCC1] | pyrrolidin-1-yl(1-(4-(5-(trifluoromethyl)-1H- pyrazol-3-ylamino)-6,7-dihydro-5H- cyclopenta[d]pyrimidin-2-yl)pyrrolidin-2- yl)methanone |
| 114a | | (R)-pyrrolidin-1-yl(1-(4-(5-(trifluoromethyl)- |
| | | 1H-pyrazol-3-ylamino)-6,7-dihydro-5H- |
| | | cyclopenta[d]pyrimidin-2-yl)pyrrolidin-2- |
| | | yl)methanone |
| 114b | | (S)-pyrrolidin-1-yl(1-(4-(5-(trifluoromethyl)-1H- |
| | | pyrazol-3-ylamino)-6,7-dihydro-5H- |
| | | cyclopenta[d]pyrimidin-2-yl)pyrrolidin-2- |
| | | yl)methanone |
| 115 | 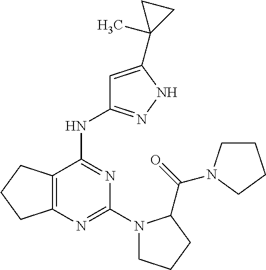 [SMILES: CC1(c2cc(Nc3nc(N4CCCC4C(=O)N4CCCC4)nc4c3CCC4)n[nH]2)CC1] | (1-(4-(5-(1-methylcyclopropyl)-1H-pyrazol-3- ylamino)-6,7-dihydro-5H- cyclopenta[d]pyrimidin-2-yl)pyrrolidin-2- yl)(pyrrolidin-1-yl)methanone |
| 115a | | (R)-(1-(4-(5-(1-methylcyclopropyl)-1H-pyrazol- |
| | | 3-ylamino)-6,7-dihydro-5H- |
| | | cyclopenta[d]pyrimidin-2-yl)pyrrolidin-2- |
| | | yl)(pyrrolidin-1-yl)methanone |
| 115b | | (S)-(1-(4-(5-(1-methylcyclopropyl)-1H-pyrazol- |
| | | 3-ylamino)-6,7-dihydro-5H- |
| | | cyclopenta[d]pyrimidin-2-yl)pyrrolidin-2- |
| | | yl)(pyrrolidin-1-yl)methanone |
| 116 | 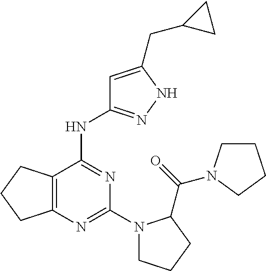 [SMILES: O=C(C1CCCN1c1nc2c(c(Nc3cc(CC4CC4)[nH]n3)n1)CCC2)N1CCCC1] | (1-(4-(5-(cyclopropylmethyl)-1H-pyrazol-3- ylamino)-6,7-dihydro-5H- cyclopenta[d]pyrimidin-2-yl)pyrrolidin-2- yl)(pyrrolidin-1-yl)methanone |
| 116a | | (R)-(1-(4-(5-(cyclopropylmethyl)-1H-pyrazol-3- |
| | | ylamino)-6,7-dihydro-5H- |
| | | cyclopenta[d]pyrimidin-2-yl)pyrrolidin-2- |
| | | yl)(pyrrolidin-1-yl)methanone |
| 116b | | (S)-(1-(4-(5-(cyclopropylmethyl)-1H-pyrazol-3- |
| | | ylamino)-6,7-dihydro-5H- |
| | | cyclopenta[d]pyrimidin-2-yl)pyrrolidin-2- |
| | | yl)(pyrrolidin-1-yl)methanone |
| 117 | 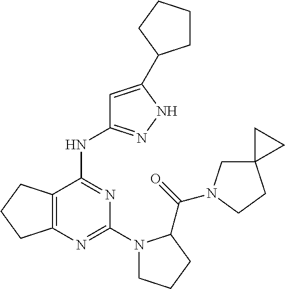 [SMILES: O=C(C1CCCN1c1nc2c(c(Nc3cc(C4CCCC4)[nH]n3)n1)CCC2)N1CCC2(CC2)C1] | (1-(4-(5-cyclopentyl-1H-pyrazol-3-ylamino)- 6,7-dihydro-5H-cyclopenta[d]pyrimidin-2- yl)pyrrolidin-2-yl)(5-azaspiro[2.4]heptan-5- yl)methanone |
| 117a | | (R)-(1-(4-(5-cyclopentyl-1H-pyrazol-3- |
| | | ylamino)-6,7-dihydro-5H- |
| | | cyclopenta[d]pyrimidin-2-yl)pyrrolidin-2-yl)(5- |
| | | azaspiro[2.4]heptan-5-yl)methanone |
| 117b | | (S)-(1-(4-(5-cyclopentyl-1H-pyrazol-3-ylamino)- |
| | | 6,7-dihydro-5H-cyclopenta[d]pyrimidin-2- |
| | | yl)pyrrolidin-2-yl)(5-azaspiro[2.4]heptan-5- |
| | | yl)methanone |
| 118 | 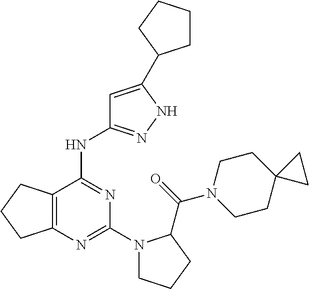 [SMILES: O=C(C1CCCN1c1nc2c(c(Nc3cc(C4CCCC4)[nH]n3)n1)CCC2)N1CCC2(CC1)CC2] | (1-(4-(5-cyclopentyl-1H-pyrazol-3-ylamino)- 6,7-dihydro-5H-cyclopenta[d]pyrimidin-2- yl)pyrrolidin-2-yl)(6-azaspiro[2.5]octan-6- yl)methanone |
| 118a | | (R)-(1-(4-(5-cyclopentyl-1H-pyrazol-3- |
| | | ylamino)-6,7-dihydro-5H- |
| | | cyclopenta[d]pyrimidin-2-yl)pyrrolidin-2-yl)(6- |
| | | azaspiro[2.5]octan-6-yl)methanone |
| 118b | | (S)-(1-(4-(5-cyclopentyl-1H-pyrazol-3-ylamino)- |
| | | 6,7-dihydro-5H-cyclopenta[d]pyrimidin-2- |
| | | yl)pyrrolidin-2-yl)(6-azaspiro[2.5]octan-6- |
| | | yl)methanone |
| 119 | 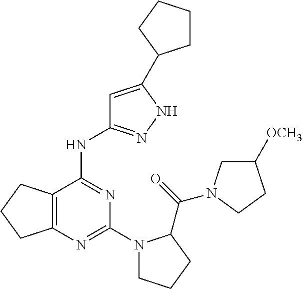 [SMILES: COC1CCN(C(=O)C2CCCN2c2nc3c(c(Nc4cc(C5CCCC5)[nH]n4)n2)CCC3)C1] | (1-(4-(5-cyclopentyl-1H-pyrazol-3-ylamino)- 6,7-dihydro-5H-cyclopenta[d]pyrimidin-2- yl)pyrrolidin-2-yl)(3-methoxypyrrolidin-1- yl)methanone |
| 119a | | (R)-(1-(4-(5-cyclopentyl-1H-pyrazol-3- |
| | | ylamino)-6,7-dihydro-5H- |
| | | cyclopenta[d]pyrimidin-2-yl)pyrrolidin-2-yl)(3- |
| | | methoxypyrrolidin-1-yl)methanone |
| 119b | | (S)-(1-(4-(5-cyclopentyl-1H-pyrazol-3-ylamino)- |
| | | 6,7-dihydro-5H-cyclopenta[d]pyrimidin-2- |
| | | yl)pyrrolidin-2-yl)(3-methoxypyrrolidin-1- |
| | | yl)methanone |
| 120 | 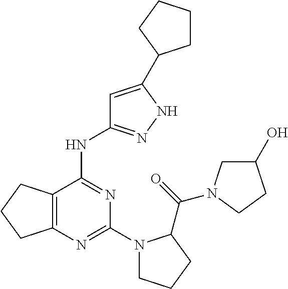 [SMILES: O=C(C1CCCN1c1nc2c(c(Nc3cc(C4CCCC4)[nH]n3)n1)CCC2)N1CCC(O)C1] | (1-(4-(5-cyclopentyl-1H-pyrazol-3-ylamino)- 6,7-dihydro-5H-cyclopenta[d]pyrimidin-2- yl)pyrrolidin-2-yl)(3-hydroxypyrrolidin-1- yl)methanone |
| 120a | | (R)-(1-(4-(5-cyclopentyl-1H-pyrazol-3- |
| | | ylamino)-6,7-dihydro-5H- |
| | | cyclopenta[d]pyrimidin-2-yl)pyrrolidin-2-yl)(3- |
| | | hydroxypyrrolidin-1-yl)methanone |
| 120b | | (S)-(1-(4-(5-cyclopentyl-1H-pyrazol-3-ylamino)- |
| | | 6,7-dihydro-5H-cyclopenta[d]pyrimidin-2- |
| | | yl)pyrrolidin-2-yl)(3-hydroxypyrrolidin-1- |
| | | yl)methanone |
| 121 | 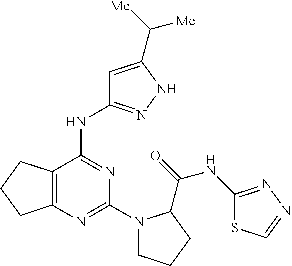 [SMILES: CC(C)c1cc(Nc2nc(N3CCCC3C(=O)Nc3nncs3)nc3c2CCC3)n[nH]1] | 1-(4-((5-isopropyl-1H-pyrazol-3-yl)amino)-6,7- dihydro-5H-cyclopenta[d]pyrimidin-2-yl)-N- (1,3,4-thiadiazol-2-yl)pyrrolidine-2-carboxamide |
| 121a | | (R)-1-(4-((5-isopropyl-1H-pyrazol-3-yl)amino)- |
| | | 6,7-dihydro-5H-cyclopenta[d]pyrimidin-2-yl)- |
| | | N-(1,3,4-thiadiazol-2-yl)pyrrolidine-2- |
| | | carboxamide |
| 121b | | (S)-1-(4-((5-isopropyl-1H-pyrazol-3-yl)amino)- |
| | | 6,7-dihydro-5H-cyclopenta[d]pyrimidin-2-yl)- |
| | | N-(1,3,4-thiadiazol-2-yl)pyrrolidine-2- |
| | | carboxamide |
| 122 | 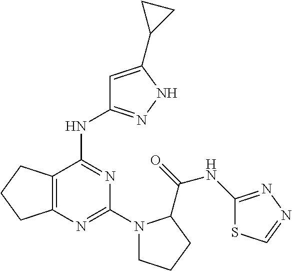 [SMILES: O=C(Nc1nncs1)C1CCCN1c1nc2c(c(Nc3cc(C4CC4)[nH]n3)n1)CCC2] | 1-(4-((5-cyclopropyl-1H-pyrazol-3-yl)amino)- 6,7-dihydro-5H-cyclopenta[d]pyrimidin-2-yl)- N-(1,3,4-thiadiazol-2-yl)pyrrolidine-2- carboxamide |
| 122a | | (R)-1-(4-((5-cyclopropyl-1H-pyrazol-3- |
| | | yl)amino)-6,7-dihydro-5H- |
| | | cyclopenta[d]pyrimidin-2-yl)-N-(1,3,4- |
| | | thiadiazol-2-yl)pyrrolidine-2-carboxamide |
| 122b | | (S)-1-(4-((5-cyclopropyl-1H-pyrazol-3- |
| | | yl)amino)-6,7-dihydro-5H- |
| | | cyclopenta[d]pyrimidin-2-yl)-N-(1,3,4- |
| | | thiadiazol-2-yl)pyrrolidine-2-carboxamide |
| 123 | 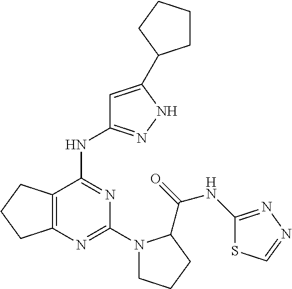 [SMILES: O=C(Nc1nncs1)C1CCCN1c1nc2c(c(Nc3cc(C4CCCC4)[nH]n3)n1)CCC2] | 1-(4-((5-cyclopentyl-1H-pyrazol-3-yl)amino)- 6,7-dihydro-5H-cyclopenta[d]pyrimidin-2-yl)- N-(1,3,4-thiadiazol-2-yl)pyrrolidine-2- carboxamide |
| 123a | | (R)-1-(4-(5-cyclopentyl-1H-pyrazol-3- |
| | | yl)amino)-6,7-dihydro-5H- |
| | | cyclopenta[d]pyrimidin-2-yl)-N-(1,3,4- |
| | | thiadiazol-2-yl)pyrrolidine-2-carboxamide |
| 123b | | (S)-1-(4-(5-cyclopentyl-1H-pyrazol-3- |
| | | yl)amino)-6,7-dihydro-5H- |
| | | cyclopenta[d]pyrimidin-2-yl)-N-(1,3,4- |
| | | thiadiazol-2-yl)pyrrolidine-2-carboxamide |
| 124 | 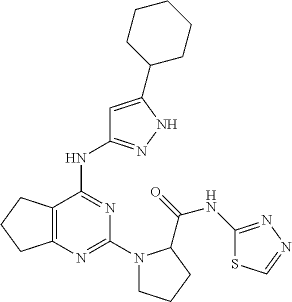 [SMILES: O=C(Nc1nncs1)C1CCCN1c1nc2c(c(Nc3cc(C4CCCCC4)[nH]n3)n1)CCC2] | 1-(4-((5-cyclohexyl-1H-pyrazol-3-yl)amino)- 6,7-dihydro-5H-cyclopenta[d]pyrimidin-2-yl)- N-(1,3,4-thiadiazol-2-yl)pyrrolidine-2- carboxamide |
| 124a | | (R)-1-(4-((5-cyclohexyl-1H-pyrazol-3- |
| | | yl)amino)-6,7-dihydro-5H- |
| | | cyclopenta[d]pyrimidin-2-yl)-N-(1,3,4- |
| | | thiadiazol-2-yl)pyrrolidine-2-carboxamide |
| 124b | | (S)-1-(4-((5-cyclohexyl-1H-pyrazol-3- |
| | | yl)amino)-6,7-dihydro-5H- |
| | | cyclopenta[d]pyrimidin-2-yl)-N-(1,3,4- |
| | | thiadiazol-2-yl)pyrrolidine-2-carboxamide |
| 125 | 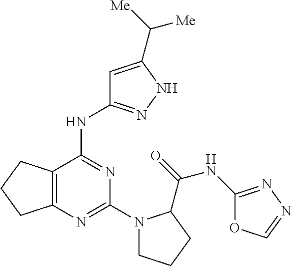 [SMILES: CC(C)c1cc(Nc2nc(N3CCCC3C(=O)Nc3nnco3)nc3c2CCC3)n[nH]1] | 1-(4-((5-isopropyl-1H-pyrazol-3-yl)amino)-6,7- dihydro-5H-cyclopenta[d]pyrimidin-2-yl)-N- (1,3,4-oxadiazol-2-yl)pyrrolidine-2-carboxamide |
| 125a | | (R)-1-(4-((5-isopropyl-1H-pyrazol-3-yl)amino)- |
| | | 6,7-dihydro-5H-cyclopenta[d]pyrimidin-2-yl)- |
| | | N-(1,3,4-oxadiazol-2-yl)pyrrolidine-2- |
| | | carboxamide |
| 125b | | (S)-1-(4-((5-isopropyl-1H-pyrazol-3-yl)amino)- |
| | | 6,7-dihydro-5H-cyclopenta[d]pyrimidin-2-yl)- |
| | | N-(1,3,4-oxadiazol-2-yl)pyrrolidine-2- |
| | | carboxamide |
| 126 | 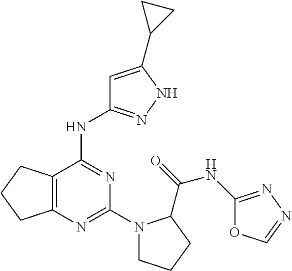 [SMILES: O=C(Nc1nnco1)C1CCCN1c1nc2c(c(Nc3cc(C4CC4)[nH]n3)n1)CCC2] | 1-(4-((5-cyclopropyl-1H-pyrazol-3-yl)amino)- 6,7-dihydro-5H-cyclopenta[d]pyrimidin-2-yl)- N-(1,3,4-oxadiazol-2-yl)pyrrolidine-2- carboxamide |
| 126a | | (R)-1-(4-((5-cyclopropyl-1H-pyrazol-3- |
| | | yl)amino)-6,7-dihydro-5H- |
| | | cyclopenta[d]pyrimidin-2-yl)-N-(1,3,4- |
| | | oxadiazol-2-yl)pyrrolidine-2-carboxamide |
| 126b | | (S)-1-(4-((5-cyclopropyl-1H-pyrazol-3- |
| | | yl)amino)-6,7-dihydro-5H- |
| | | cyclopenta[d]pyrimidin-2-yl)-N-(1,3,4- |
| | | oxadiazol-2-yl)pyrrolidine-2-carboxamide |
| 127 | 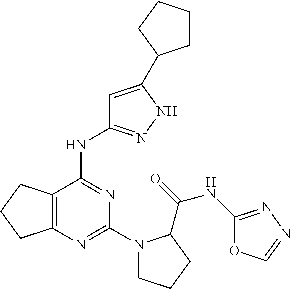 [SMILES: O=C(Nc1nnco1)C1CCCN1c1nc2c(c(Nc3cc(C4CCCC4)[nH]n3)n1)CCC2] | 1-(4-((5-cyclopentyl-1H-pyrazol-3-yl)amino)- 6,7-dihydro-5H-cyclopenta[d]pyrimidin-2-yl)- N-(1,3,4-oxadiazol-2-yl)pyrrolidine-2- carboxamide |
| 127a | | (R)-1-(4-(5-cyclopentyl-1H-pyrazol-3- |
| | | yl)amino)-6,7-dihydro-5H- |
| | | cyclopenta[d]pyrimidin-2-yl)-N-(1,3,4- |
| | | oxadiazol-2-yl)pyrrolidine-2-carboxamide |
| 127b | | (S)-1-(4-(5-cyclopentyl-1H-pyrazol-3- |
| | | yl)amino)-6,7-dihydro-5H- |
| | | cyclopenta[d]pyrimidin-2-yl)-N-(1,3,4- |
| | | oxadiazol-2-yl)pyrrolidine-2-carboxamide |
| 128 | 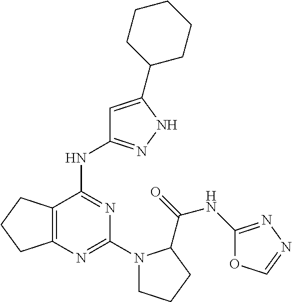 [SMILES: O=C(Nc1nnco1)C1CCCN1c1nc2c(c(Nc3cc(C4CCCCC4)[nH]n3)n1)CCC2] | 1-(4-((5-cyclohexyl-1H-pyrazol-3-yl)amino)- 6,7-dihydro-5H-cyclopenta[d]pyrimidin-2-yl)- N-(1,3,4-oxadiazol-2-yl)pyrrolidine-2- carboxamide |
| 128a | | (R)-1-(4-((5-cyclohexyl-1H-pyrazol-3- |
| | | yl)amino)-6,7-dihydro-5H- |
| | | cyclopenta[d]pyrimidin-2-yl)-N-(1,3,4- |
| | | oxadiazol-2-yl)pyrrolidine-2-carboxamide |
| 128b | | (S)-1-(4-((5-cyclohexyl-1H-pyrazol-3- |
| | | yl)amino)-6,7-dihydro-5H- |
| | | cyclopenta[d]pyrimidin-2-yl)-N-(1,3,4- |
| | | oxadiazol-2-yl)pyrrolidine-2-carboxamide |
| 129 | 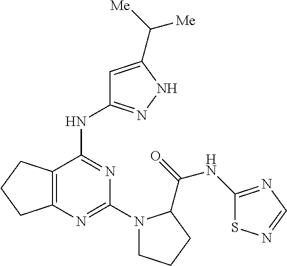 [SMILES: CC(C)c1cc(Nc2nc(N3CCCC3C(=O)Nc3ncns3)nc3c2CCC3)n[nH]1] | 1-(4-((5-isopropyl-1H-pyrazol-3-yl)amino)-6,7- dihydro-5H-cyclopenta[d]pyrimidin-2-yl)-N- (1,2,4-thiadiazol-2-yl)pyrrolidine-2-carboxamide |
| 129a | | (R)-1-(4-((5-isopropyl-1H-pyrazol-3-yl)amino)- |
| | | 6,7-dihydro-5H-cyclopenta[d]pyrimidin-2-yl)- |
| | | N-(1,2,4-thiadiazol-2-yl)pyrrolidine-2- |
| | | carboxamide |
| 129b | | (S)-1-(4-((5-isopropyl-1H-pyrazol-3-yl)amino)- |
| | | 6,7-dihydro-5H-cyclopenta[d]pyrimidin-2-yl)- |
| | | N-(1,2,4-thiadiazol-2-yl)pyrrolidine-2- |
| | | carboxamide |
| 130 | 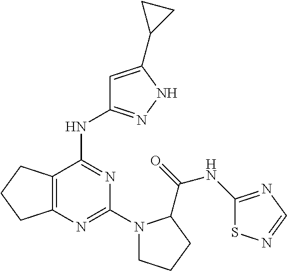 [SMILES: O=C(Nc1ncns1)C1CCCN1c1nc2c(c(Nc3cc(C4CC4)[nH]n3)n1)CCC2] | 1-(4-((5-cyclopropyl-1H-pyrazol-3-yl)amino)- 6,7-dihydro-5H-cyclopenta[d]pyrimidin-2-yl)- N-(1,2,4-thiadiazol-2-yl)pyrrolidine-2- carboxamide |
| 130a | | (R)-1-(4-((5-cyclopropyl-1H-pyrazol-3- |
| | | yl)amino)-6,7-dihydro-5H- |
| | | cyclopenta[d]pyrimidin-2-yl)-N-(1,2,4- |
| | | thiadiazol-2-yl)pyrrolidine-2-carboxamide |
| 130b | | (S)-1-(4-((5-cyclopropyl-1H-pyrazol-3- |
| | | yl)amino)-6,7-dihydro-5H- |
| | | cyclopenta[d]pyrimidin-2-yl)-N-(1,2,4- |
| | | thiadiazol-2-yl)pyrrolidine-2-carboxamide |
| 131 | 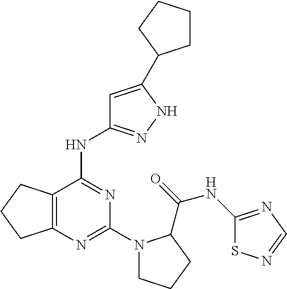 [SMILES: O=C(Nc1ncns1)C1CCCN1c1nc2c(c(Nc3cc(C4CCCC4)[nH]n3)n1)CCC2] | 1-(4-((5-cyclopentyl-1H-pyrazol-3-yl)amino)- 6,7-dihydro-5H-cyclopenta[d]pyrimidin-2-yl)- N-(1,2,4-thiadiazol-2-yl)pyrrolidine-2- carboxamide |
| 131a | | (R)-1-(4-(5-cyclopentyl-1H-pyrazol-3- |
| | | yl)amino)-6,7-dihydro-5H- |
| | | cyclopenta[d]pyrimidin-2-yl)-N-(1,2,4- |
| | | thiadiazol-2-yl)pyrrolidine-2-carboxamide |
| 131b | | (S)-1-(4-(5-cyclopentyl-1H-pyrazol-3- |
| | | yl)amino)-6,7-dihydro-5H- |
| | | cyclopenta[d]pyrimidin-2-yl)-N-(1,2,4- |
| | | thiadiazol-2-yl)pyrrolidine-2-carboxamide |
| 132 | 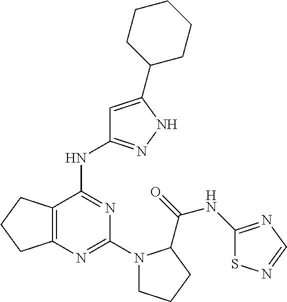 [SMILES: O=C(Nc1ncns1)C1CCCN1c1nc2c(c(Nc3cc(C4CCCCC4)[nH]n3)n1)CCC2] | 1-(4-((5-cyclohexyl-1H-pyrazol-3-yl)amino)- 6,7-dihydro-5H-cyclopenta[d]pyrimidin-2-yl)- N-(1,2,4-thiadiazol-5-yl)pyrrolidine-2- carboxamide |
| 132a | | (R)-1-(4-((5-cyclohexyl-1H-pyrazol-3- |
| | | yl)amino)-6,7-dihydro-5H- |
| | | cyclopenta[d]pyrimidin-2-yl)-N-(1,2,4- |
| | | thiadiazol-5-yl)pyrrolidine-2-carboxamide |
| 132b | | (S)-1-(4-((5-cyclohexyl-1H-pyrazol-3- |
| | | yl)amino)-6,7-dihydro-5H- |
| | | cyclopenta[d]pyrimidin-2-yl)-N-(1,2,4- |
| | | thiadiazol-5-yl)pyrrolidine-2-carboxamide |
| 133 | 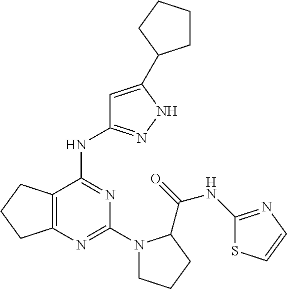 [SMILES: O=C(Nc1nccs1)C1CCCN1c1nc2c(c(Nc3cc(C4CCCC4)[nH]n3)n1)CCC2] | 1-(4-(5-cyclopentyl-1H-pyrazol-3-ylamino)-6,7- dihydro-5H-cyclopenta[d]pyrimidin-2-yl)-N- (thiazol-2-yl)pyrrolidine-2-carboxamide |
| 133a | | (R)-1-(4-(5-cyclopentyl-1H-pyrazol-3-ylamino)- |
| | | 6,7-dihydro-5H-cyclopenta[d]pyrimidin-2-yl)- |
| | | N-(thiazol-2-yl)pyrrolidine-2-carboxamide |
| 133b | | (S)-1-(4-(5-cyclopentyl-1H-pyrazol-3-ylamino)- |
| | | 6,7-dihydro-5H-cyclopenta[d]pyrimidin-2-yl)- |
| | | N-(thiazol-2-yl)pyrrolidine-2-carboxamide |
| 134 | 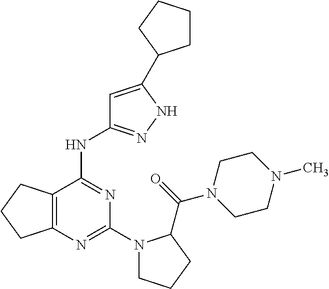 [SMILES: CN1CCN(C(=O)C2CCCN2c2nc3c(c(Nc4cc(C5CCCC5)[nH]n4)n2)CCC3)CC1] | (1-(4-(5-cyclopentyl-1H-pyrazol-3-ylamino)- 6,7-dihydro-5H-cyclopenta[d]pyrimidin-2- yl)pyrrolidin-2-yl)(4-methylpiperazin-1- yl)methanone |
| 134a | | (R)-(1-(4-(5-cyclopentyl-1H-pyrazol-3- |
| | | ylamino)-6,7-dihydro-5H- |
| | | cyclopenta[d]pyrimidin-2-yl)pyrrolidin-2-yl)(4- |
| | | methylpiperazin-1-yl)methanone |
| 134b | | (S)-(1-(4-(5-cyclopentyl-1H-pyrazol-3-ylamino)- |
| | | 6,7-dihydro-5H-cyclopenta[d]pyrimidin-2- |
| | | yl)pyrrolidin-2-yl)(4-methylpiperazin-1- |
| | | yl)methanone |
| 135 | 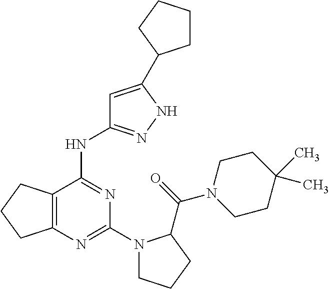 [SMILES: CC1(C)CCN(C(=O)C2CCCN2c2nc3c(c(Nc4cc(C5CCCC5)[nH]n4)n2)CCC3)CC1] | (1-(4-(5-cyclopentyl-1H-pyrazol-3-ylamino)- 6,7-dihydro-5H-cyclopenta[d]pyrimidin-2- yl)pyrrolidin-2-yl)(4,4-dimethylpiperidin-1- yl)methanone |
| 135a | | (R)-(1-(4-(5-cyclopentyl-1H-pyrazol-3- |
| | | ylamino)-6,7-dihydro-5H- |
| | | cyclopenta[d]pyrimidin-2-yl)pyrrolidin-2- |
| | | yl)(4,4-dimethylpiperidin-1-yl)methanone |
| 135b | | (S)-(1-(4-(5-cyclopentyl-1H-pyrazol-3-ylamino)- |
| | | 6,7-dihydro-5H-cyclopenta[d]pyrimidin-2- |
| | | yl)pyrrolidin-2-yl)(4,4-dimethylpiperidin-1- |
| | | yl)methanone |
| 136 | 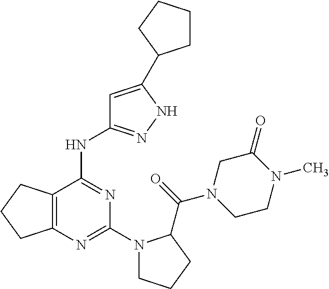 [SMILES: CN1CCN(C(=O)C2CCCN2c2nc3c(c(Nc4cc(C5CCCC5)[nH]n4)n2)CCC3)CC1=O] | 4-(1-(4-(5-cyclopentyl-1H-pyrazol-3-ylamino)- 6,7-dihydro-5H-cyclopenta[d]pyrimidin-2- yl)pyrrolidine-2-carbonyl)-1-methylpiperazin-2- one |
| 136a | | (R)-4-(1-(4-(5-cyclopentyl-1H-pyrazol-3- |
| | | ylamino)-6,7-dihydro-5H- |
| | | cyclopenta[d]pyrimidin-2-yl)pyrrolidine-2- |
| | | carbonyl)-1-methylpiperazin-2-one |
| 136b | | (S)-4-(1-(4-(5-cyclopentyl-1H-pyrazol-3- |
| | | ylamino)-6,7-dihydro-5H- |
| | | cyclopenta[d]pyrimidin-2-yl)pyrrolidine-2- |
| | | carbonyl)-1-methylpiperazin-2-one |
| 137 | 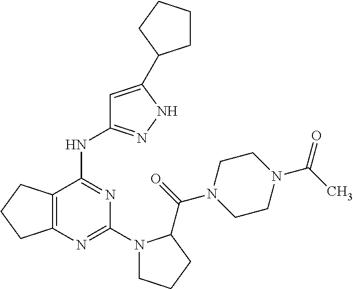 [SMILES: CC(=O)N1CCN(C(=O)C2CCCN2c2nc3c(c(Nc4cc(C5CCCC5)[nH]n4)n2)CCC3)CC1] | 1-(4-(1-(4-(5-cyclopentyl-1H-pyrazol-3- ylamino)-6,7-dihydro-5H- cyclopenta[d]pyrimidin-2-yl)pyrrolidine-2- carbonyl)piperazin-1-yl)ethanone |
| 137a | | (R)-1-(4-(1-(4-(5-cyclopentyl-1H-pyrazol-3- |
| | | ylamino)-6,7-dihydro-5H- |
| | | cyclopenta[d]pyrimidin-2-yl)pyrrolidine-2- |
| | | carbonyl)piperazin-1-yl)ethanone |
| 137b | | (S)-1-(4-(1-(4-(5-cyclopentyl-1H-pyrazol-3- |
| | | ylamino)-6,7-dihydro-5H- |
| | | cyclopenta[d]pyrimidin-2-yl)pyrrolidine-2- |
| | | carbonyl)piperazin-1-yl)ethanone |
| 138 | 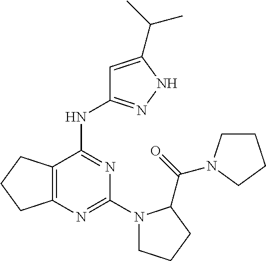 [SMILES: CC(C)c1cc(Nc2nc(N3CCCC3C(=O)N3CCCC3)nc3c2CCC3)n[nH]1] | (1-(4-((5-isopropyl-1H-pyrazol-3-yl)amino)-6,7- dihydro-5H-cyclopenta[d]pyrimidin-2- yl)pyrrolidin-2-yl)(pyrrolidin-1-yl)methanone |
| 138a | | (R)-(1-(4-((5-isopropyl-1H-pyrazol-3-yl)amino)- |
| | | 6,7-dihydro-5H-cyclopenta[d]pyrimidin-2- |
| | | yl)pyrrolidin-2-yl)(pyrrolidin-1-yl)methanone |
| 138b | | (S)-(1-(4-((5-isopropyl-1H-pyrazol-3-yl)amino)- |
| | | 6,7-dihydro-5H-cyclopenta[d]pyrimidin-2- |
| | | yl)pyrrolidin-2-yl)(pyrrolidin-1-yl)methanone |
| 139 | 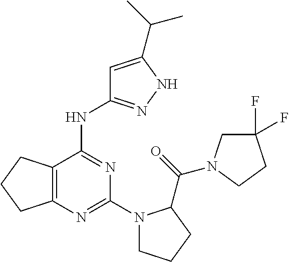 [SMILES: CC(C)c1cc(Nc2nc(N3CCCC3C(=O)N3CCC(F)(F)C3)nc3c2CCC3)n[nH]1] | (3,3-difluoropyrrolidin-1-yl)(1-(4-(5-isopropyl- 1H-pyrazol-3-yl)amino)-6,7-dihydro-5H- cyclopenta[d]pyrimidin-2-yl)pyrrolidin-2- yl)methanone |
| 139a | | (R)-(3,3-difluoropyrrolidin-1-yl)(1-(4-((5- |
| | | isopropyl-1H-pyrazol-3-yl)amino)-6,7-dihydro- |
| | | 5H-cyclopenta[d]pyrimidin-2-yl)pyrrolidin-2- |
| | | yl)methanone |
| 139b | | (S)-(3,3-difluoropyrrolidin-1-yl)(1-(4-((5- |
| | | isopropyl-1H-pyrazol-3-yl)amino)-6,7-dihydro- |
| | | 5H-cyclopenta[d]pyrimidin-2-yl)pyrrolidin-2- |
| | | yl)methanone |
| 140 | 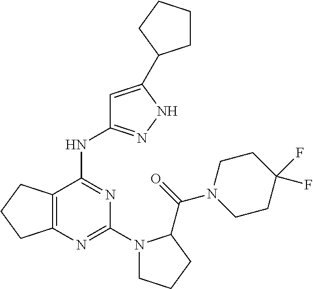 [SMILES: O=C(C1CCCN1c1nc2c(c(Nc3cc(C4CCCC4)[nH]n3)n1)CCC2)N1CCC(F)(F)CC1] | (1-(4-((5-cyclopentyl-1H-pyrazol-3-yl)amino)- 6,7-dihydro-5H-cyclopenta[d]pyrimidin-2- yl)pyrrolidin-2-yl)(4,4-difluoropiperidin-1- yl)methanone |
| 140a | | (R)-(1-(4-((5-cyclopentyl-1H-pyrazol-3- |
| | | yl)amino)-6,7-dihydro-5H- |
| | | cyclopenta[d]pyrimidin-2-yl)pyrrolidin-2- |
| | | yl)(4,4-difluoropiperidin-1-yl)methanone |
| 140b | | (S)-(1-(4-((5-cyclopentyl-1H-pyrazol-3- |
| | | yl)amino)-6,7-dihydro-5H- |
| | | cyclopenta[d]pyrimidin-2-yl)pyrrolidin-2- |
| | | yl)(4,4-difluoropiperidin-1-yl)methanone |
| 141 | 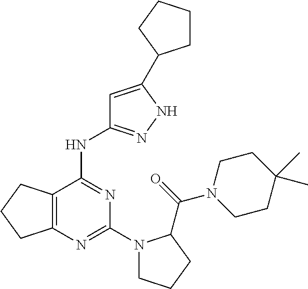 [SMILES: CC1(C)CCN(C(=O)C2CCCN2c2nc3c(c(Nc4cc(C5CCCC5)[nH]n4)n2)CCC3)CC1] | (1-(4-((5-cyclopentyl-1H-pyrazol-3-yl)amino)- 6,7-dihydro-5H-cyclopenta[d]pyrimidin-2- yl)pyrrolidin-2-yl)(4,4-dimethylpiperidin-1- yl)methanone |
| 141a | | (R)-(1-(4-((5-cyclopentyl-1H-pyrazol-3- |
| | | yl)amino)-6,7-dihydro-5H- |
| | | cyclopenta[d]pyrimidin-2-yl)pyrrolidin-2- |
| | | yl)(4,4-dimethylpiperidin-1-yl)methanone |
| 141b | | (S)-(1-(4-((5-cyclopentyl-1H-pyrazol-3- |
| | | yl)amino)-6,7-dihydro-5H- |
| | | cyclopenta[d]pyrimidin-2-yl)pyrrolidin-2- |
| | | yl)(4,4-dimethylpiperidin-1-yl)methanone |
| 142 | 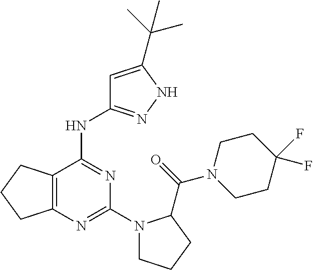 [SMILES: CC(C)(C)c1cc(Nc2nc(N3CCCC3C(=O)N3CCC(F)(F)CC3)nc3c2CCC3)n[nH]1] | (1-(4-((5-(tert-butyl)-1H-pyrazol-3-yl)amino)- 6,7-dihydro-5H-cyclopenta[d]pyrimidin-2- yl)pyrrolidin-2-yl)(4,4-difluoropiperidin-1- yl)methanone |
| 142a | | (R)-(1-(4-((5-(tert-butyl)-1H-pyrazol-3- |
| | | yl)amino)-6,7-dihydro-5H- |
| | | cyclopenta[d]pyrimidin-2-yl)pyrrolidin-2- |
| | | yl)(4,4-difluoropiperidin-1-yl)methanone |
| 142b | | (S)-(1-(4-((5-(tert-butyl)-1H-pyrazol-3- |
| | | yl)amino)-6,7-dihydro-5H- |
| | | cyclopenta[d]pyrimidin-2-yl)pyrrolidin-2- |
| | | yl)(4,4-difluoropiperidin-1-yl)methanone |
| 143 | 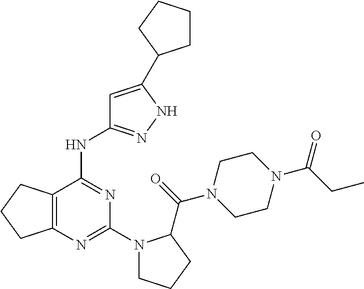 [SMILES: CCC(=O)N1CCN(C(=O)C2CCCN2c2nc3c(c(Nc4cc(C5CCCC5)[nH]n4)n2)CCC3)CC1] | 1-(4-((4-((5-cyclopentyl-1H-pyrazol-3- yl)amino)-6,7-dihydro-5H- cyclopenta[d]pyrimidin-2-yl)prolyl)piperazin-1- yl)propan-1-one |
| 143a | | 1-(4-((4-((5-cyclopentyl-1H-pyrazol-3- |
| | | yl)amino)-6,7-dihydro-5H- |
| | | cyclopenta[d]pyrimidin-2-yl)-D- |
| | | prolyl)piperazin-1-yl)propan-1-one |
| 143b | | 1-(4-((4-((5-cyclopentyl-1H-pyrazol-3- |
| | | yl)amino)-6,7-dihydro-5H- |
| | | cyclopenta[d]pyrimidin-2-yl)-L- |
| | | prolyl)piperazin-1-yl)propan-1-one |
| 144 | 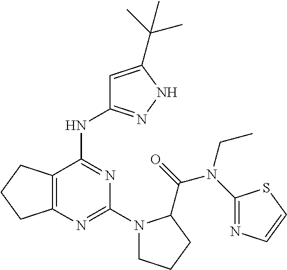 [SMILES: CCN(C(=O)C1CCCN1c1nc2c(c(Nc3cc(C(C)(C)C)[nH]n3)n1)CCC2)c1nccs1] | 1-(4-(5-(tert-butyl)-1H-pyrazol-3-yl)amino)- 6,7-dihydro-5H-cyclopenta[d]pyrimidin-2-yl)- N-ethyl-N-(thiazol-2-yl)pyrrolidine-2- carboxamide |
| 144a | | (R)-1-(4-(5-(tert-butyl)-1H-pyrazol-3- |
| | | yl)amino)-6,7-dihydro-5H- |
| | | cyclopenta[d]pyrimidin-2-yl)-N-ethyl-N- |
| | | (thiazol-2-yl)pyrrolidine-2-carboxamide |
| 144b | | (S)-1-(4-((5-(tert-butyl)-1H-pyrazol-3- |
| | | yl)amino)-6,7-dihydro-5H- |
| | | cyclopenta[d]pyrimidin-2-yl)-N-ethyl-N- |
| | | (thiazol-2-yl)pyrrolidine-2-carboxamide |
| 145 | 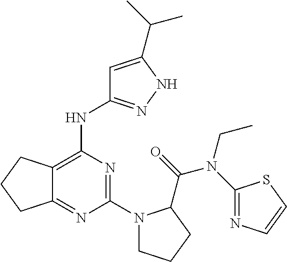 [SMILES: CCN(C(=O)C1CCCN1c1nc2c(c(Nc3cc(C(C)C)[nH]n3)n1)CCC2)c1nccs1] | N-ethyl-1-(4-((5-isopropyl-1H-pyrazol-3- yl)amino)-6,7-dihydro-5H- cyclopenta[d]pyrimidin-2-yl)-N-(thiazol-2- yl)pyrrolidine-2-carboxamide |
| 145a | | (R)-N-ethyl-1-(4-((5-isopropyl-1H-pyrazol-3- |
| | | yl)amino)-6,7-dihydro-5H- |
| | | cyclopenta[d]pyrimidin-2-yl)-N-(thiazol-2- |
| | | yl)pyrrolidine-2-carboxamide |
| 145b | | (S)-N-ethyl-1-(4-((5-isopropyl-1H-pyrazol-3- |
| | | yl)amino)-6,7-dihydro-5H- |
| | | cyclopenta[d]pyrimidin-2-yl)-N-(thiazol-2- |
| | | yl)pyrrolidine-2-carboxamide |
| 146 | 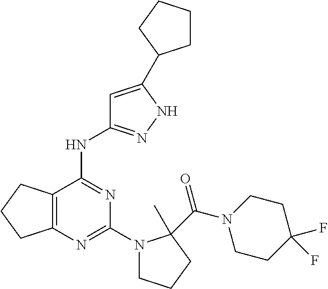 [SMILES: CC1(C(=O)N2CCC(F)(F)CC2)CCCN1c1nc2c(c(Nc3cc(C4CCCC4)[nH]n3)n1)CCC2] | (1-(4-((5-cyclopentyl-1H-pyrazol-3-yl)amino)- 6,7-dihydro-5H-cyclopenta[d]pyrimidin-2-yl)-2- methylpyrrolidin-2-yl)(4,4-difluoropiperidin-1- yl)methanone |
| 146a | | (R)-(1-(4-((5-cyclopentyl-1H-pyrazol-3- |
| | | yl)amino)-6,7-dihydro-5H- |
| | | cyclopenta[d]pyrimidin-2-yl)-2- |
| | | methylpyrrolidin-2-yl)(4,4-difluoropiperidin-1- |
| | | yl)methaone |
| 146b | | (S)-(1-(4-((5-cyclopentyl-1H-pyrazol-3- |
| | | yl)amino)-6,7-dihydro-5H- |
| | | cyclopenta[d]pyrimidin-2-yl)-2- |
| | | methylpyrrolidin-2-yl)(4,4-difluoropiperidin-1- |
| | | yl)methanone |

In some embodiments, the invention relates to compounds described in Table 1, including pharmaceutically acceptable salts thereof, and uses thereof.

In some embodiments, the invention relates to Compounds 1-137 as described in Table 1, including pharmaceutically acceptable salts thereof, and uses thereof. In one embodiment, provided is a compound selected from Compound Nos. 1-137, or a pharmaceutically acceptable salt thereof, and uses thereof.

In another embodiment, the invention relates to Compounds 1-137, and stereoisomers thereof, and uses thereof. In one embodiment, provided is a compound selected from Compound Nos. 1a, 1b, 2a, 2b, 3a, 3b, 4a, 4b, 5a, 5b, 6a, 6b, 7a, 7b, 8a, 8b, 9a, 9b, 10a, 10b, 11a, 11b, 12a, 12b, 13a, 13b, 14a, 14b, 15a, 15b, 16a, 16b, 17a, 17b, 18a, 18b, 19a, 19b, 20a, 20b, 21a, 21b, 22a, 22b, 23a, 23b, 24a, 24b, 25a, 25b, 26a, 26b, 27b, 27b, 28a, 28b, 29a, 29b, 30a, 30b, 31a, 31b, 32a, 32b, 33a, 33b, 34a, 34b, 35a, 35b, 36a, 36b, 37a, 37b, 38a, 38b, 39a, 39b, 40a, 40b, 41a, 41b, 42a, 42b, 43a, 43b, 44a, 44b, 45a, 45b, 46a, 46b, 47a, 47b, 48a, 48b, 49a, 49b, 50a, 50b, 51a, 51b, 52a, 52b, 53a, 53b, 54a, 54b, 55a, 55b, 56a, 56b, 57a, 57b, 58a, 58b, 59a, 59b, 60a, 60b, 61a, 61b, 62a, 62b, 63a, 63b, 64a, 64b, 65a, 65b, 66a, 66b, 67a, 67b, 68a, 68b, 69a, 69b, 70a, 70b, 71a, 71b, 72a, 72b, 73a, 73b, 74a, 74b, 75a, 75b, 76a, 76b, 76c, 76d, 77a, 77b, 77c, 77d, 78a, 78b, 78c, 78d, 79a, 79b, 79c, 79d, 80a, 80b, 80c, 80d, 81a, 81b, 81c, 81d, 82a, 82b, 82c, 82d, 83a, 83b, 83c, 83d, 84a, 84b, 85a, 85b, 86a, 86b, 87a, 87b, 88a, 88b, 89a, 89b, 90a, 90b, 91a, 91b, 92a, 92b, 93a, 93b, 94a, 94b, 95a, 95b, 95c, 95d, 96a, 96b, 96c, 96d, 97a, 97b, 97c, 97d, 98a, 98b, 98c, 98d, 99a, 99b, 99c, 99d, 100a, 100b, 100c, 100d, 101a, 101b, 101c, 101d, 102a, 102b, 102c, 102d, 103a, 103b, 104a, 104b, 105a, 105b, 106a, 106b, 107a, 107b, 108a, 108b, 109a, 109b, 110a, 110b, 111a, 111b, 112a, 112b, 113a, 113b, 114a, 114b, 115a, 115b, 116a, 116b, 117a, 117b, 118a, 118b, 119a, 119b, 120a, 120b, 121a, 121b, 122a, 122b, 123a, 123b, 124a, 124b, 125a, 125b, 126a, 126b, 127a, 127b, 128a, 128b, 129a, 129b, 130a, 130b, 131a, 131b, 132a, 132b, 133a, 133b, 134a, 134b, 135a, 135b, 136a, 136b, 137a and 137b, or a pharmaceutically acceptable salt thereof, and uses thereof.

In another embodiment, the invention relates to Compounds 138-146, and stereoisomers thereof, and uses thereof. In one embodiment, provided is a compound selected from Compound Nos. 138a, 138b, 139a, 139b, 140a, 140b, 141a, 141b, 142a, 142b, 143a, 143b, 144a, 144b, 145a, 145b, 146a, 146b or a pharmaceutically acceptable salt thereof, and uses thereof.

The embodiments and variations described herein are suitable for compounds of any formulae detailed herein, where applicable.

It is understood that compounds of the invention include enantiomers and/or diastereomers, if applicable, in isomerically pure form or in a composition comprising mixtures of compounds of the invention in any ratio, including two or more stereochemical forms, such as in a racemic or non-racemic mixture or a mixture of one or more diastereomers. A structure or name is intended to embrace all possible stereoisomers of a compound depicted, and mixtures thereof. Each unique stereoisomer has a compound number bearing a suffix “a”, “b”, etc. Compositions comprising a compound of the invention are also contemplated, such as a composition of substantially pure compound, including a specific stereochemical form thereof, or a composition comprising mixtures of compounds of the invention in any ratio, including two or more stereochemical forms, such as in a racemic or non-racemic mixture.

Where tautomeric forms may be present for any of the compounds described herein, each and every tautomeric form is intended even though only one or some of the tautomeric forms may be explicitly depicted. For example, when a 5-cyclopropyl-1H-pyrazol-3-yl moiety is depicted, the corresponding 3-cyclopropyl-1H-pyrazol-5-yl tautomer is also intended. In addition, if a 2-hydroxy-pyridine moiety is described, the corresponding 2-oxo-1,2-dihydropyridine tautomer is also intended, and vice versa. The tautomeric forms specifically depicted may or may not be the predominant forms in solution or when used according to the methods described herein.

The compounds depicted herein may be present as salts even if salt forms are not explicitly depicted and it is understood that the invention embraces all salts and solvates of the compounds depicted here, as well as the non-salt and non-solvate form of the compound, as is well understood by the skilled artisan.

All forms of the compounds are also embraced by the invention, such as crystalline or non-crystalline forms of the compounds.

Where a tertiary amine or pyridyl moiety is present in the compound, the N-oxides of the nitrogens in those groups are also provided and described.

The invention also intends isotopically-labeled and/or isotopically-enriched forms of compounds described herein. The compounds herein may contain unnatural proportions of atomic isotopes at one or more of the atoms that constitute such compounds. In some embodiments, the compound is isotopically-labeled, such as an isotopically-labeled compound of the formula (I) or variations thereof described herein, where a fraction of one or more atoms are replaced by an isotope of the same element. Exemplary isotopes that can be incorporated into compounds of the invention include isotopes of hydrogen, carbon, nitrogen, oxygen, phosphorus, sulfur, or chlorine, such as ²H, ³H, ¹¹C, ¹³C, ¹⁴C ¹³N, ¹⁵O, ¹⁷O, ³²P, ³⁵S, ¹⁸F, or ³⁶Cl. Certain isotope labeled compounds (e.g. ³H and ¹⁴C) are useful in compound or substrate tissue distribution study. Incorporation of heavier isotopes such as deuterium (²H) can afford certain therapeutic advantages resulting from greater metabolic stability, for example, increased in vivo half-life, or reduced dosage requirements and, hence may be preferred in some instances. Isotopically-labeled compounds of the present invention can generally be prepared by standard methods and techniques known to those skilled in the art or by procedures similar to those described in the accompanying Examples substituting appropriate isotopically-labeled reagents in place of the corresponding non-labeled reagent.

The invention also includes any or all metabolites of any of the compounds described. The metabolites may include any chemical species generated by a biotransformation of any of the compounds described, such as intermediates and products of metabolism of the compound. Typical examples of metabolites of a compound presented herein are depicted below.

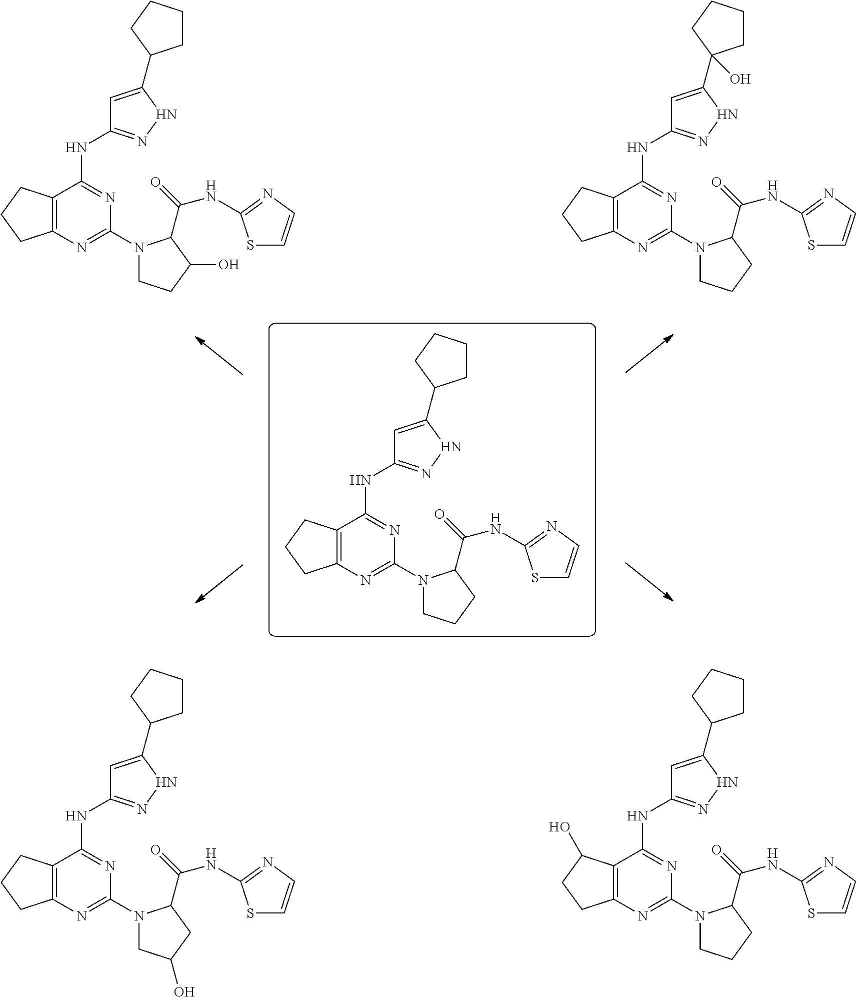 [SMILES: O=C(Nc1nccs1)C1C(O)CCN1c1nc2c(c(Nc3cc(C4CCCC4)[nH]n3)n1)CCC2 ; O=C(Nc1nccs1)C1CCCN1c1nc2c(c(Nc3cc(C4(O)CCCC4)[nH]n3)n1)CCC2 ; O=C(Nc1nccs1)C1CCCN1c1nc2c(c(Nc3cc(C4CCCC4)[nH]n3)n1)CCC2 ; O=C(Nc1nccs1)C1CC(O)CN1c1nc2c(c(Nc3cc(C4CCCC4)[nH]n3)n1)CCC2 ; O=C(Nc1nccs1)C1CCCN1c1nc2c(c(Nc3cc(C4CCCC4)[nH]n3)n1)C(O)CC2]

Representative examples of compounds detailed herein, including intermediates and final compounds according to the invention are depicted below. It is understood that in one aspect, any of the compounds may be used in the methods detailed herein, including, where applicable, intermediate compounds that may be isolated and administered to an individual.

### Pharmaceutical Compositions and Formulations

Pharmaceutical compositions of any of the compounds detailed herein are embraced by this invention. Thus, the invention includes pharmaceutical compositions comprising a compound of the invention or a pharmaceutically acceptable salt thereof and a pharmaceutically acceptable carrier or excipient. In one aspect, the pharmaceutically acceptable salt is an acid addition salt, such as a salt formed with an inorganic or organic acid.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form. In one variation, “substantially pure” intends a composition that contains no more than 35% impurity, wherein the impurity denotes a compound other than the compound comprising the majority of the composition or a salt thereof. Taking compound 1 as an example, a composition of substantially pure compound 1 intends a composition that contains no more than 35% impurity, wherein the impurity denotes a compound other than compound 1 or a salt thereof. In one variation, a composition of substantially pure compound or a salt thereof is provided wherein the composition contains no more than 25% impurity. In another variation, a composition of substantially pure compound or a salt thereof is provided wherein the composition contains or no more than 20% impurity. In still another variation, a composition of substantially pure compound or a salt thereof is provided wherein the composition contains or no more than 10% impurity. In a further variation, a composition of substantially pure compound or a salt thereof is provided wherein the composition contains or no more than 5% impurity. In another variation, a composition of substantially pure compound or a salt thereof is provided wherein the composition contains or no more than 3% impurity. In still another variation, a composition of substantially pure compound or a salt thereof is provided wherein the composition contains or no more than 1% impurity. In a further variation, a composition of substantially pure compound or a salt thereof is provided wherein the composition contains or no more than 0.5% impurity. In yet other variations, a composition of substantially pure compound means that the composition contains no more than 15% or preferably no more than 10% or more preferably no more than 5% or even more preferably no more than 3% and most preferably no more than 1% impurity, which impurity may be the compound in a different stereochemical form. For instance, a composition of substantially pure (S) compound means that the composition contains no more than 15% or no more than 10% or no more than 5% or no more than 3% or no more than 1% of the (R) form of the compound.

In one variation, the compounds herein are synthetic compounds prepared for administration to an individual. In another variation, compositions are provided containing a compound in substantially pure form. In another variation, the invention embraces pharmaceutical compositions comprising a compound detailed herein and a pharmaceutically acceptable carrier. In another variation, methods of administering a compound are provided. The purified forms, pharmaceutical compositions and methods of administering the compounds are suitable for any compound or form thereof detailed herein.

The compound may be formulated for any available delivery route, including an oral, mucosal (e.g., nasal, sublingual, vaginal, buccal or rectal), parenteral (e.g., intramuscular, subcutaneous or intravenous), topical or transdermal delivery form. In some embodiments, the compounds detailed herein are orally bioavailable. A compound may be formulated with suitable carriers to provide delivery forms that include, but are not limited to, tablets, caplets, capsules (such as hard gelatin capsules or soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

One or several compounds described herein can be used in the preparation of a formulation, such as a pharmaceutical formulation, by combining the compound or compounds as an active ingredient with a pharmaceutically acceptable carrier, such as those mentioned above. Depending on the therapeutic form of the system (e.g., transdermal patch vs. oral tablet), the carrier may be in various forms. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants. Formulations comprising the compound may also contain other substances which have valuable therapeutic properties. Pharmaceutical formulations may be prepared by known pharmaceutical methods. Suitable formulations and preparation methods can be found, e.g., in Remington's Pharmaceutical Sciences, Mack Publishing Company, Philadelphia, Pa., 20^{th} ed. (2000), which is incorporated herein by reference.

Compounds as described herein may be administered to individuals in a form of generally accepted oral compositions, such as tablets, coated tablets, and gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shell are, for instance, plant oils, wax, fats, semisolid and liquid poly-ols, and so on. Any of the compounds described herein can be formulated in a tablet in any dosage form described, for example, a compound as described herein or a pharmaceutically acceptable salt thereof can be formulated as a 10 mg tablet.

One or several compounds described herein can be used in the preparation of a medicament by combining the compound or compounds as an active ingredient with a pharmacologically acceptable carrier, which are known in the art. Depending on the therapeutic form of the medication, the carrier may be in various forms. In one variation, the manufacture of a medicament is for use in any of the methods disclosed herein, e.g., for the treatment of cancer.

Pharmaceutical compositions as described herein may also comprise one or more additional active ingredients in the treatment of the disease and disorders described herein. Further additional active ingredients include other therapeutics or agents that mitigate adverse effects of therapies for the intended disease targets. Such combinations may serve to increase efficacy, ameliorate other disease symptoms, decrease one or more side effects, or decrease the required dose of an inventive compound. The additional active ingredients may be administered in a separate pharmaceutical composition from a compound of the present invention or may be included with a compound of the present invention in a single pharmaceutical composition. The additional active ingredients may be administered simultaneously with, prior to, or after administration of a compound of the present invention.

Combination agents include additional active ingredients are those that are known or discovered to be effective in treating the diseases and disorders described herein, including those active against another target associated with the disease. For example, compositions and formulations of the invention, as well as methods of treatment, can further comprise other drugs or pharmaceuticals, e.g., other active agents useful for treating or palliative for the target diseases or related symptoms or conditions. For cancer indications, additional such agents include, but are not limited to, kinase inhibitors, such as EGFR inhibitors (e.g., erlotinib, gefitinib), Raf inhibitors (e.g., vemurafenib), VEGFR inhibitors (e.g., sunitinib), standard chemotherapy agents such as alkylating agents, antimetabolites, anti-tumor antibiotics, topoisomerase inhibitors, platinum drugs, mitotic inhibitors, antibodies, hormone therapies, or corticosteroids. The pharmaceutical compositions of the invention may additional comprise one or more of such active agents, and methods of treatment may additionally comprise administering an effective amount of one or more of such active agents.

Articles of manufacture include articles comprising a compound of the invention, or a salt or solvate thereof, or a composition comprising a compound of the invention or a salt or solvate thereof, optionally in a unit dosage form, and presented in a suitable container for storage or for use in the methods described herein. Suitable packaging is known in the art and includes, for example, vials, vessels, ampules, bottles, jars, reloaded syringes, i.v. bags, flexible packaging and the like. An article of manufacture may be sterilized and/or sealed.

### Methods of Use

Compounds and compositions of the invention, such as a pharmaceutical composition containing a compound of any formula provided herein or a salt thereof and a pharmaceutically acceptable carrier or excipient, may be used in methods of administration and treatment as provided herein. The compounds and compositions may also be used in in vitro methods, such as in vitro methods of administering a compound or composition to cells for screening purposes and/or for conducting quality control assays.

In another aspect, the invention provides a method of inhibiting a FMS-like kinase (e.g., FLT3, FLT3(D835Y) and FLT3(ITD)) comprising administering to an individual an effective amount of one or more compounds of the invention, or a compound in Table 1, or a salt thereof (e.g., a pharmaceutically acceptable salt).

In one aspect, the invention provides a method of inhibiting a tropomyosin-receptor kinase receptor (e.g., Trk A, Trk B and Trk C) comprising administering to an individual an effective amount of one or more compounds of the invention, or a salt thereof (e.g., a pharmaceutically acceptable salt). In one such method, the method is further characterized by little or no inhibition of IGR (e.g., IGR-1R) due to administration of one or more compounds of the invention. In one such method, the compounds exhibit one or more of the following structural features: (1) R¹ is C₅₋₈cycloalkyl (e.g., cyclopentyl, cyclohexyl or bicyclo[3.1.0]hexan-3-yl) or 5- to 7-membered heterocyclyl (e.g., tetrahydro-2H-pyran-4-yl), each optionally substituted as described for Formula (I); (2) R² is H or C₁₋₄alkyl (e.g., methyl or ethyl), and R³ is a 5-membered heteroaryl (e.g., thiophenyl, thiazolyl, oxazolyl, pyrazolyl, imidazolyl, oxadiazolyl, thiadizaolyl, or triazolyl) optionally substituted as described for Formula (I), or R² and R³ taken together with the nitrogen to which they are attached form a 5- to 8-membered heterocyclyl (e.g., pyrrolidinyl, piperidinyl, morpholinyl, thiomorpholinyl, piperazinyl, 5-azaspiro[2.4]heptan-5-yl or 6-azaspiro[2.5]octan-6-yl) optionally substituted with C₁₋₄alkyl, —OH, —OC₁₋₄alkyl, oxo, halo, —CF₃, —CN, —C(O)C₁₋₄alkyl, —CO₂H, —CO₂C₁₋₄alkyl, —C(O)NR^{g}R^{h}, or —NR^{g}R^{h}; (3) R¹ is C₅₋₈cycloalkyl (e.g., cyclopentyl, cyclohexyl or bicyclo[3.1.0]hexan-3-yl) or 5- to 7-membered heterocyclyl (e.g., tetrahydro-2H-pyran-4-yl), each optionally substituted as described for Formula (I), R² is H or C₁₋₄alkyl (e.g., methyl or ethyl), and R³ is a 5-membered heteroaryl (e.g., thiophenyl, thiazolyl, oxazolyl, pyrazolyl, imidazolyl, oxadiazolyl, thiadizaolyl, or triazolyl) optionally substituted as described for Formula (I); or (4) R¹ is C₅₋₈cycloalkyl (e.g., cyclopentyl, cyclohexyl or bicyclo[3.1.0]hexan-3-yl) or 5- to 7-membered heterocyclyl (e.g., tetrahydro-2H-pyran-4-yl), each optionally substituted as described for Formula (I), and R² and R³ taken together with the nitrogen to which they are attached form a 5- to 8-membered heterocyclyl (e.g., pyrrolidinyl, piperidinyl, morpholinyl, thiomorpholinyl, piperazinyl, 5-azaspiro[2.4]heptan-5-yl or 6-azaspiro[2.5]octan-6-yl) optionally substituted with C₁₋₄alkyl, —OH, —OC₁₋₄alkyl, oxo, halo, —CF₃, —CN, —C(O)C₁₋₄alkyl, —CO₂H, —CO₂C₁₋₄alkyl, —C(O)NR^{g}R^{h}, or —NR^{g}R^{h}. In one aspect of the method, a compound of the invention or salt thereof inhibits binding of a ligand to the Trk receptor (e.g., Trk A, Trk B and/or Trk C) and/or reduces or eliminates or increases or enhances or mimics an activity of the Trk receptor in a reversible or irreversible manner. In some aspects, a compound of the invention inhibits binding of a ligand to the Trk receptor (e.g., Trk A, Trk B and/or Trk C) by at least about or by about any one of 10%, 20%, 30%, 40%, 50%, 60%, 70%, 80%, 90%, 95% or 100% as determined by an assay described herein. In some aspects, a compound of the invention reduces an activity of the Trk receptor (e.g., Trk A, Trk B and/or Trk C) by at least about or about any of 10%, 20%, 30%, 40%, 50%, 60%, 70%, 80%, 90%, 95% or 100% as compared to the corresponding activity in the same subject prior to treatment with the receptor modulator or compared to the corresponding activity in other subjects not receiving the compound. In one aspect, the individual has or is believed to have a disorder in which a Trk receptor (e.g., Trk A, Trk B and/or Trk C) is implicated. In some embodiments, the compound or salt thereof inhibits one or two or three of Trk A, Trk B and Trk C. In some embodiments, the compound or salt thereof selectively inhibits Trk A, Trk B or Trk C. In some embodiments, the compound or salt thereof selectively inhibits Trk A. In some embodiments, the compound or salt thereof selectively inhibits Trk B. In some embodiments, the compound or salt thereof selectively inhibits Trk C. In certain embodiments, the compound or salt thereof preferentially inhibits two of Trk A, Trk B and Trk C. In certain embodiments, the compound or salt thereof inhibits Trk A, Trk B and Trk C. In certain variations, a compound or composition of the invention is used to treat or prevent an Trk receptor related disorder, such as cancer (e.g., neuroblastoma, pancreatic cancer and colon cancer).

In one aspect of the method, a compound of the invention or salt thereof inhibits binding of a ligand to a FMS-like tyrosine kinase receptor such as an FLT3 receptor (e.g., FLT3, FLT3(D835Y) and FLT3(ITD)) and/or reduces or eliminates or increases or enhances or mimics an activity of the FLT3 receptor in a reversible or irreversible manner. In some aspects, a compound of the invention inhibits binding of a ligand to the FLT3 receptor (e.g., FLT3, FLT3(D835Y) and FLT3(ITD)) by at least about or by about any one of 10%, 20%, 30%, 40%, 50%, 60%, 70%, 80%, 90%, 95% or 100% as determined by an assay described herein. In some aspects, a compound of the invention reduces an activity of the FLT3 receptor (e.g., FLT3, FLT3(D835Y) and FLT3(ITD)) by at least about or about any of 10%, 20%, 30%, 40%, 50%, 60%, 70%, 80%, 90%, 95% or 100% as compared to the corresponding activity in the same subject prior to treatment with the receptor modulator or compared to the corresponding activity in other subjects not receiving the compound. In one aspect, the individual has or is believed to have a disorder in which a FLT3 receptor (e.g., FLT3, FLT3(D835Y) and FLT3(ITD)) is implicated. In some embodiments, the compound or salt thereof inhibits one or two or three of FLT3, FLT3(D835Y) and FLT3(ITD). In some embodiments, the compound or salt thereof selectively inhibits FLT3, FLT3(D835Y) or FLT3(ITD). In some embodiments, the compound or salt thereof selectively inhibits FLT3. In some embodiments, the compound or salt thereof selectively inhibits FLT3(D835Y). In some embodiments, the compound or salt thereof selectively inhibits FLT3(ITD). In certain embodiments, the compound or salt thereof preferentially inhibits two of FLT3, FLT3(D835Y) and FLT3(ITD). In certain embodiments, the compound or salt thereof inhibits FLT3, FLT3(D835Y) and FLT3(ITD). In certain variations, a compound or composition of the invention is used to treat or prevent an FLT3 receptor related disorder, such as cancer (e.g., acute myeloid leukemia and acute lymphoblastic leukemia).

In one aspect, the method comprises administering to the individual a compound provided herein, or a pharmaceutically acceptable salt thereof, including but not limited to a compound of the invention such as a compound according to any of the formulae provided herein; or a compound of Table 1 or an isomer thereof, or a salt (such as a pharmaceutically acceptable salt) of any of the foregoing. In one aspect, the individual is a human in need of cancer treatment.

Inhibitory activity of protein kinase inhibitors may be assessed by methods known in the art and methods detailed herein. In one aspect, compounds provided herein are selective protein kinase inhibitors that inhibit strongly certain protein kinases detailed herein but do not inhibit appreciably certain other protein kinases. For example, in some embodiments, the compounds inhibit strongly one or more FMS-like tyrosine kinases (e.g., FLT3, FLT3(D835Y) and FLT3(ITD)). In some embodiments, the compounds inhibit strongly the Trk family kinases (e.g., Trk A, Trk B and/or Trk C). In some embodiments, the compounds do not inhibit appreciably the activity of IGF-1R and/or IR receptors. In some embodiments, the compounds inhibit strongly the activity of one or more Trk receptors and do not inhibit appreciably the activity of IGF-1R and/or IR receptors. In some embodiments, the compounds inhibit the activity of one or more Trk receptors and do not inhibit the activity of IGF-1R and/or IR receptors. In some embodiments, the compounds bind to Trk and have no efficacy against IGF-1R and/or IR. Compounds do not inhibit or do not inhibit appreciably the activity of IGF-1R and/or IR receptors may lack or have diminished toxicity associated with inhibition of IGF-1R and/or IR. In one variation, a selective Trk family kinase inhibitor exhibits (i) equal to or greater than about any of 60%, 65%, 70%, 75%, 80%, 85%, 90%, or 95%, or between about 60% to about 100%, or between about 60% to about 80%, or between about 70% to about 90%, or between about 80% to about 100% inhibition of Trk (e.g., Trk A, Trk B and/or Trk C) at 100 nM; and (ii) equal to or less than about any of 30%, 25%, 20%, 15%, 10%, or 5%, or between about 0% to about 30%, or between about 10% to about 30%, or between about 20% to about 30% inhibition of IGF-1R and/or IR at 100 nM. In one variation, a selective Trk family kinase inhibitor exhibits (i) equal to or lower than about any of 0.1 nM, 1 nM, 10 nM, 100 nM, or 1 μM IC₅₀ for Trk (e.g., Trk A, Trk B and/or Trk C); and (ii) equal to or higher than about any of 100 nM, 1 μM, or 10 μM IC₅₀ for IGF-1R and/or IR. In one variation, a selective Trk family kinase inhibitor exhibits (i) equal to or lower than about any of 0.1 nM, 1 nM, 10 nM, or 100 nM IC₅₀ for Trk (e.g., Trk A, Trk B and/or Trk C); and (ii) equal to or higher than about any of 1 μM, or 10 μM IC₅₀ for IGF-1R and/or IR. In one variation, a selective Trk family kinase inhibitor exhibits equal to or lower than about any of 0.1 nM, 1 nM, 10 nM, 100 nM, or 1 μM IC₅₀ for Trk (e.g., Trk A, Trk B and/or Trk C); and does not inhibit IGF-1R and/or IR.

In some embodiments, an individual is a human. In some embodiments, an individual is a non-human primate such as a chimpanzee, or other primate from ape or monkey species. In some embodiments, an individual is a farm animal such as a cattle, horse, sheep, goat, or swine; a pet such as a rabbit, dog or cat; a laboratory animal including a rodent, such as rats, mice, and guinea pigs; and the like. The invention may find use in both human medicine and in the veterinary context.

The invention also provides methods for modulating the activity of a protein kinase comprising administering an effective amount of one or more compounds of the invention, or a salt thereof, to an individual. In one aspect, a method of modulating a protein kinase selected from the kinases of Examples B1-B7 (e.g., kinases of Examples B1, B4, B5, B6 or B7) is provided. In some embodiments, the protein kinase comprises one or more protein serine/threonine kinases or one or more protein tyrosine kinases. In some embodiments, the protein kinase comprises one or more protein kinase provided in the accompanying Examples, e.g., one or more protein kinase of Example B4 and B7. In some embodiments, the protein tyrosine kinase is selected from the group consisting of IGF-1R, IR, AXL; c-Met; c-Mer; DDR1; DDR2, and MUSK. In some embodiments, the protein serine/threonine kinase is selected from the group consisting of AURA, AURB, and AURC. In some embodiments, the protein tyrosine kinase is selected from the group consisting of ABL1, ABL2/ARG and ROS/ROS1. In one aspect the protein kinase comprises Trk but excludes IGF (e.g., IGR-1R). In one aspect the protein kinase comprises FLT3 but excludes IGF (e.g., IGR-1R). In one aspect, the method comprises administering to the individual a compound provided herein, or a pharmaceutically acceptable salt thereof, including but not limited to a compound of the invention such as a compound according to any one or more of the formulae herein; or a compound of Table 1 or an isomer thereof, or a salt (such as a pharmaceutically acceptable salt) of any of the foregoing.

The invention additionally provides methods for treating cancer in an individual (e.g., human) comprising administering to the individual an effective amount of a compound of the invention or a pharmaceutically acceptable salt thereof or a composition comprising the compound or salt thereof. In one aspect, the method comprises administering to the individual a compound provided herein, or a pharmaceutically acceptable salt thereof, including but not limited to a compound of the invention such as a compound according to any one or more of the formulae herein; or a compound of Table 1 or an isomer thereof, or a salt (such as a pharmaceutically acceptable salt) of any of the foregoing. Trk C and the members of the TAM family of receptor tyrosine kinases (AXL, Mer and Met) have been widely implicated in tumorigenesis and progression of several cancers and proposed as therapeutic targets for the treatment of prostate, breast, lung, renal, pancreatic cancers, neuroblastoma, medulloblastoma, head and neck carcinoma and acute myeloid leukemia. In contrast, inhibition of aurora kinase family members AURA, AURB and AURC has been associated with adverse effects like grade 3 neutropenia, a significant drop in the white blood cell count. In some embodiments, the invention also provides methods for modulating selectively the activity of protein tyrosine kinase receptors of IGF-1R, IR, AXL, FAK2, Mer, Met, Trk B, and Trk C with lower levels or absence of inhibition of members of Aurora kinase family. The invention also provides methods for preventing, and/or delaying the onset and/or development of cancer in an individual (e.g., human) comprising administering to the individual an effective amount of a compound of the invention or a pharmaceutically acceptable salt thereof or a composition comprising the compound or salt thereof.

In one variation, a method of treating cancer in an individual (e.g., human) is provided comprising administering to the individual an effective amount of a compound of the invention or a pharmaceutically acceptable salt thereof, wherein the cancer is dependent on a kinase signaling pathway (e.g., a signaling pathway of any of the kinases of Examples B1-B7 (e.g., kinases of Examples B1, B4, B5, B6 or B7)). In one variation, a method of treating cancer in an individual (e.g., human) is provided comprising administering to the individual an effective amount of a compound of the invention or a pharmaceutically acceptable salt thereof, wherein the cancer is dependent on the Trk family kinases (e.g., Trk A, Trk B and/or Trk C). In another variation, a method of treating cancer in an individual (e.g., human) is provided comprising administering to the individual an effective amount of a compound of the invention or a pharmaceutically acceptable salt thereof, wherein the cancer is characterized by depending on Trk family kinases (e.g., Trk A, Trk B and/or Trk C) for survival and/or proliferation. In yet another variation, a method of treating cancer in an individual (e.g., human) is provided comprising administering to the individual an effective amount of a compound of the invention or a pharmaceutically acceptable salt thereof, wherein the cancer cells overexpress one or more of the Trk family kinases (e.g., Trk A, Trk B and/or Trk C) as compared to non-cancerous cells, e.g., as compared to non-cancerous cells of the same cell type.

In one variation, a method of treating cancer in an individual (e.g., human) is provided comprising administering to the individual an effective amount of a compound of the invention or a pharmaceutically acceptable salt thereof, wherein the cancer is dependent on a FMS-like tyrosine kinase such as a FLT3 family kinase (e.g., FLT3, FLT3(D835Y) and/or FLT3(ITD)). In another variation, a method of treating cancer in an individual (e.g., human) is provided comprising administering to the individual an effective amount of a compound of the invention or a pharmaceutically acceptable salt thereof, wherein the cancer is characterized by depending on FLT3 family kinases (e.g., FLT3, FLT3(D835Y) and/or FLT3(ITD)) for survival and/or proliferation. In yet another variation, a method of treating cancer in an individual (e.g., human) is provided comprising administering to the individual an effective amount of a compound of the invention or a pharmaceutically acceptable salt thereof, wherein the cancer cells overexpress one or more of the FLT3 family kinases (e.g., FLT3, FLT3(D835Y) and/or FLT3(ITD)) as compared to non-cancerous cells, e.g., as compared to non-cancerous cells of the same cell type. In some embodiments, the method is for the treatment of an FLT3 receptor related cancer (e.g., acute myeloid leukemia and acute lymphoblastic leukemia).

In some embodiments, the amount of the compound or pharmaceutically acceptable salt thereof that is administered to an individual is an amount sufficient to decrease the size of a tumor, decrease the number of cancer cells, or decrease the growth rate of a tumor by at least about any of 10%, 20%, 30%, 40%, 50%, 60%, 70%, 80%, 90%, 95% or 100% compared to the corresponding tumor size, number of cancer cells, or tumor growth rate in the same subject prior to treatment or compared to the corresponding activity in other subjects not receiving the treatment. Standard methods can be used to measure the magnitude of this effect, such as in vitro assays with purified enzyme, cell-based assays, animal models, or human testing.

In some embodiments, the cancer that may be treated is a solid tumor such as sarcomas and carcinomas. In some embodiments, the cancer that may be treated is a liquid tumor such as leukemia. Examples of cancers that may be treated by methods of the invention include, but are not limited to, breast cancer, prostate cancer, ovarian cancer, lung cancer, colon cancer, brain tumors, gastric cancer, liver cancer, thyroid cancer, endometrial cancer, gallbladder cancer, kidney cancer, adrenocortical cancer, sarcoma, skin cancer, head and neck cancer, leukemia, bladder cancer, colorectal cancer, hematopoietic cancer and pancreatic cancer. In some embodiments, the breast cancer is breast carcinoma (ER negative or ER positive), primary breast ductal carcinoma, mammary adenocarcinoma, mammary ductal carcinoma (ER positive, ER negative or HER2 positive), HER2 positive breast cancer, luminal breast cancer or triple negative breast cancer (TNBC). In some embodiments, the breast cancer is unclassified. In some embodiments, the triple negative breast cancer is a basal-like TNBC, a mesenchymal TNBC (mesenchymal or mesenchymal stem-like), an immunomodulatory TNBC, or a luminal androgen receptor TNBC. In some embodiments, the prostate cancer is prostate adenocarcinoma. In some embodiments, the ovarian cancer is ovary adenocarcinoma. In some embodiments, the lung cancer is lung carcinoma, non-small lung carcinoma, adenocarcinoma, mucoepidermoid, anaplastic, large cell, or unclassified. In some embodiments, the colon cancer is colon adenocarcinoma, colon adenocarcinoma from a metastatic site lymph node, metastatic colorectal cancer, or colon carcinoma. In some embodiments, a brain tumor is glioblastoma, astrocytoma, meduloblastoma, meningioma or neuroblastoma. In some embodiments, gastric cancer is stomach cancer. In some embodiments, liver cancer is hepatocellular carcinoma, hepatoblastoma or cholangiocarcinoma. In some embodiments, liver cancer is hepatitis B virus-derived. In some embodiments, liver cancer is virus negative. In some embodiments, thyroid cancer is papillary thyroid carcinoma, follicular thyroid cancer or medullary thyroid cancer. In some embodiments, endometrial cancer is high grade endometroid cancer, uterine papillary serous carcinoma or uterine clear cell carcinoma. In some embodiments, gallbladder cancer is gallbladder adenocarcinoma or squamous cell gallbladder carcinoma. In some embodiments, kidney cancer is renal cell carcinoma or urothelial cell carcinoma. In some embodiments, adrenocortical cancer is adrenal cortical carcinoma. In some embodiments, sarcoma is synovial sarcoma, osteosarcoma, rhabdomiosarcoma, fibrosarcoma or Ewing's sarcoma. In some embodiments, skin cancer is basal cell carcinoma, squamous carcinoma or melanoma. In some embodiments, head and neck cancer is oropharyngeal cancer, nasopharyngeal cancer, laryngeal cancer and cancer of the trachea. In some embodiments, the leukemia is acute promyelocytic leukemia, acute lymphoblastic leukemia, chronic lymphocytic leukemia, chronic myelogenous leukemia, mantle cell lymphoma or multiple myeloma. In some embodiments, the leukemia is acute promyelocytic leukemia, acute lymphoblastic leukemia, chronic lymphocytic leukemia, chronic myelogenous leukemia, mantle cell lymphoma or multiple myeloma.

The invention additionally provides a method for treating a tumor comprising contacting the tumor with an effective amount of one or more compounds of the invention, or a salt thereof. In one aspect of the method, a compound or salt thereof is administered to an individual in need of tumor treatment. Exemplary tumors are derived from carcinomas of the breast, prostate, ovary, lung, or colon. In one aspect, the treatment results in a reduction of the tumor size. In another aspect, the treatment slows or prevents tumor growth and/or metastasis.

The invention further provides methods for treating a hematopoietic malignancy comprising administering an effective amount of one or more compounds of the invention to an individual in need thereof. In some embodiments, the hematopoietic malignancy is acute promyelocytic leukemia.

Any of the methods of treatment provided herein may be used to treat a primary tumor. Any of the methods of treatment provided herein may also be used to treat a metastatic cancer (that is, cancer that has metastasized from the primary tumor). Any of the methods of treatment provided herein may be used to treat cancer at an advanced stage. Any of the methods of treatment provided herein may be used to treat cancer at a locally advanced stage. Any of the methods of treatment provided herein may be used to treat early stage cancer. Any of the methods of treatment provided herein may be used to treat cancer in remission. In some of the embodiments of any of the methods of treatment provided herein, the cancer has reoccurred after remission. In some embodiments of any of the methods of treatment provided herein, the cancer is progressive cancer.

Any of the methods of treatment provided herein may be used to treat an individual (e.g., human) who has been diagnosed with or is suspected of having cancer. In some embodiments, the individual may be a human who exhibits one or more symptoms associated with cancer. In some embodiments, the individual may have advanced disease or a lesser extent of disease, such as low tumor burden. In some embodiments, the individual is at an early stage of a cancer. In some embodiments, the individual is at an advanced stage of cancer. In some of the embodiments of any of the methods of treatment provided herein, the individual may be a human who is genetically or otherwise predisposed (e.g., has one or more so-called risk factors) to developing cancer who has or has not been diagnosed with cancer. In some embodiments, these risk factors include, but are not limited to, age, sex, race, diet, history of previous disease, presence of precursor disease, genetic (e.g., hereditary) considerations, and environmental exposure. In some embodiments, the individuals at risk for cancer include, e.g., those having relatives who have experienced this disease, and those whose risk is determined by analysis of genetic or biochemical markers. In some embodiments, the individual does not have type I diabetes. In some embodiments, the individual does not have type II diabetes with sustained hyperglycemia or type II diabetes with hyperglycemia for prolonged duration (e.g., for several years).

Any of the methods of treatment provided herein may be practiced in an adjuvant setting. In some embodiments, any of the methods of treatment provided herein may be used to treat an individual who has previously been treated for cancer, e.g., with one or more other therapies such as radiation, surgery or chemotherapy. Any of the methods of treatment provided herein may be used to treat an individual who has not previously been treated for cancer. Any of the methods of treatment provided herein may be used to treat an individual at risk for developing cancer, but who has not been diagnosed with cancer. Any of the methods of treatment provided herein may be used as a first line therapy. Any of the methods of treatment provided herein may be used as a second line therapy.

Any of the methods of treatment provided herein in one aspect reduce the severity of one or more symptoms associated with cancer by at least about any of 10%, 20%, 30%, 40%, 50%, 60%, 70%, 80%, 90%, 95% or 100% compared to the corresponding symptom in the same subject prior to treatment or compared to the corresponding symptom in other subjects not receiving a compound or composition of the invention.

Any of the methods of treatment provided herein may be used to treat, stabilize, prevent, and/or delay any type or stage of cancer. In some embodiments, the individual is at least about any of 40, 45, 50, 55, 60, 65, 70, 75, 80, or 85 years old. In some embodiments, one or more symptoms of the cancer are ameliorated or eliminated. In some embodiments, the size of a tumor, the number of cancer cells, or the growth rate of a tumor decreases by at least about any of 10%, 20%, 30%, 40%, 50%, 60%, 70%, 80%, 90%, 95% or 100%. In some embodiments, the cancer is delayed or prevented.

In some embodiments, a compound or composition of the invention may be used to treat or prevent cancer in conjunction with a second therapy useful to reduce one or more side effects associated with administering the compound or composition of the invention. In some embodiments, the second compound for such combination therapy is selected from agents used for the treatment of glucose-related disorders such as Type 2 diabetes mellitus, impaired glucose tolerance, Insulin Resistance Syndrome and hyperglycemia. Examples of such agents include oral antidiabetic compounds from the classes of sulfonylureas, biguanides, thiazolidinediones, alpha-glucosidase inhibitors, meglitinides, other insulin-sensitizing compounds and/or other antidiabetic agents. Particular examples comprise Metformin (N,N-dimethylimidodicarbonimidic diamide), sulfonylureas and the like, or a salt of the foregoing. Testing of glucose concentration levels in an individual receiving a compound of the present invention may be followed by the co-administration of such a second agent (e.g., Metformin) as part of a combination therapy where appropriate (e.g., where the results of a glucose concentration level test in an individual indicate that such combination therapy will be or is expected to be beneficial for the individual).

In some embodiments, the compounds and compositions of the invention may be used to treat or prevent cancer in conjunction with a second therapy useful for cancer treatment. The second therapy includes, but is not limited to, surgery, radiation, and/or chemotherapy.

### Dosing and Method of Administration

The dose of a compound administered to an individual (such as a human) may vary with the particular compound or salt thereof, the method of administration, and the particular stage of cancer being treated. The amount should be sufficient to produce a desirable response, such as a therapeutic or prophylactic response against cancer. In some embodiments, the amount of the compound or salt thereof is a therapeutically effective amount. In some embodiments, the amount of the compound or salt thereof is a prophylactically effective amount. In some embodiments, the amount of compound or salt thereof is below the level that induces a toxicological effect (e.g., an effect above a clinically acceptable level of toxicity) or is at a level where a potential side effect can be controlled or tolerated when the composition is administered to the individual.

In some embodiments, the amount of compound or salt thereof is an amount sufficient to inhibit a Trk family kinase (e.g., Trk A, Trk B and/or Trk C), inhibit cancer cell growth and/or proliferation or increase apoptosis of cancer cells.

In some embodiments, the amount of compound or salt thereof is an amount sufficient to inhibit a FLT3 family kinase (e.g., FLT3, FLT3(D835Y) and/or FLT3(ITD)), inhibit cancer cell growth and/or proliferation or increase apoptosis of cancer cells.

In treating an individual as described herein, effective amounts or doses of the compounds of the invention may be ascertained by routine methods, such as modeling, dose escalation, or clinical trials, taking into account routine factors, e.g., the mode or route of administration or drug delivery, the pharmacokinetics of the agent, the severity and course of the infection, the subject's health status, condition, and weight, and the judgment of the treating physician. The effective amount of the compound may in one aspect be a dose of between about 0.01 and about 100 mg/kg. Effective amounts or doses of the compounds of the invention may be ascertained by routine methods, such as modeling, dose escalation, or clinical trials, taking into account routine factors, e.g., the mode or route of administration or drug delivery, the pharmacokinetics of the agent, the severity and course of the infection, the subject's health status, condition, and weight, and the judgment of the treating physician. An exemplary dose is in the range of about from about 0.1 mg to 1 g daily, or about 1 mg to 50 mg daily, or about 50 to 250 mg daily, or about 250 mg to 1 g daily. The total dosage may be given in single or divided dosage units (e.g., BID, TID, QID).

Any of the methods provided herein may in one aspect comprise administering to an individual a pharmaceutical composition that contains an effective amount of a compound provided herein or a salt thereof and a pharmaceutically acceptable excipient.

A compound or composition of the invention may be administered to an individual in accordance with an effective dosing regimen for a desired period of time or duration, such as at least about one month, at least about 2 months, at least about 3 months, at least about 6 months, or at least about 12 months or longer, which in some variations may be for the duration of the individual's life. In one variation, the compound is administered on a daily or intermittent schedule. The compound can be administered to an individual continuously (for example, at least once daily) over a period of time. The dosing frequency can also be less than once daily, e.g., about a once weekly dosing. The dosing frequency can be more than once daily, e.g., twice or three times daily. The dosing frequency can also be intermittent, including a “drug holiday” (e.g., once daily dosing for 7 days followed by no doses for 7 days, repeated for any 14 day time period, such as about 2 months, about 4 months, about 6 months or more). Any of the dosing frequencies can employ any of the compounds described herein together with any of the dosages described herein.

The compounds provided herein or a salt thereof may be administered to an individual via various routes, including, e.g., intravenous, intramuscular, subcutaneous, oral and transdermal. A compound provided herein can be administered frequently at low doses, known as “metronomic therapy,” or as part of a maintenance therapy using compound alone or in combination with one or more additional drugs. Metronomic therapy or maintenance therapy can comprise administration of a compound provided herein in cycles. Metronomic therapy or maintenance therapy can comprise intra-tumoral administration of a compound provided herein.

In one aspect, the invention provides a method of treating cancer in an individual by parenterally administering to the individual (e.g., a human) an effective amount of a compound or salt thereof. In some embodiments, the route of administration is intravenous, intra-arterial, intramuscular, or subcutaneous. In some embodiments, the route of administration is oral. In still other embodiments, the route of administration is transdermal.

The invention also provides compositions (including pharmaceutical compositions) as described herein for the use in treating, preventing, and/or delaying the onset and/or development of cancer and other methods described herein. In certain embodiments, the composition comprises a pharmaceutical formulation which is present in a unit dosage form.

### Kits

The invention further provides kits for carrying out the methods of the invention, which comprises one or more compounds described herein or a pharmacological composition comprising a compound described herein. The kits may employ any of the compounds disclosed herein. In one variation, the kit employs a compound described herein or a pharmaceutically acceptable salt thereof. The kits may be used for any one or more of the uses described herein, and, accordingly, may contain instructions for the treatment of cancer.

Kits generally comprise suitable packaging. The kits may comprise one or more containers comprising any compound described herein. Each component (if there is more than one component) can be packaged in separate containers or some components can be combined in one container where cross-reactivity and shelf life permit.

The kits may be in unit dosage forms, bulk packages (e.g., multi-dose packages) or sub-unit doses. For example, kits may be provided that contain sufficient dosages of a compound as disclosed herein and/or a second pharmaceutically active compound useful for a disease detailed herein (e.g., hypertension) to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present invention. The instructions included with the kit generally include information as to the components and their administration to an individual.

### General Synthetic Methods

The compounds of the invention may be prepared by a number of processes as generally described below and more specifically in the Examples hereinafter. In the following process descriptions, the symbols when used in the formulae depicted are to be understood to represent those groups described above in relation to the formulae herein.

Where it is desired to obtain a particular enantiomer of a compound, this may be accomplished from a corresponding mixture of enantiomers using any suitable conventional procedure for separating or resolving enantiomers. Thus, for example, diastereomeric derivatives may be produced by reaction of a mixture of enantiomers, e.g., a racemate, and an appropriate chiral compound. The diastereomers may then be separated by any convenient means, for example by crystallization and the desired enantiomer recovered. In another resolution process, a racemate may be separated using chiral High Performance Liquid Chromatography. Alternatively, if desired a particular enantiomer may be obtained by using an appropriate chiral intermediate in one of the processes described.

Chromatography, recrystallization and other conventional separation procedures may also be used with intermediates or final products where it is desired to obtain a particular isomer of a compound or to otherwise purify a product of a reaction.

Solvates and/or polymorphs of a compound provided herein or a pharmaceutically acceptable salt thereof are also contemplated. Solvates contain either stoichiometric or non-stoichiometric amounts of a solvent, and are often formed during the process of crystallization. Hydrates are formed when the solvent is water, or alcoholates are formed when the solvent is alcohol. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and/or solubility. Various factors such as the recrystallization solvent, rate of crystallization, and storage temperature may cause a single crystal form to dominate.

### General Synthesis of Compounds of Formula (I)

Compounds of Formula (I) may be prepared according to the Schemes and representative Examples below.

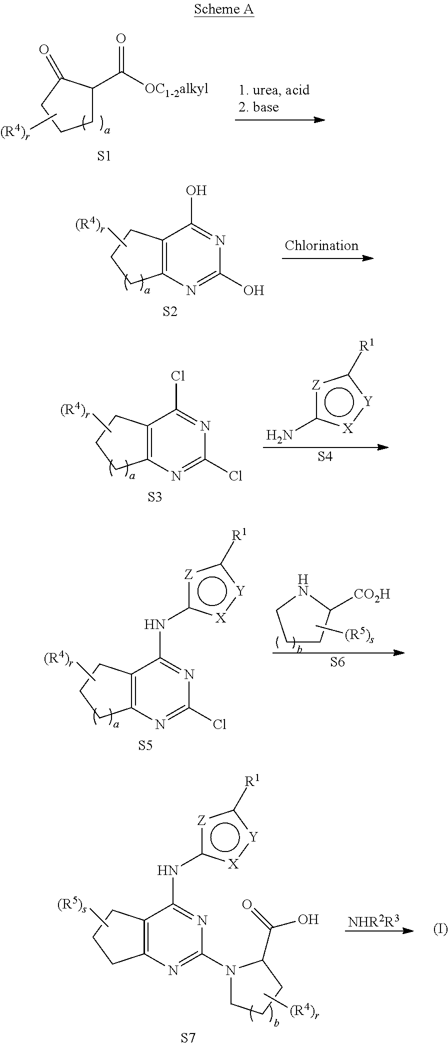

To prepare compounds of Formula (I), keto-esters S1, which are commercially available or readily prepared by methods known to one of ordinary skill in the art, are condensed with urea in the presence of an acid, then exposed to a base to form pyrimidine compounds S2. Chlorination using a suitable chlorinating agent such as POCl₃ provides dichloropyrimidines S3. Displacement of the 4-chloro substituent with suitably substituted heteroaromatic amines S4, in the presence of a tertiary amine base, or by palladium-mediated coupling with a suitable palladium catalyst such as Pd₂(dba)₃ optionally in the presence of a phosphine ligand and a base such as Cs₂CO₃, yields amino-pyrimidines S5. Displacement of the 2-chloro substituent with suitably substituted proline derivatives S6, in the presence of a base such as sodium hydroxide, generates diaminopyrimidines S7. If the reaction is performed in the presence of an acid such as TFA and an alkyl alcohol, the alkyl ester analogs of S7 are produced. Coupling with amines HNR²R³ with acids S7 under standard peptide coupling conditions, or use of or conversion to analogous alkyl esters, followed by reaction with conjugate base forms of HNR²R³ (generated by reaction with a strong base such as, for example, an alkyl Grignard), provides compounds of Formula (I). Such compounds are readily converted to their corresponding salt forms by reaction with at least one equivalent of a suitable acid as described herein.

Additional suitable methods for the synthesis of compounds of Formula (I) are described in PCT Application No. PCT/US2013/077817.

### EXAMPLES

The following Examples are provided to illustrate but not to limit the invention.

### General Protocol for Chiral Preparative HPLC Separation of Racemic Compounds

For chiral separations, samples are dissolved in MeOH and EtOH according to the solubility of sample and filtered through 0.22μ PTFE filters. The columns used are CHIRALPAK-AD; 20*250 mm, 10μ and CHIRALCEL-ODH; 20*250 mm, 5μ. A flow rate of 12 mL/min-17 mL/min is used according to the resolution. Alkanes such as n-pentane, hexane, and heptane (40%-95%) and alcohols such as EtOH, isopropyl alcohol, and t-butanol (5%-60%) are used as mobile phase. In some cases alcohol combinations, e.g., (EtOH+MeOH), (EtOH+IPA), (IPA+MeOH), (t-Butanol+MeOH), (t-Butanol+EtOH), are used instead of a single alcohol. Diethyl amine (up to 0.3%) may be used as modifier in the mobile phase.

A: General Method for the Chiral HPLC Separation and Characterization of Compounds that are Synthesized Initially as a Mixture of Enantiomers:

Crude or in some cases partially purified (normal or reverse phase HPLC) mixtures of enantiomers are analyzed by analytical chiral HPLC methods. Once adequate separation is achieved, larger quantities of the mixtures are separated using preparative scale columns. Separation is followed by removal of solvents on a rotary evaporator to accomplish the isolation of the individual single enantiomers. In some cases where appropriate, after removal of solvent, the samples are lyophilized. After isolation, each individual enantiomer is further analyzed by analytical (reverse phase and chiral) HPLC, LCMS and NMR. When final products are converted to salts, final characterization of the compounds is carried out after conversion to the salt for each enantiomer.

### Analytical Chiral HPLC of Compounds of the Invention.

### Column: Chiralcel OD-H; Column ID: 4.6*250 mm, 5μ. Mobile Phase:

Hexane:(EtOH:MeOH 80:20)—93:7. Flow rate: 1 mL/min.

### Chiral Preparative Data of Compounds of the Invention.

### Column: Chiralcel OD-H. Column ID: 20*250 mm, 5μ. Mobile Phase: Hexane:

(EtOH:MeOH 80:20)—95:5. Flow rate: 15 mL/min.
B: General Method for the Chiral HPLC Separation and Characterization of Compounds that are Synthesized Initially as a Mixture of Diastereomers:

Crude or in some cases partially purified (normal or reverse phase HPLC) mixtures of diastereomers are analyzed by analytical chiral HPLC methods. Once adequate separation is achieved, larger quantities of the mixtures are separated using preparative scale columns. Separation is followed by removal of solvents on a rotary evaporator to accomplish the isolation of the individual single diastereomers. In some cases where appropriate, after removal of solvent, the samples are lyophilized. Once each individual diastereomer is isolated they are further analyzed by analytical (reverse phase and chiral) HPLC, LCMS and NMR. When final products are converted to salts, final characterization of the compounds is carried out after conversion to the salt for each diastereomer.

### Analytical Chiral HPLC Data of Compounds of the Invention.

Column: Chiral Pak AD-H. Column ID: 4.6*250 mm, 5μ. Mobile Phase: Hexane (0.2% diethylamine):Isopropanol—93:7. Flow rate: 1 mL/min.

### Chiral Preparative Data of Compounds of the Invention.

Column: Chiral PAK-AD-H. Column ID: 20*250 mm, 5μ. Mobile Phase: Hexane (0.2% diethylamine): Isopropanol—93:7. Flow rate: 15 mL/min.

The following abbreviations are used herein: hour (h); minute (min); second (sec); ethanol (EtOH); dimethylsulfoxide (DMSO); N,N-dimethylformamide (DMF); trifluoroacetic acid (TFA); tetrahydrofuran (THF); Normal (N); aqueous (aq.); methanol (MeOH); dichloromethane (DCM); ethyl acetate (EtOAc); room temperature (RT); high performance liquid chromatography (HPLC); nuclear magnetic resonance (NMR); N,N-diisopropylethylamine (DIPEA); triethylamine (TEA); liquid chromatography/mass spectrometry (LCMS).

### Example 1: Synthesis of Compounds S2

Compound S1 and urea are combined in a suitable solvent such as EtOH, cooled in an ice-bath, and concentrated HCl is added dropwise. After completion of the addition, the ice bath is removed and the reaction mixture stirred at RT for 30 min. The reaction mixture is then heated at reflux for 5 h. The reaction mixture is cooled to RT, and the EtOH is decanted to give a solid. The solid is heated at reflux with aqueous 5% NaOH solution for 2 h. The reaction mixture is cooled to RT and the precipitate collected by filtration. The precipitate is washed with water and dried to afford compound S2.

### Example 2: Synthesis of Compounds S3

A suspension of compound S2 in POCl₃ is stirred at 100° C. for 3 h. The reaction mixture is cooled to RT and poured slowly with constant shaking into crushed ice to quench the excess of POCl₃. The aqueous layer is extracted with EtOAc. The combined organic layer is washed with water and dried over anhydrous Na₂SO₄. Removal of EtOAc under reduced pressure affords compound S3.

### Example 3: Synthesis of Compounds S5

DIPEA is added to a solution of compound S3 in isopropanol followed by addition of compound S4 (5.89 g, 47 mmol). The reaction mixture is heated to reflux at 100° C. for 16 h. The reaction mixture is cooled to RT. The precipitated product is filtered and washed with hexane to afford compound S5.

### Example 4: Synthesis of Compounds S7

Compound S6 is added to a suspension of compound S5 in dioxane followed by 5 N NaOH and DIPEA. The reaction mixture is allowed to stir at 80° C. for 16 h. The solvent is removed under reduced pressure and the residue is acidified with 1 N HCl solution to pH 4. The product is suspended in water, filtered, washed with ether and dried to afford compound S7.

### Example 5: Syntheses for Conversion of S7 to Formula (I)

Step 1.

To a solution of compound S7 in MeOH (120 mL) is added HOBt.H₂O, N-methylmorpholine and EDC.HCl. The reaction mixture is stirred at RT overnight. The MeOH is removed under reduced pressure and the residue is dissolved in EtOAc. The solution is washed with water followed by brine and dried over anhydrous sodium sulfate. Removal of EtOAc under reduced pressure affords the methyl ester of compound S7.

Step 2.

To a solution of HNR²R³ in dry THF is added a 2 M solution of isopropylmagnesium chloride in THF dropwise under nitrogen at 0° C. The resultant mixture is stirred at 0° C. for 20 min. To this solution is added a solution of the alkyl ester of compound S7 in THF (20 mL) dropwise at 0° C. and the reaction mixture is stirred at RT for 2 h. The reaction is quenched with saturated ammonium chloride solution (50 mL). The product is extracted with EtOAc and the organic layer is dried over anhydrous sodium sulfate. Removal of EtOAc under reduced pressure gives a solid residue that is purified by column chromatography to afford a compound of Formula (I).

### Example 6. Preparation of Compound Nos. 68, 68a and 68b

To a solution of 2-amino thioazole (369.7 mg, 3.24 mmol) in THF (15 ml) was added isopropyl magnesium chloride (1.4 ml, 2.70 mmol) drop wise at 0 deg C. and the resultant mixture was allowed to stir at the same temperature for 30 minutes. To this reaction mixture a solution of methyl (2S)-1-[4-[(5-isopropyl-1H-pyrazol-3-yl)amino]-6,7-dihydro-5H-cyclopenta[d]pyrimidin-2-yl]pyrrolidine-2-carboxylate (200 mg, 0.54 mmol) in THF (5 ml) was added drop wise. The reaction mixture was allowed to stir at room temperature for 24 h. The reaction was quenched with saturated ammonium chloride solution (5 ml) and extracted with ethyl acetate (3×20 ml). The combined organic layers were washed with brine (20 ml) and dried over anhydrous sodium sulfate. Removal of ethyl acetate under reduced pressure gave solid crude which was purified by reverse phase HPLC followed by chiral HPLC to afford 29 mg of(S)-1-(4-((5-isopropyl-1H-pyrazol-3-yl)amino)-6,7-dihydro-5H-cyclopenta[d]pyrimidin-2-yl)-N-methyl-N-(thiazol-2-yl)pyrrolidine-2-carboxamide (Compound No. 68b). 1HNMR (CD3OD, 400 MHz) δ 1.00-1.40 (m, 8H), 2.08-2.30 (m, 4H), 2.43-2.59 (m, 1H), 2.68-2.90 (m, 3H), 2.95-3.05 (m, 2H), 360-3.82 (m, 4H), 5.40 (d, 1H), 5.85 (brs, 1H), 7.16 (brs, 1H), 7.48 (brs, 1H).

The racemate (Compound No. 68), and the enantiomer (Compound No. 68a) are prepared in a similar fashion using the appropriate racemic or enantiomeric starting materials, respectively.

### Example 7. Preparation of Compound Nos. 70, 70a and 70b

To a solution of N-methylthiazol-2-amine (115.1 mg, 1.01 mmol) in THF (3 mL) was added isopropyl magnesium chloride (2 M) in THF (0.5 mL, 1.10 mmol) dropwise at 0° C. and the resultant mixture was allowed to stir at the same temperature for 30 min. To this reaction mixture a solution of methyl (2S)-1-[4-[(3-cyclopentyl-1H-pyrazol-5-yl)amino]-6,7-dihydro 5H-cyclopenta[d]pyrimidin-2-yl]pyrrolidine-2-carboxylate (100 mg, 0.25 mmol) in THF (1 mL) was added dropwise. The reaction mixture was allowed to stir at RT for 16 h. The reaction was quenched with saturated ammonium chloride solution (2 mL) and extracted with EtOAc (2×20 mL). The combined organic layers were washed with brine (20 mL) and dried over anhydrous sodium sulfate. Concentration under reduced pressure gave a solid crude product that was purified by reverse phase HPLC to afford 6 mg of (2S)-1-[4-[(3-cyclopentyl-1H-pyrazol-5-yl)amino]-6,7-dihydro-5H-cyclopenta[d]pyrimidin-2-yl]-N-methyl-N-thiazol-2-yl-pyrrolidine-2-carboxamide (Compound No. 70b). ¹H NMR (CDCl₃, 400 MHz) 1.42-1.82 (m, 4H), 1.90-2.42 (m, 7H), 2.50-3.20 (m, 6H), 3.87-4.02 (m, 6H), 5.22 (d, 1H), 6.20 (brs, 1H), 6.66 (brs, 1H), 6.98 (d, 1H), 7.51 (d, 1H).

The racemate (Compound No. 70), and the enantiomer (Compound No. 70a) are prepared in a similar fashion using the appropriate racemic or enantiomeric starting materials, respectively.

### Example 8. Preparation of Compound Nos. 91, 91a and 91b

To a solution of 1-[4-[(5-cyclopentyl-1H-pyrazol-3-yl)amino]-6,7-dihydro-5H-cyclopenta[d]pyrimidin-2-yl]pyrrolidine-2-carboxylic acid hydrochloride (500 mg, 1.25 mmol) in DMF (10 ml) was added N-methylmethanamine (203 mg, 2.51 mmol), HOBt (16 mg, 0.12 mmol), EDC.HCl (335 mg, 1.75 mmol), N,N-diisopropylethylamine (483 mg, 3.76 mmol) and the reaction mixture was stirred at room temperature for 48 h. Progress of reaction was monitored by LCMS. Reaction mixture was diluted with water (20 ml) and extracted with ethyl acetate (3×20 ml). The combined organic layers were washed with water (8×15 ml) and dried over anhydrous sodium sulfate. Removal of solvent under reduced pressure gave crude product which was purified by reverse phase HPLC followed by chiral HPLC to afford 30 mg of (R)—N,N-dimethyl-1-(4-((5-(tetrahydro-2H-pyran-4-yl)-1H-pyrazol-3-yl)amino)-6,7-dihydro-5H-cyclopenta[d]pyrimidin-2-yl)pyrrolidine-2-carboxamide (Compound No. 91a). 1HNMR (CDCl3, 400 MHz) δ 1.76-2.20 (m, 10H), 2.40 (brs, 1H), 2.65-2.81 (m, 4H), 2.96 (s, 3H), 3.21 (s, 3H), 3.50-3.90 (m, 4H), 4.00-4.10 (m, 2H), 5.03 (dd, 1H), 5.80 (brs, 1H).

The racemate (Compound No. 91) and the enantiomer (Compound No. 91b) are prepared in a similar fashion by either omitting the chiral HPLC separation step or by isolating the fraction corresponding to the enantiomer, respectively.

### Example 9. Preparation of Compound Nos. 109, 109a and 109b

To a solution of 1-[4-[(5-cyclopentyl-1H-pyrazol-3-yl)amino]-6,7-dihydro-5H-cyclopenta[d]pyrimidin-2-yl]pyrrolidine-2-carboxylic acid hydrochloride (1 g, 2.39 mmol) in DMF (10 ml) was added 3,3-difluropyrrolidine (511 mg, 4.77 mmol), HOBt (32.2 mg, 0.23 mmol), EDC.HCl (638 mg, 3.34 mmol), N,N-diisopropylethylamine (924.1 mg, 7.16 mmol) and the reaction mixture was stirred at room temperature for 48 h. Progress of reaction was monitored by LCMS. Reaction mixture was diluted with water (30 ml) and extracted with ethyl acetate (3×30 ml). The combined organic layers were washed with water (8×30 ml) and dried over anhydrous sodium sulfate. Removal of solvent under reduced pressure gave crude product which was purified by reverse phase HPLC followed by chiral HPLC to afford 30 mg of (S)-(1-(4-((5-cyclopentyl-1H-pyrazol-3-yl)amino)-6,7-dihydro-5H-cyclopenta[d]pyrimidin-2-yl)pyrrolidin-2-yl)(3,3-difluoropyrrolidin-1-yl)methanone (Compound No. 109b). 1HNMR (CDCl3, 400 MHz) δ 1.40-1.90 (m, 10H), 1.95-2.2.59 (m, 6H), 2.60-2.90 (m, 4H), 2.99-3.16 (m, 1H), 3.60-4.00 (m, 4H), 4.50 (brs, 1H), 4.70 (brs, 1H), 6.20 (brs, 1H), 6.60 (brs, 1H).

The racemate (Compound No. 109) and the enantiomer (Compound No. 109a) are prepared in a similar fashion by either omitting the chiral HPLC separation step or by isolating the fraction corresponding to the enantiomer, respectively.

### Example 10. Preparation of Compound Nos. 110, 110a and 110b

To a solution of 1-[4-[(5-cyclopentyl-1H-pyrazol-3-yl)amino]-6,7-dihydro-5H-cyclopenta[d]pyrimidin-2-yl]pyrrolidine-2-carboxylic acid hydrochloride (1 g, 2.39 mmol) in DMF (10 ml) was added 3,3,4,4-tetrafluropyrrolidine (500 mg, 4.77 mmol), HOBt (32.2 mg, 0.23 mmol), EDC.HCl (638 mg, 3.34 mmol), N,N-diisopropylethylamine (924.1 mg, 7.16 mmol) and the reaction mixture was stirred at room temperature for 48 h. Progress of reaction was monitored by LCMS. Reaction mixture was diluted with water (30 ml) and extracted with ethyl acetate (3×30 ml). The combined organic layers were washed with water (8×30 ml) and dried over anhydrous sodium sulfate. Removal of solvent under reduced pressure gave crude product which was purified by reverse phase HPLC followed by chiral HPLC to afford 50 mg of (R)-(1-(4-((5-cyclopentyl-1H-pyrazol-3-yl)amino)-6,7-dihydro-5H-cyclopenta[d]pyrimidin-2-yl)pyrrolidin-2-yl)(3,3,4,4-tetrafluoropyrrolidin-1-yl)methanone (Compound No. 110a). 1HNMR (CDCl3, 400 MHz) δ 1.30-1.90 (m, 10H), 1.92-2.40 (m, 6H), 2.48-2.78 (m, 4H), 3.00-3.15 (m, 1H), 3.75-3.97 (m, 2H), 3.98-4.18 (m, 2H), 4.50 (brs, 1H), 5.05 (brs, 1H), 6.22 (brs, 1H), 6.62 (brs, 1H).

The racemate (Compound No. 110) and the enantiomer (Compound No. 110b) are prepared in a similar fashion by either omitting the chiral HPLC separation step or by isolating the fraction corresponding to the enantiomer, respectively.

### Example 11. Preparation of Compound Nos. 111, 111a and 111b

To a solution of 1-[4-[(3-isopropyl-1H-pyrazol-5-yl)amino]-6,7-dihydro-5H-cyclopenta[d]pyrimidin-2-yl]pyrrolidine-2-carboxylic acid hydrochloride (1.00 g, 2.55 mmol) in DMF (10 ml) was added 3,3-difluoropyrrolidine (464 mg, 4.33 mmol), HOBt (34 mg, 0.255 mmol), EDC.HCl (682 mg, 3.57 mmol), N,N-diisopropylethylamine (1.316 g, 10.204 mmol) and the reaction mixture was stirred at room temperature for 18 h. Progress of reaction was monitored by LCMS. Reaction mixture was diluted with water (50 ml) and extracted with ethyl acetate (2×50 ml). The combined organic layers were washed with water (8×50 ml) and dried over anhydrous sodium sulfate. Removal of solvent under reduced pressure gave crude product which was purified by reverse phase HPLC followed by chiral HPLC to afford 20 mg of (S)-(3,3-difluoropyrrolidin-1-yl)(1-(4-((5-isopropyl-1H-pyrazol-3-yl)amino)-6,7-dihydro-5H-cyclopenta[d]pyrimidin-2-yl)pyrrolidin-2-yl)methanone (Compound 111b). 1HNMR 400 MHz (CD3OD): 1.20-1.40 (m, 6H), 1.80-2.20 (m, 6H), 2.28-2.41 (m, 2H), 2.30-2.60 (m, 2H), 2.61-2.81 (m, 4H), 2.82-3.03 (m, 1H0, 3.50-4.00 (m, 6H), 4.65 (brs, 1H), 5.78 (brs, 1H).

The racemate (Compound No. 111) and the enantiomer (Compound No. 111a) are prepared in a similar fashion by either omitting the chiral HPLC separation step or by isolating the fraction corresponding to the enantiomer, respectively.

### Example 12. Preparation of Compound Nos. 112, 112a and 112b

To a solution of 1-[4-[(3-isopropyl-1H-pyrazol-5-yl)amino]-6,7-dihydro-5H-cyclopenta[d]pyrimidin-2-yl]pyrrolidine-2-carboxylic acid hydrochloride (1.00 g, 2.55 mmol) in DMF (10 ml) was added 3,3,4,4-tetrafluoropyrrolidine (510 mg, 3.57 mmol), HOBt (34 mg, 0.255 mmol), EDC.HCl (682 mg, 3.57 mmol), N,N-diisopropylethylamine (1.316 g, 10.204 mmol) and the reaction mixture was stirred at room temperature for 18 h. Progress of reaction was monitored by LCMS. Reaction mixture was diluted with water (50 ml) and extracted with ethyl acetate (2×50 ml). The combined organic layers were washed with water (8×50 ml) and dried over anhydrous sodium sulfate. Removal of solvent under reduced pressure gave crude product which was purified by reverse phase HPLC followed by chiral HPLC to afford 10 mg of (R)-(1-(4-((5-isopropyl-1H-pyrazol-3-yl)amino)-6,7-dihydro-5H-cyclopenta[d]pyrimidin-2-yl)pyrrolidin-2-yl)(3,3,4,4-tetrafluoropyrrolidin-1-yl)methanone (Compound No. 112a). 1HNMR 400 MHz (CD3OD): 1.20-1.40 (m, 6H), 1.95-2.23 (m, 5H), 2.24-2.41 (m, 1H), 2.60-2.80 (m, 4H), 2.81-3.02 (m, 1H), 3.70-4.10 (m, 5H), 4.20-4.40 (m, 1H), 4.65 (brs, 1H), 5.80 (brs, 1H).

The racemate (Compound No. 112) and the enantiomer (Compound No. 112b) are prepared in a similar fashion by either omitting the chiral HPLC separation step or by isolating the fraction corresponding to the enantiomer, respectively.

### Example 13. Preparation of Compound Nos. 133, 133a, and 133b

To a solution of 2-aminothiazole (403 mg, 4.00 mmol) in THF (10 mL) was added isopropyl magnesium chloride (2 mL, 4.00 mmol) dropwise at 0° C. and the resultant mixture was allowed to stir at the same temperature for 30 min. To this reaction mixture a solution of methyl (2S)-1-[4-[(3-cyclopentyl-1H-pyrazol-5-yl)amino]-6,7-dihydro 5H-cyclopenta[d]pyrimidin-2-yl]pyrrolidine-2-carboxylate (400 mg, 1.00 mmol) in THF (10 mL) was added dropwise. The reaction mixture was allowed to stir at RT for 18 h. The reaction was quenched with saturated ammonium chloride solution (5 mL) and extracted with EtOAc (3×20 mL). The combined organic layers were washed with brine (20 mL) and dried over anhydrous sodium sulfate. Concentration under reduced pressure gave a solid crude product that was purified by reverse phase HPLC to afford 40 mg of (2S)-1-[4-[(3-cyclopentyl-1H-pyrazol-5-yl)amino]-6,7-dihydro-5H-cyclopenta[d]pyrimidin-2-yl]-N-thiazol-2-yl-pyrrolidine-2-carboxamide (Compound No. 133b). ¹HNMR (CD₃OD, 400 MHz) 1.45-1.94 (m, 6H), 1.96-2.40 (m, 8H), 2.70-2.98 (m, 5H), 3.60-3.90 (m, 2H), 4.81 (dd, 1H), 6.20 (brs, 1H), 7.10 (d, 1H), 7.41 (d, 1H).

The racemate (Compound No. 133) and the enantiomer (Compound No. 133a) are prepared in a similar fashion using the appropriate racemic or enantiomeric starting materials, respectively.

### Example 14. Preparation of Compound Nos. 134, 134a and 134b

To a solution of 1-[4-[(3-cyclopentyl-1H-pyrazol-5-yl)amino]-6,7-dihydro-5H-cyclopenta[d]pyrimidin-2-yl]pyrrolidine-2-carboxylic acid hydrochloride (500 mg, 1.19 mmol) in DMF (6 mL) was added 1-methylpiperazine (239 mg, 2.39 mmol), 1-hydroxybenzotriazole (HOBt; 16 mg, 0.119 mmol), 1-ethyl-3-(3-dimethylaminopropyl)carbodiimide hydrochloride (EDC.HCl; 320 mg, 1.67 mmol), and DIPEA (464 mg, 3.59 mmol), and the reaction mixture was stirred at RT for 24 h. The reaction mixture was diluted with water (30 mL) and extracted with EtOAc (3×30 mL). The combined organic layers were washed with water (8×30 mL) and dried over anhydrous sodium sulfate. Removal of solvent under reduced pressure gave a crude product that was purified by reverse phase HPLC followed by chiral HPLC to afford 10 mg of [(2R)-1-[4-[(3-cyclopentyl-1H-pyrazol-5-yl)amino]-6,7-dihydro-5H-cyclopenta[d]pyrimidin-2-yl]pyrrolidin-2-yl]-(4-methylpiperazin-1-yl)methanone (Compound No. 134a). ¹H NMR (CD₃OD, 400 MHz) 1.25-1.38 (m, 2H), 1.60-1.85 (m, 6H), 1.90-2.15 (m, 8H), 2.23-2.60 (m, 6H), 3.00-3.20 (m, 1H), 3.60-3.85 (m, 4H), 5.02 (dd, 1H).

To a solution of 1-[4-[(3-cyclopentyl-1H-pyrazol-5-yl)amino]-6,7-dihydro-5H-cyclopenta[d]pyrimidin-2-yl]pyrrolidine-2-carboxylic acid hydrochloride (500 mg, 1.19 mmol) in DMF (6 mL) was added 1-methylpiperazine (239 mg, 2.39 mmol), HOBt (16 mg, 0.119 mmol), EDC*HCl (320 mg, 1.67 mmol), and DIPEA (464 mg, 3.59 mmol), and the reaction mixture was stirred at RT for 24 h. The reaction mixture was diluted with water (30 mL) and extracted with EtOAc (3×30 mL). The combined organic layers were washed with water (8×30 mL) and dried over anhydrous sodium sulfate. Removal of solvent under reduced pressure gave a crude product that was purified by reverse phase HPLC followed by chiral HPLC to afford 10 mg of [(2S)-1-[4-[(3-cyclopentyl-1H-pyrazol-5-yl)amino]-6,7-dihydro-5H-cyclopenta[d]pyrimidin-2-yl]pyrrolidin-2-yl]-(4-methylpiperazin-1-yl)methanone (Compound No. 134b). ¹H NMR (CD₃OD, 400 MHz) 1.25-1.38 (m, 2H), 1.60-1.85 (m, 6H), 1.90-2.15 (m, 8H), 2.23-2.60 (m, 6H), 3.00-3.20 (m, 1H), 3.60-3.85 (m, 4H), 5.02 (dd, 1H).

The racemate (Compound No. 134) is prepared in a similar fashion by omitting the chiral HPLC separation step.

### Example 15. Preparation of Compound Nos. 135, 135a and 135b

To a solution of 1-[4-[(3-cyclopentyl-1H-pyrazol-5-yl)amino]-6,7-dihydro-5H-cyclopenta[d]pyrimidin-2-yl]pyrrolidine-2-carboxylic acid hydrochloride (500 mg, 1.19 mmol) in DMF (6 mL) was added 4,4-dimethylpiperidine hydrochloride (269 mg, 1.79 mmol), HOBt (16 mg, 0.11 mmol), EDC*HCl (320 mg, 1.67 mmol), and DIPEA (618 mg, 4.79 mmol), and the reaction mixture was stirred at RT for 18 h. The reaction mixture was diluted with water (50 mL) and extracted with EtOAc (2×50 mL). The combined organic layers were washed with water (8×50 mL) and dried over anhydrous sodium sulfate. Removal of solvent under reduced pressure gave a crude product that was purified by reverse phase HPLC followed by chiral HPLC to afford 20 mg of [(2R)-1-[4-[(3-cyclopentyl-1H-pyrazol-5-yl)amino]-6,7-dihydro-5H-cyclopenta[d]pyrimidin-2-yl]pyrrolidin-2-yl]-(4,4-dimethyl-1-piperidyl)methanone (Compound No. 135a). ¹H NMR (CD₃OD, 400 MHz) 0.98-1.18 (m, 8H), 1.21-1.42 (m, 4H), 1.60-1.88 (m, 6H), 1.90-2.20 (m, 6H), 2.30-2.45 (m, 1H), 2.61-2.81 (m, 4H), 2.98-3.20 (m, 1H), 3.60-3.90 (m, 4H), 5.01-5.10 (m, 1H), 5.71 (brs, 1H).

To a solution of 1-[4-[(3-cyclopentyl-1H-pyrazol-5-yl)amino]-6,7-dihydro-5H-cyclopenta[d]pyrimidin-2-yl]pyrrolidine-2-carboxylic acid hydrochloride (500 mg, 1.19 mmol) in DMF (6 mL) was added 4,4-dimethylpiperidine hydrochloride (269 mg, 1.79 mmol), HOBt (16 mg, 0.11 mmol), EDC*HCl (320 mg, 1.67 mmol), and DIPEA\(618 mg, 4.79 mmol), and the reaction mixture was stirred at RT for 18 h. The reaction mixture was diluted with water (50 mL) and extracted with EtOAc (2×50 mL). The combined organic layers were washed with water (8×50 mL) and dried over anhydrous sodium sulfate. Removal of solvent under reduced pressure gave a crude product that was purified by reverse phase HPLC followed by chiral HPLC to afford 40 mg of [(2S)-1-[4-[(3-cyclopentyl-1H-pyrazol-5-yl)amino]-6,7-dihydro-5H-cyclopenta[d]pyrimidin-2-yl]pyrrolidin-2-yl]-(4,4-dimethyl-1-piperidyl)methanone (Compound No. 135b). ¹H NMR (CD₃OD, 400 MHz) 0.98-1.18 (m, 8H), 1.21-1.42 (m, 4H), 1.60-1.88 (m, 6H), 1.90-2.20 (m, 6H), 2.30-2.45 (m, 1H), 2.61-2.81 (m, 4H), 2.98-3.20 (m, 1H), 3.60-3.90 (m, 4H), 5.01-5.10 (m, 1H), 5.71 (brs, 1H).

The racemate (Compound No. 135) is prepared in a similar fashion by omitting the chiral HPLC separation step.

### Example 16. Preparation of Compound Nos. 136, 136a and 136b

To a solution of 1-[4-[(3-cyclopentyl-1H-pyrazol-5-yl)amino]-6,7-dihydro-5H-cyclopenta[d]pyrimidin-2-yl]pyrrolidine-2-carboxylic acid hydrochloride (500 mg, 1.19 mmol) in DMF (6 mL) was added 1-methylpiperazin-2-one (205 mg, 1.79 mmol), HOBt (16 mg, 0.11 mmol), EDC*HCl (320 mg, 1.67 mmol), and DIPEA (618 mg, 4.79 mmol), and the reaction mixture was stirred at RT for 18 h. The reaction mixture was diluted with water (50 mL) and extracted with EtOAc (2×50 mL). The combined organic layers were washed with water (8×50 mL) and dried over anhydrous sodium sulfate. Removal of solvent under reduced pressure gave a crude product that was purified by reverse phase HPLC followed by chiral HPLC to afford 20 mg of 4-[(2R)-1-[4-[(3-cyclopentyl-1H-pyrazol-5-yl)amino]-6,7-dihydro-5H-cyclopenta[d]pyrimidin-2-yl]pyrrolidine-2-carbonyl]-1-methyl-piperazin-2-one (Compound No. 136a). ¹H NMR (CD₃OD, 400 MHz) 1.60-1.90 (m, 8H), 1.92-2.20 (m, 8H), 2.30-2.42 (m, 1H), 2.60-2.80 (m, 6H), 2.97-3.12 (m, 1H), 3.22-3.56 (m, 3.61-4.30 (m, 4H), 5.02 (brs, 1H), 5.75 (brs, 1H).

To a solution of 1-[4-[(3-cyclopentyl-1H-pyrazol-5-yl)amino]-6,7-dihydro-5H-cyclopenta[d]pyrimidin-2-yl]pyrrolidine-2-carboxylic acid hydrochloride (500 mg, 1.19 mmol) in DMF (6 mL) was added 1-methylpiperazin-2-one (205 mg, 1.79 mmol), HOBt (16 mg, 0.11 mmol), EDC*HCl (320 mg, 1.67 mmol), and DIPEA (618 mg, 4.79 mmol), and the reaction mixture was stirred at RT for 18 h. The reaction mixture was diluted with water (50 mL) and extracted with EtOAc (2×50 mL). The combined organic layers were washed with water (8×50 mL) and dried over anhydrous sodium sulfate. Removal of solvent under reduced pressure gave a crude product that was purified by reverse phase HPLC followed by chiral HPLC to afford 20 mg of 4-[(2S)-1-[4-[(3-cyclopentyl-1H-pyrazol-5-yl)amino]-6,7-dihydro-5H-cyclopenta[d]pyrimidin-2-yl]pyrrolidine-2-carbonyl]-1-methyl-piperazin-2-one (Compound No. 136b). ¹H NMR (CD₃OD, 400 MHz) 1.60-1.90 (m, 8H), 1.92-2.20 (m, 8H), 2.30-2.42 (m, 1H), 2.60-2.80 (m, 6H), 2.97-3.12 (m, 1H), 3.22-3.56 (m, 3.61-4.30 (m, 4H), 5.02 (brs, 1H), 5.75 (brs, 1H).

The racemate (Compound No. 136) is prepared in a similar fashion by omitting the chiral HPLC separation step.

### Example 17. Preparation of Compound Nos. 137, 137a and 137b

Step 1.

To a solution of tert-butyl piperazine-1-carboxylate (1.00 g, 5.37 mmol) in dichloromethane (10 mL) at 0° C. was added TEA (1.357 g, 13.44 mmol) and acetyl chloride (0.629 g, 8.06 mmol). The reaction mixture was stirred at RT for 18 h. The reaction mixture was diluted with water (20 mL) and extracted with DCM (20 mL). The organic layer was dried over sodium sulfate and concentrated under reduced pressure to afford 900 mg of tert-butyl 4-acetylpiperazine-1-carboxylate.

Step 2.

To a solution of tert-butyl 4-acetylpiperazine-1-carboxylate (900 mg, 3.94 mmol) in DCM (10 mL) was added TFA (5 mL) and the reaction mixture was stirred at 50° C. for 3 h. The reaction solvent was evaporated under reduced pressure to afford 1.120 g of 1-piperazin-1-ylethanone as the TFA salt.

Step 3.

To a solution of (S)-1-[4-[(3-cyclopentyl-1H-pyrazol-5-yl)amino]-6,7-dihydro-5H-cyclopenta[d]pyrimidin-2-yl]pyrrolidine-2-carboxylic acid hydrochloride (1.00 g, 2.39 mmol) in DMF (10 mL) was added 1-piperazin-1-ylethanone trifluoracetate (1.160 g, 4.79 mmol), HOBt (32 mg, 0.239 mmol), EDC*HCl (641 mg, 3.35 mmol), and DIPEA (1.546 g, 11.99 mmol), and the reaction mixture was stirred at RT for 18 h. The reaction mixture was diluted with water (50 mL) and extracted with EtOAc (2×50 mL). The combined organic layers were washed with water (8×50 mL) and dried over anhydrous sodium sulfate. Removal of solvent under reduced pressure gave a crude product that was purified by reverse phase HPLC followed by chiral HPLC to afford 10 mg of 1-[4-[(2S)-1-[4-[(3-cyclopentyl-1H-pyrazol-5-yl)amino]-6,7-dihydro-5H-cyclopenta[d]pyrimidin-2-yl]pyrrolidine-2-carbonyl]piperazin-1-yl]ethanone (Compound No. 137b). ¹H NMR (CD₃OD, 400 MHz) 1.60-2.20 (m, 18H), 2.30-2.45 (m, 1H), 2.60-2.82 (m, 4H), 2.95-3.20 (m, 1H), 3.40-4.00 (m, 8H), 5.03 (brs, 1H), 5.75 (brs, 1H).

The racemate (Compound No. 137) and the enantiomer (Compound No. 137a) are prepared in a similar fashion using the appropriate racemic or enantiomeric starting materials, respectively.

### Example 18. Preparation of Compound Nos. 138, 138a and 138b

To a solution of 1-[4-[(3-isopropyl-1H-pyrazol-5-yl)amino]-6,7-dihydro-5H-cyclopenta[d]pyrimidin-2-yl]pyrrolidine-2-carboxylic acid hydrochloride (1.00 g, 2.55 mmol) in DMF (10 ml) was added pyrrolidine (363 mg, 5.11 mmol), HOBt (34 mg, 0.255 mmol), EDC.HCl (682 mg, 3.57 mmol), N,N-diisopropylethylamine (0.989 g, 7.67 mmol) and the reaction mixture was stirred at room temperature for 18 h. Progress of reaction was monitored by LCMS. Reaction mixture was diluted with water (50 ml) and extracted with ethyl acetate (2×50 ml). The combined organic layers were washed with water (8×50 ml) and dried over anhydrous sodium sulfate. Removal of solvent under reduced pressure gave crude product which was purified by reverse phase HPLC followed by chiral HPLC to afford 20 mg of (R)-(1-(4-((5-isopropyl-1H-pyrazol-3-yl)amino)-6,7-dihydro-5H-cyclopenta[d]pyrimidin-2-yl)pyrrolidin-2-yl)(pyrrolidin-1-yl)methanone (Compound No. 138a). 1HNMR 400 MHz (CD3OD) 1.20-1.40 (m, 6H), 1.80-2.20 (m, 8H), 2.27-2.41 (m, 1H), 2.60-3.10 (m, 6H), 3.20-3.60 (m, 4H), 3.62-3.90 (m, 2H), 4.80 (brs, 1H), 5.80 (brs, 1H).

To a solution of 1-[4-[(3-isopropyl-1H-pyrazol-5-yl)amino]-6,7-dihydro-5H-cyclopenta[d]pyrimidin-2-yl]pyrrolidine-2-carboxylic acid hydrochloride (1.00 g, 2.55 mmol) in DMF (10 ml) was added pyrrolidine (363 mg, 5.11 mmol), HOBt (34 mg, 0.255 mmol), EDC.HCl (682 mg, 3.57 mmol), N,N-diisopropylethylamine (0.989 g, 7.67 mmol) and the reaction mixture was stirred at room temperature for 18 h. Progress of reaction was monitored by LCMS. Reaction mixture was diluted with water (50 ml) and extracted with ethyl acetate (2×50 ml). The combined organic layers were washed with water (8×50 ml) and dried over anhydrous sodium sulfate. Removal of solvent under reduced pressure gave crude product which was purified by reverse phase HPLC followed by chiral HPLC to afford 30 mg of (S)-(1-(4-((5-isopropyl-1H-pyrazol-3-yl)amino)-6,7-dihydro-5H-cyclopenta[d]pyrimidin-2-yl)pyrrolidin-2-yl)(pyrrolidin-1-yl)methanone (Compound No. 138b). 1HNMR 400 MHz (CD3OD) 1.20-1.40 (m, 6H), 1.80-2.20 (m, 8H), 2.27-2.41 (m, 1H), 2.60-3.10 (m, 6H), 3.20-3.60 (m, 4H), 3.62-3.90 (m, 2H), 4.80 (brs, 1H), 5.80 (brs, 1H).

The racemate (Compound No. 138) is prepared in a similar fashion by omitting the chiral HPLC separation step.

### Example 19. Preparation of Compound Nos. 139, 139a and 139b

To a solution of 1-[4-[(3-isopropyl-1H-pyrazol-5-yl)amino]-6,7-dihydro-5H-cyclopenta[d]pyrimidin-2-yl]pyrrolidine-2-carboxylic acid hydrochloride (1.00 g, 2.55 mmol) in DMF (10 ml) was added 3,3-difluoropyrrolidine (464 mg, 4.33 mmol), HOBt (34 mg, 0.255 mmol), EDC.HCl (682 mg, 3.57 mmol), N,N-diisopropylethylamine (1.316 g, 10.204 mmol) and the reaction mixture was stirred at room temperature for 18 h. Progress of reaction was monitored by LCMS. Reaction mixture was diluted with water (50 ml) and extracted with ethyl acetate (2×50 ml). The combined organic layers were washed with water (8×50 ml) and dried over anhydrous sodium sulfate. Removal of solvent under reduced pressure gave crude product which was purified by reverse phase HPLC followed by chiral HPLC to afford 20 mg of (R)-(3,3-difluoropyrrolidin-1-yl)(1-(4-((5-isopropyl-1H-pyrazol-3-yl)amino)-6,7-dihydro-5H-cyclopenta[d]pyrimidin-2-yl)pyrrolidin-2-yl)methanone (Compound No. 139a). 1HNMR 400 MHz (CD3OD): 1.20-1.40 (m, 6H), 1.80-2.20 (m, 6H), 2.28-2.41 (m, 2H), 2.30-2.60 (m, 2H), 2.61-2.81 (m, 4H), 2.82-3.03 (m, 1H0, 3.50-4.00 (m, 6H), 4.65 (brs, 1H), 5.78 (brs, 1H).

The racemate (Compound No. 139) and the enantiomer (Compound No. 139b) are prepared in a similar fashion by either omitting the chiral HPLC separation step or by isolating the fraction corresponding to the enantiomer, respectively.

### Example 20. Preparation of Compound Nos. 140, 140a and 140b

To a solution of 1-[4-[(3-cyclopentyl-1H-pyrazol-5-yl)amino]-6,7-dihydro-5H-cyclopenta[d]pyrimidin-2-yl]pyrrolidine-2-carboxylic acid hydrochloride (1.00 g, 2.39 mmol) in DMF (10 ml) was added 4,4-difluoropiperidine hydrochloride (755 mg, 4.79 mmol), HOBt (32 mg, 0.239 mmol), EDC.HCl (641 mg, 3.35 mmol), N,N-diisopropylethylamine (1.237 g, 9.59 mmol) and the reaction mixture was stirred at room temperature for 18 h. Progress of reaction was monitored by LCMS. Reaction mixture was diluted with water (50 ml) and extracted with ethyl acetate (2×50 ml). The combined organic layers were washed with water (8×50 ml) and dried over anhydrous sodium sulfate. Removal of solvent under reduced pressure gave crude product which was purified by reverse phase HPLC followed by chiral HPLC to afford 90 mg of (S)-(1-(4-((5-cyclopentyl-1H-pyrazol-3-yl)amino)-6,7-dihydro-5H-cyclopenta[d]pyrimidin-2-yl)pyrrolidin-2-yl)(4,4-difluoropiperidin-1-yl)methanone (Compound No. 140b). 1HNMR (CD3OD, 400 MHz) 1.60-2.20 (m, 18H), 2.30-2.42 (m, 1H), 2.6-2.80 (m, 5H), 3.0-3.15 (m, 1H), 3.60-3.90 (m, 4H), 5.0-5.1 (brs, 1H), 6.20 (brs, 1H).

The racemate (Compound No. 140) and the enantiomer (Compound No. 140a) are prepared in a similar fashion by either omitting the chiral HPLC separation step or by isolating the fraction corresponding to the enantiomer, respectively.

### Example 21. Preparation of Compound Nos. 141, 141a and 141b

To a solution of 1-[4-[(3-cyclopentyl-1H-pyrazol-5-yl)amino]-6,7-dihydro-5H-cyclopenta[d]pyrimidin-2-yl]pyrrolidine-2-carboxylic acid hydrochloride (500 mg, 1.19 mmol) in DMF (6 ml) was added 4,4-dimethylpiperidine hydrochloride (269 mg, 1.79 mmol), HOBt (16 mg, 0.11 mmol), EDC.HCl (320 mg, 1.67 mmol), N,N-diisopropylethylamine (618 mg, 4.79 mmol) and the reaction mixture was stirred at room temperature for 18 h. Progress of reaction was monitored by LCMS. Reaction mixture was diluted with water (50 ml) and extracted with ethyl acetate (2×50 ml). The combined organic layers were washed with water (8×50 ml) and dried over anhydrous sodium sulfate. Removal of solvent under reduced pressure gave crude product which was purified by reverse phase HPLC followed by chiral HPLC to afford 20 mg of (R)-(1-(4-((5-cyclopentyl-1H-pyrazol-3-yl)amino)-6,7-dihydro-5H-cyclopenta[d]pyrimidin-2-yl)pyrrolidin-2-yl)(4,4-dimethylpiperidin-1-yl)methanone (Compound No. 141a). 1H NMR (CD3OD, 400 MHz) δ 0.98-1.18 (m, 8H), 1.21-1.42 (m, 4H), 1.60-1.88 (m, 6H), 1.90-2.20 (m, 6H), 2.30-2.45 (m, 1H), 2.61-2.81 (m, 4H), 2.98-3.20 (m, 1H), 3.60-3.90 (m, 4H), 5.01-5.10 (m, 1H), 5.71 (brs, 1H).

The racemate (Compound No. 141) and the enantiomer (Compound No. 141b) are prepared in a similar fashion by either omitting the chiral HPLC separation step or by isolating the fraction corresponding to the enantiomer, respectively.

### Example 22. Preparation of Compound Nos. 142, 142a and 142b

Step 1.

Acetonitrile (1.07 g, 26.32 mmol) was added dropwise to an ice cold solution of NaH (2.05 g, 17.55 mmol) in THF (25 mL). The reaction mixture was stirred at r. t. for 1 h. Methyl pivalate (2.05 g, 17.55 mmol) was added dropwise at 0° C. The reaction mixture was heated to reflux at 100° C. for 16 h. The reaction mixture was concentrated to remove the solvent, diluted with water, extracted with EtOAc (100 mL). Aqueous layer was acidified with HCl which was extracted with DCM (300 mL×3). The combined organic layers were dried over anhydrous sodium sulfate, concentrated under reduced pressure to afford 4,4-dimethyl-3-oxopentanenitrile (2.22 g, crude).

Step 2.

To a solution of 4,4-dimethyl-3-oxopentanenitrile (2.22 g, 19.79 mmol) in ethanol (25 mL) was added hydrazine hydrate (1.06 g, 21.37 mmol) at 0° C. The reaction mixture was stirred at 100° C. for 16 h. The reaction mixture was concentrated to remove the solvent, diluted with water, extracted with EtOAc (300 mL×3). The combined organic layers were dried over anhydrous sodium sulfate, concentrated under reduced pressure to afford 5-(tert-butyl)-1H-pyrazol-3-amine (1.3 g, crude).

Step 3.

To a solution of 2,4-dichloro-6,7-dihydro-5H-cyclopenta[d]pyrimidine (1.5 g, 7.90 mmol) in isopropanol (15 mL) was added N,N-diisopropylethyl amine (2.18 mL, 12.64 mmol) followed by 5-(tert-butyl)-1H-pyrazol-3-amine (1.32 g, 9.48 mmol). The reaction mixture was refluxed at 100° C. for 16 h. The reaction mixture was concentrated under vacuum to get oily residue which was diluted with water (50 mL) and extracted with ethyl acetate (2×50 mL). The combined organic layers were washed with water (3×60 mL) and dried over anhydrous sodium sulfate. Removal of solvent afforded solid crude was triturated with ether (20 mL) to get N-(5-(tert-butyl)-1H-pyrazol-3-yl)-2-chloro-6,7-dihydro-5H-cyclopenta[d]pyrimidin-4-amine (720 mg, 31.44%).

Step 4.

To a suspension of N-(5-(tert-butyl)-1H-pyrazol-3-yl)-2-chloro-6,7-dihydro-5H-cyclopenta[d]pyrimidin-4-amine (0.720 g, 2.46 mmol) and L-proline (1.41 g, 12.33 mmol) in methanol (10 mL) was added trifluoroacetic acid (2.0 mL, 24.6 mmol) drop wise. The reaction mixture was allowed to stir 80° C. for 48 h. Methanol was removed under reduced pressure, residue was basified with saturated sodium bicarbonate solution (50 mL) and product was extracted with ethyl acetate (2×100 mL). The combined organic layers were washed with water (100 mL) and dried over anhydrous sodium sulfate. Removal of ethyl acetate under reduced pressure afforded methyl-(4-((5-(tert-butyl)-1H-pyrazol-3-yl)amino)-6,7-dihydro-5H-cyclopenta[d]pyrimidin-2-yl)-L-prolinate (0.9 g, 94.93%).

Step 5.

A solution of (4-((5-(tert-butyl)-1H-pyrazol-3-yl)amino)-6,7-dihydro-5H-cyclopenta[d]pyrimidin-2-yl)-L-prolinate (600 mg, 1.56 mmol) in 10 mL of 6N HCl was heated at 100° C. for 24 h. The reaction mixture was concentrated, dried using toluene azeotrope to afford the compound (4-((5-(tert-butyl)-1H-pyrazol-3-yl)amino)-6,7-dihydro-5H-cyclopenta[d]pyrimidin-2-yl)-L-proline (700 mg, crude) as light brown solid.

Step 6.

To a solution of (4-((5-(tert-butyl)-1H-pyrazol-3-yl)amino)-6,7-dihydro-5H-cyclopenta[d]pyrimidin-2-yl)-L-proline (700 mg, 1.89 mmol) in DMF (6 mL) was added 4,4-difluoropiperidine hydrochloride (380 mg, 2.46 mmol), HOBt (380 mg, 2.83 mmol), EDC.HCl (540 mg, 2.83 mmol), N,N-diisopropylethylamine (1.6 mL, 9.45 mmol) and the reaction mixture was stirred at r. t. for 16 h. Progress of reaction was monitored by LCMS. Reaction mixture was diluted with water (30 mL) and extracted with ethyl acetate (3×30 mL). The combined organic layers were washed with water (8×30 mL) and dried over anhydrous sodium sulfate. Removal of solvent under reduced pressure afforded crude product which was purified by reverse phase HPLC to afford (S)-(1-(4-((5-(tert-butyl)-1H-pyrazol-3-yl)amino)-6,7-dihydro-5H-cyclopenta[d]pyrimidin-2-yl)pyrrolidin-2-yl)(4,4-difluoropiperidin-1-yl)methanone (Compound No. 142b, 350 mg, 39.13%) as an off white solid.

The racemate (Compound No. 142) and the enantiomer (Compound No. 142a) are prepared in a similar fashion using the appropriate racemic or enantiomeric starting materials, respectively. 1H NMR (400 MHz, DMSO-d6): δ 11.95 (brs, 1H), 8.76 (brs, 1H), 6.65 (brs, 1H), 4.95 (brs, 1H), 4.18-4.05 (m, 2H), 3.72-3.58 (m, 3H), 3.42-3.38 (m, 1H), 3.05-2.98 (m, 1H), 2.60-2.59 (m, 3H), 2.30-2.20 (m, 1H), 1.96-1.60 (m, 9H), 1.25 (s, 9H). LCMS: 474.3 (M+1). UPLC: At Max Plot: 94.81%, At 220 nm: 96.84%.

### Example 23. Preparation of Compound Nos. 143, 143a and 144b

Step 1.

To a solution of tert-butyl piperazine-1-carboxylate (1.0 g, 5.36 mmol) in THF (10 mL) was added propionyl chloride (0.93 mL, 10.73 mmol) and TEA (1.5 mL, 10.73 mmol) dropwise at 0° C. The reaction mixture was stirred at room temperature for 3 h. The reaction mixture was diluted with water, extracted with EtOAc (100 mL×2). The combined organic layers were dried over anhydrous sodium sulfate, concentrated under reduced pressure to afford tert-butyl-4-propionylpiperazine-1-carboxylate (1.3 g, 99.95%) as colorless liquid.

Step 2.

To a solution of tert-butyl 4-propionylpiperazine-1-carboxylate (1.3 g, 5.36 mmol) in dioxane (16 mL) was added 4M HCl in dioxane (8.0 mL). The reaction mixture was stirred at room temperature for 16 h. The reaction mixture was concentrated to afford 1-(piperazin-1-yl)propan-1-one hydrochloride (500 mg, 95.23%) as white solid.

Step 3.

To a solution of (4-((5-cyclopentyl-1H-pyrazol-3-yl)amino)-6,7-dihydro-5H-cyclopenta[d]pyrimidin-2-yl)-L-proline (433 mg, 1.13 mmol) in DMF (10 mL) was added 1-(piperazin-1-yl)prop-2-en-1-one hydrochloride (400 mg, 2.26 mmol), HOBt (228 mg, 1.69 mmol), EDC.HCl (324 mg, 1.69 mmol), N,N-diisopropylethylamine (0.97 mL, 5.65 mmol) and the reaction mixture was stirred at room temperature for 1 h. Progress of reaction was monitored by LCMS. The reaction mixture was diluted with water (30 mL) and extracted with ethyl acetate (3×30 mL). The combined organic layers were washed with water (30 mL×2) and dried over anhydrous sodium sulfate. Removal of solvent under reduced pressure gave crude product which was purified by reverse phase HPLC to afford (S)-1-(4-((4-((5-cyclopentyl-1H-pyrazol-3-yl)amino)-6,7-dihydro-5H-cyclopenta[d]pyrimidin-2-yl)prolyl)piperazin-1-yl)propan-1-one (Compound No. 143b, 284 mg, 28.60%) as an off white solid.

The racemate (Compound No. 143) and the enantiomer (Compound No. 143a) are prepared in a similar fashion using the appropriate racemic or enantiomeric starting materials, respectively. 1H NMR (400 MHz, DMSO-d6): δ 11.90 (brs, 1H), 8.69 (brs, 1H), 6.60 (brs, 1H), 4.93 (s, 2H), 3.76-3.40 (m, 10H), 2.65 (s, 6H), 2.40-2.20 (m, 5H), 1.99-1.80 (m, 8H), 1.05-0.96 (m, 6H). LCMS: 507.4 (M+1). UPLC: At Max Plot: 98.76%, At 220 nm: 98.88%.

### Example 24. Preparation of Compound Nos. 144, 144a and 144b

To a stirred solution of N-ethylthiazol-2-amine (400 mg, 3.12 mmol) in THF (20 mL) was added isopropyl magnesium chloride (3.12 mL, 6.24 mmol) dropwise at 0° C. The resulting mixture was stirred at same temperature for 45 minutes, followed by addition of methyl (4-((5-(tert-butyl)-1H-pyrazol-3-yl)amino)-6,7-dihydro-5H-cyclopenta[d]pyrimidin-2-yl)-L-prolinate (300 mg, 0.78 mmol). The resulting mixture was stirred at r. t. for 2 h. The reaction mixture was quenched with ammonium chloride solution, extracted with EtOAc (100 mL×2). The combined organic layers were dried over sodium sulphate, concentrated under reduced pressure purified by chiral HPLC to afford the compound (S)-1-(4-((5-(tert-butyl)-1 H-pyrazol-3-yl)amino)-6,7-dihydro-5H-cyclopenta[d]pyrimidin-2-yl)-N-ethyl-N-(thiazol-2-yl)pyrrolidine-2-carboxamide (Compound No. 144b, 111 mg, 22.20%) as an off white solid. 1H NMR: (400 MHz, DMSO-d6): δ 11.90 (brs, 1H), 8.80 (brs, 1H), 7.53 (s, 1H), 7.20 (s, 1H), 6.65 (brs, 1H), 5.19-5.15 (m, 1H), 4.62-4.58 (m, 1H), 3.80-3.65 (m, 2H), 2.70-2.60 (m, 2H), 2.50-2.44 (m, 2H), 2.15-1.90 (m, 3H), 1.89-1.84 (m, 2H), 1.53-1.49 (m, 3H), 1.25 (s, 9H). LCMS: 481.3 (M+1). UPLC: At Max Plot: 97.96%, At 220 nm: 98.96%.

The racemate (Compound No. 144) and the enantiomer (Compound No. 144a) are prepared in a similar fashion using the appropriate racemic or enantiomeric starting materials, respectively.

### Example 25. Preparation of Compound Nos. 145, 145a and 145b

Step 1.

To a stirred solution of tert-butyl thiazol-2-ylcarbamate (5.0 g, 24.97 mmol) in DMF (50 mL) was added NaH (5.0 g, 124.85 mmol) portionwise at 0° C. followed by addition of ethyl iodide (6.0 mL, 74.92 mmol) dropwise. The resulting mixture was stirred at room temperature for 5 h. The reaction mixture was diluted with water (300 mL), extracted with EtOAc (300 mL×2). The combined organic layers were dried over sodium sulphate, concentrated under reduced, washed with ether to afford the compound tert-butylethyl(thiazol-2-yl)carbamate (4.8 g, 84.21%) as an off white solid.

Step 2.

A solution of tert-butyl ethyl(thiazol-2-yl)carbamate (4.8 g, 21.02 mmol) in TFA (20 mL) was stirred at r. t. for 5 h. The reaction mixture was concentrated to remove TFA, neutralized with sodium bicarbonate solution (˜50 mL), extracted with EtOAc (300 mL×2). The combined organic layers were dried over sodium sulphate, concentrated under reduced, washed with ether to afford the compound N-ethylthiazol-2-amine (2.5 g, 92.93%) as an off white solid.

Step 3.

To a stirred solution of N-ethylthiazol-2-amine (505 mg, 3.94 mmol) in THF (30 mL) was added isopropyl magnesium chloride (3.94 mL, 7.88 mmol) dropwise at 0° C. The resulting mixture was stirred at same temperature for 45 minutes, followed by addition of methyl (4-((5-isopropyl-1H-pyrazol-3-yl)amino)-6,7-dihydro-5H-cyclopenta[d]pyrimidin-2-yl)-L-prolinate (365 mg, 0.98 mmol). The resulting mixture was stirred at r. t. for 2 h. The reaction mixture was quenched with ammonium chloride solution, extracted with EtOAc (100 mL×2). The combined organic layers were dried over sodium sulphate, concentrated under reduced pressure purified by chiral HPLC to afford the compound (S)—N-ethyl-1-(4-((5-isopropyl-1H-pyrazol-3-yl)amino)-6,7-dihydro-5H-cyclopenta[d]pyrimidin-2-yl)-N-(thiazol-2-yl)pyrrolidine-2-carboxamide (Compound No. 145b, 250 mg, crude) as an off white solid. 1H NMR (400 MHz, DMSO-d6): δ 11.90 (brs, 1H), 8.81 (brs, 1H), 7.58 (s, 1H), 7.22 (s, 1H), 5.16-5.10 (m, 1H), 4.62-4.58 (m, 1H), 4.25-4.18 (m, 1H), 3.80-3.60 (m, 2H), 2.89-2.86 (m, 1H), 2.72-2.63 (m, 3H), 2.44-2.26 (m, 3H), 2.18-1.90 (m, 3H), 1.82-1.60 (m, 2H), 1.46-1.41 (m, 2H), 1.20 (d, 6H). LCMS: 467.3 (M+1). UPLC: At Max Plot: 98.57%, At 220 nm: 98.53%.

The racemate (Compound No. 145) and the enantiomer (Compound No. 145a) are prepared in a similar fashion using the appropriate racemic or enantiomeric starting materials, respectively.

### Example 26. Preparation of Compound Nos. 146, 146a and 146b

Step 1.

To a suspension of 2-chloro-N-(5-cyclopentyl-1H-pyrazol-3-yl)-6,7-dihydro-5H-cyclopenta[d]pyrimidin-4-amine (1.0 g, 3.29 mmol) and 2-methylproline (2.72 g, 16.45 mmol) in methanol (20 mL) was added trifluoroacetic acid (2.51 mL, 32.9 mmol) drop wise. The reaction mixture was allowed to stir 80° C. for 48 h. Methanol was removed under reduced pressure, residue was basified with saturated sodium bicarbonate solution (˜50 mL) and product was extracted with ethyl acetate (2×200 mL). The combined organic layers were washed with water (100 mL) and dried over anhydrous sodium sulfate. Removal of ethyl acetate under reduced pressure afforded methyl 1-(4-((5-cyclopentyl-1H-pyrazol-3-yl)amino)-6,7-dihydro-5H-cyclopenta[d]pyrimidin-2-yl)-2-methylpyrrolidine-2-carboxylate (600 mg, 44.44%).

Step 2.

A solution of methyl 1-(4-((5-cyclopentyl-1H-pyrazol-3-yl)amino)-6,7-dihydro-5H-cyclopenta[d]pyrimidin-2-yl)-2-methylpyrrolidine-2-carboxylate (600 mg, 1.46 mmol) in 10 mL of 6N HCl was heated at 100° C. in microwave for 1 h. The reaction mixture was concentrated, dried using toluene azeotrope to afford the compound 1-(4-((5-cyclopentyl-1H-pyrazol-3-yl)amino)-6,7-dihydro-5H-cyclopenta[d]pyrimidin-2-yl)-2-methylpyrrolidine-2-carboxylic acid (350 mg, 60.39%) as light brown solid.

Step 3.

To a solution of 1-(4-((5-cyclopentyl-1H-pyrazol-3-yl)amino)-6,7-dihydro-5H-cyclopenta[d]pyrimidin-2-yl)-2-methylpyrrolidine-2-carboxylic acid (350 mg, 0.882 mmol) in DMF (6 mL) was added 4,4-difluoropiperidine hydrochloride (278 mg, 1.76 mmol), HOBt (178 mg, 1.32 mmol), EDC.HCl (254 mg, 1.32 mmol), N,N-diisopropylethylamine (0.76 mL, 4.41 mmol) and the reaction mixture was stirred at r. t. for 16 h. Progress of reaction was monitored by LCMS. Reaction mixture was diluted with water (50 mL) and extracted with EtOAc (3×50 mL). The combined organic layers were washed with water (6×50 mL) and dried over anhydrous sodium sulfate. Removal of solvent under reduced pressure afforded crude product which was purified by reverse phase HPLC to afford (1-(4-((5-cyclopentyl-1H-pyrazol-3-yl)amino)-6,7-dihydro-5H-cyclopenta[d]pyrimidin-2-yl)-2-methylpyrrolidin-2-yl)(4,4-difluoropiperidin-1-yl)methanone (Compound No. 146, 150 mg, 34.01%) as pink solid. 1H NMR (400 MHz, DMSO-d6): δ 8.10 (s, 1H), 6.30 (s, 2H), 4.19-4.00 (m, 1H), 3.80-3.70 (m, 1H), 3.60-3.40 (m, 1H), 3.00 (m, 1H), 2.70-2.60 (m, 4H), 2.40-2.25 (m, 4H), 2.15-1.90 (m, 10H), 1.80-1.40 (m, 10H). LCMS: 500.3 (M+1). UPLC: At Max Plot: 93.67%, At 220 nm: 92.20%.

Both enantiomers are prepared in a similar fashion using the appropriate enantiomeric starting materials or by resolution of the racemic mixture by chiral HPLC.

### Example B1. Screening of Compounds Against Trk Kinases

Kinase assays were performed using test compounds against a panel of Trk kinases: Trk A; Trk B; and Trk C. Each compound was tested at a concentration of 0.1 μM in single duplicate mode against the three kinases. Compounds were prepared as 10 mM stock solutions in DMSO prior to use in the assay. The control compound, staurosporine, was tested at 10 concentrations with three-fold serial dilution starting at 20 μM. IC₅₀ values and curve fits were obtained using Prism (GraphPad Software). IC₅₀ values were determined for staurosporine for comparison. Data is presented in Table B1.

The screening was performed using the “HotSpot” assay platform. The assay measures the conversion of a peptide substrate to ³³P-labelled phosphorylated product. Briefly, specific kinase/substrate pairs along with required cofactors were prepared in reaction buffer. Specifically, the substrate was prepared in reaction buffer (20 mM Hepes, pH 7.5; 10 mM MgCl₂; 1 mM EGTA; 0.02% Brij35; 0.02 mg/mL BSA; 0.1 mM Na₃VO₄; 2 mM DTT; 1% DMSO), followed by delivery of any required cofactor to the resulting substrate solution. The corresponding kinase was delivered into the substrate solution and the reaction mixture was mixed gently. Compound was delivered into the reaction mixture, followed 15-20 min later by addition of a mixture of ATP (Sigma, St. Louis Mo.) and ³³P ATP (specific activity 0.01 μCi/μL final; Perkin Elmer, Waltham, Mass.) to a final concentration of 10 μM-30 μM to initiate the reaction. Reactions were carried out at RT for 120 min, followed by spotting of the reactions onto P81 ion exchange filter paper (Whatman #3698-915; Whatman Inc., Piscataway, N.J.). Unbound phosphate was removed by extensive washing of filters in 0.75% phosphoric acid. After subtraction of background derived from control reactions containing inactive enzyme, kinase activity data was expressed as the percent of remaining kinase activity in test samples compared to vehicle (DMSO) reactions. The percent of enzyme inhibition was calculated based on the percent of remaining kinase activity.

TABLE B1
Activity of Test Compounds against Trk Kinases

| Compound | Trk A IC₅₀ | Trk B IC₅₀ | Trk C IC₅₀ |
|---|---|---|---|
| No. | (μM) | (μM) | (μM) |
| 70b | 0.026 | 0.002 | 0.0012 |
| 91a | 1.44 | 0.403 | 0.18 |
| 91b | 6.31 | 0.902 | 0.362 |
| 109b | 0.011 | 0.001 | 0.0003 |
| 110a | 0.189 | 0.171 | 0.0804 |
| 110b | 0.041 | 0.002 | 0.0008 |
| 111b | 0.053 | 0.002 | 0.0007 |
| 112a | 0.67 | 0.628 | 0.279 |
| 112b | 0.109 | 0.004 | 0.0013 |
| 133b | 0.000515 | 0.000038 | 0.000243 |
| 134a | 0.032 | 0.09 | 0.0444 |
| 134b | 0.337 | 0.031 | 0.0048 |
| 135b | 0.012 | 0.001 | 0.0025 |
| 136a | 0.237 | 0.113 | 0.083 |
| 136b | 1.73 | 0.106 | 0.0424 |
| 137b | 1.91 | 0.265 | 0.11 |
| 138a | 0.878 | 0.246 | 0.105 |
| 138b | 0.141 | 0.005 | 0.0014 |
| 139a | 0.669 | 0.258 | 0.144 |
| 141a | 0.135 | 0.231 | 0.146 |

### Example B2. Screening of Compounds in a 192 Kinase Panel

A kinase profiling study of test compounds is conducted by a suitable testing organization such as Caliper LifeSciences Services (Hanover, Md.). The compound is tested at concentrations of 1.0×10⁻⁸ and 1.0×10⁻⁹ M in the RapidKinase192™ panel in duplicate. One or more of the following kinases are assessed in the panel: ABL; Abl(H396P); Abl(Q252H); Abl(T315I); ABL1(E255K); ABL1(G250E); ABL1(Y253F); AKT1; AKT2; AKT3; ALK; AMPK; AMPK-alpha2/beta1/gamma1; Arg; AurA; AurB; AurC; AXL; BLK; BMX; BRSK1; BRSK2; BTK; CaMK1a; CamK1d; CAMK2; CaMK2a; CAMK4; CaMKII beta; CaMKII gamma; Casein kinase 1g2; CDK1/Cycline B1; CDK2; CDK3; CDK5/p25; CHK1; CHK2; CK1d; CK1-epsilon; CK1g3 (CSNK1G3); CK1-gamma1; CLK2; c-Raf; CSNK1A1; c-TAK1; DAPK1; DCAMKL1; DCAMKL2; DDR2; DYRK1a; DYRK1B; DYRK3; DYRK4; EGFR; EGFR (ErbB1) T790M L858R; EGFR(T790M); EPHA1; EPHA2; EPHA3; EPHA4; EPHA5; EPHA8; EPHB1; EPHB2; EPHB3; EPHB4; Erk1; Erk2; Fer; FES; FGFR1; FGFR1 (V561M); FGFR2; FGFR2(N549H); FGFR3; FGFR3 [K650E]; FGFR4; FGR; FLT1; FLT3; Flt3(D835Y); FLT4; FMS; FRK; FYN; GCK; GSK3-alpha; GSK3b; Hck; HER4; HGK; HIPK1; HIPK2; IGF1R; IKBKE (IKK epsilon); IKK-beta; INSR; IRAK4; ITK; JAK2; KDR; KIT; KIT[T670I]; LCK; LOK; LTK; LYN; LYNB; MAPKAPK2; MAPKAPK3; MARK1; MARK2; MARK4; MELK; Mer; MET; MET M1250T; MINK; MNK1 (MKNK1); MSK1; MSK2; MST1; MST1R; MST2; MST3 (STK24); NEK1; NEK2; NTRK2 (TRKB); NuaK1; p38a; p38alpha/SAPK2a (T106M); p38-beta2; p38-delta; p38-gamma; p70S6K; PAK2; PAK3; PAK4; PAK5 (PAK7); PASK; PDGFR beta; PDGFR_alpha; PDGFRA (D842V); PDGFR-alpha (V561D); PhKg1; PhKg2; PIM1; PIM2; PIM3; PKA; PKC-alpha; PKCb2; PKC-beta1; PKC-delta; PKC-epsilon; PKC-eta; PKC-gamma; PKC-theta; PKCz; PKD1; PKD2; PKD3; PKG1-beta; PKGa; PRAK; PRKCI (PKC-iota); PRKX; PYK2; RET; Ret (V804L); RET; Y791F; ROCK1; ROCK2; ROS (ROS1); RSK1; RSK2; RSK3; RSK4; SGK1; SGK2; SGK3; SRC; SRM (SRMS); SYK; TEC; TRKC (NTRK3); TSSK1; TSSK2; TXK; TYRO3; YES; and ZIPK (DAPK3). The screening is performed using the Caliper EZReader2, a 4-sipper LabChip, and ProfilerPro Kinase Selectivity Assay Kits 1 and 2. The assay measures the conversion of a fluorescent peptide substrate to a phosphorylated product. Briefly, the reaction mixture is introduced through a capillary sipper onto the microfluidic chip, where the substrate and product are electrophoretically separated and detected by laser-induced fluorescence. The time-dependent fluorescence signal indicates the extent of the reaction. The extent of binding is measured.

### Example B3. Screening of Compounds Against a Panel of 372 Kinases

A kinase profiling study of test compounds can be conducted by, for example, Reaction Biology Corporation (San Francisco, Calif.). The compounds can be tested at a concentration of 1 μM in a single dose duplicate mode against a panel of 372 kinases: ABL1, ABL2/ARG, ACK1, AKT1, AKT2, AKT3, ALK, ALK1/ACVRL1, ALK2/ACVR1, ALK3/BMPR1A, ALK4/ACVR1B, ALK5/TGFBR1, ALK6/BMPR1B, ARAF, ARK5/NUAK1, ASK1/MAP3K5, Aurora A, Aurora B, Aurora C, AXL, BLK, BMPR2, BMX/ETK, BRAF, BRK, BRSK1, BRSK2, BTK, c-Kit, c-MER, c-MET, c-Src, CAMK1a, CAMK1b, CAMK1d, CAMK1g, CAMK2a, CAMK2b, CAMK2d, CAMK2g, CAMK4, CAMKK1, CAMKK2, CDC7/DBF4, CDK1/cyclin A, CDK1/cyclin B, CDK1/cyclin E, CDK14/cyclin Y (PFTK1), CDK16/cyclin Y (PCTAIRE), CDK18/cyclin Y (PCTK3), CDK2/cyclin A, CDK2/Cyclin A1, CDK2/cyclin E, CDK2/cyclin O, CDK3/cyclin E, CDK4/cyclin D1, CDK4/cyclin D3, CDK5/p25, CDK5/p35, CDK6/cyclin D1, CDK6/cyclin D3, CDK7/cyclin H, CDK9/cyclin K, CDK9/cyclin T1, CHK1, CHK2, CK1a1, CK1d, CK1epsilon, CK1g1, CK1g2, CK1g3, CK2a, CK2a2, CLK1, CLK2, CLK3, CLK4, COT1/MAP3K8, CSK, CTK/MATK, DAPK1, DAPK2, DCAMKL1, DCAMKL2, DDR1, DDR2, DLK/MAP3K12, DMPK, DMPK2, DRAK1/STK17A, DYRK1/DYRK1A, DYRK1B, DYRK2, DYRK3, DYRK4, EGFR, EPHA1, EPHA2, EPHA3, EPHA4, EPHA5, EPHA6, EPHA7, EPHA8, EPHB1, EPHB2, EPHB3, EPHB4, ERBB2/HER2, ERBB4/HER4, ERK1, ERK2/MAPK1, ERK5/MAPK7, ERK7/MAPK15, FAK/PTK2, FER, FES/FPS, FGFR1, FGFR2, FGFR3, FGFR4, FGR, FLT1/VEGFR1, FLT3, FLT4/VEGFR3, FMS, FRK/PTK5, FYN, GCK/MAP4K2, GLK/MAP4K3, GRK1, GRK2, GRK3, GRK4, GRK5, GRK6, GRK7, GSK3a, GSK3b, Haspin, HCK, HGK/MAP4K4, HIPK1, HIPK2, HIPK3, HIPK4, HPK1/MAP4K1, IGF1R, IKKa/CHUK, IKKb/IKBKB, IKKe/IKBKE, IR, IRAK1, IRAK4, IRR/INSRR, ITK, JAK1, JAK2, JAK3, JNK1, JNK2, JNK3, KDR/VEGFR2, KHS/MAP4K5, KSR2, LATS1, LATS2, LCK, LCK2/ICK, LIMK1, LIMK2, LKB1, LOK/STK10, LRRK2, LYN, LYN B, MAPKAPK2, MAPKAPK3, MAPKAPK5/PRAK, MARK1, MARK2/PAR-1Ba, MARK3, MARK4, MEK1, MEK2, MEK3, MEKK1, MEKK2, MEKK3, MEKK6, MELK, MINK/MINK1, MKK4, MKK6, MKK7, MLCK/MYLK, MLCK2/MYLK2, MLK1/MAP3K9, MLK2/MAP3K10, MLK3/MAP3K11, MLK4, MNK1, MNK2, MRCKa/CDC42BPA, MRCKb/CDC42BPB, MSK1/RPS6KA5, MSK2/RPS6KA4, MSSK1/STK23, MST1/STK4, MST2/STK3, MST3/STK24, MST4, MUSK, MYLK3, MYO3b, NEK1, NEK11, NEK2, NEK3, NEK4, NEK5, NEK6, NEK7, NEK9, NIM1, NLK, OSR1/OXSR1, P38a/MAPK14, P38b/MAPK11, P38d/MAPK13, P38g, p70S6K/RPS6KB1, p70S6Kb/RPS6KB2, PAK1, PAK2, PAK3, PAK4, PAK5, PAK6, PASK, PBK/TOPK, PDGFRa, PDGFRb, PDK1/PDPK1, PEAK1, PHKg1, PHKg2, PIM1, PIM2, PIM3, PKA, PKAcb, PKAcg, PKCa, PKCb1, PKCb2, PKCd, PKCepsilon, PKCeta, PKCg, PKCiota, PKCmu/PRKD1, PKCnu/PRKD3, PKCtheta, PKCzeta, PKD2/PRKD2, PKG1a, PKG1b, PKG2/PRKG2, PKN1/PRK1, PKN2/PRK2, PKN3/PRK3, PLK1, PLK2, PLK3, PLK4/SAK, PRKX, PYK2, RAF1, RET, RIPK2, RIPK3, RIPK5, ROCK1, ROCK2, RON/MST1R, ROS/ROS1, RSK1, RSK2, RSK3, RSK4, SGK1, SGK2, SGK3/SGKL, SIK1, SIK2, SIK3, SLK/STK2, SNARK/NUAK2, SRMS, SRPK1, SRPK2, SSTK/TSSK6, STK16, STK21/CIT, STK22D/TSSK1, STK25/YSK1, STK32B/YANK2, STK32C/YANK3, STK33, STK38/NDR1, STK38L/NDR2, STK39/STLK3, SYK, TAK1, TAOK1, TAOK2/TAO1, TAOK3/JIK, TBK1, TEC, TESK1, TGFBR2, TIE2/TEK, TLK1, TLK2, TNIK, TNK1, TRKA, TRKB, TRKC, TSSK2, TSSK3/STK22C, TTBK1, TTBK2, TXK, TYK1/LTK, TYK2, TYRO3/SKY, ULK1, ULK2, ULK3, VRK1, VRK2, WEE1, WNK1, WNK2, WNK3, YES/YES1, ZAK/MLTK, ZAP70, ZIPK/DAPK3, AMPK(A1/B1/G1), AMPK(A1/B1/G2), AMPK(A1/B1/G3), AMPK(A1/B2/G1), AMPK(A2/B1/G1), AMPK(A2/B2/G1), AMPK(A2/B2/G2), AMPK(A2/B2/G3), DNA-PK, EEF2K, EIF2AK1, EIF2AK2, EIF2AK3, EIF2AK4, mTOR/FRAP1, PDK1/PDHK1, PDK2/PDHK2, PDK3/PDHK3, PDK4/PDKH4 and TRPM7/CHAK1.

In vitro profiling protein kinases are performed using the “HotSpot” assay platform. Briefly, specific kinase/substrate pairs along with required cofactors are prepared in reaction buffer (for example, 20 mM Hepes (pH 7.5), 10 mM MgCl₂, 1 mM EGTA, 0.02% Brij35, 0.02 mg/ml BSA, 0.1 mM Na₃VO₄, 2 mM DTT and 1% DMSO). Compounds are delivered into the reaction, followed 15-20 minutes later by addition of a mixture of ATP (Sigma, St. Louis Mo.) and ³³P ATP (Perkin Elmer, Waltham Mass.) to a final concentration of 10 μM. Reactions are carried out at room temperature for 120 min, followed by spotting of the reactions onto P81 ion exchange filter paper (Whatman Inc., Piscataway, N.J.). Unbound phosphate are removed by extensive washing of filters in 0.75% Phosphoric acid. After subtraction of background derived from control reactions containing inactive enzyme, kinase activity data are expressed as the percent of remaining kinase activity in test samples compared to vehicle (dimethyl sulfoxide) reactions. IC₅₀ values and curve fits are obtained using Prism (GraphPad Software).

### Example B4. In Vitro IGF-1R and IR Enzymatic Assays

Inhibition of IGF-1R and IR kinases by compounds described herein were tested. The IC₅₀ values were measured in vitro using an ADP-Glo kinase assay Kit (Promega). For the IGF-1R assay, active recombinant IGF-1R (Millipore #14-802) was used at 1 ng/reaction, the substrate IGF-1R tide (Millipore #12-527) was used at 125 μM and ATP was used at 100 μM. For the IR assay, active recombinant IR (Millipore #14-803) was used at 1 ng/reaction, the substrate Axl tide (Millipore #12-516) was used at 125 μM and ATP at 50 μM. Kinase reactions in both assays were conducted at 30° C. for 20 min with increasing concentrations of the test compounds.

The compounds were screened against activated IGF-1R and activated IR at a concentration of 3.00×10⁻⁸ M in duplicates. The assay results as the percent inhibition of binding are presented in Tables B2 and B3 for IGF-1R and IR respectively.

TABLE B2
Percent Inhibition of Binding against Activated
IGF-1R, and selected IC₅₀ values

| Compound | % Inhibition | | | | IC₅₀ |
|---|---|---|---|---|---|
| No. | @ 30 μM | @ 10 μM | @ 3 μM | @ 1 μM | (μM) |
| 68b | | | | 84 | 0.243 |
| 70b | 95 | 83 | 57 | 35 | |
| 91a | 38 | 14 | 14 | 6 | |
| 91b | 21 | −2 | 4 | 0 | |
| 109b | 84 | 61 | 33 | 12 | |
| 110a | 96 | 83 | 61 | 31 | |
| 110b | 68 | 34 | 18 | 1 | |
| 112b | 86 | 65 | 36 | | |
| 133b | | | | 95 | |
| 134a | | | | 73 | |
| 134b | 77 | 55 | 26 | 16 | |
| 135b | 82 | 43 | 14 | 12 | |
| 136a | | | | 72 | |
| 136b | 63 | 28 | 12 | 15 | |
| 137b | 43 | 15 | −1 | 10 | |
| 138a | 95 | 83 | 62 | | |
| 138b | 94 | 80 | 57 | | |
| 141a | 96 | 74 | 47 | 27 | |
| 142a | | | | | 4.850 |
| 142b | | | | | >10.000 |
| 143a | | | | | 0.740 |
| 143b | | | | | >30.000 |
| 144b | | | | | 10.350 |
| 145b | | | | | 0.470 |
| 146 | | | | | >30.000 |

TABLE B3
Percent Inhibition of Binding against
Activated IR, and selected IC₅₀ values.

| Compound | % Inhibition | | | | IC₅₀ |
|---|---|---|---|---|---|
| No. | @ 30 μM | @ 10 μM | @ 3 μM | @ 1 μM | (μM) |
| 68b | | 0.243 | | 77 | 0.364 |
| 70b | 97 | | 60 | 30 | |
| 91a | 45 | | 16 | 5 | |
| 91b | 7 | | −2 | 1 | |
| 109b | 86 | | 39 | 12 | |
| 110a | 97 | | 64 | 32 | |
| 110b | 74 | | 21 | 3 | |
| 111b | | | | 35 | |
| 112a | | | | 35 | |
| 112b | 89 | | 36 | 12 | |
| 133b | | | | 97 | |
| 134a | | | | 80 | |
| 134b | 85 | | 36 | 26 | |
| 135b | 88 | | 26 | 11 | |
| 136a | | | | 64 | |
| 136b | 72 | | 21 | −2 | |
| 137b | 60 | | 9 | 19 | |
| 138a | 96 | | 61 | 37 | |
| 138b | 92 | | 46 | 21 | |
| 139a | | | | 42 | |
| 141a | 97 | | 47 | 17 | |
| 142a | | 4.850 | | | 2.940 |
| 142b | | >10.000 | | | 11.650 |
| 143a | | 0.740 | | | 0.650 |
| 143b | | >30.000 | | | 17.150 |
| 144b | | 10.350 | | | 5.960 |
| 145b | | 0.470 | | | 0.670 |
| 146 | | >30.000 | | | >30.000 |

### Example B5. Screening of Compounds Against FLT3 Kinases

Kinase assays were performed using test compounds against a panel of FLT3 kinases: FLT3; FLT3 (D835Y); and FLT3 (ITD). Compounds were prepared as 10 mM stock solutions in DMSO prior to use in the assay. Each compound was tested at 10 concentrations with three-fold serial dilution starting at 1 μM against the three kinases. The control compound, staurosporine, was tested at 10 concentrations with four-fold serial dilution starting at 20 μM. IC₅₀ values and curve fits were obtained using Prism (GraphPad Software). The screening was performed using the “HotSpot” assay platform. The assay measures the conversion of a peptide substrate to ³³P-labelled phosphorylated product. Briefly, specific kinase/substrate pairs along with required cofactors were prepared in reaction buffer. Specifically, the substrate was prepared in reaction buffer (20 mM Hepes, pH 7.5; 10 mM MgCl₂; 1 mM EGTA; 0.02% Brij35; 0.02 mg/mL BSA; 0.1 mM Na₃VO₄; 2 mM DTT; 1% DMSO), followed by delivery of any required cofactor to the resulting substrate solution. The corresponding kinase was delivered into the substrate solution and the reaction mixture was mixed gently. Compound was delivered into the reaction mixture, followed 15-20 min later by addition of a mixture of ATP (Sigma, St. Louis Mo.) and ³³P ATP (specific activity 0.01 μCi/μL final; Perkin Elmer, Waltham, Mass.) to a final concentration of 10 μM to initiate the reaction. Reactions were carried out at RT for 120 min, followed by spotting of the reactions onto P81 ion exchange filter paper (Whatman #3698-915; Whatman Inc., Piscataway, N.J.). Unbound phosphate was removed by extensive washing of filters in 0.75% phosphoric acid. After subtraction of background derived from control reactions containing inactive enzyme, kinase activity data was expressed as the percent of remaining kinase activity in test samples compared to vehicle (DMSO) reactions. The percent of enzyme inhibition was calculated based on the percent of remaining kinase activity. IC₅₀ values were determined. Data is presented in Table B5.

TABLE B5
Activity of Test Compounds against FLT3 Kinases

| Compound | FLT3 IC₅₀ | FLT3 (D835Y) IC₅₀ | FLT3 (ITD) IC₅₀ |
|---|---|---|---|
| No. | (μM) | (μM) | (μM) |
| 68b | 0.112 | 0.0838 | 0.167 |
| 142a | >1 | >1 | >1 |
| 142b | >1 | >1 | >1 |
| 143a | 0.0139 | 0.014 | 0.0137 |
| 143b | 0.058 | 0.187 | 0.31 |
| 144b | >1 | >1 | >1 |
| 145b | 0.155 | 0.262 | 0.435 |
| 146 | 0.798 | 1.57 | 1.48 |

### Example B6. Screening of Compounds Against ABL1, ABL2/ARG and ROS/ROS1 Kinases

The screening was performed using the “HotSpot” assay platform. The assay measures the conversion of a peptide substrate to ³³P-labelled phosphorylated product. Briefly, specific kinase/substrate pairs along with required cofactors were prepared in reaction buffer. Specifically, the substrate was prepared in reaction buffer (20 mM Hepes, pH 7.5; 10 mM MgCl₂; 1 mM EGTA; 0.02% Brij35; 0.02 mg/mL BSA; 0.1 mM Na₃VO₄; 2 mM DTT; 1% DMSO), followed by delivery of any required cofactor to the resulting substrate solution. The corresponding kinase was delivered into the substrate solution and the reaction mixture was mixed gently. Compound was delivered into the reaction mixture, followed 15-20 minutes later by addition of a mixture of ATP (Sigma, St. Louis Mo.) and ³³P ATP (specific activity 0.01 μCi/μL final; Perkin Elmer, Waltham, Mass.) to a final concentration of 10 μM-30 μM to initiate the reaction. Reactions were carried out at RT for 120 min, followed by spotting of the reactions onto P81 ion exchange filter paper (Whatman #3698-915; Whatman Inc., Piscataway, N.J.). Unbound phosphate was removed by extensive washing of filters in 0.75% phosphoric acid. After subtraction of background derived from control reactions containing inactive enzyme, kinase activity data was expressed as the percent of remaining kinase activity in test samples compared to vehicle (DMSO) reactions. The percent of enzyme inhibition was calculated based on the percent of remaining kinase activity. IC₅₀ values and curve fits were obtained using Prism (GraphPad Software).

TABLE B6
Activity of Test Compounds against
ABL1, ABL2/ARG and ROS/ROS1 Kinases

| Compound | ABL1 IC₅₀ | ABL2/ARG IC₅₀ | ROS/ROS1 IC₅₀ |
|---|---|---|---|
| No. | (μM) | (μM) | (μM) |
| 68b | 0.316 | 0.041 | 0.00441 |
| 142a | >1.000000 | >1.000000 | >1.000000 |
| 142b | >1.000000 | >1.000000 | >1.000000 |
| 143a | >1.000000 | 0.398 | 0.0522 |
| 143b | >1.000000 | >1.000000 | >1.000000 |
| 144b | >1.000000 | >1.000000 | >1.000000 |

### Example B7. Screening of Compounds in Additional Kinase Panels

Kinase assays are performed using test compounds against panels of kinases including AXL; c-Met; c-Mer; DDR1; DDR2, and MUSK. Each compound is tested at a concentration of 0.1 μM in single duplicate mode against the four kinases. Compounds are prepared as 10 mM stock solutions in DMSO prior to use in the assay. The control compound, staurosporine, is tested at 10 concentrations with four-fold serial dilution starting at 20 μM. IC₅₀ values and curve fits are obtained using Prism (GraphPad Software). IC₅₀ values are determined for staurosporine for comparison.

The screening is performed using the “HotSpot” assay platform. The assay measures the conversion of a peptide substrate to ³³P-labelled phosphorylated product. Briefly, specific kinase/substrate pairs along with required cofactors are prepared in reaction buffer. Specifically, the substrate is prepared in reaction buffer (20 mM Hepes, pH 7.5; 10 mM MgCl₂; 1 mM EGTA; 0.02% Brij35; 0.02 mg/mL BSA; 0.1 mM Na₃VO₄; 2 mM DTT; 1% DMSO), followed by delivery of any required cofactor to the resulting substrate solution. The corresponding kinase is delivered into the substrate solution and the reaction mixture was mixed gently. Test compound is delivered into the reaction mixture, followed 15-20 min later by addition of a mixture of ATP (Sigma, St. Louis Mo.) and ³³P ATP (specific activity 0.01 μCi/μL final; Perkin Elmer, Waltham, Mass.) to a final concentration of 10 μM-30 μM to initiate the reaction. Reactions are carried out at RT for 120 min, followed by spotting of the reactions onto P81 ion exchange filter paper (Whatman #3698-915; Whatman Inc., Piscataway, N.J.). Unbound phosphate is removed by extensive washing of filters in 0.75% phosphoric acid. After subtraction of background derived from control reactions containing inactive enzyme, kinase activity data is expressed as the percent of remaining kinase activity in test samples compared to vehicle (DMSO) reactions. The percent of enzyme inhibition is calculated based on the percent of remaining kinase activity.

### Example B8. Efficacy in Xenograft Models

To evaluate the efficacy of tumor growth inhibition, test compounds are studied using human cancer mouse models. The models are prepared by subcutaneously or orthotopically implanting mice with human cancer cells. When the tumor size of the mice develops (e.g., reaches 100 mm³), the mice are divided into groups for treatment with the test compound (where different groups receiving the test compound may be administered different amounts of the test compound) or are provided no compound as a control. The mice are treated (e.g., orally administered a compound of the invention) for a period of time. During the course of the study, the tumor is measured and the weight of the mice is determined. After the treatment period, the mice are sacrificed shortly (e.g. 1 or 2 hours) after the final dose. Blood and tissue are collected for biochemical analysis.

### Example B9. Cell Screens for Cancer Types and Subtypes

Cancer cells are grown according to recommended culture conditions. To evaluate cell viability, cells are plated and allowed to attach overnight. The cell density is adjusted so that the cells will be ˜70-80% confluent at the end of the assay. After cell attachment, the medium is removed and replaced with fresh medium containing test compound at different concentrations. Test compound is diluted from a DMSO stock such that the final DMSO concentration in the assay is 0.2%. Seventy-two hours after exposing cells to test compound, cell viability is evaluated using a cell viability assay such as CellTiter Glo™ (Promega, Madison, Wis.), and IC₅₀ values are determined.

Cancer cell types that may be used in this assay include, without limitation, bladder cancer, breast cancer, colorectal cancer, endometrial cancer, hematopoietic cancer, liver cancer, lung cancer, ovarian cancer, pancreatic cancer, and prostate cancer cells. Breast cancer cell types include, without limitation, HER2 positive breast cancer, luminal breast cancer, triple negative breast cancer (e.g., basal, mesenchymal, mesenchymal stem-like, immunomodulatory, and luminal androgen receptor subtypes), and unclassified breast cancer cells. Liver cancer cell types include, without limitation, hepatitis B virus-derived liver cancer and virus-negative liver cancer cells. Lung cancer cell types include, without limitation, non-small cell lung carcinoma, small cell lung carcinoma, adenocarcinoma, mucoepidermoid, anaplastic, large cell, and unclassified lung cancer cells.

All references throughout, such as publications, patents, patent applications and published patent applications, are incorporated herein by reference in their entireties.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

1: A compound of Formula (I): 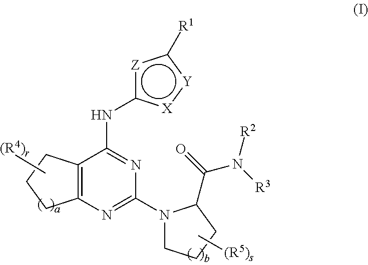
wherein:
X is N;
Y is NH or CH; and
Z is N, NH, N(C₁₋₄alkyl), or CH;
wherein the X-, Y-, and Z-containing ring is a 5-membered heteroaryl with at least two nitrogen ring atoms;
R¹ is C₃₋₈alkyl, C₃₋₈cycloalkyl, or 3- to 8-membered heterocyclyl;
wherein the C₃₋₈alkyl of R¹ is optionally substituted with 1, 2, or 3 substituents selected from the group consisting of —OH, —OC₁₋₄alkyl, —F, —CN, —NR^{a}R^{b}, and C₃₋₈cycloalkyl; and
the C₃₋₈cycloalkyl and 3- to 8-membered heterocyclyl of R¹ are optionally substituted with 1, 2, 3, or 4 substituents selected from the group consisting of C₁₋₄alkyl, —OH, —OC₁₋₄alkyl, —F, —CF₃, —CN, —CO₂H, —CO₂C₁₋₄alkyl, —C(O)NR^{a}R^{b}, and —NR^{a}R^{b};
wherein R^{a} and R^{b} are each independently H or C₁₋₄alkyl;
R² is H or C₁₋₄alkyl optionally substituted with —OH, —OC₁₋₄alkyl, —F, —CF₃, —CN, or —NR^{c}R^{d};
wherein R^{c} and R^{d} are each independently H or C₁₋₄alkyl;
R³ is (a) C₁₋₄alkyl optionally substituted with —OH, —OC₁₋₄alkyl, —F, —CF₃, —CN, or —NR^{e}R^{f}; or (b) a 5- or 6-membered heteroaryl optionally substituted with one or more substitutents selected from the group consisting of C₁₋₄alkyl, —OH, —OC₁₋₄alkyl, halo, —CF₃, —CN, —CO₂H, —CO₂C₁₋₄alkyl, —C(O)NR^{e}R^{f}, and —NR^{e}R^{f};
wherein R^{e} and R^{f} are each independently H or C₁₋₄alkyl;
or R² and R³ taken together with the nitrogen to which they are attached form a 5- to 10-membered heterocyclyl optionally substituted with 1, 2, 3, or 4 substituents selected from the group consisting of C₁₋₄alkyl, —OH, —OC₁₋₄alkyl, oxo, halo, —CF₃, —CN, —C(O)C₁₋₄alkyl, —CO₂H, —CO₂C₁₋₄alkyl, —C(O)NR^{g}R^{h}, and —NR^{g}R^{h};
wherein R^{g} and R^{h} are each independently H or C₁₋₄alkyl;
a and b are each independently 1 or 2;
r and s are each independently 0, 1, 2, 3, or 4;
each R⁴ and R⁵ is independently C₁₋₄alkyl, —OH, —OC₁₋₄alkyl, —F, —CF₃, —CN, —NRⁱR^{j}, or oxo;
wherein Rⁱ and R^{j} are each independently H or C₁₋₄alkyl;
or a pharmaceutically acceptable salt thereof;
wherein the compound is not a compound in Table X or a pharmaceutically acceptable salt thereof.

2: The compound of claim 1, wherein R¹ is propyl, isopropyl, butyl, isobutyl, tert-butyl, pentyl, isopentyl, hexyl, isohexyl, heptyl, or octyl, each optionally substituted as described for Formula (I).

3: The compound of claim 1, wherein R¹ is cyclopropyl, cyclobutyl, cyclopentyl, cyclohexyl, cycloheptyl, or cyclooctyl, each optionally substituted as described for Formula (I).

4: The compound of claim 1, wherein R¹ is C₅₋₈alkyl or C₅₋₈cycloalkyl, each optionally substituted as described for Formula (I).

5: The compound of claim 1, wherein R¹ is cyclopentyl or cyclohexyl, each optionally substituted as described for Formula (I).

6: The compound of claim 1, wherein R¹ is cyclopentyl or cyclohexyl.

7: The compound of claim 1, wherein R¹ is 3- to 8-membered heterocyclyl, optionally substituted as described for Formula (I).

8: The compound of claim 1, wherein R¹ is a 5- to 7-membered heterocyclyl, optionally substituted as described for Formula (I).

9: The compound of claim 1, wherein R¹ is C₃₋₈alkyl substituted with 1, 2, or 3 substituents independently selected from —OH, methoxy, ethoxy, propyloxy, isopropoxy, —F, —CN, amino, methylamino, dimethylamino, and C₃₋₈cycloalkyl.

10: The compound of claim 1, wherein R¹ is C₃₋₈cycloalkyl or 3- to 8-membered heterocyclyl, optionally substituted with 1 to 4 substituents independently selected from methyl, ethyl, propyl, isopropyl, —OH, methoxy, —F, and —CF₃.

11: The compound of claim 1, wherein R² is H or C₁₋₄alkyl.

12: The compound of claim 1, wherein R² is C₂₋₄alkyl substituted as described for Formula (I).

13: The compound of claim 1, wherein R² is H, methyl, or ethyl.

14: The compound of claim 1, wherein R² is H.

15: The compound of claim 1, wherein R² is C₁₋₄alkyl.

16: The compound of claim 1, wherein R² is methyl.

17: The compound of claim 1, wherein R³ is C₁₋₄alkyl optionally substituted as described for Formula (I).

18: The compound of claim 1, wherein R³ is methyl or ethyl.

19: The compound of claim 1, wherein R³ is a 5-membered heteroaryl optionally substituted as described for Formula (I).

20: The compound of claim 19, wherein the 5-membered heteroaryl is selected from the group consisting of thiophenyl, thiazolyl, oxazolyl, pyrazolyl, imidazolyl, oxadiazolyl, thiadizaolyl, and triazolyl.

21: The compound of claim 1, wherein R³ is unsubstituted oxazolyl, pyrazolyl, imidazolyl, oxadiazolyl, thiadiazolyl, or triazolyl.

22: The compound of claim 21, wherein the 5-membered heteroaryl is thiophenyl or thiazolyl.

23: The compound of claim 1, wherein R³ is thiophenyl or thiazolyl, each substituted with C₁₋₄alkyl, —OH, —OC₁₋₄alkyl, —CF₃, —CN, —CO₂H, —CO₂C₁₋₄alkyl, —C(O)NR^{e}R^{f}, or —NR^{e}R^{f}.

24: The compound of claim 1, wherein R³ is a 6-membered heteroaryl optionally substituted as described for Formula (I).

25: The compound of claim 1, wherein R³ is pyridinyl, optionally substituted as described for Formula (I).

26: The compound of claim 1, wherein R³ is pyridinyl substituted with —OH or —F.

27: The compound of claim 1, wherein R² and R³ taken together with the nitrogen to which they are attached form a 5- to 8-membered heterocyclyl ring optionally substituted with C₁₋₄alkyl, —OH, —OC₁₋₄alkyl, oxo, halo, —CF₃, —CN, —C(O)C₁₋₄alkyl, —CO₂H, —CO₂C₁₋₄alkyl, —C(O)NR^{g}R^{h}, or —NR^{g}R^{h}.

28: The compound of claim 1, wherein R² and R³ taken together with the nitrogen to which they are attached form a pyrrolidinyl or piperidinyl ring substituted with one or two substituents selected from the group consisting of —OH, —OC₁₋₄alkyl, oxo, halo, —CF₃, —CN, —CO₂H, —CO₂C₁₋₄alkyl, —C(O)NR^{g}R^{h}, and —NR^{g}R^{h}.

29: The compound of claim 28, wherein the piperidinyl ring is substituted with one or two substituents selected from the group consisting of —OH, —OC₁₋₄alkyl, oxo, —CF₃, —CN, —CO₂H, —CO₂C₁₋₄alkyl, —C(O)NR^{g}R^{h}, and —NR^{g}R^{h}.

30: The compound of claim 1, wherein R² and R³ taken together with the nitrogen to which they are attached form a morpholinyl, thiomorpholinyl, 1,1-dioxo-thiomorpholinyl, or piperazinyl ring, each optionally substituted as described for Formula (I).

31: The compound of claim 1, wherein R² and R³ taken together with the nitrogen to which they are attached form a piperazinyl ring, optionally substituted as described for Formula (I).

32: The compound of claim 1, wherein a is 1.

33: The compound of claim 1, wherein a is 2.

34: The compound of claim 1, wherein b is 1.

35: The compound of claim 1, wherein b is 2.

36: The compound of claim 1, wherein r is 0.

37: The compound of claim 1, wherein r is 1.

38: The compound of claim 1, wherein r is 2.

39: The compound of claim 1, wherein r is 3 or 4.

40: The compound of claim 1, wherein s is 0.

41: The compound of claim 1, wherein s is 1.

42: The compound of claim 1, wherein s is 2.

43: The compound of claim 1, wherein s is 3 or 4.

44: The compound of claim 1, wherein each of R⁴ and R⁵ is independently methyl, —OH, methoxy, —F, —CF₃, —CN, amino, methylamino, dimethylamino, or oxo.

45: The compound of claim 1, wherein R¹ is C₅₋₈cycloalkyl, R² is C₁₋₄alkyl, or R² and R³ taken together with the nitrogen to which they are attached form a 5- to 7-membered heterocyclyl optionally substituted with one or two substituents selected from the group consisting of C₁₋₄alkyl, —OH, —OC₁₋₄alkyl, oxo, halo, —CF₃, —CN, —CO₂H, —CO₂C₁₋₄alkyl, —C(O)NR^{g}R^{h}, and —NR^{g}R^{h}; wherein R^{g} and R^{h} are each independently H or C₁₋₄alkyl.

46: A compound selected from the group consisting of compounds of Formula (II), Formula (III), Formula (IV), and Formula (V), or a pharmaceutically acceptable salt thereof, wherein the compound is not a compound from Table X or a pharmaceutically acceptable salt thereof.

47: A compound selected from the group consisting of:
1-(4-(5-isobutyl-1H-pyrazol-3-ylamino)-6,7-dihydro-5H-cyclopenta[d]pyrimidin-2-yl)-N-(6-oxo-1,6-dihydropyridin-3-yl)pyrrolidine-2-carboxamide;
(R)-1-(4-(5-isobutyl-1H-pyrazol-3-ylamino)-6,7-dihydro-5H-cyclopenta[d]pyrimidin-2-yl)-N-(6-oxo-1,6-dihydropyridin-3-yl)pyrrolidine-2-carboxamide;
(S)-1-(4-(5-isobutyl-1H-pyrazol-3-ylamino)-6,7-dihydro-5H-cyclopenta[d]pyrimidin-2-yl)-N-(6-oxo-1,6-dihydropyridin-3-yl)pyrrolidine-2-carboxamide;
1-(4-(5-cyclopropyl-1H-pyrazol-3-ylamino)-6,7-dihydro-5H-cyclopenta[d]pyrimidin-2-yl)-N-(6-oxo-1,6-dihydropyridin-3-yl)pyrrolidine-2-carboxamide;
(R)-1-(4-(5-cyclopropyl-1H-pyrazol-3-ylamino)-6,7-dihydro-5H-cyclopenta[d]pyrimidin-2-yl)-N-(6-oxo-1,6-dihydropyridin-3-yl)pyrrolidine-2-carboxamide;
(S)-1-(4-(5-cyclopropyl-1H-pyrazol-3-ylamino)-6,7-dihydro-5H-cyclopenta[d]pyrimidin-2-yl)-N-(6-oxo-1,6-dihydropyridin-3-yl)pyrrolidine-2-carboxamide;
1-(4-(5-cyclopentyl-1H-pyrazol-3-ylamino)-6,7-dihydro-5H-cyclopenta[d]pyrimidin-2-yl)-N-(6-oxo-1, 6-dihydropyridin-3-yl)pyrrolidine-2-carboxamide;
(R)-1-(4-(5-cyclopentyl-1H-pyrazol-3-ylamino)-6,7-dihydro-5H-cyclopenta[d]pyrimidin-2-yl)-N-(6-oxo-1,6-dihydropyridin-3-yl)pyrrolidine-2-carboxamide;
(S)-1-(4-(5-cyclopentyl-1H-pyrazol-3-ylamino)-6,7-dihydro-5H-cyclopenta[d]pyrimidin-2-yl)-N-(6-oxo-1,6-dihydropyridin-3-yl)pyrrolidine-2-carboxamide;
1-(4-(5-cyclohexyl-1H-pyrazol-3-ylamino)-6,7-dihydro-5H-cyclopenta[d]pyrimidin-2-yl)-N-(6-oxo-1,6-dihydropyridin-3-yl)pyrrolidine-2-carboxamide;
(R)-1-(4-(5-cyclohexyl-1H-pyrazol-3-ylamino)-6,7-dihydro-5H-cyclopenta[d]pyrimidin-2-yl)-N-(6-oxo-1,6-dihydropyridin-3-yl)pyrrolidine-2-carboxamide;
(S)-1-(4-(5-cyclohexyl-1H-pyrazol-3-ylamino)-6,7-dihydro-5H-cyclopenta[d]pyrimidin-2-yl)-N-(6-oxo-1,6-dihydropyridin-3-yl)pyrrolidine-2-carboxamide;
N-(5-chlorothiophen-2-yl)-1-(4-(5-isopropyl-1H-pyrazol-3-ylamino)-6,7-dihydro-5H-cyclopenta[d]pyrimidin-2-yl)pyrrolidine-2-carboxamide;
(R)—N-(5-chlorothiophen-2-yl)-1-(4-(5-isopropyl-1H-pyrazol-3-ylamino)-6,7-dihydro-5H-cyclopenta[d]pyrimidin-2-yl)pyrrolidine-2-carboxamide;
(S)—N-(5-chlorothiophen-2-yl)-1-(4-(5-isopropyl-1H-pyrazol-3-ylamino)-6,7-dihydro-5H-cyclopenta[d]pyrimidin-2-yl)pyrrolidine-2-carboxamide;
N-(5-chlorothiophen-2-yl)-1-(4-(5-cyclopropyl-1H-pyrazol-3-ylamino)-6,7-dihydro-5H-cyclopenta[d]pyrimidin-2-yl)pyrrolidine-2-carboxamide;
(R)—N-(5-chlorothiophen-2-yl)-1-(4-(5-cyclopropyl-1H-pyrazol-3-ylamino)-6,7-dihydro-5H-cyclopenta[d]pyrimidin-2-yl)pyrrolidine-2-carboxamide;
(S)—N-(5-chlorothiophen-2-yl)-1-(4-(5-cyclopropyl-1H-pyrazol-3-ylamino)-6,7-dihydro-5H-cyclopenta[d]pyrimidin-2-yl)pyrrolidine-2-carboxamide;
N-(5-chlorothiophen-2-yl)-1-(4-(5-cyclopentyl-1H-pyrazol-3-ylamino)-6,7-dihydro-5H-cyclopenta[d]pyrimidin-2-yl)pyrrolidine-2-carboxamide;
(R)—N-(5-chlorothiophen-2-yl)-1-(4-(5-cyclopentyl-1H-pyrazol-3-ylamino)-6,7-dihydro-5H-cyclopenta[d]pyrimidin-2-yl)pyrrolidine-2-carboxamide;
(S)—N-(5-chlorothiophen-2-yl)-1-(4-(5-cyclopentyl-1H-pyrazol-3-ylamino)-6,7-dihydro-5H-cyclopenta[d]pyrimidin-2-yl)pyrrolidine-2-carboxamide;
N-(5-chlorothiophen-2-yl)-1-(4-(5-cyclohexyl-1H-pyrazol-3-ylamino)-6,7-dihydro-5H-cyclopenta[d]pyrimidin-2-yl)pyrrolidine-2-carboxamide;
(R)—N-(5-chlorothiophen-2-yl)-1-(4-(5-cyclohexyl-1H-pyrazol-3-ylamino)-6,7-dihydro-5H-cyclopenta[d]pyrimidin-2-yl)pyrrolidine-2-carboxamide;
(S)—N-(5-chlorothiophen-2-yl)-1-(4-(5-cyclohexyl-1H-pyrazol-3-ylamino)-6,7-dihydro-5H-cyclopenta[d]pyrimidin-2-yl)pyrrolidine-2-carboxamide;
N-(5-chlorothiazol-2-yl)-1-(4-(5-isopropyl-1H-pyrazol-3-ylamino)-6,7-dihydro-5H-cyclopenta[d]pyrimidin-2-yl)pyrrolidine-2-carboxamide;
(R)—N-(5-chlorothiazol-2-yl)-1-(4-(5-isopropyl-1H-pyrazol-3-ylamino)-6,7-dihydro-5H-cyclopenta[d]pyrimidin-2-yl)pyrrolidine-2-carboxamide;
(S)—N-(5-chlorothiazol-2-yl)-1-(4-(5-isopropyl-1H-pyrazol-3-ylamino)-6,7-dihydro-5H-cyclopenta[d]pyrimidin-2-yl)pyrrolidine-2-carboxamide;
N-(5-chlorothiazol-2-yl)-1-(4-(5-cyclopropyl-1H-pyrazol-3-ylamino)-6,7-dihydro-5H-cyclopenta[d]pyrimidin-2-yl)pyrrolidine-2-carboxamide;
(R)—N-(5-chlorothiazol-2-yl)-1-(4-(5-cyclopropyl-1H-pyrazol-3-ylamino)-6,7-dihydro-5H-cyclopenta[d]pyrimidin-2-yl)pyrrolidine-2-carboxamide;
(S)—N-(5-chlorothiazol-2-yl)-1-(4-(5-cyclopropyl-1H-pyrazol-3-ylamino)-6,7-dihydro-5H-cyclopenta[d]pyrimidin-2-yl)pyrrolidine-2-carboxamide;
N-(5-chlorothiazol-2-yl)-1-(4-(5-cyclopentyl-1H-pyrazol-3-ylamino)-6,7-dihydro-5H-cyclopenta[d]pyrimidin-2-yl)pyrrolidine-2-carboxamide;
(R)—N-(5-chlorothiazol-2-yl)-1-(4-(5-cyclopentyl-1H-pyrazol-3-ylamino)-6,7-dihydro-5H-cyclopenta[d]pyrimidin-2-yl)pyrrolidine-2-carboxamide;
(S)—N-(5-chlorothiazol-2-yl)-1-(4-(5-cyclopentyl-1H-pyrazol-3-ylamino)-6,7-dihydro-5H-cyclopenta[d]pyrimidin-2-yl)pyrrolidine-2-carboxamide;
N-(5-chlorothiazol-2-yl)-1-(4-(5-cyclohexyl-1H-pyrazol-3-ylamino)-6,7-dihydro-5H-cyclopenta[d]pyrimidin-2-yl)pyrrolidine-2-carboxamide;
(R)—N-(5-chlorothiazol-2-yl)-1-(4-(5-cyclohexyl-1H-pyrazol-3-ylamino)-6,7-dihydro-5H-cyclopenta[d]pyrimidin-2-yl)pyrrolidine-2-carboxamide;
(S)—N-(5-chlorothiazol-2-yl)-1-(4-(5-cyclohexyl-1H-pyrazol-3-ylamino)-6,7-dihydro-5H-cyclopenta[d]pyrimidin-2-yl)pyrrolidine-2-carboxamide;
1-(4-(5-isopropyl-1H-pyrazol-3-ylamino)-6,7-dihydro-5H-cyclopenta[d]pyrimidin-2-yl)-N-(oxazol-2-yl)pyrrolidine-2-carboxamide;
(R)-1-(4-(5-isopropyl-1H-pyrazol-3-ylamino)-6,7-dihydro-5H-cyclopenta[d]pyrimidin-2-yl)-N-(oxazol-2-yl)pyrrolidine-2-carboxamide;
(S)-1-(4-(5-isopropyl-1H-pyrazol-3-ylamino)-6,7-dihydro-5H-cyclopenta[d]pyrimidin-2-yl)-N-(oxazol-2-yl)pyrrolidine-2-carboxamide;
1-(4-(5-cyclopropyl-1H-pyrazol-3-ylamino)-6,7-dihydro-5H-cyclopenta[d]pyrimidin-2-yl)-N-(oxazol-2-yl)pyrrolidine-2-carboxamide;
(R)-1-(4-(5-cyclopropyl-1H-pyrazol-3-ylamino)-6,7-dihydro-5H-cyclopenta[d]pyrimidin-2-yl)-N-(oxazol-2-yl)pyrrolidine-2-carboxamide;
(S)-1-(4-(5-cyclopropyl-1H-pyrazol-3-ylamino)-6,7-dihydro-5H-cyclopenta[d]pyrimidin-2-yl)-N-(oxazol-2-yl)pyrrolidine-2-carboxamide;
1-(4-(5-cyclopentyl-1H-pyrazol-3-ylamino)-6,7-dihydro-5H-cyclopenta[d]pyrimidin-2-yl)-N-(oxazol-2-yl)pyrrolidine-2-carboxamide;
(R)-1-(4-(5-cyclopentyl-1H-pyrazol-3-ylamino)-6,7-dihydro-5H-cyclopenta[d]pyrimidin-2-yl)-N-(oxazol-2-yl)pyrrolidine-2-carboxamide;
(S)-1-(4-(5-cyclopentyl-1H-pyrazol-3-ylamino)-6,7-dihydro-5H-cyclopenta[d]pyrimidin-2-yl)-N-(oxazol-2-yl)pyrrolidine-2-carboxamide;
1-(4-(5-cyclohexyl-1H-pyrazol-3-ylamino)-6,7-dihydro-5H-cyclopenta[d]pyrimidin-2-yl)-N-(oxazol-2-yl)pyrrolidine-2-carboxamide;
(R)-1-(4-(5-cyclohexyl-1H-pyrazol-3-ylamino)-6,7-dihydro-5H-cyclopenta[d]pyrimidin-2-yl)-N-(oxazol-2-yl)pyrrolidine-2-carboxamide;
(S)-1-(4-(5-cyclohexyl-1H-pyrazol-3-ylamino)-6,7-dihydro-5H-cyclopenta[d]pyrimidin-2-yl)-N-(oxazol-2-yl)pyrrolidine-2-carboxamide;
N-(1H-imidazol-2-yl)-1-(4-(5-isopropyl-1H-pyrazol-3-ylamino)-6,7-dihydro-5H-cyclopenta[d]pyrimidin-2-yl)pyrrolidine-2-carboxamide;
(R)—N-(1H-imidazol-2-yl)-1-(4-(5-isopropyl-1H-pyrazol-3-ylamino)-6,7-dihydro-5H-cyclopenta[d]pyrimidin-2-yl)pyrrolidine-2-carboxamide;
(S)—N-(1H-imidazol-2-yl)-1-(4-(5-isopropyl-1H-pyrazol-3-ylamino)-6,7-dihydro-5H-cyclopenta[d]pyrimidin-2-yl)pyrrolidine-2-carboxamide;
1-(4-(5-cyclopropyl-1H-pyrazol-3-ylamino)-6,7-dihydro-5H-cyclopenta[d]pyrimidin-2-yl)-N-(1H-imidazol-2-yl)pyrrolidine-2-carboxamide;
(R)-1-(4-(5-cyclopropyl-1H-pyrazol-3-ylamino)-6,7-dihydro-5H-cyclopenta[d]pyrimidin-2-yl)-N-(1H-imidazol-2-yl)pyrrolidine-2-carboxamide;
(S)-1-(4-(5-cyclopropyl-1H-pyrazol-3-ylamino)-6,7-dihydro-5H-cyclopenta[d]pyrimidin-2-yl)-N-(1H-imidazol-2-yl)pyrrolidine-2-carboxamide;
1-(4-(5-cyclopentyl-1H-pyrazol-3-ylamino)-6,7-dihydro-5H-cyclopenta[d]pyrimidin-2-yl)-N-(1H-imidazol-2-yl)pyrrolidine-2-carboxamide;
(R)-1-(4-(5-cyclopentyl-1H-pyrazol-3-ylamino)-6,7-dihydro-5H-cyclopenta[d]pyrimidin-2-yl)-N-(1H-imidazol-2-yl)pyrrolidine-2-carboxamide;
(S)-1-(4-(5-cyclopentyl-1H-pyrazol-3-ylamino)-6,7-dihydro-5H-cyclopenta[d]pyrimidin-2-yl)-N-(1H-imidazol-2-yl)pyrrolidine-2-carboxamide;
1-(4-(5-cyclohexyl-1H-pyrazol-3-ylamino)-6,7-dihydro-5H-cyclopenta[d]pyrimidin-2-yl)-N-(1H-imidazol-2-yl)pyrrolidine-2-carboxamide;
(R)-1-(4-(5-cyclohexyl-1H-pyrazol-3-ylamino)-6,7-dihydro-5H-cyclopenta[d]pyrimidin-2-yl)-N-(1H-imidazol-2-yl)pyrrolidine-2-carboxamide;
(S)-1-(4-(5-cyclohexyl-1H-pyrazol-3-ylamino)-6,7-dihydro-5H-cyclopenta[d]pyrimidin-2-yl)-N-(1H-imidazol-2-yl)pyrrolidine-2-carboxamide;
1-(4-(5-isopropyl-1H-pyrazol-3-ylamino)-6,7-dihydro-5H-cyclopenta[d]pyrimidin-2-yl)-N-(1H-1,2,4-triazol-5-yl)pyrrolidine-2-carboxamide;
(R)-1-(4-(5-isopropyl-1H-pyrazol-3-ylamino)-6,7-dihydro-5H-cyclopenta[d]pyrimidin-2-yl)-N-(1H-1,2,4-triazol-5-yl)pyrrolidine-2-carboxamide;
(S)-1-(4-(5-isopropyl-1H-pyrazol-3-ylamino)-6,7-dihydro-5H-cyclopenta[d]pyrimidin-2-yl)-N-(1H-1,2,4-triazol-5-yl)pyrrolidine-2-carboxamide;
1-(4-(5-cyclopropyl-1H-pyrazol-3-ylamino)-6,7-dihydro-5H-cyclopenta[d]pyrimidin-2-yl)-N-(1H-1,2,4-triazol-5-yl)pyrrolidine-2-carboxamide;
(R)-1-(4-(5-cyclopropyl-1H-pyrazol-3-ylamino)-6,7-dihydro-5H-cyclopenta[d]pyrimidin-2-yl)-N-(1H-1,2,4-triazol-5-yl)pyrrolidine-2-carboxamide;
(S)-1-(4-(5-cyclopropyl-1H-pyrazol-3-ylamino)-6,7-dihydro-5H-cyclopenta[d]pyrimidin-2-yl)-N-(1H-1,2,4-triazol-5-yl)pyrrolidine-2-carboxamide;
1-(4-(5-cyclopentyl-1H-pyrazol-3-ylamino)-6,7-dihydro-5H-cyclopenta[d]pyrimidin-2-yl)-N-(1H-1,2,4-triazol-5-yl)pyrrolidine-2-carboxamide;
(R)-1-(4-(5-cyclopentyl-1H-pyrazol-3-ylamino)-6,7-dihydro-5H-cyclopenta[d]pyrimidin-2-yl)-N-(1H-1,2,4-triazol-5-yl)pyrrolidine-2-carboxamide;
(S)-1-(4-(5-cyclopentyl-1H-pyrazol-3-ylamino)-6,7-dihydro-5H-cyclopenta[d]pyrimidin-2-yl)-N-(1H-1,2,4-triazol-5-yl)pyrrolidine-2-carboxamide;
1-(4-(5-cyclohexyl-1H-pyrazol-3-ylamino)-6,7-dihydro-5H-cyclopenta[d]pyrimidin-2-yl)-N-(1H-1,2,4-triazol-5-yl)pyrrolidine-2-carboxamide;
(R)-1-(4-(5-cyclohexyl-1H-pyrazol-3-ylamino)-6,7-dihydro-5H-cyclopenta[d]pyrimidin-2-yl)-N-(1H-1,2,4-triazol-5-yl)pyrrolidine-2-carboxamide;
(S)-1-(4-(5-cyclohexyl-1H-pyrazol-3-ylamino)-6,7-dihydro-5H-cyclopenta[d]pyrimidin-2-yl)-N-(1H-1,2,4-triazol-5-yl)pyrrolidine-2-carboxamide;
1-(4-(5-isopropyl-1H-pyrazol-3-ylamino)-6,7-dihydro-5H-cyclopenta[d]pyrimidin-2-yl)-N-(1H-pyrazol-5-yl)pyrrolidine-2-carboxamide;
(R)-1-(4-(5-isopropyl-1H-pyrazol-3-ylamino)-6,7-dihydro-5H-cyclopenta[d]pyrimidin-2-yl)-N-(1H-pyrazol-5-yl)pyrrolidine-2-carboxamide;
(S)-1-(4-(5-isopropyl-1H-pyrazol-3-ylamino)-6,7-dihydro-5H-cyclopenta[d]pyrimidin-2-yl)-N-(1H-pyrazol-5-yl)pyrrolidine-2-carboxamide;
1-(4-(5-cyclopropyl-1H-pyrazol-3-ylamino)-6,7-dihydro-5H-cyclopenta[d]pyrimidin-2-yl)-N-(1H-pyrazol-5-yl)pyrrolidine-2-carboxamide;
(R)-1-(4-(5-cyclopropyl-1H-pyrazol-3-ylamino)-6,7-dihydro-5H-cyclopenta[d]pyrimidin-2-yl)-N-(1H-pyrazol-5-yl)pyrrolidine-2-carboxamide;
(S)-1-(4-(5-cyclopropyl-1H-pyrazol-3-ylamino)-6,7-dihydro-5H-cyclopenta[d]pyrimidin-2-yl)-N-(1H-pyrazol-5-yl)pyrrolidine-2-carboxamide;
1-(4-(5-cyclopentyl-1H-pyrazol-3-ylamino)-6,7-dihydro-5H-cyclopenta[d]pyrimidin-2-yl)-N-(1H-pyrazol-5-yl)pyrrolidine-2-carboxamide;
(R)-1-(4-(5-cyclopentyl-1H-pyrazol-3-ylamino)-6,7-dihydro-5H-cyclopenta[d]pyrimidin-2-yl)-N-(1H-pyrazol-5-yl)pyrrolidine-2-carboxamide;
(S)-1-(4-(5-cyclopentyl-1H-pyrazol-3-ylamino)-6,7-dihydro-5H-cyclopenta[d]pyrimidin-2-yl)-N-(1H-pyrazol-5-yl)pyrrolidine-2-carboxamide;
1-(4-(5-cyclohexyl-1H-pyrazol-3-ylamino)-6,7-dihydro-5H-cyclopenta[d]pyrimidin-2-yl)-N-(1H-pyrazol-5-yl)pyrrolidine-2-carboxamide;
(R)-1-(4-(5-cyclohexyl-1H-pyrazol-3-ylamino)-6,7-dihydro-5H-cyclopenta[d]pyrimidin-2-yl)-N-(1H-pyrazol-5-yl)pyrrolidine-2-carboxamide;
(S)-1-(4-(5-cyclohexyl-1H-pyrazol-3-ylamino)-6,7-dihydro-5H-cyclopenta[d]pyrimidin-2-yl)-N-(1H-pyrazol-5-yl)pyrrolidine-2-carboxamide;
1-(4-(5-isopropyl-1H-pyrazol-3-ylamino)-6,7-dihydro-5H-cyclopenta[d]pyrimidin-2-yl)-N-(thiazol-2-yl)piperidine-2-carboxamide;
(R)-1-(4-(5-isopropyl-1H-pyrazol-3-ylamino)-6,7-dihydro-5H-cyclopenta[d]pyrimidin-2-yl)-N-(thiazol-2-yl)piperidine-2-carboxamide;
(S)-1-(4-(5-isopropyl-1H-pyrazol-3-ylamino)-6,7-dihydro-5H-cyclopenta[d]pyrimidin-2-yl)-N-(thiazol-2-yl)piperidine-2-carboxamide;
1-(4-(5-cyclopropyl-1H-pyrazol-3-ylamino)-6,7-dihydro-5H-cyclopenta[d]pyrimidin-2-yl)-N-(thiazol-2-yl)piperidine-2-carboxamide;
(R)-1-(4-(5-cyclopropyl-1H-pyrazol-3-ylamino)-6,7-dihydro-5H-cyclopenta[d]pyrimidin-2-yl)-N-(thiazol-2-yl)piperidine-2-carboxamide;
(S)-1-(4-(5-cyclopropyl-1H-pyrazol-3-ylamino)-6,7-dihydro-5H-cyclopenta[d]pyrimidin-2-yl)-N-(thiazol-2-yl)piperidine-2-carboxamide;
1-(4-(5-cyclopentyl-1H-pyrazol-3-ylamino)-6,7-dihydro-5H-cyclopenta[d]pyrimidin-2-yl)-N-(thiazol-2-yl)piperidine-2-carboxamide;
(R)-1-(4-(5-cyclopentyl-1H-pyrazol-3-ylamino)-6,7-dihydro-5H-cyclopenta[d]pyrimidin-2-yl)-N-(thiazol-2-yl)piperidine-2-carboxamide;
(S)-1-(4-(5-cyclopentyl-1H-pyrazol-3-ylamino)-6,7-dihydro-5H-cyclopenta[d]pyrimidin-2-yl)-N-(thiazol-2-yl)piperidine-2-carboxamide;
1-(4-(5-cyclohexyl-1H-pyrazol-3-ylamino)-6,7-dihydro-5H-cyclopenta[d]pyrimidin-2-yl)-N-(thiazol-2-yl)piperidine-2-carboxamide;
(R)-1-(4-(5-cyclohexyl-1H-pyrazol-3-ylamino)-6,7-dihydro-5H-cyclopenta[d]pyrimidin-2-yl)-N-(thiazol-2-yl)piperidine-2-carboxamide;
(S)-1-(4-(5-cyclohexyl-1H-pyrazol-3-ylamino)-6,7-dihydro-5H-cyclopenta[d]pyrimidin-2-yl)-N-(thiazol-2-yl)piperidine-2-carboxamide;
N-(6-fluoropyridin-3-yl)-1-(4-(5-isopropyl-1H-1,2,4-triazol-3-ylamino)-6,7-dihydro-5H-cyclopenta[d]pyrimidin-2-yl)pyrrolidine-2-carboxamide;
(R)—N-(6-fluoropyridin-3-yl)-1-(4-(5-isopropyl-1H-1,2,4-triazol-3-ylamino)-6,7-dihydro-5H-cyclopenta[d]pyrimidin-2-yl)pyrrolidine-2-carboxamide;
(S)—N-(6-fluoropyridin-3-yl)-1-(4-(5-isopropyl-1H-1,2,4-triazol-3-ylamino)-6,7-dihydro-5H-cyclopenta[d]pyrimidin-2-yl)pyrrolidine-2-carboxamide;
1-(4-(5-cyclopropyl-1H-1,2,4-triazol-3-ylamino)-6,7-dihydro-5H-cyclopenta[d]pyrimidin-2-yl)-N-(6-fluoropyridin-3-yl)pyrrolidine-2-carboxamide;
(R)-1-(4-(5-cyclopropyl-1H-1,2,4-triazol-3-ylamino)-6,7-dihydro-5H-cyclopenta[d]pyrimidin-2-yl)-N-(6-fluoropyridin-3-yl)pyrrolidine-2-carboxamide;
(S)-1-(4-(5-cyclopropyl-1H-1,2,4-triazol-3-ylamino)-6,7-dihydro-5H-cyclopenta[d]pyrimidin-2-yl)-N-(6-fluoropyridin-3-yl)pyrrolidine-2-carboxamide;
1-(4-(5-cyclopentyl-1H-1,2,4-triazol-3-ylamino)-6,7-dihydro-5H-cyclopenta[d]pyrimidin-2-yl)-N-(6-fluoropyridin-3-yl)pyrrolidine-2-carboxamide;
(R)-1-(4-(5-cyclopentyl-1H-1,2,4-triazol-3-ylamino)-6,7-dihydro-5H-cyclopenta[d]pyrimidin-2-yl)-N-(6-fluoropyridin-3-yl)pyrrolidine-2-carboxamide;
(S)-1-(4-(5-cyclopentyl-1H-1,2,4-triazol-3-ylamino)-6,7-dihydro-5H-cyclopenta[d]pyrimidin-2-yl)-N-(6-fluoropyridin-3-yl)pyrrolidine-2-carboxamide;
1-(4-(5-cyclohexyl-1H-1,2,4-triazol-3-ylamino)-6,7-dihydro-5H-cyclopenta[d]pyrimidin-2-yl)-N-(6-fluoropyridin-3-yl)pyrrolidine-2-carboxamide;
(R)-1-(4-(5-cyclohexyl-1H-1,2,4-triazol-3-ylamino)-6,7-dihydro-5H-cyclopenta[d]pyrimidin-2-yl)-N-(6-fluoropyridin-3-yl)pyrrolidine-2-carboxamide;
(S)-1-(4-(5-cyclohexyl-1H-1,2,4-triazol-3-ylamino)-6,7-dihydro-5H-cyclopenta[d]pyrimidin-2-yl)-N-(6-fluoropyridin-3-yl)pyrrolidine-2-carboxamide;
N-(6-fluoropyridin-3-yl)-1-(4-(5-isopropyl-1H-imidazol-2-ylamino)-6,7-dihydro-5H-cyclopenta[d]pyrimidin-2-yl)pyrrolidine-2-carboxamide;
(R)—N-(6-fluoropyridin-3-yl)-1-(4-(5-isopropyl-1H-imidazol-2-ylamino)-6,7-dihydro-5H-cyclopenta[d]pyrimidin-2-yl)pyrrolidine-2-carboxamide;
(S)—N-(6-fluoropyridin-3-yl)-1-(4-(5-isopropyl-1H-imidazol-2-ylamino)-6,7-dihydro-5H-cyclopenta[d]pyrimidin-2-yl)pyrrolidine-2-carboxamide;
1-(4-(5-cyclopropyl-1H-imidazol-2-ylamino)-6,7-dihydro-5H-cyclopenta[d]pyrimidin-2-yl)-N-(6-fluoropyridin-3-yl)pyrrolidine-2-carboxamide;
(R)-1-(4-(5-cyclopropyl-1H-imidazol-2-ylamino)-6,7-dihydro-5H-cyclopenta[d]pyrimidin-2-yl)-N-(6-fluoropyridin-3-yl)pyrrolidine-2-carboxamide;
(S)-1-(4-(5-cyclopropyl-1H-imidazol-2-ylamino)-6,7-dihydro-5H-cyclopenta[d]pyrimidin-2-yl)-N-(6-fluoropyridin-3-yl)pyrrolidine-2-carboxamide;
1-(4-(5-cyclopentyl-1H-imidazol-2-ylamino)-6,7-dihydro-5H-cyclopenta[d]pyrimidin-2-yl)-N-(6-fluoropyridin-3-yl)pyrrolidine-2-carboxamide;
(R)-1-(4-(5-cyclopentyl-1H-imidazol-2-ylamino)-6,7-dihydro-5H-cyclopenta[d]pyrimidin-2-yl)-N-(6-fluoropyridin-3-yl)pyrrolidine-2-carboxamide;
(S)-1-(4-(5-cyclopentyl-1H-imidazol-2-ylamino)-6,7-dihydro-5H-cyclopenta[d]pyrimidin-2-yl)-N-(6-fluoropyridin-3-yl)pyrrolidine-2-carboxamide;
1-(4-(5-cyclohexyl-1H-imidazol-2-ylamino)-6,7-dihydro-5H-cyclopenta[d]pyrimidin-2-yl)-N-(6-fluoropyridin-3-yl)pyrrolidine-2-carboxamide;
(R)-1-(4-(5-cyclohexyl-1H-imidazol-2-ylamino)-6,7-dihydro-5H-cyclopenta[d]pyrimidin-2-yl)-N-(6-fluoropyridin-3-yl)pyrrolidine-2-carboxamide;
(S)-1-(4-(5-cyclohexyl-1H-imidazol-2-ylamino)-6,7-dihydro-5H-cyclopenta[d]pyrimidin-2-yl)-N-(6-fluoropyridin-3-yl)pyrrolidine-2-carboxamide;
1-(4-(5-isopropyl-1H-imidazol-2-ylamino)-6,7-dihydro-5H-cyclopenta[d]pyrimidin-2-yl)-N-(thiazol-2-yl)pyrrolidine-2-carboxamide;
(R)-1-(4-(5-isopropyl-1H-imidazol-2-ylamino)-6,7-dihydro-5H-cyclopenta[d]pyrimidin-2-yl)-N-(thiazol-2-yl)pyrrolidine-2-carboxamide;
(S)-1-(4-(5-isopropyl-1H-imidazol-2-ylamino)-6,7-dihydro-5H-cyclopenta[d]pyrimidin-2-yl)-N-(thiazol-2-yl)pyrrolidine-2-carboxamide;
1-(4-(5-cyclopropyl-1H-imidazol-2-ylamino)-6,7-dihydro-5H-cyclopenta[d]pyrimidin-2-yl)-N-(thiazol-2-yl)pyrrolidine-2-carboxamide;
(R)-1-(4-(5-cyclopropyl-1H-imidazol-2-ylamino)-6,7-dihydro-5H-cyclopenta[d]pyrimidin-2-yl)-N-(thiazol-2-yl)pyrrolidine-2-carboxamide;
(S)-1-(4-(5-cyclopropyl-1H-imidazol-2-ylamino)-6,7-dihydro-5H-cyclopenta[d]pyrimidin-2-yl)-N-(thiazol-2-yl)pyrrolidine-2-carboxamide;
1-(4-(5-cyclopentyl-1H-imidazol-2-ylamino)-6,7-dihydro-5H-cyclopenta[d]pyrimidin-2-yl)-N-(thiazol-2-yl)pyrrolidine-2-carboxamide;
(R)-1-(4-(5-cyclopentyl-1H-imidazol-2-ylamino)-6,7-dihydro-5H-cyclopenta[d]pyrimidin-2-yl)-N-(thiazol-2-yl)pyrrolidine-2-carboxamide;
(S)-1-(4-(5-cyclopentyl-1H-imidazol-2-ylamino)-6,7-dihydro-5H-cyclopenta[d]pyrimidin-2-yl)-N-(thiazol-2-yl)pyrrolidine-2-carboxamide;
1-(4-(5-cyclohexyl-1H-imidazol-2-ylamino)-6,7-dihydro-5H-cyclopenta[d]pyrimidin-2-yl)-N-(thiazol-2-yl)pyrrolidine-2-carboxamide;
(R)-1-(4-(5-cyclohexyl-1H-imidazol-2-ylamino)-6,7-dihydro-5H-cyclopenta[d]pyrimidin-2-yl)-N-(thiazol-2-yl)pyrrolidine-2-carboxamide;
(S)-1-(4-(5-cyclohexyl-1H-imidazol-2-ylamino)-6,7-dihydro-5H-cyclopenta[d]pyrimidin-2-yl)-N-(thiazol-2-yl)pyrrolidine-2-carboxamide;
(1-(4-(5-isobutyl-1H-imidazol-2-ylamino)-6,7-dihydro-5H-cyclopenta[d]pyrimidin-2-yl)pyrrolidin-2-yl)(morpholino)methanone;
(R)-(1-(4-(5-isobutyl-1H-imidazol-2-ylamino)-6,7-dihydro-5H-cyclopenta[d]pyrimidin-2-yl)pyrrolidin-2-yl)(morpholino)methanone;
(S)-(1-(4-(5-isobutyl-1H-imidazol-2-ylamino)-6,7-dihydro-5H-cyclopenta[d]pyrimidin-2-yl)pyrrolidin-2-yl)(morpholino)methanone;
(1-(4-(5-cyclopropyl-1H-imidazol-2-ylamino)-6,7-dihydro-5H-cyclopenta[d]pyrimidin-2-yl)pyrrolidin-2-yl)(morpholino)methanone;
(R)-(1-(4-(5-cyclopropyl-1H-imidazol-2-ylamino)-6,7-dihydro-5H-cyclopenta[d]pyrimidin-2-yl)pyrrolidin-2-yl)(morpholino)methanone;
(S)-(1-(4-(5-cyclopropyl-1H-imidazol-2-ylamino)-6,7-dihydro-5H-cyclopenta[d]pyrimidin-2-yl)pyrrolidin-2-yl)(morpholino)methanone;
(1-(4-(5-cyclohexyl-1H-imidazol-2-ylamino)-6,7-dihydro-5H-cyclopenta[d]pyrimidin-2-yl)pyrrolidin-2-yl)(morpholino)methanone;
(R)-(1-(4-(5-cyclohexyl-1H-imidazol-2-ylamino)-6,7-dihydro-5H-cyclopenta[d]pyrimidin-2-yl)pyrrolidin-2-yl)(morpholino)methanone;
(S)-(1-(4-(5-cyclohexyl-1H-imidazol-2-ylamino)-6,7-dihydro-5H-cyclopenta[d]pyrimidin-2-yl)pyrrolidin-2-yl)(morpholino)methanone;
(1-(4-(5-isopropyl-1H-imidazol-2-ylamino)-6,7-dihydro-5H-cyclopenta[d]pyrimidin-2-yl)pyrrolidin-2-yl)(4-methylpiperazin-1-yl)methanone;
(R)-(1-(4-(5-isopropyl-1H-imidazol-2-ylamino)-6,7-dihydro-5H-cyclopenta[d]pyrimidin-2-yl)pyrrolidin-2-yl)(4-methylpiperazin-1-yl)methanone;
(S)-(1-(4-(5-isopropyl-1H-imidazol-2-ylamino)-6,7-dihydro-5H-cyclopenta[d]pyrimidin-2-yl)pyrrolidin-2-yl)(4-methylpiperazin-1-yl)methanone;
(1-(4-(5-cyclopropyl-1H-imidazol-2-ylamino)-6,7-dihydro-5H-cyclopenta[d]pyrimidin-2-yl)pyrrolidin-2-yl)(4-methylpiperazin-1-yl)methanone;
(R)-(1-(4-(5-cyclopropyl-1H-imidazol-2-ylamino)-6,7-dihydro-5H-cyclopenta[d]pyrimidin-2-yl)pyrrolidin-2-yl)(4-methylpiperazin-1-yl)methanone;
(S)-(1-(4-(5-cyclopropyl-1H-imidazol-2-ylamino)-6,7-dihydro-5H-cyclopenta[d]pyrimidin-2-yl)pyrrolidin-2-yl)(4-methylpiperazin-1-yl)methanone;
(1-(4-(5-cyclopentyl-1H-imidazol-2-ylamino)-6,7-dihydro-5H-cyclopenta[d]pyrimidin-2-yl)pyrrolidin-2-yl)(4-methylpiperazin-1-yl)methanone;
(R)-(1-(4-(5-cyclopentyl-1H-imidazol-2-ylamino)-6,7-dihydro-5H-cyclopenta[d]pyrimidin-2-yl)pyrrolidin-2-yl)(4-methylpiperazin-1-yl)methanone;
(S)-(1-(4-(5-cyclopentyl-1H-imidazol-2-ylamino)-6,7-dihydro-5H-cyclopenta[d]pyrimidin-2-yl)pyrrolidin-2-yl)(4-methylpiperazin-1-yl)methanone;
(1-(4-(5-cyclohexyl-1H-imidazol-2-ylamino)-6,7-dihydro-5H-cyclopenta[d]pyrimidin-2-yl)pyrrolidin-2-yl)(4-methylpiperazin-1-yl)methanone;
(R)-(1-(4-(5-cyclohexyl-1H-imidazol-2-ylamino)-6,7-dihydro-5H-cyclopenta[d]pyrimidin-2-yl)pyrrolidin-2-yl)(4-methylpiperazin-1-yl)methanone;
(S)-(1-(4-(5-cyclohexyl-1H-imidazol-2-ylamino)-6,7-dihydro-5H-cyclopenta[d]pyrimidin-2-yl)pyrrolidin-2-yl)(4-methylpiperazin-1-yl)methanone;
4-(1-{4-[(4-isopropyl-3H-imidazol-2-yl)-amino]-5H,6H,7H-cyclopenta[d]-pyrimidin-2-yl}pyrrolidine-2-carbonyl)-1λ⁶-thiomorpholine-1,1-dione;
(R)-4-(1-{4-[(4-isopropyl-3H-imidazol-2-yl)-amino]-5H,6H,7H-cyclopenta[d]-pyrimidin-2-yl}pyrrolidine-2-carbonyl)-1λ⁶-thiomorpholine-1,1-dione;
(S)-4-(1-{4-[(4-isopropyl-3H-imidazol-2-yl)-amino]-5H,6H,7H-cyclopenta[d]-pyrimidin-2-yl}pyrrolidine-2-carbonyl)-1λ⁶-thiomorpholine-1,1-dione;
4-(1-{4-[(4-cyclopropyl-3H-imidazol-2-yl)amino]-5H,6H,7H-cyclopenta[d]-pyrimidin-2-yl}pyrrolidine-2-carbonyl)-1λ⁶-thiomorpholine-1,1-dione;
(R)-4-(1-{4-[(4-cyclopropyl-3H-imidazol-2-yl)amino]-5H,6H,7H-cyclopenta[d]-pyrimidin-2-yl}pyrrolidine-2-carbonyl)-1λ⁶-thiomorpholine-1,1-dione;
(S)-4-(1-{4-[(4-cyclopropyl-3H-imidazol-2-yl)amino]-5H,6H,7H-cyclopenta[d]-pyrimidin-2-yl}pyrrolidine-2-carbonyl)-1λ⁶-thiomorpholine-1,1-dione;
4-[1-(4-{[4-(2-methylpropyl)-3H-imidazol-2-yl]amino}-5H,6H,7H-cyclopenta[d]-pyrimidin-2-yl)pyrrolidine-2-carbonyl]-1λ⁶-thiomorpholine-1,1-dione;
(R)-4-[1-(4-{[4-(2-methylpropyl)-3H-imidazol-2-yl]amino}-5H,6H,7H-cyclopenta[d]-pyrimidin-2-yl)pyrrolidine-2-carbonyl]-1λ⁶-thiomorpholine-1,1-dione;
(S)-4-[1-(4-{[4-(2-methylpropyl)-3H-imidazol-2-yl]amino}-5H,6H,7H-cyclopenta[d]-pyrimidin-2-yl)pyrrolidine-2-carbonyl]-1λ⁶-thiomorpholine-1,1-dione;
4-(1-{4-[(4-cyclohexyl-3H-imidazol-2-yl)amino]-5H,6H,7H-cyclopenta[d]-pyrimidin-2-yl}pyrrolidine-2-carbonyl)-1λ⁶-thiomorpholine-1,1-dione;
(R)-4-(1-{4-[(4-cyclohexyl-3H-imidazol-2-yl)amino]-5H,6H,7H-cyclopenta[d]-pyrimidin-2-yl}pyrrolidine-2-carbonyl)-1λ⁶-thiomorpholine-1,1-dione;
(S)-4-(1-{4-[(4-cyclohexyl-3H-imidazol-2-yl)amino]-5H,6H,7H-cyclopenta[d]-pyrimidin-2-yl}pyrrolidine-2-carbonyl)-1λ⁶-thiomorpholine-1,1-dione;
(4,4-dimethylpiperidin-1-yl)(1-(4-(5-isopropyl-1H-imidazol-2-ylamino)-6,7-dihydro-5H-cyclopenta[d]pyrimidin-2-yl)pyrrolidin-2-yl)methanone;
(R)-(4,4-dimethylpiperidin-1-yl)(1-(4-(5-isopropyl-1H-imidazol-2-ylamino)-6,7-dihydro-5H-cyclopenta[d]pyrimidin-2-yl)pyrrolidin-2-yl)methanone;
(S)-(4,4-dimethylpiperidin-1-yl)(1-(4-(5-isopropyl-1H-imidazol-2-ylamino)-6,7-dihydro-5H-cyclopenta[d]pyrimidin-2-yl)pyrrolidin-2-yl)methanone;
(1-(4-(5-cyclopropyl-1H-imidazol-2-ylamino)-6,7-dihydro-5H-cyclopenta[d]pyrimidin-2-yl)pyrrolidin-2-yl)(4,4-dimethylpiperidin-1-yl)methanone;
(R)-(1-(4-(5-cyclopropyl-1H-imidazol-2-ylamino)-6,7-dihydro-5H-cyclopenta[d]pyrimidin-2-yl)pyrrolidin-2-yl)(4,4-dimethylpiperidin-1-yl)methanone;
(S)-(1-(4-(5-cyclopropyl-1H-imidazol-2-ylamino)-6,7-dihydro-5H-cyclopenta[d]pyrimidin-2-yl)pyrrolidin-2-yl)(4,4-dimethylpiperidin-1-yl)methanone;
(1-(4-(5-cyclopentyl-1H-imidazol-2-ylamino)-6,7-dihydro-5H-cyclopenta[d]pyrimidin-2-yl)pyrrolidin-2-yl)(4,4-dimethylpiperidin-1-yl)methanone;
(R)-(1-(4-(5-cyclopentyl-1H-imidazol-2-ylamino)-6,7-dihydro-5H-cyclopenta[d]pyrimidin-2-yl)pyrrolidin-2-yl)(4,4-dimethylpiperidin-1-yl)methanone;
(S)-(1-(4-(5-cyclopentyl-1H-imidazol-2-ylamino)-6,7-dihydro-5H-cyclopenta[d]pyrimidin-2-yl)pyrrolidin-2-yl)(4,4-dimethylpiperidin-1-yl)methanone;
(1-(4-(5-cyclohexyl-1H-imidazol-2-ylamino)-6,7-dihydro-5H-cyclopenta[d]pyrimidin-2-yl)pyrrolidin-2-yl)(4,4-dimethylpiperidin-1-yl)methanone;
(R)-(1-(4-(5-cyclohexyl-1H-imidazol-2-ylamino)-6,7-dihydro-5H-cyclopenta[d]pyrimidin-2-yl)pyrrolidin-2-yl)(4,4-dimethylpiperidin-1-yl)methanone;
(S)-(1-(4-(5-cyclohexyl-1H-imidazol-2-ylamino)-6,7-dihydro-5H-cyclopenta[d]pyrimidin-2-yl)pyrrolidin-2-yl)(4,4-dimethylpiperidin-1-yl)methanone;
(4,4-difluoropiperidin-1-yl)(1-(4-(5-isopropyl-1H-imidazol-2-ylamino)-6,7-dihydro-5H-cyclopenta[d]pyrimidin-2-yl)pyrrolidin-2-yl)methanone;
(R)-(4,4-difluoropiperidin-1-yl)(1-(4-(5-isopropyl-1H-imidazol-2-ylamino)-6,7-dihydro-5H-cyclopenta[d]pyrimidin-2-yl)pyrrolidin-2-yl)methanone;
(S)-(4,4-difluoropiperidin-1-yl)(1-(4-(5-isopropyl-1H-imidazol-2-ylamino)-6,7-dihydro-5H-cyclopenta[d]pyrimidin-2-yl)pyrrolidin-2-yl)methanone;
(1-(4-(5-cyclopropyl-1H-imidazol-2-ylamino)-6,7-dihydro-5H-cyclopenta[d]pyrimidin-2-yl)pyrrolidin-2-yl)(4,4-difluoropiperidin-1-yl)methanone;
(R)-(1-(4-(5-cyclopropyl-1H-imidazol-2-ylamino)-6,7-dihydro-5H-cyclopenta[d]pyrimidin-2-yl)pyrrolidin-2-yl)(4,4-difluoropiperidin-1-yl)methanone;
(S)-(1-(4-(5-cyclopropyl-1H-imidazol-2-ylamino)-6,7-dihydro-5H-cyclopenta[d]pyrimidin-2-yl)pyrrolidin-2-yl)(4,4-difluoropiperidin-1-yl)methanone;
(1-(4-(5-cyclopentyl-1H-imidazol-2-ylamino)-6,7-dihydro-5H-cyclopenta[d]pyrimidin-2-yl)pyrrolidin-2-yl)(4,4-difluoropiperidin-1-yl)methanone;
(R)-(1-(4-(5-cyclopentyl-1H-imidazol-2-ylamino)-6,7-dihydro-5H-cyclopenta[d]pyrimidin-2-yl)pyrrolidin-2-yl)(4,4-difluoropiperidin-1-yl)methanone;
(S)-(1-(4-(5-cyclopentyl-1H-imidazol-2-ylamino)-6,7-dihydro-5H-cyclopenta[d]pyrimidin-2-yl)pyrrolidin-2-yl)(4,4-difluoropiperidin-1-yl)methanone;
(1-(4-(5-cyclohexyl-1H-imidazol-2-ylamino)-6,7-dihydro-5H-cyclopenta[d]pyrimidin-2-yl)pyrrolidin-2-yl)(4,4-difluoropiperidin-1-yl)methanone;
(R)-(1-(4-(5-cyclohexyl-1H-imidazol-2-ylamino)-6,7-dihydro-5H-cyclopenta[d]pyrimidin-2-yl)pyrrolidin-2-yl)(4,4-difluoropiperidin-1-yl)methanone;
(S)-(1-(4-(5-cyclohexyl-1H-imidazol-2-ylamino)-6,7-dihydro-5H-cyclopenta[d]pyrimidin-2-yl)pyrrolidin-2-yl)(4,4-difluoropiperidin-1-yl)methanone;
4-(1-(4-(5-isopropyl-1H-imidazol-2-ylamino)-6,7-dihydro-5H-cyclopenta[d]pyrimidin-2-yl)pyrrolidine-2-carbonyl)-1-methylpiperazin-2-one;
(R)-4-(1-(4-(5-isopropyl-1H-imidazol-2-ylamino)-6,7-dihydro-5H-cyclopenta[d]pyrimidin-2-yl)pyrrolidine-2-carbonyl)-1-methylpiperazin-2-one;
(S)-4-(1-(4-(5-isopropyl-1H-imidazol-2-ylamino)-6,7-dihydro-5H-cyclopenta[d]pyrimidin-2-yl)pyrrolidine-2-carbonyl)-1-methylpiperazin-2-one;
4-(1-(4-(5-cyclopropyl-1H-imidazol-2-ylamino)-6,7-dihydro-5H-cyclopenta[d]pyrimidin-2-yl)pyrrolidine-2-carbonyl)-1-methylpiperazin-2-one;
(R)-4-(1-(4-(5-cyclopropyl-1H-imidazol-2-ylamino)-6,7-dihydro-5H-cyclopenta[d]pyrimidin-2-yl)pyrrolidine-2-carbonyl)-1-methylpiperazin-2-one;
(S)-4-(1-(4-(5-cyclopropyl-1H-imidazol-2-ylamino)-6,7-dihydro-5H-cyclopenta[d]pyrimidin-2-yl)pyrrolidine-2-carbonyl)-1-methylpiperazin-2-one;
4-(1-(4-(5-cyclopentyl-1H-imidazol-2-ylamino)-6,7-dihydro-5H-cyclopenta[d]pyrimidin-2-yl)pyrrolidine-2-carbonyl)-1-methylpiperazin-2-one;
(R)-4-(1-(4-(5-cyclopentyl-1H-imidazol-2-ylamino)-6,7-dihydro-5H-cyclopenta[d]pyrimidin-2-yl)pyrrolidine-2-carbonyl)-1-methylpiperazin-2-one;
(S)-4-(1-(4-(5-cyclopentyl-1H-imidazol-2-ylamino)-6,7-dihydro-5H-cyclopenta[d]pyrimidin-2-yl)pyrrolidine-2-carbonyl)-1-methylpiperazin-2-one;
4-(1-(4-(5-cyclohexyl-1H-imidazol-2-ylamino)-6,7-dihydro-5H-cyclopenta[d]pyrimidin-2-yl)pyrrolidine-2-carbonyl)-1-methylpiperazin-2-one;
(R)-4-(1-(4-(5-cyclohexyl-1H-imidazol-2-ylamino)-6,7-dihydro-5H-cyclopenta[d]pyrimidin-2-yl)pyrrolidine-2-carbonyl)-1-methylpiperazin-2-one;
(S)-4-(1-(4-(5-cyclohexyl-1H-imidazol-2-ylamino)-6,7-dihydro-5H-cyclopenta[d]pyrimidin-2-yl)pyrrolidine-2-carbonyl)-1-methylpiperazin-2-one;
1-(4-(5-isopropyl-1H-pyrazol-3-ylamino)-6,7-dihydro-5H-cyclopenta[d]pyrimidin-2-yl)-N-methyl-N-(thiazol-2-yl)pyrrolidine-2-carboxamide;
(R)-1-(4-(5-isopropyl-1H-pyrazol-3-ylamino)-6,7-dihydro-5H-cyclopenta[d]pyrimidin-2-yl)-N-methyl-N-(thiazol-2-yl)pyrrolidine-2-carboxamide;
(S)-1-(4-(5-isopropyl-1H-pyrazol-3-ylamino)-6,7-dihydro-5H-cyclopenta[d]pyrimidin-2-yl)-N-methyl-N-(thiazol-2-yl)pyrrolidine-2-carboxamide;
1-(4-(5-cyclopropyl-1H-pyrazol-3-ylamino)-6,7-dihydro-5H-cyclopenta[d]pyrimidin-2-yl)-N-methyl-N-(thiazol-2-yl)pyrrolidine-2-carboxamide;
(R)-1-(4-(5-cyclopropyl-1H-pyrazol-3-ylamino)-6,7-dihydro-5H-cyclopenta[d]pyrimidin-2-yl)-N-methyl-N-(thiazol-2-yl)pyrrolidine-2-carboxamide;
(S)-1-(4-(5-cyclopropyl-1H-pyrazol-3-ylamino)-6,7-dihydro-5H-cyclopenta[d]pyrimidin-2-yl)-N-methyl-N-(thiazol-2-yl)pyrrolidine-2-carboxamide;
1-(4-(5-cyclopentyl-1H-pyrazol-3-ylamino)-6,7-dihydro-5H-cyclopenta[d]pyrimidin-2-yl)-N-methyl-N-(thiazol-2-yl)pyrrolidine-2-carboxamide;
(R)-1-(4-(5-cyclopentyl-1H-pyrazol-3-ylamino)-6,7-dihydro-5H-cyclopenta[d]pyrimidin-2-yl)-N-methyl-N-(thiazol-2-yl)pyrrolidine-2-carboxamide;
(S)-1-(4-(5-cyclopentyl-1H-pyrazol-3-ylamino)-6,7-dihydro-5H-cyclopenta[d]pyrimidin-2-yl)-N-methyl-N-(thiazol-2-yl)pyrrolidine-2-carboxamide;
1-(4-(5-cyclohexyl-1H-pyrazol-3-ylamino)-6,7-dihydro-5H-cyclopenta[d]pyrimidin-2-yl)-N-methyl-N-(thiazol-2-yl)pyrrolidine-2-carboxamide;
(R)-1-(4-(5-cyclohexyl-1H-pyrazol-3-ylamino)-6,7-dihydro-5H-cyclopenta[d]pyrimidin-2-yl)-N-methyl-N-(thiazol-2-yl)pyrrolidine-2-carboxamide;
(S)-1-(4-(5-cyclohexyl-1H-pyrazol-3-ylamino)-6,7-dihydro-5H-cyclopenta[d]pyrimidin-2-yl)-N-methyl-N-(thiazol-2-yl)pyrrolidine-2-carboxamide;
(1-(4-(5-(bicyclo[3.1.0]hexan-3-yl)-1H-pyrazol-3-ylamino)-6,7-dihydro-5H-cyclopenta[d]pyrimidin-2-yl)pyrrolidin-2-yl)(piperidin-1-yl)methanone;
(R)-(1-(4-(5-(bicyclo[3.1.0]hexan-3-yl)-1H-pyrazol-3-ylamino)-6,7-dihydro-5H-cyclopenta[d]pyrimidin-2-yl)pyrrolidin-2-yl)(piperidin-1-yl)methanone;
(S)-(1-(4-(5-(bicyclo[3.1.0]hexan-3-yl)-1H-pyrazol-3-ylamino)-6,7-dihydro-5H-cyclopenta[d]pyrimidin-2-yl)pyrrolidin-2-yl)(piperidin-1-yl)methanone;
(1-(4-(5-(bicyclo[3.1.0]hexan-3-yl)-1H-pyrazol-3-ylamino)-6,7-dihydro-5H-cyclopenta[d]pyrimidin-2-yl)pyrrolidin-2-yl)(pyrrolidin-1-yl)methanone;
(R)-(1-(4-(5-(bicyclo[3.1.0]hexan-3-yl)-1H-pyrazol-3-ylamino)-6,7-dihydro-5H-cyclopenta[d]pyrimidin-2-yl)pyrrolidin-2-yl)(pyrrolidin-1-yl)methanone;
(S)-(1-(4-(5-(bicyclo[3.1.0]hexan-3-yl)-1H-pyrazol-3-ylamino)-6,7-dihydro-5H-cyclopenta[d]pyrimidin-2-yl)pyrrolidin-2-yl)(pyrrolidin-1-yl)methanone;
1-(4-(5-(bicyclo[3.1.0]hexan-3-yl)-1H-pyrazol-3-ylamino)-6,7-dihydro-5H-cyclopenta[d]pyrimidin-2-yl)-N,N-diethylpyrrolidine-2-carboxamide;
(R)-1-(4-(5-(bicyclo[3.1.0]hexan-3-yl)-1H-pyrazol-3-ylamino)-6,7-dihydro-5H-cyclopenta[d]pyrimidin-2-yl)-N,N-diethylpyrrolidine-2-carboxamide;
(S)-1-(4-(5-(bicyclo[3.1.0]hexan-3-yl)-1H-pyrazol-3-ylamino)-6,7-dihydro-5H-cyclopenta[d]pyrimidin-2-yl)-N,N-diethylpyrrolidine-2-carboxamide;
1-(4-(5-(bicyclo[3.1.0]hexan-3-yl)-1H-pyrazol-3-ylamino)-6,7-dihydro-5H-cyclopenta[d]pyrimidin-2-yl)-N,N-dimethylpyrrolidine-2-carboxamide;
(R)-1-(4-(5-(bicyclo[3.1.0]hexan-3-yl)-1H-pyrazol-3-ylamino)-6,7-dihydro-5H-cyclopenta[d]pyrimidin-2-yl)-N,N-dimethylpyrrolidine-2-carboxamide;
(S)-1-(4-(5-(bicyclo[3.1.0]hexan-3-yl)-1H-pyrazol-3-ylamino)-6,7-dihydro-5H-cyclopenta[d]pyrimidin-2-yl)-N,N-dimethylpyrrolidine-2-carboxamide;
(1-(4-(5-(3,3-dimethylcyclopentyl)-1H-pyrazol-3-ylamino)-6,7-dihydro-5H-cyclopenta[d]pyrimidin-2-yl)pyrrolidin-2-yl)(piperidin-1-yl)methanone;
(R,R)-(1-(4-(5-(3,3-dimethylcyclopentyl)-1H-pyrazol-3-ylamino)-6,7-dihydro-5H-cyclopenta[d]pyrimidin-2-yl)pyrrolidin-2-yl)(piperidin-1-yl)methanone;
(R,S)-(1-(4-(5-(3,3-dimethylcyclopentyl)-1H-pyrazol-3-ylamino)-6,7-dihydro-5H-cyclopenta[d]pyrimidin-2-yl)pyrrolidin-2-yl)(piperidin-1-yl)methanone;
(S,R)-(1-(4-(5-(3,3-dimethylcyclopentyl)-1H-pyrazol-3-ylamino)-6,7-dihydro-5H-cyclopenta[d]pyrimidin-2-yl)pyrrolidin-2-yl)(piperidin-1-yl)methanone;
(S,S)-(1-(4-(5-(3,3-dimethylcyclopentyl)-1H-pyrazol-3-ylamino)-6,7-dihydro-5H-cyclopenta[d]pyrimidin-2-yl)pyrrolidin-2-yl)(piperidin-1-yl)methanone;
(1-(4-(5-(3,3-dimethylcyclopentyl)-1H-pyrazol-3-ylamino)-6,7-dihydro-5H-cyclopenta[d]pyrimidin-2-yl)pyrrolidin-2-yl)(pyrrolidin-1-yl)methanone;
(R,R)-(1-(4-(5-(3,3-dimethylcyclopentyl)-1H-pyrazol-3-ylamino)-6,7-dihydro-5H-cyclopenta[d]pyrimidin-2-yl)pyrrolidin-2-yl)(pyrrolidin-1-yl)methanone;
(R,S)-(1-(4-(5-(3,3-dimethylcyclopentyl)-1H-pyrazol-3-ylamino)-6,7-dihydro-5H-cyclopenta[d]pyrimidin-2-yl)pyrrolidin-2-yl)(pyrrolidin-1-yl)methanone;
(S,R)-(1-(4-(5-(3,3-dimethylcyclopentyl)-1H-pyrazol-3-ylamino)-6,7-dihydro-5H-cyclopenta[d]pyrimidin-2-yl)pyrrolidin-2-yl)(pyrrolidin-1-yl)methanone;
(S,S)-(1-(4-(5-(3,3-dimethylcyclopentyl)-1H-pyrazol-3-ylamino)-6,7-dihydro-5H-cyclopenta[d]pyrimidin-2-yl)pyrrolidin-2-yl)(pyrrolidin-1-yl)methanone;
1-(4-(5-(3,3-dimethylcyclopentyl)-1H-pyrazol-3-ylamino)-6,7-dihydro-5H-cyclopenta[d]pyrimidin-2-yl)-N,N-diethylpyrrolidine-2-carboxamide;
(R,R)-1-(4-(5-(3,3-dimethylcyclopentyl)-1H-pyrazol-3-ylamino)-6,7-dihydro-5H-cyclopenta[d]pyrimidin-2-yl)-N,N-diethylpyrrolidine-2-carboxamide;
(R,S)-1-(4-(5-(3,3-dimethylcyclopentyl)-1H-pyrazol-3-ylamino)-6,7-dihydro-5H-cyclopenta[d]pyrimidin-2-yl)-N,N-diethylpyrrolidine-2-carboxamide;
(S,R)-1-(4-(5-(3,3-dimethylcyclopentyl)-1H-pyrazol-3-ylamino)-6,7-dihydro-5H-cyclopenta[d]pyrimidin-2-yl)-N,N-diethylpyrrolidine-2-carboxamide;
(S,S)-1-(4-(5-(3,3-dimethylcyclopentyl)-1H-pyrazol-3-ylamino)-6,7-dihydro-5H-cyclopenta[d]pyrimidin-2-yl)-N,N-diethylpyrrolidine-2-carboxamide;
1-(4-(5-(3,3-dimethylcyclopentyl)-1H-pyrazol-3-ylamino)-6,7-dihydro-5H-cyclopenta[d]pyrimidin-2-yl)-N,N-dimethylpyrrolidine-2-carboxamide;
(R,R)-1-(4-(5-(3,3-dimethylcyclopentyl)-1H-pyrazol-3-ylamino)-6,7-dihydro-5H-cyclopenta[d]pyrimidin-2-yl)-N,N-dimethylpyrrolidine-2-carboxamide;
(R,S)-1-(4-(5-(3,3-dimethylcyclopentyl)-1H-pyrazol-3-ylamino)-6,7-dihydro-5H-cyclopenta[d]pyrimidin-2-yl)-N,N-dimethylpyrrolidine-2-carboxamide;
(S,R)-1-(4-(5-(3,3-dimethylcyclopentyl)-1H-pyrazol-3-ylamino)-6,7-dihydro-5H-cyclopenta[d]pyrimidin-2-yl)-N,N-dimethylpyrrolidine-2-carboxamide;
(S,S)-1-(4-(5-(3,3-dimethylcyclopentyl)-1H-pyrazol-3-ylamino)-6,7-dihydro-5H-cyclopenta[d]pyrimidin-2-yl)-N,N-dimethylpyrrolidine-2-carboxamide;
(1-(4-(5-(3,3-difluorocyclopentyl)-1H-pyrazol-3-ylamino)-6,7-dihydro-5H-cyclopenta[d]pyrimidin-2-yl)pyrrolidin-2-yl)(piperidin-1-yl)methanone;
(R,R)-(1-(4-(5-(3,3-difluorocyclopentyl)-1H-pyrazol-3-ylamino)-6,7-dihydro-5H-cyclopenta[d]pyrimidin-2-yl)pyrrolidin-2-yl)(piperidin-1-yl)methanone;
(R,S)-(1-(4-(5-(3,3-difluorocyclopentyl)-1H-pyrazol-3-ylamino)-6,7-dihydro-5H-cyclopenta[d]pyrimidin-2-yl)pyrrolidin-2-yl)(piperidin-1-yl)methanone;
(S,R)-(1-(4-(5-(3,3-difluorocyclopentyl)-1H-pyrazol-3-ylamino)-6,7-dihydro-5H-cyclopenta[d]pyrimidin-2-yl)pyrrolidin-2-yl)(piperidin-1-yl)methanone;
(S,S)-(1-(4-(5-(3,3-difluorocyclopentyl)-1H-pyrazol-3-ylamino)-6,7-dihydro-5H-cyclopenta[d]pyrimidin-2-yl)pyrrolidin-2-yl)(piperidin-1-yl)methanone;
(1-(4-(5-(3,3-difluorocyclopentyl)-1H-pyrazol-3-ylamino)-6,7-dihydro-5H-cyclopenta[d]pyrimidin-2-yl)pyrrolidin-2-yl)(pyrrolidin-1-yl)methanone;
(R,R)-(1-(4-(5-(3,3-difluorocyclopentyl)-1H-pyrazol-3-ylamino)-6,7-dihydro-5H-cyclopenta[d]pyrimidin-2-yl)pyrrolidin-2-yl)(pyrrolidin-1-yl)methanone;
(R,S)-(1-(4-(5-(3,3-difluorocyclopentyl)-1H-pyrazol-3-ylamino)-6,7-dihydro-5H-cyclopenta[d]pyrimidin-2-yl)pyrrolidin-2-yl)(pyrrolidin-1-yl)methanone;
(S,R)-(1-(4-(5-(3,3-difluorocyclopentyl)-1H-pyrazol-3-ylamino)-6,7-dihydro-5H-cyclopenta[d]pyrimidin-2-yl)pyrrolidin-2-yl)(pyrrolidin-1-yl)methanone;
(S,S)-(1-(4-(5-(3,3-difluorocyclopentyl)-1H-pyrazol-3-ylamino)-6,7-dihydro-5H-cyclopenta[d]pyrimidin-2-yl)pyrrolidin-2-yl)(pyrrolidin-1-yl)methanone;
1-(4-(5-(3,3-difluorocyclopentyl)-1H-pyrazol-3-ylamino)-6,7-dihydro-5H-cyclopenta[d]pyrimidin-2-yl)-N,N-diethylpyrrolidine-2-carboxamide;
(R,R)-1-(4-(5-(3,3-difluorocyclopentyl)-1H-pyrazol-3-ylamino)-6,7-dihydro-5H-cyclopenta[d]pyrimidin-2-yl)-N,N-diethylpyrrolidine-2-carboxamide;
(R,S)-1-(4-(5-(3,3-difluorocyclopentyl)-1H-pyrazol-3-ylamino)-6,7-dihydro-5H-cyclopenta[d]pyrimidin-2-yl)-N,N-diethylpyrrolidine-2-carboxamide;
(S,R)-1-(4-(5-(3,3-difluorocyclopentyl)-1H-pyrazol-3-ylamino)-6,7-dihydro-5H-cyclopenta[d]pyrimidin-2-yl)-N,N-diethylpyrrolidine-2-carboxamide;
(S,S)-1-(4-(5-(3,3-difluorocyclopentyl)-1H-pyrazol-3-ylamino)-6,7-dihydro-5H-cyclopenta[d]pyrimidin-2-yl)-N,N-diethylpyrrolidine-2-carboxamide;
1-(4-(5-(3,3-difluorocyclopentyl)-1H-pyrazol-3-ylamino)-6,7-dihydro-5H-cyclopenta[d]pyrimidin-2-yl)-N,N-dimethylpyrrolidine-2-carboxamide;
(R,R)-1-(4-(5-(3,3-difluorocyclopentyl)-1H-pyrazol-3-ylamino)-6,7-dihydro-5H-cyclopenta[d]pyrimidin-2-yl)-N,N-dimethylpyrrolidine-2-carboxamide;
(R,S)-1-(4-(5-(3,3-difluorocyclopentyl)-1H-pyrazol-3-ylamino)-6,7-dihydro-5H-cyclopenta[d]pyrimidin-2-yl)-N,N-dimethylpyrrolidine-2-carboxamide;
(S,R)-1-(4-(5-(3,3-difluorocyclopentyl)-1H-pyrazol-3-ylamino)-6,7-dihydro-5H-cyclopenta[d]pyrimidin-2-yl)-N,N-dimethylpyrrolidine-2-carboxamide;
(S,S)-1-(4-(5-(3,3-difluorocyclopentyl)-1H-pyrazol-3-ylamino)-6,7-dihydro-5H-cyclopenta[d]pyrimidin-2-yl)-N,N-dimethylpyrrolidine-2-carboxamide;
(1-(4-(5-tert-butyl-1H-pyrazol-3-ylamino)-6,7-dihydro-5H-cyclopenta[d]pyrimidin-2-yl)pyrrolidin-2-yl)(piperidin-1-yl)methanone;
(R)-(1-(4-(5-tert-butyl-1H-pyrazol-3-ylamino)-6,7-dihydro-5H-cyclopenta[d]pyrimidin-2-yl)pyrrolidin-2-yl)(piperidin-1-yl)methanone;
(S)-(1-(4-(5-tert-butyl-1H-pyrazol-3-ylamino)-6,7-dihydro-5H-cyclopenta[d]pyrimidin-2-yl)pyrrolidin-2-yl)(piperidin-1-yl)methanone;
(1-(4-(5-tert-butyl-1H-pyrazol-3-ylamino)-6,7-dihydro-5H-cyclopenta[d]pyrimidin-2-yl)pyrrolidin-2-yl)(pyrrolidin-1-yl)methanone;
(R)-(1-(4-(5-tert-butyl-1H-pyrazol-3-ylamino)-6,7-dihydro-5H-cyclopenta[d]pyrimidin-2-yl)pyrrolidin-2-yl)(pyrrolidin-1-yl)methanone;
(S)-(1-(4-(5-tert-butyl-1H-pyrazol-3-ylamino)-6,7-dihydro-5H-cyclopenta[d]pyrimidin-2-yl)pyrrolidin-2-yl)(pyrrolidin-1-yl)methanone;
1-(4-(5-tert-butyl-1H-pyrazol-3-ylamino)-6,7-dihydro-5H-cyclopenta[d]pyrimidin-2-yl)-N,N-diethylpyrrolidine-2-carboxamide;
(R)-1-(4-(5-tert-butyl-1H-pyrazol-3-ylamino)-6,7-dihydro-5H-cyclopenta[d]pyrimidin-2-yl)-N,N-diethylpyrrolidine-2-carboxamide;
(S)-1-(4-(5-tert-butyl-1H-pyrazol-3-ylamino)-6,7-dihydro-5H-cyclopenta[d]pyrimidin-2-yl)-N,N-diethylpyrrolidine-2-carboxamide;
1-(4-(5-tert-butyl-1H-pyrazol-3-ylamino)-6,7-dihydro-5H-cyclopenta[d]pyrimidin-2-yl)-N,N-dimethylpyrrolidine-2-carboxamide;
(R)-1-(4-(5-tert-butyl-1H-pyrazol-3-ylamino)-6,7-dihydro-5H-cyclopenta[d]pyrimidin-2-yl)-N,N-dimethylpyrrolidine-2-carboxamide;
(S)-1-(4-(5-tert-butyl-1H-pyrazol-3-ylamino)-6,7-dihydro-5H-cyclopenta[d]pyrimidin-2-yl)-N,N-dimethylpyrrolidine-2-carboxamide;
piperidin-1-yl(1-(4-(5-(tetrahydro-2H-pyran-4-yl)-1H-pyrazol-3-ylamino)-6,7-dihydro-5H-cyclopenta[d]pyrimidin-2-yl)pyrrolidin-2-yl)methanone;
(R)-piperidin-1-yl(1-(4-(5-(tetrahydro-2H-pyran-4-yl)-1H-pyrazol-3-ylamino)-6,7-dihydro-5H-cyclopenta[d]pyrimidin-2-yl)pyrrolidin-2-yl)methanone;
(S)-piperidin-1-yl(1-(4-(5-(tetrahydro-2H-pyran-4-yl)-1H-pyrazol-3-ylamino)-6,7-dihydro-5H-cyclopenta[d]pyrimidin-2-yl)pyrrolidin-2-yl)methanone;
pyrrolidin-1-yl(1-(4-(5-(tetrahydro-2H-pyran-4-yl)-1H-pyrazol-3-ylamino)-6,7-dihydro-5H-cyclopenta[d]pyrimidin-2-yl)pyrrolidin-2-yl)methanone;
(R)-pyrrolidin-1-yl(1-(4-(5-(tetrahydro-2H-pyran-4-yl)-1H-pyrazol-3-ylamino)-6,7-dihydro-5H-cyclopenta[d]pyrimidin-2-yl)pyrrolidin-2-yl)methanone;
(S)-pyrrolidin-1-yl(1-(4-(5-(tetrahydro-2H-pyran-4-yl)-1H-pyrazol-3-ylamino)-6,7-dihydro-5H-cyclopenta[d]pyrimidin-2-yl)pyrrolidin-2-yl)methanone;
N,N-diethyl-1-(4-(5-(tetrahydro-2H-pyran-4-yl)-1H-pyrazol-3-ylamino)-6,7-dihydro-5H-cyclopenta[d]pyrimidin-2-yl)pyrrolidine-2-carboxamide;
(R)—N,N-diethyl-1-(4-(5-(tetrahydro-2H-pyran-4-yl)-1H-pyrazol-3-ylamino)-6,7-dihydro-5H-cyclopenta[d]pyrimidin-2-yl)pyrrolidine-2-carboxamide;
(S)—N,N-diethyl-1-(4-(5-(tetrahydro-2H-pyran-4-yl)-1H-pyrazol-3-ylamino)-6,7-dihydro-5H-cyclopenta[d]pyrimidin-2-yl)pyrrolidine-2-carboxamide;
N,N-dimethyl-1-(4-(5-(tetrahydro-2H-pyran-4-yl)-1H-pyrazol-3-ylamino)-6,7-dihydro-5H-cyclopenta[d]pyrimidin-2-yl)pyrrolidine-2-carboxamide;
(R)—N,N-dimethyl-1-(4-(5-(tetrahydro-2H-pyran-4-yl)-1H-pyrazol-3-ylamino)-6,7-dihydro-5H-cyclopenta[d]pyrimidin-2-yl)pyrrolidine-2-carboxamide;
(S)—N,N-dimethyl-1-(4-(5-(tetrahydro-2H-pyran-4-yl)-1H-pyrazol-3-ylamino)-6,7-dihydro-5H-cyclopenta[d]pyrimidin-2-yl)pyrrolidine-2-carboxamide;
1-(4-(5-cyclopentyl-1H-pyrazol-3-ylamino)-5,5-difluoro-6,7-dihydro-5H-cyclopenta[d]pyrimidin-2-yl)-N-(thiazol-2-yl)pyrrolidine-2-carboxamide;
(R)-1-(4-(5-cyclopentyl-1H-pyrazol-3-ylamino)-5,5-difluoro-6,7-dihydro-5H-cyclopenta[d]pyrimidin-2-yl)-N-(thiazol-2-yl)pyrrolidine-2-carboxamide;
(S)-1-(4-(5-cyclopentyl-1H-pyrazol-3-ylamino)-5,5-difluoro-6,7-dihydro-5H-cyclopenta[d]pyrimidin-2-yl)-N-(thiazol-2-yl)pyrrolidine-2-carboxamide;
1-(4-(5-cyclopentyl-1H-pyrazol-3-ylamino)-6,6-difluoro-6,7-dihydro-5H-cyclopenta[d]pyrimidin-2-yl)-N-(thiazol-2-yl)pyrrolidine-2-carboxamide;
(R)-1-(4-(5-cyclopentyl-1H-pyrazol-3-ylamino)-6,6-difluoro-6,7-dihydro-5H-cyclopenta[d]pyrimidin-2-yl)-N-(thiazol-2-yl)pyrrolidine-2-carboxamide;
(S)-1-(4-(5-cyclopentyl-1H-pyrazol-3-ylamino)-6,6-difluoro-6,7-dihydro-5H-cyclopenta[d]pyrimidin-2-yl)-N-(thiazol-2-yl)pyrrolidine-2-carboxamide;
1-(4-(5-cyclopentyl-1H-pyrazol-3-ylamino)-7,7-difluoro-6,7-dihydro-5H-cyclopenta[d]pyrimidin-2-yl)-N-(thiazol-2-yl)pyrrolidine-2-carboxamide;
(R)-1-(4-(5-cyclopentyl-1H-pyrazol-3-ylamino)-7,7-difluoro-6,7-dihydro-5H-cyclopenta[d]pyrimidin-2-yl)-N-(thiazol-2-yl)pyrrolidine-2-carboxamide;
(S)-1-(4-(5-cyclopentyl-1H-pyrazol-3-ylamino)-7,7-difluoro-6,7-dihydro-5H-cyclopenta[d]pyrimidin-2-yl)-N-(thiazol-2-yl)pyrrolidine-2-carboxamide;
1-(4-(5-cyclopentyl-1H-pyrazol-3-ylamino)-5-hydroxy-6,7-dihydro-5H-cyclopenta[d]pyrimidin-2-yl)-N-(thiazol-2-yl)pyrrolidine-2-carboxamide;
(R,R)-1-(4-(5-cyclopentyl-1H-pyrazol-3-ylamino)-5-hydroxy-6,7-dihydro-5H-cyclopenta[d]pyrimidin-2-yl)-N-(thiazol-2-yl)pyrrolidine-2-carboxamide;
(R,S)-1-(4-(5-cyclopentyl-1H-pyrazol-3-ylamino)-5-hydroxy-6,7-dihydro-5H-cyclopenta[d]pyrimidin-2-yl)-N-(thiazol-2-yl)pyrrolidine-2-carboxamide;
(S,R)-1-(4-(5-cyclopentyl-1H-pyrazol-3-ylamino)-5-hydroxy-6,7-dihydro-5H-cyclopenta[d]pyrimidin-2-yl)-N-(thiazol-2-yl)pyrrolidine-2-carboxamide;
(S,S)-1-(4-(5-cyclopentyl-1H-pyrazol-3-ylamino)-5-hydroxy-6,7-dihydro-5H-cyclopenta[d]pyrimidin-2-yl)-N-(thiazol-2-yl)pyrrolidine-2-carboxamide;
1-(4-(5-cyclopentyl-1H-pyrazol-3-ylamino)-6-hydroxy-6,7-dihydro-5H-cyclopenta[d]pyrimidin-2-yl)-N-(thiazol-2-yl)pyrrolidine-2-carboxamide;
(R,R)-1-(4-(5-cyclopentyl-1H-pyrazol-3-ylamino)-6-hydroxy-6,7-dihydro-5H-cyclopenta[d]pyrimidin-2-yl)-N-(thiazol-2-yl)pyrrolidine-2-carboxamide;
(R,S)-1-(4-(5-cyclopentyl-1H-pyrazol-3-ylamino)-6-hydroxy-6,7-dihydro-5H-cyclopenta[d]pyrimidin-2-yl)-N-(thiazol-2-yl)pyrrolidine-2-carboxamide;
(S,R)-1-(4-(5-cyclopentyl-1H-pyrazol-3-ylamino)-6-hydroxy-6,7-dihydro-5H-cyclopenta[d]pyrimidin-2-yl)-N-(thiazol-2-yl)pyrrolidine-2-carboxamide;
(S,S)-1-(4-(5-cyclopentyl-1H-pyrazol-3-ylamino)-6-hydroxy-6,7-dihydro-5H-cyclopenta[d]pyrimidin-2-yl)-N-(thiazol-2-yl)pyrrolidine-2-carboxamide;
1-(4-(5-cyclopentyl-1H-pyrazol-3-ylamino)-7-hydroxy-6,7-dihydro-5H-cyclopenta[d]pyrimidin-2-yl)-N-(thiazol-2-yl)pyrrolidine-2-carboxamide;
(R,R)-1-(4-(5-cyclopentyl-1H-pyrazol-3-ylamino)-7-hydroxy-6,7-dihydro-5H-cyclopenta[d]pyrimidin-2-yl)-N-(thiazol-2-yl)pyrrolidine-2-carboxamide;
(R,S)-1-(4-(5-cyclopentyl-1H-pyrazol-3-ylamino)-7-hydroxy-6,7-dihydro-5H-cyclopenta[d]pyrimidin-2-yl)-N-(thiazol-2-yl)pyrrolidine-2-carboxamide;
(S,R)-1-(4-(5-cyclopentyl-1H-pyrazol-3-ylamino)-7-hydroxy-6,7-dihydro-5H-cyclopenta[d]pyrimidin-2-yl)-N-(thiazol-2-yl)pyrrolidine-2-carboxamide;
(S,S)-1-(4-(5-cyclopentyl-1H-pyrazol-3-ylamino)-7-hydroxy-6,7-dihydro-5H-cyclopenta[d]pyrimidin-2-yl)-N-(thiazol-2-yl)pyrrolidine-2-carboxamide;
1-(4-(5-cyclopentyl-1H-pyrazol-3-ylamino)-5-methyl-6,7-dihydro-5H-cyclopenta[d]pyrimidin-2-yl)-N-(thiazol-2-yl)pyrrolidine-2-carboxamide;
(R,R)-1-(4-(5-cyclopentyl-1H-pyrazol-3-ylamino)-5-methyl-6,7-dihydro-5H-cyclopenta[d]pyrimidin-2-yl)-N-(thiazol-2-yl)pyrrolidine-2-carboxamide;
(R,S)-1-(4-(5-cyclopentyl-1H-pyrazol-3-ylamino)-5-methyl-6,7-dihydro-5H-cyclopenta[d]pyrimidin-2-yl)-N-(thiazol-2-yl)pyrrolidine-2-carboxamide;
(S,R)-1-(4-(5-cyclopentyl-1H-pyrazol-3-ylamino)-5-methyl-6,7-dihydro-5H-cyclopenta[d]pyrimidin-2-yl)-N-(thiazol-2-yl)pyrrolidine-2-carboxamide;
(S,S)-1-(4-(5-cyclopentyl-1H-pyrazol-3-ylamino)-5-methyl-6,7-dihydro-5H-cyclopenta[d]pyrimidin-2-yl)-N-(thiazol-2-yl)pyrrolidine-2-carboxamide;
1-(4-(5-cyclopentyl-1H-pyrazol-3-ylamino)-6-methyl-6,7-dihydro-5H-cyclopenta[d]pyrimidin-2-yl)-N-(thiazol-2-yl)pyrrolidine-2-carboxamide;
(R,R)-1-(4-(5-cyclopentyl-1H-pyrazol-3-ylamino)-6-methyl-6,7-dihydro-5H-cyclopenta[d]pyrimidin-2-yl)-N-(thiazol-2-yl)pyrrolidine-2-carboxamide;
(R,S)-1-(4-(5-cyclopentyl-1H-pyrazol-3-ylamino)-6-methyl-6,7-dihydro-5H-cyclopenta[d]pyrimidin-2-yl)-N-(thiazol-2-yl)pyrrolidine-2-carboxamide;
(S,R)-1-(4-(5-cyclopentyl-1H-pyrazol-3-ylamino)-6-methyl-6,7-dihydro-5H-cyclopenta[d]pyrimidin-2-yl)-N-(thiazol-2-yl)pyrrolidine-2-carboxamide;
(S,S)-1-(4-(5-cyclopentyl-1H-pyrazol-3-ylamino)-6-methyl-6,7-dihydro-5H-cyclopenta[d]pyrimidin-2-yl)-N-(thiazol-2-yl)pyrrolidine-2-carboxamide;
1-(4-(5-cyclopentyl-1H-pyrazol-3-ylamino)-7-methyl-6,7-dihydro-5H-cyclopenta[d]pyrimidin-2-yl)-N-(thiazol-2-yl)pyrrolidine-2-carboxamide;
(R,R)-1-(4-(5-cyclopentyl-1H-pyrazol-3-ylamino)-7-methyl-6,7-dihydro-5H-cyclopenta[d]pyrimidin-2-yl)-N-(thiazol-2-yl)pyrrolidine-2-carboxamide;
(R,S)-1-(4-(5-cyclopentyl-1H-pyrazol-3-ylamino)-7-methyl-6,7-dihydro-5H-cyclopenta[d]pyrimidin-2-yl)-N-(thiazol-2-yl)pyrrolidine-2-carboxamide;
(S,R)-1-(4-(5-cyclopentyl-1H-pyrazol-3-ylamino)-7-methyl-6,7-dihydro-5H-cyclopenta[d]pyrimidin-2-yl)-N-(thiazol-2-yl)pyrrolidine-2-carboxamide;
(S,S)-1-(4-(5-cyclopentyl-1H-pyrazol-3-ylamino)-7-methyl-6,7-dihydro-5H-cyclopenta[d]pyrimidin-2-yl)-N-(thiazol-2-yl)pyrrolidine-2-carboxamide;
1-(4-(5-cyclopentyl-1H-pyrazol-3-ylamino)-6,7-dihydro-5H-cyclopenta[d]pyrimidin-2-yl)-4-hydroxy-N-(thiazol-2-yl)pyrrolidine-2-carboxamide;
(R,R)-1-(4-(5-cyclopentyl-1H-pyrazol-3-ylamino)-6,7-dihydro-5H-cyclopenta[d]pyrimidin-2-yl)-4-hydroxy-N-(thiazol-2-yl)pyrrolidine-2-carboxamide;
(R,S)-1-(4-(5-cyclopentyl-1H-pyrazol-3-ylamino)-6,7-dihydro-5H-cyclopenta[d]pyrimidin-2-yl)-4-hydroxy-N-(thiazol-2-yl)pyrrolidine-2-carboxamide;
(S,R)-1-(4-(5-cyclopentyl-1H-pyrazol-3-ylamino)-6,7-dihydro-5H-cyclopenta[d]pyrimidin-2-yl)-4-hydroxy-N-(thiazol-2-yl)pyrrolidine-2-carboxamide;
(S,S)-1-(4-(5-cyclopentyl-1H-pyrazol-3-ylamino)-6,7-dihydro-5H-cyclopenta[d]pyrimidin-2-yl)-4-hydroxy-N-(thiazol-2-yl)pyrrolidine-2-carboxamide;
1-(4-(5-cyclopentyl-1H-pyrazol-3-ylamino)-6,7-dihydro-5H-cyclopenta[d]pyrimidin-2-yl)-4-methoxy-N-(thiazol-2-yl)pyrrolidine-2-carboxamide;
(R,R)-1-(4-(5-cyclopentyl-1H-pyrazol-3-ylamino)-6,7-dihydro-5H-cyclopenta[d]pyrimidin-2-yl)-4-methoxy-N-(thiazol-2-yl)pyrrolidine-2-carboxamide;
(R,S)-1-(4-(5-cyclopentyl-1H-pyrazol-3-ylamino)-6,7-dihydro-5H-cyclopenta[d]pyrimidin-2-yl)-4-methoxy-N-(thiazol-2-yl)pyrrolidine-2-carboxamide;
(S,R)-1-(4-(5-cyclopentyl-1H-pyrazol-3-ylamino)-6,7-dihydro-5H-cyclopenta[d]pyrimidin-2-yl)-4-methoxy-N-(thiazol-2-yl)pyrrolidine-2-carboxamide;
(S,S)-1-(4-(5-cyclopentyl-1H-pyrazol-3-ylamino)-6,7-dihydro-5H-cyclopenta[d]pyrimidin-2-yl)-4-methoxy-N-(thiazol-2-yl)pyrrolidine-2-carboxamide;
1-(4-(5-cyclopentyl-1H-pyrazol-3-ylamino)-6,7-dihydro-5H-cyclopenta[d]pyrimidin-2-yl)-4,4-difluoro-N-(thiazol-2-yl)pyrrolidine-2-carboxamide;
(R)-1-(4-(5-cyclopentyl-1H-pyrazol-3-ylamino)-6,7-dihydro-5H-cyclopenta[d]pyrimidin-2-yl)-4,4-difluoro-N-(thiazol-2-yl)pyrrolidine-2-carboxamide;
(S)-1-(4-(5-cyclopentyl-1H-pyrazol-3-ylamino)-6,7-dihydro-5H-cyclopenta[d]pyrimidin-2-yl)-4,4-difluoro-N-(thiazol-2-yl)pyrrolidine-2-carboxamide;
1-(4-(5-cyclopentyl-1H-pyrazol-3-ylamino)-6,7-dihydro-5H-cyclopenta[d]pyrimidin-2-yl)-4-oxo-N-(thiazol-2-yl)pyrrolidine-2-carboxamide;
(R)-1-(4-(5-cyclopentyl-1H-pyrazol-3-ylamino)-6,7-dihydro-5H-cyclopenta[d]pyrimidin-2-yl)-4-oxo-N-(thiazol-2-yl)pyrrolidine-2-carboxamide;
(S)-1-(4-(5-cyclopentyl-1H-pyrazol-3-ylamino)-6,7-dihydro-5H-cyclopenta[d]pyrimidin-2-yl)-4-oxo-N-(thiazol-2-yl)pyrrolidine-2-carboxamide;
pyrrolidin-1-yl(1-(4-(5-(3,3,4,4-tetrafluorocyclopentyl)-1H-pyrazol-3-ylamino)-6,7-dihydro-5H-cyclopenta[d]pyrimidin-2-yl)pyrrolidin-2-yl)methanone;
(R)-pyrrolidin-1-yl(1-(4-(5-(3,3,4,4-tetrafluorocyclopentyl)-1H-pyrazol-3-ylamino)-6,7-dihydro-5H-cyclopenta[d]pyrimidin-2-yl)pyrrolidin-2-yl)methanone;
(S)-pyrrolidin-1-yl(1-(4-(5-(3,3,4,4-tetrafluorocyclopentyl)-1H-pyrazol-3-ylamino)-6,7-dihydro-5H-cyclopenta[d]pyrimidin-2-yl)pyrrolidin-2-yl)methanone;
(1-(4-(5-(pentan-3-yl)-1H-pyrazol-3-ylamino)-6,7-dihydro-5H-cyclopenta[d]pyrimidin-2-yl)pyrrolidin-2-yl)(pyrrolidin-1-yl)methanone;
(R)-(1-(4-(5-(pentan-3-yl)-1H-pyrazol-3-ylamino)-6,7-dihydro-5H-cyclopenta[d]pyrimidin-2-yl)pyrrolidin-2-yl)(pyrrolidin-1-yl)methanone;
(S)-(1-(4-(5-(pentan-3-yl)-1H-pyrazol-3-ylamino)-6,7-dihydro-5H-cyclopenta[d]pyrimidin-2-yl)pyrrolidin-2-yl)(pyrrolidin-1-yl)methanone;
(1-(4-(5-cyclobutyl-1H-pyrazol-3-ylamino)-6,7-dihydro-5H-cyclopenta[d]pyrimidin-2-yl)pyrrolidin-2-yl)(pyrrolidin-1-yl)methanone;
(R)-(1-(4-(5-cyclobutyl-1H-pyrazol-3-ylamino)-6,7-dihydro-5H-cyclopenta[d]pyrimidin-2-yl)pyrrolidin-2-yl)(pyrrolidin-1-yl)methanone;
(S)-(1-(4-(5-cyclobutyl-1H-pyrazol-3-ylamino)-6,7-dihydro-5H-cyclopenta[d]pyrimidin-2-yl)pyrrolidin-2-yl)(pyrrolidin-1-yl)methanone;
(1-(4-(5-(1-methylcyclobutyl)-1H-pyrazol-3-ylamino)-6,7-dihydro-5H-cyclopenta[d]pyrimidin-2-yl)pyrrolidin-2-yl)(pyrrolidin-1-yl)methanone;
(R)-(1-(4-(5-(1-methylcyclobutyl)-1H-pyrazol-3-ylamino)-6,7-dihydro-5H-cyclopenta[d]pyrimidin-2-yl)pyrrolidin-2-yl)(pyrrolidin-1-yl)methanone;
(S)-(1-(4-(5-(1-methylcyclobutyl)-1H-pyrazol-3-ylamino)-6,7-dihydro-5H-cyclopenta[d]pyrimidin-2-yl)pyrrolidin-2-yl)(pyrrolidin-1-yl)methanone;
(1-(4-(5-cyclopentyl-1H-pyrazol-3-ylamino)-6,7-dihydro-5H-cyclopenta[d]pyrimidin-2-yl)pyrrolidin-2-yl)(3,3-difluoropyrrolidin-1-yl)methanone;
(R)-(1-(4-(5-cyclopentyl-1H-pyrazol-3-ylamino)-6,7-dihydro-5H-cyclopenta[d]pyrimidin-2-yl)pyrrolidin-2-yl)(3,3-difluoropyrrolidin-1-yl)methanone;
(S)-(1-(4-(5-cyclopentyl-1H-pyrazol-3-ylamino)-6,7-dihydro-5H-cyclopenta[d]pyrimidin-2-yl)pyrrolidin-2-yl)(3,3-difluoropyrrolidin-1-yl)methanone;
(1-(4-(5-cyclopentyl-1H-pyrazol-3-ylamino)-6,7-dihydro-5H-cyclopenta[d]pyrimidin-2-yl)pyrrolidin-2-yl)(3,3,4,4-tetrafluoropyrrolidin-1-yl)methanone;
(R)-(1-(4-(5-cyclopentyl-1H-pyrazol-3-ylamino)-6,7-dihydro-5H-cyclopenta[d]pyrimidin-2-yl)pyrrolidin-2-yl)(3,3,4,4-tetrafluoropyrrolidin-1-yl)methanone;
(S)-(1-(4-(5-cyclopentyl-1H-pyrazol-3-ylamino)-6,7-dihydro-5H-cyclopenta[d]pyrimidin-2-yl)pyrrolidin-2-yl)(3,3,4,4-tetrafluoropyrrolidin-1-yl)methanone;
(3,3-difluoropyrrolidin-1-yl)(1-(4-(5-isopropyl-1H-pyrazol-3-ylamino)-6,7-dihydro-5H-cyclopenta[d]pyrimidin-2-yl)pyrrolidin-2-yl)methanone;
(R)-(3,3-difluoropyrrolidin-1-yl)(1-(4-(5-isopropyl-1H-pyrazol-3-ylamino)-6,7-dihydro-5H-cyclopenta[d]pyrimidin-2-yl)pyrrolidin-2-yl)methanone;
(S)-(3,3-difluoropyrrolidin-1-yl)(1-(4-(5-isopropyl-1H-pyrazol-3-ylamino)-6,7-dihydro-5H-cyclopenta[d]pyrimidin-2-yl)pyrrolidin-2-yl)methanone;
(1-(4-(5-isopropyl-1H-pyrazol-3-ylamino)-6,7-dihydro-5H-cyclopenta[d]pyrimidin-2-yl)pyrrolidin-2-yl)(3,3,4,4-tetrafluoropyrrolidin-1-yl)methanone;
(R)-(1-(4-(5-isopropyl-1H-pyrazol-3-ylamino)-6,7-dihydro-5H-cyclopenta[d]pyrimidin-2-yl)pyrrolidin-2-yl)(3,3,4,4-tetrafluoropyrrolidin-1-yl)methanone;
(S)-(1-(4-(5-isopropyl-1H-pyrazol-3-ylamino)-6,7-dihydro-5H-cyclopenta[d]pyrimidin-2-yl)pyrrolidin-2-yl)(3,3,4,4-tetrafluoropyrrolidin-1-yl)methanone;
N,N-dimethyl-1-(4-(5-(trifluoromethyl)-1H-pyrazol-3-ylamino)-6,7-dihydro-5H-cyclopenta[d]pyrimidin-2-yl)pyrrolidine-2-carboxamide;
(R)—N,N-dimethyl-1-(4-(5-(trifluoromethyl)-1H-pyrazol-3-ylamino)-6,7-dihydro-5H-cyclopenta[d]pyrimidin-2-yl)pyrrolidine-2-carboxamide;
(S)—N,N-dimethyl-1-(4-(5-(trifluoromethyl)-1H-pyrazol-3-ylamino)-6,7-dihydro-5H-cyclopenta[d]pyrimidin-2-yl)pyrrolidine-2-carboxamide;
pyrrolidin-1-yl(1-(4-(5-(trifluoromethyl)-1H-pyrazol-3-ylamino)-6,7-dihydro-5H-cyclopenta[d]pyrimidin-2-yl)pyrrolidin-2-yl)methanone;
(R)-pyrrolidin-1-yl(1-(4-(5-(trifluoromethyl)-1H-pyrazol-3-ylamino)-6,7-dihydro-5H-cyclopenta[d]pyrimidin-2-yl)pyrrolidin-2-yl)methanone;
(S)-pyrrolidin-1-yl(1-(4-(5-(trifluoromethyl)-1H-pyrazol-3-ylamino)-6,7-dihydro-5H-cyclopenta[d]pyrimidin-2-yl)pyrrolidin-2-yl)methanone;
(1-(4-(5-(1-methylcyclopropyl)-1H-pyrazol-3-ylamino)-6,7-dihydro-5H-cyclopenta[d]pyrimidin-2-yl)pyrrolidin-2-yl)(pyrrolidin-1-yl)methanone;
(R)-(1-(4-(5-(1-methylcyclopropyl)-1H-pyrazol-3-ylamino)-6,7-dihydro-5H-cyclopenta[d]pyrimidin-2-yl)pyrrolidin-2-yl)(pyrrolidin-1-yl)methanone;
(S)-(1-(4-(5-(1-methylcyclopropyl)-1H-pyrazol-3-ylamino)-6,7-dihydro-5H-cyclopenta[d]pyrimidin-2-yl)pyrrolidin-2-yl)(pyrrolidin-1-yl)methanone;
(1-(4-(5-(cyclopropylmethyl)-1H-pyrazol-3-ylamino)-6,7-dihydro-5H-cyclopenta[d]pyrimidin-2-yl)pyrrolidin-2-yl)(pyrrolidin-1-yl)methanone;
(R)-(1-(4-(5-(cyclopropylmethyl)-1H-pyrazol-3-ylamino)-6,7-dihydro-5H-cyclopenta[d]pyrimidin-2-yl)pyrrolidin-2-yl)(pyrrolidin-1-yl)methanone;
(S)-(1-(4-(5-(cyclopropylmethyl)-1H-pyrazol-3-ylamino)-6,7-dihydro-5H-cyclopenta[d]pyrimidin-2-yl)pyrrolidin-2-yl)(pyrrolidin-1-yl)methanone;
(1-(4-(5-cyclopentyl-1H-pyrazol-3-ylamino)-6,7-dihydro-5H-cyclopenta[d]pyrimidin-2-yl)pyrrolidin-2-yl)(5-azaspiro[2.4]heptan-5-yl)methanone;
(R)-(1-(4-(5-cyclopentyl-1H-pyrazol-3-ylamino)-6,7-dihydro-5H-cyclopenta[d]pyrimidin-2-yl)pyrrolidin-2-yl)(5-azaspiro[2.4]heptan-5-yl)methanone;
(S)-(1-(4-(5-cyclopentyl-1H-pyrazol-3-ylamino)-6,7-dihydro-5H-cyclopenta[d]pyrimidin-2-yl)pyrrolidin-2-yl)(5-azaspiro[2.4]heptan-5-yl)methanone;
(1-(4-(5-cyclopentyl-1H-pyrazol-3-ylamino)-6,7-dihydro-5H-cyclopenta[d]pyrimidin-2-yl)pyrrolidin-2-yl)(6-azaspiro[2.5]octan-6-yl)methanone;
(R)-(1-(4-(5-cyclopentyl-1H-pyrazol-3-ylamino)-6,7-dihydro-5H-cyclopenta[d]pyrimidin-2-yl)pyrrolidin-2-yl)(6-azaspiro[2.5]octan-6-yl)methanone;
(S)-(1-(4-(5-cyclopentyl-1H-pyrazol-3-ylamino)-6,7-dihydro-5H-cyclopenta[d]pyrimidin-2-yl)pyrrolidin-2-yl)(6-azaspiro[2.5]octan-6-yl)methanone;
(1-(4-(5-cyclopentyl-1H-pyrazol-3-ylamino)-6,7-dihydro-5H-cyclopenta[d]pyrimidin-2-yl)pyrrolidin-2-yl)(3-methoxypyrrolidin-1-yl)methanone;
(R)-(1-(4-(5-cyclopentyl-1H-pyrazol-3-ylamino)-6,7-dihydro-5H-cyclopenta[d]pyrimidin-2-yl)pyrrolidin-2-yl)(3-methoxypyrrolidin-1-yl)methanone;
(S)-(1-(4-(5-cyclopentyl-1H-pyrazol-3-ylamino)-6,7-dihydro-5H-cyclopenta[d]pyrimidin-2-yl)pyrrolidin-2-yl)(3-methoxypyrrolidin-1-yl)methanone;
(1-(4-(5-cyclopentyl-1H-pyrazol-3-ylamino)-6,7-dihydro-5H-cyclopenta[d]pyrimidin-2-yl)pyrrolidin-2-yl)(3-hydroxypyrrolidin-1-yl)methanone;
(R)-(1-(4-(5-cyclopentyl-1H-pyrazol-3-ylamino)-6,7-dihydro-5H-cyclopenta[d]pyrimidin-2-yl)pyrrolidin-2-yl)(3-hydroxypyrrolidin-1-yl)methanone;
(S)-(1-(4-(5-cyclopentyl-1H-pyrazol-3-ylamino)-6,7-dihydro-5H-cyclopenta[d]pyrimidin-2-yl)pyrrolidin-2-yl)(3-hydroxypyrrolidin-1-yl)methanone;
1-(4-((5-isopropyl-1H-pyrazol-3-yl)amino)-6,7-dihydro-5H-cyclopenta[d]pyrimidin-2-yl)-N-(1,3,4-thiadiazol-2-yl)pyrrolidine-2-carboxamide;
(R)-1-(4-((5-isopropyl-1H-pyrazol-3-yl)amino)-6,7-dihydro-5H-cyclopenta[d]pyrimidin-2-yl)-N-(1,3,4-thiadiazol-2-yl)pyrrolidine-2-carboxamide;
(S)-1-(4-((5-isopropyl-1H-pyrazol-3-yl)amino)-6,7-dihydro-5H-cyclopenta[d]pyrimidin-2-yl)-N-(1,3,4-thiadiazol-2-yl)pyrrolidine-2-carboxamide;
1-(4-((5-cyclopropyl-1H-pyrazol-3-yl)amino)-6,7-dihydro-5H-cyclopenta[d]pyrimidin-2-yl)-N-(1,3,4-thiadiazol-2-yl)pyrrolidine-2-carboxamide;
(R)-1-(4-((5-cyclopropyl-1H-pyrazol-3-yl)amino)-6,7-dihydro-5H-cyclopenta[d]pyrimidin-2-yl)-N-(1,3,4-thiadiazol-2-yl)pyrrolidine-2-carboxamide;
(S)-1-(4-((5-cyclopropyl-1H-pyrazol-3-yl)amino)-6,7-dihydro-5H-cyclopenta[d]pyrimidin-2-yl)-N-(1,3,4-thiadiazol-2-yl)pyrrolidine-2-carboxamide;
1-(4-((5-cyclopentyl-1H-pyrazol-3-yl)amino)-6,7-dihydro-5H-cyclopenta[d]pyrimidin-2-yl)-N-(1,3,4-thiadiazol-2-yl)pyrrolidine-2-carboxamide;
(R)-1-(4-((5-cyclopentyl-1H-pyrazol-3-yl)amino)-6,7-dihydro-5H-cyclopenta[d]pyrimidin-2-yl)-N-(1,3,4-thiadiazol-2-yl)pyrrolidine-2-carboxamide;
(S)-1-(4-((5-cyclopentyl-1H-pyrazol-3-yl)amino)-6,7-dihydro-5H-cyclopenta[d]pyrimidin-2-yl)-N-(1,3,4-thiadiazol-2-yl)pyrrolidine-2-carboxamide;
1-(4-((5-cyclohexyl-1H-pyrazol-3-yl)amino)-6,7-dihydro-5H-cyclopenta[d]pyrimidin-2-yl)-N-(1,3,4-thiadiazol-2-yl)pyrrolidine-2-carboxamide;
(R)-1-(4-((5-cyclohexyl-1H-pyrazol-3-yl)amino)-6,7-dihydro-5H-cyclopenta[d]pyrimidin-2-yl)-N-(1,3,4-thiadiazol-2-yl)pyrrolidine-2-carboxamide;
(S)-1-(4-((5-cyclohexyl-1H-pyrazol-3-yl)amino)-6,7-dihydro-5H-cyclopenta[d]pyrimidin-2-yl)-N-(1,3,4-thiadiazol-2-yl)pyrrolidine-2-carboxamide;
1-(4-((5-isopropyl-1H-pyrazol-3-yl)amino)-6,7-dihydro-5H-cyclopenta[d]pyrimidin-2-yl)-N-(1,3,4-oxadiazol-2-yl)pyrrolidine-2-carboxamide;
(R)-1-(4-((5-isopropyl-1H-pyrazol-3-yl)amino)-6,7-dihydro-5H-cyclopenta[d]pyrimidin-2-yl)-N-(1,3,4-oxadiazol-2-yl)pyrrolidine-2-carboxamide;
(S)-1-(4-((5-isopropyl-1H-pyrazol-3-yl)amino)-6,7-dihydro-5H-cyclopenta[d]pyrimidin-2-yl)-N-(1,3,4-oxadiazol-2-yl)pyrrolidine-2-carboxamide;
1-(4-((5-cyclopropyl-1H-pyrazol-3-yl)amino)-6,7-dihydro-5H-cyclopenta[d]pyrimidin-2-yl)-N-(1,3,4-oxadiazol-2-yl)pyrrolidine-2-carboxamide;
(R)-1-(4-((5-cyclopropyl-1H-pyrazol-3-yl)amino)-6,7-dihydro-5H-cyclopenta[d]pyrimidin-2-yl)-N-(1,3,4-oxadiazol-2-yl)pyrrolidine-2-carboxamide;
(S)-1-(4-((5-cyclopropyl-1H-pyrazol-3-yl)amino)-6,7-dihydro-5H-cyclopenta[d]pyrimidin-2-yl)-N-(1,3,4-oxadiazol-2-yl)pyrrolidine-2-carboxamide;
1-(4-((5-cyclopentyl-1H-pyrazol-3-yl)amino)-6,7-dihydro-5H-cyclopenta[d]pyrimidin-2-yl)-N-(1,3,4-oxadiazol-2-yl)pyrrolidine-2-carboxamide;
(R)-1-(4-((5-cyclopentyl-1H-pyrazol-3-yl)amino)-6,7-dihydro-5H-cyclopenta[d]pyrimidin-2-yl)-N-(1,3,4-oxadiazol-2-yl)pyrrolidine-2-carboxamide;
(S)-1-(4-((5-cyclopentyl-1H-pyrazol-3-yl)amino)-6,7-dihydro-5H-cyclopenta[d]pyrimidin-2-yl)-N-(1,3,4-oxadiazol-2-yl)pyrrolidine-2-carboxamide;
1-(4-((5-cyclohexyl-1H-pyrazol-3-yl)amino)-6,7-dihydro-5H-cyclopenta[d]pyrimidin-2-yl)-N-(1,3,4-oxadiazol-2-yl)pyrrolidine-2-carboxamide;
(R)-1-(4-((5-cyclohexyl-1H-pyrazol-3-yl)amino)-6,7-dihydro-5H-cyclopenta[d]pyrimidin-2-yl)-N-(1,3,4-oxadiazol-2-yl)pyrrolidine-2-carboxamide;
(S)-1-(4-((5-cyclohexyl-1H-pyrazol-3-yl)amino)-6,7-dihydro-5H-cyclopenta[d]pyrimidin-2-yl)-N-(1,3,4-oxadiazol-2-yl)pyrrolidine-2-carboxamide;
1-(4-((5-isopropyl-1H-pyrazol-3-yl)amino)-6,7-dihydro-5H-cyclopenta[d]pyrimidin-2-yl)-N-(1,2,4-thiadiazol-2-yl)pyrrolidine-2-carboxamide;
(R)-1-(4-((5-isopropyl-1H-pyrazol-3-yl)amino)-6,7-dihydro-5H-cyclopenta[d]pyrimidin-2-yl)-N-(1,2,4-thiadiazol-2-yl)pyrrolidine-2-carboxamide;
(S)-1-(4-((5-isopropyl-1H-pyrazol-3-yl)amino)-6,7-dihydro-5H-cyclopenta[d]pyrimidin-2-yl)-N-(1,2,4-thiadiazol-2-yl)pyrrolidine-2-carboxamide;
1-(4-((5-cyclopropyl-1H-pyrazol-3-yl)amino)-6,7-dihydro-5H-cyclopenta[d]pyrimidin-2-yl)-N-(1,2,4-thiadiazol-2-yl)pyrrolidine-2-carboxamide;
(R)-1-(4-((5-cyclopropyl-1H-pyrazol-3-yl)amino)-6,7-dihydro-5H-cyclopenta[d]pyrimidin-2-yl)-N-(1,2,4-thiadiazol-2-yl)pyrrolidine-2-carboxamide;
(S)-1-(4-((5-cyclopropyl-1H-pyrazol-3-yl)amino)-6,7-dihydro-5H-cyclopenta[d]pyrimidin-2-yl)-N-(1,2,4-thiadiazol-2-yl)pyrrolidine-2-carboxamide;
1-(4-((5-cyclopentyl-1H-pyrazol-3-yl)amino)-6,7-dihydro-5H-cyclopenta[d]pyrimidin-2-yl)-N-(1,2,4-thiadiazol-2-yl)pyrrolidine-2-carboxamide;
(R)-1-(4-((5-cyclopentyl-1H-pyrazol-3-yl)amino)-6,7-dihydro-5H-cyclopenta[d]pyrimidin-2-yl)-N-(1,2,4-thiadiazol-2-yl)pyrrolidine-2-carboxamide;
(S)-1-(4-((5-cyclopentyl-1H-pyrazol-3-yl)amino)-6,7-dihydro-5H-cyclopenta[d]pyrimidin-2-yl)-N-(1,2,4-thiadiazol-2-yl)pyrrolidine-2-carboxamide;
1-(4-((5-cyclohexyl-1H-pyrazol-3-yl)amino)-6,7-dihydro-5H-cyclopenta[d]pyrimidin-2-yl)-N-(1,2,4-thiadiazol-5-yl)pyrrolidine-2-carboxamide;
(R)-1-(4-((5-cyclohexyl-1H-pyrazol-3-yl)amino)-6,7-dihydro-5H-cyclopenta[d]pyrimidin-2-yl)-N-(1,2,4-thiadiazol-5-yl)pyrrolidine-2-carboxamide;
(S)-1-(4-((5-cyclohexyl-1H-pyrazol-3-yl)amino)-6,7-dihydro-5H-cyclopenta[d]pyrimidin-2-yl)-N-(1,2,4-thiadiazol-5-yl)pyrrolidine-2-carboxamide;
1-(4-(5-cyclopentyl-1H-pyrazol-3-ylamino)-6,7-dihydro-5H-cyclopenta[d]pyrimidin-2-yl)-N-(thiazol-2-yl)pyrrolidine-2-carboxamide;
(R)-1-(4-(5-cyclopentyl-1H-pyrazol-3-ylamino)-6,7-dihydro-5H-cyclopenta[d]pyrimidin-2-yl)-N-(thiazol-2-yl)pyrrolidine-2-carboxamide;
(S)-1-(4-(5-cyclopentyl-1H-pyrazol-3-ylamino)-6,7-dihydro-5H-cyclopenta[d]pyrimidin-2-yl)-N-(thiazol-2-yl)pyrrolidine-2-carboxamide;
(1-(4-(5-cyclopentyl-1H-pyrazol-3-ylamino)-6,7-dihydro-5H-cyclopenta[d]pyrimidin-2-yl)pyrrolidin-2-yl)(4-methylpiperazin-1-yl)methanone;
(R)-(1-(4-(5-cyclopentyl-1H-pyrazol-3-ylamino)-6,7-dihydro-5H-cyclopenta[d]pyrimidin-2-yl)pyrrolidin-2-yl)(4-methylpiperazin-1-yl)methanone;
(S)-(1-(4-(5-cyclopentyl-1H-pyrazol-3-ylamino)-6,7-dihydro-5H-cyclopenta[d]pyrimidin-2-yl)pyrrolidin-2-yl)(4-methylpiperazin-1-yl)methanone;
(1-(4-(5-cyclopentyl-1H-pyrazol-3-ylamino)-6,7-dihydro-5H-cyclopenta[d]pyrimidin-2-yl)pyrrolidin-2-yl)(4,4-dimethylpiperidin-1-yl)methanone;
(R)-(1-(4-(5-cyclopentyl-1H-pyrazol-3-ylamino)-6,7-dihydro-5H-cyclopenta[d]pyrimidin-2-yl)pyrrolidin-2-yl)(4,4-dimethylpiperidin-1-yl)methanone;
(S)-(1-(4-(5-cyclopentyl-1H-pyrazol-3-ylamino)-6,7-dihydro-5H-cyclopenta[d]pyrimidin-2-yl)pyrrolidin-2-yl)(4,4-dimethylpiperidin-1-yl)methanone;
4-(1-(4-(5-cyclopentyl-1H-pyrazol-3-ylamino)-6,7-dihydro-5H-cyclopenta[d]pyrimidin-2-yl)pyrrolidine-2-carbonyl)-1-methylpiperazin-2-one;
(R)-4-(1-(4-(5-cyclopentyl-1H-pyrazol-3-ylamino)-6,7-dihydro-5H-cyclopenta[d]pyrimidin-2-yl)pyrrolidine-2-carbonyl)-1-methylpiperazin-2-one;
(S)-4-(1-(4-(5-cyclopentyl-1H-pyrazol-3-ylamino)-6,7-dihydro-5H-cyclopenta[d]pyrimidin-2-yl)pyrrolidine-2-carbonyl)-1-methylpiperazin-2-one;
1-(4-(1-(4-(5-cyclopentyl-1H-pyrazol-3-ylamino)-6,7-dihydro-5H-cyclopenta[d]pyrimidin-2-yl)pyrrolidine-2-carbonyl)piperazin-1-yl)ethanone;
(R)-1-(4-(1-(4-(5-cyclopentyl-1H-pyrazol-3-ylamino)-6,7-dihydro-5H-cyclopenta[d]pyrimidin-2-yl)pyrrolidine-2-carbonyl)piperazin-1-yl)ethanone; and
(S)-1-(4-(1-(4-(5-cyclopentyl-1H-pyrazol-3-ylamino)-6,7-dihydro-5H-cyclopenta[d]pyrimidin-2-yl)pyrrolidine-2-carbonyl)piperazin-1-yl)ethanone;
(1-(4-((5-isopropyl-1H-pyrazol-3-yl)amino)-6,7-dihydro-5H-cyclopenta[d]pyrimidin-2-yl)pyrrolidin-2-yl)(pyrrolidin-1-yl)methanone;
(R)-(1-(4-((5-isopropyl-1H-pyrazol-3-yl)amino)-6,7-dihydro-5H-cyclopenta[d]pyrimidin-2-yl)pyrrolidin-2-yl)(pyrrolidin-1-yl)methanone;
(S)-(1-(4-((5-isopropyl-1H-pyrazol-3-yl)amino)-6,7-dihydro-5H-cyclopenta[d]pyrimidin-2-yl)pyrrolidin-2-yl)(pyrrolidin-1-yl)methanone;
(3,3-difluoropyrrolidin-1-yl)(1-(4-((5-isopropyl-1H-pyrazol-3-yl)amino)-6,7-dihydro-5H-cyclopenta[d]pyrimidin-2-yl)pyrrolidin-2-yl)methanone;
(R)-(3,3-difluoropyrrolidin-1-yl)(1-(4-((5-isopropyl-1H-pyrazol-3-yl)amino)-6,7-dihydro-5H-cyclopenta[d]pyrimidin-2-yl)pyrrolidin-2-yl)methanone;
(S)-(3,3-difluoropyrrolidin-1-yl)(1-(4-((5-isopropyl-1H-pyrazol-3-yl)amino)-6,7-dihydro-5H-cyclopenta[d]pyrimidin-2-yl)pyrrolidin-2-yl)methanone;
(1-(4-((5-cyclopentyl-1H-pyrazol-3-yl)amino)-6,7-dihydro-5H-cyclopenta[d]pyrimidin-2-yl)pyrrolidin-2-yl)(4,4-difluoropiperidin-1-yl)methanone;
(R)-(1-(4-((5-cyclopentyl-1H-pyrazol-3-yl)amino)-6,7-dihydro-5H-cyclopenta[d]pyrimidin-2-yl)pyrrolidin-2-yl)(4,4-difluoropiperidin-1-yl)methanone;
(S)-(1-(4-((5-cyclopentyl-1H-pyrazol-3-yl)amino)-6,7-dihydro-5H-cyclopenta[d]pyrimidin-2-yl)pyrrolidin-2-yl)(4,4-difluoropiperidin-1-yl)methanone;
(1-(4-((5-cyclopentyl-1H-pyrazol-3-yl)amino)-6,7-dihydro-5H-cyclopenta[d]pyrimidin-2-yl)pyrrolidin-2-yl)(4,4-dimethylpiperidin-1-yl)methanone;
(R)-(1-(4-((5-cyclopentyl-1H-pyrazol-3-yl)amino)-6,7-dihydro-5H-cyclopenta[d]pyrimidin-2-yl)pyrrolidin-2-yl)(4,4-dimethylpiperidin-1-yl)methanone;
(S)-(1-(4-((5-cyclopentyl-1H-pyrazol-3-yl)amino)-6,7-dihydro-5H-cyclopenta[d]pyrimidin-2-yl)pyrrolidin-2-yl)(4,4-dimethylpiperidin-1-yl)methanone;
(1-(4-((5-(tert-butyl)-1H-pyrazol-3-yl)amino)-6,7-dihydro-5H-cyclopenta[d]pyrimidin-2-yl)pyrrolidin-2-yl)(4,4-difluoropiperidin-1-yl)methanone;
(R)-(1-(4-((5-(tert-butyl)-1H-pyrazol-3-yl)amino)-6,7-dihydro-5H-cyclopenta[d]pyrimidin-2-yl)pyrrolidin-2-yl)(4,4-difluoropiperidin-1-yl)methanone;
(S)-(1-(4-((5-(tert-butyl)-1H-pyrazol-3-yl)amino)-6,7-dihydro-5H-cyclopenta[d]pyrimidin-2-yl)pyrrolidin-2-yl)(4,4-difluoropiperidin-1-yl)methanone;
1-(4-((4-((5-cyclopentyl-1H-pyrazol-3-yl)amino)-6,7-dihydro-5H-cyclopenta[d]pyrimidin-2-yl)prolyl)piperazin-1-yl)propan-1-one;
1-(4-((4-((5-cyclopentyl-1H-pyrazol-3-yl)amino)-6,7-dihydro-5H-cyclopenta[d]pyrimidin-2-yl)-D-prolyl)piperazin-1-yl)propan-1-one;
1-(4-((4-((5-cyclopentyl-1H-pyrazol-3-yl)amino)-6,7-dihydro-5H-cyclopenta[d]pyrimidin-2-yl)-L-prolyl)piperazin-1-yl)propan-1-one;
1-(4-((5-(tert-butyl)-1H-pyrazol-3-yl)amino)-6,7-dihydro-5H-cyclopenta[d]pyrimidin-2-yl)-N-ethyl-N-(thiazol-2-yl)pyrrolidine-2-carboxamide;
(R)-1-(4-((5-(tert-butyl)-1H-pyrazol-3-yl)amino)-6,7-dihydro-5H-cyclopenta[d]pyrimidin-2-yl)-N-ethyl-N-(thiazol-2-yl)pyrrolidine-2-carboxamide;
(S)-1-(4-((5-(tert-butyl)-1H-pyrazol-3-yl)amino)-6,7-dihydro-5H-cyclopenta[d]pyrimidin-2-yl)-N-ethyl-N-(thiazol-2-yl)pyrrolidine-2-carboxamide;
N-ethyl-1-(4-((5-isopropyl-1H-pyrazol-3-yl)amino)-6,7-dihydro-5H-cyclopenta[d]pyrimidin-2-yl)-N-(thiazol-2-yl)pyrrolidine-2-carboxamide;
(R)—N-ethyl-1-(4-((5-isopropyl-1H-pyrazol-3-yl)amino)-6,7-dihydro-5H-cyclopenta[d]pyrimidin-2-yl)-N-(thiazol-2-yl)pyrrolidine-2-carboxamide;
(S)—N-ethyl-1-(4-((5-isopropyl-1H-pyrazol-3-yl)amino)-6,7-dihydro-5H-cyclopenta[d]pyrimidin-2-yl)-N-(thiazol-2-yl)pyrrolidine-2-carboxamide;
(1-(4-((5-cyclopentyl-1H-pyrazol-3-yl)amino)-6,7-dihydro-5H-cyclopenta[d]pyrimidin-2-yl)-2-methylpyrrolidin-2-yl)(4,4-difluoropiperidin-1-yl)methanone;
(R)-(1-(4-((5-cyclopentyl-1H-pyrazol-3-yl)amino)-6,7-dihydro-5H-cyclopenta[d]pyrimidin-2-yl)-2-methylpyrrolidin-2-yl)(4,4-difluoropiperidin-1-yl)methanone; and
(S)-(1-(4-((5-cyclopentyl-1H-pyrazol-3-yl)amino)-6,7-dihydro-5H-cyclopenta[d]pyrimidin-2-yl)-2-methylpyrrolidin-2-yl)(4,4-difluoropiperidin-1-yl)methanone;
and pharmaceutically acceptable salts thereof.

48: The compound of claim 1, or a pharmaceutically acceptable salts thereof, where in the compound is selected from Compound Nos. 1 to 137 in Table 1.

49: The compound of claim 1, wherein the compound inhibits FLT3 to a greater extent than it inhibits IGF-1R.

50: The compound of claim 1, wherein the compound inhibits Trk to a greater extent than it inhibits IGF-1R.

51: A pharmaceutical composition comprising a compound of claim 1, or a pharmaceutically acceptable salt thereof, and a pharmaceutically acceptable carrier.

52: A method of treating cancer in an individual in need thereof, comprising administering to the individual a therapeutically effective amount of a compound of claim 1, or a pharmaceutically acceptable salt thereof.

53: The method of claim 52, wherein the cancer is selected from the group consisting of breast cancer, prostate cancer, ovarian cancer, lung cancer, colon cancer, pancreatic cancer, neuroblastoma and leukemia.

54: The method of claim 52, wherein the cancer is acute myeloid leukemia or acute lymphoblastic leukemia.

55: Use of a compound of claim 1, or a pharmaceutically acceptable salt thereof, in the manufacturing of a medicament for the treatment of cancer.
